(19) United States
(12) Patent Application Publication
(10) Pub. No.: US 2005/0124683 A1
(43) Pub. Date: Jun. 9, 2005

(54) HIV protease inhibitors, compositions containing the same, their pharmaceutical uses and materials for their synthesis

(75) Inventors: Larry Andrew Alegria, San Diego, CA (US); Stacie Sara Canan-Koch, La Jolla, CA (US); Klaus Ruprecht Dress, San Diego, CA (US); Buwen Huang, San Diego, CA (US); Robert Arnold Kumpf, Carlsbad, CA (US); Kathleen Kingsley Lewis, San Diego, CA (US); Jean Joo Matthews, San Diego, CA (US); Sylvie Kim Sakata, San Diego, CA (US); Scott Christopher Virgil, San Diego, CA (US)

Correspondence Address: AGOURON PHARMACEUTICALS, INC., 10350 NORTH TORREY PINES ROAD, LA JOLLA, CA, 92037, US

(73) Assignee: Agouron Pharmaceuticals, Inc.

(21) Appl. No.: 10/942,166

(22) Filed: Sep. 16, 2004

Related U.S. Application Data
(60) Provisional application No. 60/504,018, filed on Sep. 17, 2003.
(60) Provisional application No. 60/527,422, filed on Dec. 4, 2003.

(51) Int. Cl. A61K 31/401

(52) U.S. Cl. 514/423; 548/537

(57) ABSTRACT

The present invention relates to a series of chemical compounds useful as Human Immunodeficiency Virus (HIV) protease inhibitors and to the use of such compounds as antiviral agents. The invention further relates to pharmaceutical compositions containing such antiviral agents, and their uses and materials for their synthesis

This application claims priority to U.S. Provisional Application Nos. 60/504,018, filed 17 Sep. 2003, and 60/527,422, filed 4 Dec. 2003.

### FIELD OF THE INVENTION

This invention relates to a novel series of chemical compounds useful as Human Immunodeficiency Virus (HIV) protease inhibitors and to the use of such compounds as antiviral agents. The invention further relates to pharmaceutical compositions containing such antiviral agents, and their uses and materials for their synthesis.

### BACKGROUND

Acquired Immune Deficiency Syndrome (AIDS) is a relatively newly recognized disease or condition. AIDS causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotromic retrovirus III (HTLV-III), now more commonly referred to as the human immunodeficiency virus or HIV.

HIV is a member of the class of viruses known as retroviruses. The retroviral genome is composed of RNA which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell which plays a vital role in the body's immune system. HIV infection of these white blood cells depletes this white cell population. Eventually, the immune system is rendered inoperative and ineffective against various opportunistic diseases such as, among others, pneumocystic carini pneumonia, Karposis sarcoma, and cancer of the lymph system.

Although the exact mechanism of the formation and working of the HIV virus is not understood, identification of the virus has led to some progress in controlling the disease. For example, the drug azidothymidine (AZT) has been found effective for inhibiting the reverse transcription of the retroviral genome of the HIV virus, thus giving a measure of control, though not a cure, for patients afflicted with AIDS. The search continues for drugs that can cure or at least provide an improved measure of control of the deadly HIV virus.

Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

HIV protease is one of the translated products from the HIV structural protein pol gene. The HIV-protease enzyme is essential for the replication and dissemination of HIV throughout the body (Navia M. A. and McKeever B. M., Ann., New York Acad. Sci., 1990;616:73-85), and it has become an important target for the design and development of anti-HIV therapeutic agents (von der Helm K., Biol. Chem. 1996;377:756-774). This retroviral protease specifically cleaves other structural polypeptides at discrete sites to release these newly activated structural proteins and enzymes, thereby rendering the virion replication-competent. As such, inhibition of the HIV protease by potent compounds may prevent proviral integration of infected T-lymphocytes during the early phase of the HIV-1 life cycle, as well as inhibit viral proteolytic processing during its late stage. Additionally, the protease inhibitors may have the advantages of being more readily available, longer lived in virus, and less toxic than currently available drugs, possibly due to their specificity for the retroviral protease.

In accordance with this invention, there is provided a novel class of chemical compounds that can inhibit and/or block the activity of the HIV protease, which halts the proliferation of HIV virus, pharmaceutical compositions containing these compounds, and the use of the compounds as inhibitors of the HIV protease, and methods and materials for their preparation.

### SUMMARY

The invention relates to a novel series of chemical compounds useful as HIV protease inhibitors and to the use of such compounds as antiviral agents in pharmaceutical compositions. The invention further relates to methods and materials for synthesis of the antiviral agents and pharmaceutical compositions containing the same.

The present invention also relates to methods of inhibiting HIV protease activity, comprising contacting the protease with an effective amount of a compound of Formula (I), or a pharmaceutically acceptable salt, prodrug, pharmaceutically active metabolite, or solvate thereof. For example, HIV protease activity may be inhibited in mammalian tissue by administering a compound of Formula (I) or a pharmaceutically acceptable salt, prodrug, pharmaceutically active metabolite, or solvate thereof. More preferably, the present method is directed at inhibiting HIV-protease activity.

Furthermore, the present invention relates to the treatment of mammals, such as human beings, infected with HIV, suffering from acquired immunodeficiency syndrome (AIDS), AIDS-related complex (ARC), or other HIV- or AIDS-related diseases. The methods of the present invention comprise administering to a mammal an HIV-inhibiting amount of a compound of the present invention, or a pharmaceutically acceptable salt, prodrug, pharmaceutically active metabolite, or solvate thereof, in a pharmaceutically acceptable formulation, either alone or in combination with an effective amount of an additional agent, to treat said mammal suffering from infection with the HIV virus. The present invention also provides methods for inhibiting HIV replication in a mammal, such as a human, comprising administering to said mammal an HIV-replication inhibiting amount of a compound according to the invention, or a pharmaceutically acceptable salt or solvate thereof.

The present invention also relates to pharmaceutically acceptable formulations, comprising an effective amount of a compound according to the invention, or a pharmaceutically acceptable salt or solvate thereof, and at least one pharmaceutically acceptable carrier. The present invention also relates to pharmaceutical compositions, comprising an HIV-protease inhibiting amount of a compound according to the invention, or a pharmaceutically acceptable salt or solvate thereof, and at least one pharmaceutically acceptable carrier.

The present invention provides compounds of formula (I), 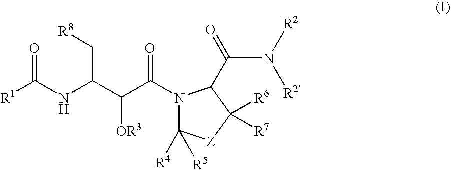
wherein:

Z is O, S, C═CH₂, or —C(R¹²)(R¹³);

R¹ is C₆₋₁₀ aryl, heteroaryl, or heterocyclyl, all of which are optionally substituted with at least one substituent chosen from C₁₋₁₀ alkyl, C₆₋₁₀ aryl, heteroaryl, heterocyclyl, hydroxyl, halogen, C₁₋₆ alkylcarbonyloxy, C₆₋₁₀ arylcarbonyloxy, heteroarylcarbonyloxy, C₆₋₁₀arylC₁₋₁₀alkyl, heteroarylC₁₋₁₀alkyl, and heterocyclylC₁₋₁₀alkyl;

R² is hydrogen, C₃₋₁₀ cycloalkyl, C₂₋₁₀ alkenyl, C₂₋₁₀ alkynyl, —(R⁴R⁵)ₙR⁹, or C₁₋₁₀ alkyl wherein any carbon atom is optionally replaced by a heteroatom chosen from O, N, and S;

n is 0 to 10;

R^{2′} is H or C₁₋₁₀ alkyl;

R³ is hydrogen or —C(O)R¹⁰;

each R⁴ and R⁵ are independently selected from hydrogen and C₁₋₁₀ alkyl;

R⁶ and R⁷ are independently chosen from hydrogen, C₁₋₁₀ alkyl, and C₆₋₁₀ aryl;

R⁸ is C₆₋₁₀ aryl optionally substituted with at least one of C₁₋₁₀ alkyl, —CF₃, halogen, hydroxyl, and —OC₁₋₁₀ alkyl;

R⁹ is —CF₃, C₆₋₁₀ aryl, heteroaryl, or heterocyclyl, wherein said C₆₋₁₀ aryl, heteroaryl, and heterocyclyl are optionally substituted with at least one substituent independently chosen from C₁₋₁₀ alkyl, C₆₋₁₀ aryl, heteroaryl, heterocyclyl, halogen, —CF₃, —OR¹⁰, —SR¹⁰, —S(O)₂R¹⁰, and —N(R¹⁰)(R¹¹);

each R¹⁰ and R¹¹ are independently chosen from hydrogen, C₁₋₁₀ alkyl, C₂₋₁₀ alkenyl, C₂₋₁₀ alkynyl, C₆₋₁₀ aryl, heteroaryl, and heterocyclyl, and C₃₋₁₀cycloalkylC₁₋₁₀alkyl; and

R¹² and R¹³ are independently selected from hydrogen, hydroxyl, —CF₃, halogen, C₁₋₁₀ alkyl, C₆₋₁₀ aryl, heteroaryl, heterocyclyl, and —OC₁₋₁₀ alkyl; or

pharmaceutically acceptable salts or solvates thereof.

In another aspect of the present invention are provided compounds of formula (I), wherein:

Z is O, S, or —C(R¹²)(R¹³);

R¹ is C₆₋₁₀ aryl or heteroaryl, which are optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is C₂₋₁₀ alkenyl, —(CH₂)ₙR⁹, or C₁₋₁₀ alkyl wherein any carbon atom is optionally replaced by a heteroatom chosen from O, N, and S;

n is 0 to 10;

R^{2′} is H;

R³ is hydrogen;

R⁴ and R⁵ are hydrogen;

R⁶ and R⁷ are C₁₋₁₀ alkyl;

R⁸ is C₆₋₁₀ aryl optionally substituted with at least one halogen;

R⁹ is —CF₃, C₆₋₁₀ aryl, or heteroaryl, wherein said C₆₋₁₀ aryl or heteroaryl are optionally substituted with at least one substituent independently chosen from C₁₋₁₀ alkyl and halogen; and

R¹² and R¹³ are independently selected from hydroxyl, halogen, and C₁₋₁₀ alkyl; or

pharmaceutically acceptable salts or solvates thereof.

In yet another aspect of the present invention are provided compounds of formula (I), wherein:

Z is O, S, or —C(R¹²)(R¹³);

R¹ is C₆₋₁₀ aryl or heteroaryl, which are optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is C₂₋₁₀ alkenyl, —(CH₂)ₙR⁹, or C₁₋₁₀ alkyl;

n is 0 to 10;

R^{2′} is H;

R³ is hydrogen;

R⁴ and R⁵ are hydrogen;

R⁶ and R⁷ are C₁₋₁₀ alkyl;

R⁸ is C₆₋₁₀ aryl optionally substituted with at least one halogen;

R⁹ is —CF₃, C₆₋₁₀ aryl, or heteroaryl, wherein said C₆₋₁₀ aryl or heteroaryl are optionally substituted with at least one substituent independently chosen from C₁₋₁₀ alkyl and halogen; and

R¹² and R¹³ are independently selected from hydroxyl, halogen, and C₁₋₁₀ alkyl; or

pharmaceutically acceptable salts or solvates thereof.

In still a further aspect of the present invention are provided compounds of formula (I), wherein n is 0 to 5, or pharmaceutically acceptable salts or solvates thereof.

In yet another aspect of the present invention are provided compounds of formula (I), wherein:

Z is S or —C(R¹²)(R¹³);

R¹ is C₆₋₁₀ aryl optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is C₁₋₁₀ alkyl;

R^{2′} is H;

R³ is hydrogen;

R⁴ and R⁵ are hydrogen;

R⁶ and R⁷ are C₆₋₁₀ alkyl;

R⁸ is C₆₋₁₀ aryl optionally substituted with at least one fluorine; and

R¹² and R¹³ are independently selected from hydroxyl, halogen, and C₁₋₁₀ alkyl; or

pharmaceutically acceptable salts or solvates thereof.

Another aspect of the present invention provides compounds of formula (I), wherein:

Z is S or —C(R¹²)(R¹³);

R¹ is C₆₋₁₀ aryl optionally substituted with at least one substituent chosen from C₁₋₁₀ alkyl, hydroxyl, and C₁₋₆ alkylcarbonyloxy;

R² is C₁₋₁₀ alkyl;

R^{2′} is H;

R³ is hydrogen;

R⁴ and R⁵ are hydrogen;

R⁶ and R⁷ are C₁₋₁₀ alkyl;

R⁸ is C₆₋₁₀ aryl optionally substituted with at least one fluorine; and

R¹² and R¹³ are independently selected from hydroxyl, halogen, and methyl; or

pharmaceutically acceptable salts or solvates thereof.

Also provided are compounds of formula (I), wherein:

Z is —C(R¹²)(R¹³);

R¹ is C₆₋₁₀ aryl optionally substituted with at least one substituent chosen from methyl, hydroxyl, and methylcarbonyloxy;

R² is methyl, ethyl, n-propyl, iso-propyl, n-butyl, iso-butyl, tert-butyl, 2-methyl-n-butyl, 3-methyl-n-butyl, n-pentyl, iso-pentyl, 2-methyl-n-pentyl, 3-methyl-n-pentyl, 4-methyl-n-pentyl, n-hexyl, 2-methyl-n-hexyl, 3-methyl-n-hexyl, 4-methyl-n-hexyl, 5-methyl-n-hexyl, n-heptyl, iso-heptyl, or —CH₂C(CH₃)₃;

R^{2′} is H;

R³ is hydrogen;

R⁴ and R⁵ are hydrogen;

R⁶ and R⁷ are methyl;

R⁸ is phenyl optionally substituted with at least one fluorine; and

R¹² and R¹³ are independently selected from halogen and methyl; or

pharmaceutically acceptable salts or solvates thereof.

In a further aspect of the present invention are provided compounds of formula (I), wherein:

Z is —C(R¹²)(R¹³);

R¹ is phenyl substituted with methyl and hydroxyl;

R² is methyl, ethyl, n-propyl, iso-propyl, n-butyl, iso-butyl, tert-butyl, 2-methyl-n-butyl, 3-methyl-n-butyl, n-pentyl, iso-pentyl, 2-methyl-n-pentyl, 3-methyl-n-pentyl, 4-methyl-n-pentyl, n-hexyl, 2-methyl-n-hexyl, 3-methyl-n-hexyl, 4-methyl-n-hexyl, 5-methyl-n-hexyl, n-heptyl, iso-heptyl, or —CH₂C(CH₃)₃;

R^{2′} is H;

R³ is hydrogen;

R⁴ and R⁵ are hydrogen;

R⁶ and R⁷ are methyl;

R⁸ is phenyl optionally substituted with at least one fluorine; and

R¹² and R¹³ are independently selected from halogen and methyl; or

pharmaceutically acceptable salts or solvates thereof.

Also provided are compounds of formula (I), wherein:

Z is —CF₂;

R¹ is phenyl substituted with methyl and hydroxyl;

R² is methyl, ethyl, n-propyl, iso-propyl, n-butyl, iso-butyl, tert-butyl, 2-methyl-n-butyl, 3-methyl-n-butyl, n-pentyl, iso-pentyl, 2-methyl-n-pentyl, 3-methyl-n-pentyl, 4-methyl-n-pentyl, n-hexyl, 2-methyl-n-hexyl, 3-methyl-n-hexyl, 4-methyl-n-hexyl, 5-methyl-n-hexyl, n-heptyl, iso-heptyl, or —CH₂C(CH₃)₃;

R^{2′} is H;

R³ is hydrogen;

R⁴ and R⁵ are hydrogen;

R⁶ and R⁷ are methyl; and

R⁸ is phenyl substituted with at least one fluorine; or

pharmaceutically acceptable salts or solvates thereof.

In another aspect of the present invention are provided compounds of formula (I), wherein:

Z is —CF₂;

R¹ is phenyl substituted with methyl and hydroxyl;

R² is ethyl;

R^{2′} is H;

R³ is hydrogen;

R⁴ and R⁵ are hydrogen;

R⁶ and R⁷ are methyl; and

R⁸ is phenyl substituted with at least one fluorine; or

pharmaceutically acceptable salts or solvates thereof.

In still a further aspect of the present invention are provided compounds of formula (I), wherein:

Z is S or —C(R¹²)(R¹³);

R¹ is C₆₋₁₀ aryl substituted with at least one substituent chosen from methyl and hydroxyl;

R² is C₂₋₁₀ alkenyl, —(CH₂)ₙR⁹, or C₁₋₁₀ alkyl;

n is 0 to 5;

R^{2′} is H;

R³ is hydrogen;

R⁴ and R⁵ are hydrogen;

R⁶ and R⁷ are methyl;

R⁸ is C₆₋₁₀ aryl optionally substituted with at least one fluorine;

R⁹ is phenyl or pyridyl, both of which are optionally substituted with at least one substituent chosen from methyl and fluorine; and

R¹² and R¹³ are independently selected from hydroxyl, fluorine, and methyl; or

pharmaceutically acceptable salts or solvates thereof.

Also provided are compounds of formula (I), wherein:

Z is S or —C(R¹²)(R¹³);

R¹ is C₆₋₁₀ aryl substituted with at least one substituent chosen from methyl and hydroxyl;

R² is —(CH₂)ₙR⁹;

n is 0 to 5;

R^{2′} is H;

R³ is hydrogen;

R⁴ and R⁵ are hydrogen;

R⁶ and R⁷ are methyl;

R⁸ is C₆₋₁₀ aryl optionally substituted with at least one fluorine;

R⁹ is phenyl or pyridyl, both of which are optionally substituted with at least one substituent chosen from methyl and fluorine; and

R¹² and R¹³ are independently selected from hydroxyl, fluorine, and methyl; or

pharmaceutically acceptable salts or solvates thereof.

In another aspect of the present invention are provided compounds of formula (I), wherein:

Z is —C(R¹²)(R¹³);

R¹ is phenyl substituted with at least one substituent chosen from methyl and hydroxyl;

R² is —(CH₂)ₙR⁹;

n is 0 to 5;

R^{2′} is H;

R³ is hydrogen;

R⁴ and R⁵ are hydrogen;

R⁶ and R⁷ are methyl;

R⁸ is phenyl optionally substituted with at least one fluorine;

R⁹ is phenyl or pyridyl, both of which are optionally substituted with at least one substituent chosen from methyl and fluorine; and

R¹² and R¹³ are independently selected from hydroxyl, fluorine, and methyl; or

pharmaceutically acceptable salts or solvates thereof.

In still a further aspect of the present invention are provided compounds of formula (I), wherein:

Z is —C(R¹²)(R¹³);

R¹ is phenyl substituted with at least one substituent chosen from methyl and hydroxyl;

R² is —(CH₂)ₙR⁹;

n is 0, 1, 2, or 3;

R^{2′} is H;

R³ is hydrogen;

R⁴ and R⁵ are hydrogen;

R⁶ and R⁷ are methyl;

R⁸ is phenyl;

R⁹is pyridyl optionally substituted with at least one methyl; and

R¹² and R¹³ are independently selected from hydroxyl, fluorine, and methyl; or

pharmaceutically acceptable salts or solvates thereof.

Another aspect of the present invention provides compounds of formula (I), wherein:

Z is —CF₂;

R¹ is phenyl substituted with at least one substituent chosen from methyl and hydroxyl;

R² is —CH₂R⁹;

R^{2′} is H;

R³ is hydrogen;

R⁴ and R⁵ are hydrogen;

R⁶ and R⁷ are methyl;

R⁸ is phenyl; and

R⁹ is pyridyl substituted with at least one methyl; or

pharmaceutically acceptable salts or solvates thereof.

A further aspect of the present invention provides compounds of formula (I), wherein:

Z is —CF₂;

R¹ is phenyl substituted with at least one substituent chosen from methyl and hydroxyl;

R² is —CH₂R⁹;

R^{2′} is H;

R³ is hydrogen;

R⁴ and R⁵ are hydrogen;

R⁶ and R⁷ are methyl;

R⁸ is phenyl substituted with at least one fluorine; and

R⁹ is phenyl substituted with at least one fluorine; or

pharmaceutically acceptable salts or solvates thereof.

In yet another aspect of the present invention are provided compounds of formula (I), wherein:

Z is —CF₂;

R¹ is phenyl substituted with at least one substituent chosen from methyl and hydroxyl;

R² is —CH₂R⁹;

R^{2′} is H;

R³ is hydrogen;

R⁴ and R⁵ are hydrogen;

R⁶ and R⁷ are methyl;

R^{8′} is phenyl substituted with at least one fluorine; and

R⁹ is phenyl substituted with at least one methyl; or

pharmaceutically acceptable salts or solvates thereof.

The present invention also provides compounds of formula (I), wherein:

Z is —CF₂;

R¹ is phenyl substituted with at least one substituent chosen from methyl and hydroxyl;

R² is —(CH₂)ₙR⁹;

n is 0 to 5;

R^{2′} is H;

R³ is hydrogen;

R⁴ and R⁵ are hydrogen;

R⁶ and R⁷ are methyl;

R⁸ is phenyl optionally substituted with at least one fluorine; and

R⁹ is phenyl or pyridyl, both of which are optionally substituted with at least one substituent chosen from methyl and fluorine; or

pharmaceutically acceptable salts or solvates thereof.

Also provided are compounds of formula (I), wherein:

Z is —CF₂;

R¹ is phenyl substituted with methyl and hydroxyl;

R² is iso-butyl;

R^{2′} is H;

R³ is hydrogen;

R⁴ and R⁵ are hydrogen;

R⁶ and R⁷ are methyl; and

R⁸ is phenyl optionally substituted with at least one fluorine; or

pharmaceutically acceptable salts or solvates thereof.

The present invention also provides compounds of formula (I), wherein:

Z is —CF₂;

R¹ is phenyl substituted with methyl and hydroxyl;

R² is —CH₂C(CH₃)₃;

R^{2′} is H;

R³ is hydrogen;

R⁴ and R⁵ are hydrogen;

R⁶ and R⁷ are methyl; and

R⁸ is phenyl optionally substituted with at least one fluorine; or

pharmaceutically acceptable salts or solvates thereof.

Also provided are compounds of formula (I), wherein:

Z is —C(R¹²)(R¹³);

R¹ is C₆₋₁₀ aryl or heteroaryl, which are optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is C₂₋₁₀ alkenyl, —(CH₂)ₙR⁹, or C₁₋₁₀ alkyl;

n is 0 to 10;

R^{2′} is H;

R³ is hydrogen;

R⁴ and R⁵ are hydrogen;

R⁶ and R⁷ are C₁₋₁₀ alkyl;

R⁸ is C₆₋₁₀ aryl optionally substituted with at least one halogen;

R⁹is —CF₃, C₆₋₁₀ aryl, or heteroaryl, wherein said C₆₋₁₀ aryl or heteroaryl are optionally substituted with at least one substituent independently chosen from C₁₋₁₀ alkyl and halogen;

R¹² is hydroxyl; and

R¹³ is C₁₋₁₀ alkyl; or

pharmaceutically acceptable salts or solvates thereof.

In still a further aspect of the present invention are provided compounds of formula (I), wherein:

Z is —C(OH)(CH₃);

R¹ is C₆₋₁₀ aryl or heteroaryl, which are optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is C₂₋₁₀ alkenyl, —(CH₂)ₙR⁹, or C₁₋₁₀ alkyl;

n is 0 to 10;

R^{2′} is H;

R³ is hydrogen;

R⁴ and R⁵ are hydrogen;

R⁶ and R⁷ are C₁₋₁₀ alkyl;

R⁸ is C₆₋₁₀ aryl optionally substituted with at least one halogen; and

R⁹ is —CF₃, C₆₋₁₀ aryl, or heteroaryl, wherein said C₆₋₁₀ aryl or heteroaryl are optionally substituted with at least one substituent independently chosen from C₁₋₁₀ alkyl and halogen; or

pharmaceutically acceptable salts or solvates thereof.

Another aspect of the present invention provides compounds of formula (I), wherein:

Z is —C(OH)(CH₃);

R¹ is C₆₋₁₀ aryl or heteroaryl, which are optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is C₂₋₁₀ alkenyl;

R^{2′} is H;

R³ is hydrogen;

R⁴ and R⁵ are hydrogen;

R⁶ and R⁷ are methyl; and

R⁸ is C₆₋₁₀ aryl optionally substituted with at least one halogen; or

pharmaceutically acceptable salts or solvates thereof.

In still a further aspect of the present invention are provided compounds of formula (I), wherein:

Z is —(OH)(CH₃);

R¹ is C₆₋₁₀ aryl or heteroaryl, which are optionally substituted with at least one substituent chosen from C₁₋₁₀ alkyl, hydroxyl, C₁₋₆ alkylcarbonyloxy, C₆₋₁₀ arylcarbonyloxy, and heteroarylcarbonyloxy;

R² is —(CH₂)ₙR⁹;

n is 0, 1, 2, or 3;

R^{2′} is H;

R³ is hydrogen;

R⁴ and R⁵ are hydrogen;

R⁶ and R⁷ are C₁₋₁₀ alkyl;

R⁸ is C₆₋₁₀ aryl optionally substituted with at least one halogen; and

R⁹ is C₆₋₁₀ aryl or heteroaryl, wherein said C₆₋₁₀ aryl or heteroaryl are optionally substituted with at least one substituent independently chosen from methyl and fluorine; or

pharmaceutically acceptable salts or solvates thereof.

Also provided in the present invention are compounds of formula (I), wherein:

Z is —C(OH)(CH₃);

R¹ is phenyl optionally substituted with at least one substituent chosen from methyl and hydroxyl;

R² is —CH₂R⁹;

R^{2′} is H;

R³ is hydrogen;

R⁴ and R⁵ are hydrogen;

R⁶ and R⁷ are methyl;

R⁸ is phenyl optionally substituted with at least one halogen; and

R⁹ is phenyl optionally substituted with at least one substituent independently chosen from methyl and fluorine; or

pharmaceutically acceptable salts or solvates thereof.

Further provided in the present invention are compounds of formula (I), wherein:

Z is —C(OH)(CH₃);

R¹ is phenyl substituted with methyl and hydroxyl;

R² is —CH₂R⁹;

R^{2′} is H;

R³ is hydrogen;

R⁴ and R⁵ are hydrogen;

R⁶ and R⁷ are methyl;

R⁸ is phenyl optionally substituted with at least one fluorine; and

R⁹ is phenyl substituted with methyl; or

pharmaceutically acceptable salts or solvates thereof.

The present invention also provides a method for preparing compounds of formula (I), 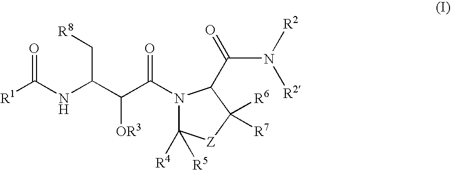
wherein:

Z is O, S, C═CH₂, or —C(R¹²)(R¹³);

R¹ is C₆₋₁₀ aryl, heteroaryl, or heterocyclyl, all of which are optionally substituted with at least one substituent chosen from C₁₋₁₀ alkyl, C₆₋₁₀ aryl, heteroaryl, heterocyclyl, hydroxyl, halogen, C₁₋₆ alkylcarbonyloxy, C₁₋₁₀ arylcarbonyloxy, heteroarylcarbonyloxy, C₆₋₁₀arylC₁₋₁₀alkyl, heteroarylC₁₋₁₀alkyl, and heterocyclylC₁₋₁₀alkyl;

R² is hydrogen, C₃₋₁₀ cycloalkyl, C₂₋₁₀ alkenyl, C₂₋₁₀ alkynyl, —(CH₂)ₙR⁹, or C₁₋₁₀ alkyl wherein any carbon atom is optionally replaced by a heteroatom chosen from O, N, and S;

n is 0 to 10;

R^{2′} is H or C₁₋₁₀ alkyl;

R³ is hydrogen or —C(O)R¹⁰;

R⁴ and R⁵ are independently selected from hydrogen and C₁₋₁₀ alkyl;

R⁶ and R⁷ are independently chosen from hydrogen, C₁₋₁₀ alkyl, and C₆₋₁₀ aryl;

R⁸ is C₆₋₁₀ aryl optionally substituted with C₁₋₁₀ alkyl, —CF₃, halogen, hydroxyl, and —OC₁₋₁₀ alkyl;

R⁹ is —CF₃, C₆₋₁₀ aryl, heteroaryl, or heterocyclyl, wherein said C₆₋₁₀ aryl, heteroaryl, and heterocyclyl are optionally substituted with at least one substituent independently chosen from C₁₋₁₀ alkyl, C₆₋₁₀ aryl, heteroaryl, heterocyclyl, halogen, —CF₃, —OR¹⁰, —SR¹⁰, —S(O)₂R¹⁰, and —N(R¹⁰)(R¹¹);

R¹⁰ and R¹¹ are independently chosen from hydrogen, C₁₋₁₀ alkyl, C₂₋₁₀ alkenyl, C₂₋₁₀ alkynyl, C₆₋₁₀ aryl, heteroaryl, and heterocyclyl, and C₃₋₁₀cycloalkylC₁₋₁₀alkyl; and

R¹² and R¹³ are independently selected from hydrogen, hydroxyl, —CF₃, halogen, C₁₋₁₀ alkyl, C₆₋₁₀ aryl, heteroaryl, heterocyclyl, and —OC₁₋₁₀ alkyl; comprising:

reacting a compound of formula (II), wherein Y¹ is hydroxyl or a leaving group and R¹, R³, and R⁸ are as described for formula (I), with a compound of formula (III), or a salt or solvate thereof,

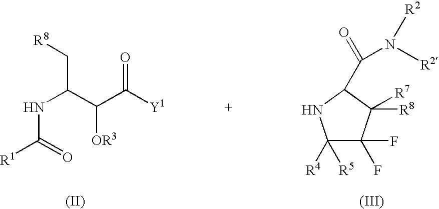

In another aspect of the present invention are provided methods for the preparation of compounds of formula (I), comprising:

(i) reacting a compound of formula (IV), wherein Y¹ is hydroxy or —OP¹, wherein P¹ is a suitable protecting group, and R³ is hydrogen, C₁-C₄ alkyl, or a suitable hydroxyl protecting group, with a compound of formula (V), wherein Y² is a leaving group, to afford a compound of formula (II);

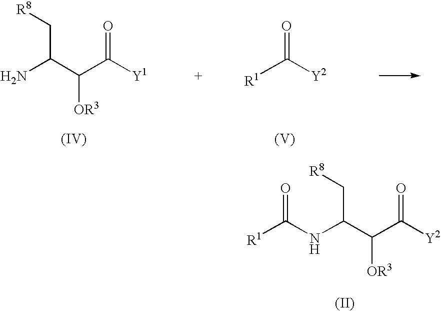

(ii) reacting the compound of formula (II) with a compound of formula (III), or a salt or solvate thereof, to afford a compound of formula (I); and

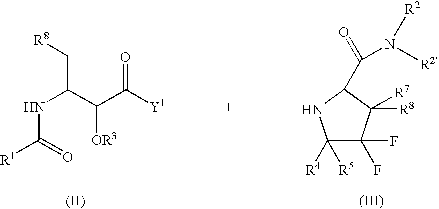

(iii) optionally deprotecting those compounds of formula (I) wherein R³ is a hydroxyl protecting group, to afford a compound of formula (I) wherein R³ is hydrogen.

The present invention also provides methods for the preparation of compounds of formula (I), wherein the compound of formula (I) is:

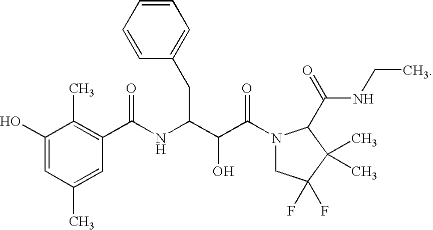

Another aspect of the present invention provides methods for the preparation of compounds of formula (I-A), 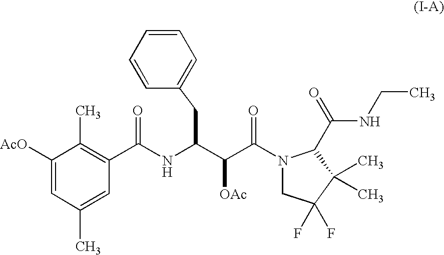
comprising:

reacting a compound of formula (II-B) with a compound of formula (III-A), or a salt or solvate thereof.

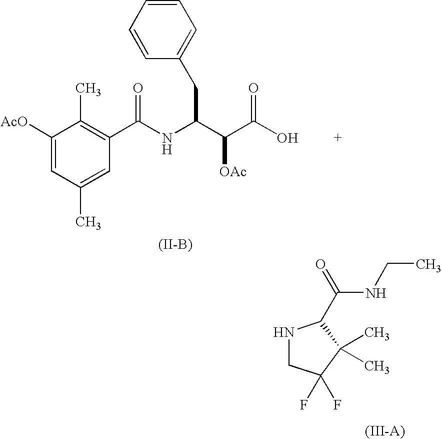

A still further aspect of the present invention provides methods for the preparation of compounds of formula (I-A), 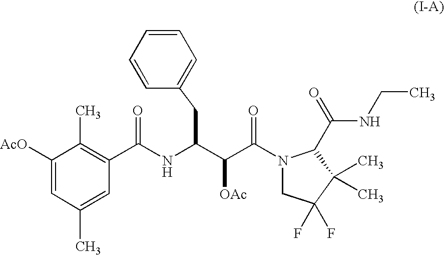
comprising:

(i) reacting a compound of formula (IV-A) with a compound of formula (V-B), 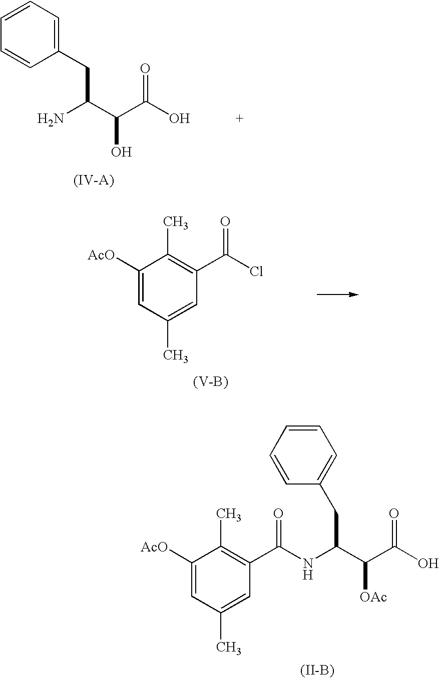
to afford a compound of formula (II-B);

(ii) treating the compound of formula (II-B) with an acetylating agent, 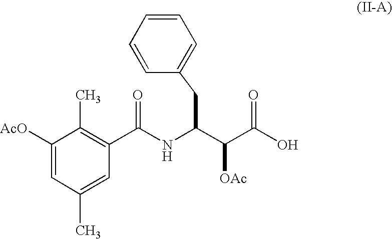
to afford a compound of formula (II-A); and

(iii) reacting the compound of formula (II-A) with a compound of formula (III-A).

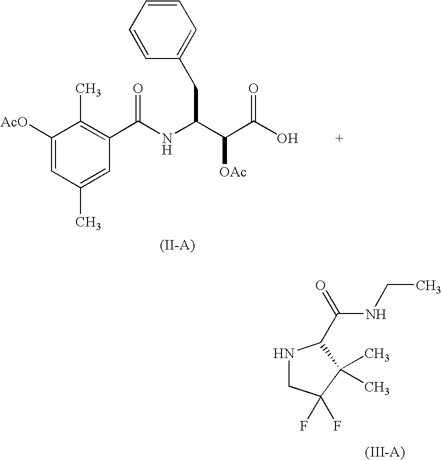

Also provided are methods for the preparation of compounds of formula (I-B), 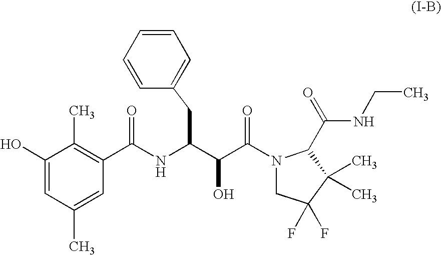
said method comprising:

(i) reacting a compound of formula (II-A) with a compound of formula (III-A), or a salt or solvate thereof, 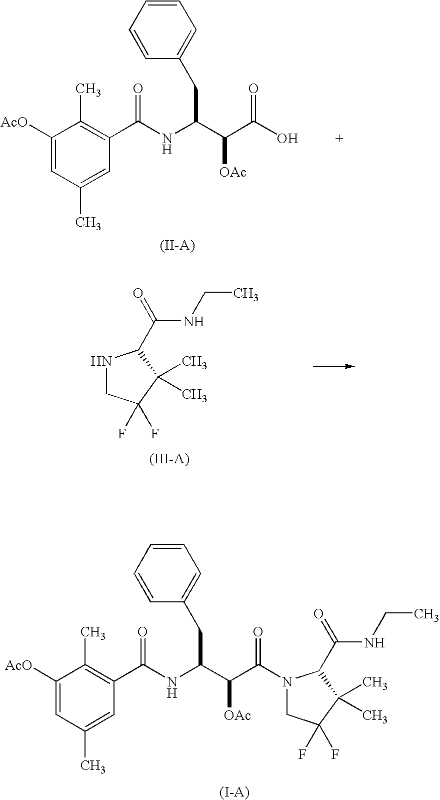
to afford a compound of formula (I-A); and

(ii) deprotecting the compound of formula (I-A).

In still a further aspect of the present invention are provided methods of preparing compounds of formula (I-B), 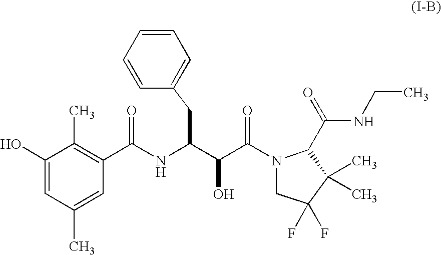
comprising:

(i) reacting a compound of formula (IV-A) with a compound of formula (V-B), 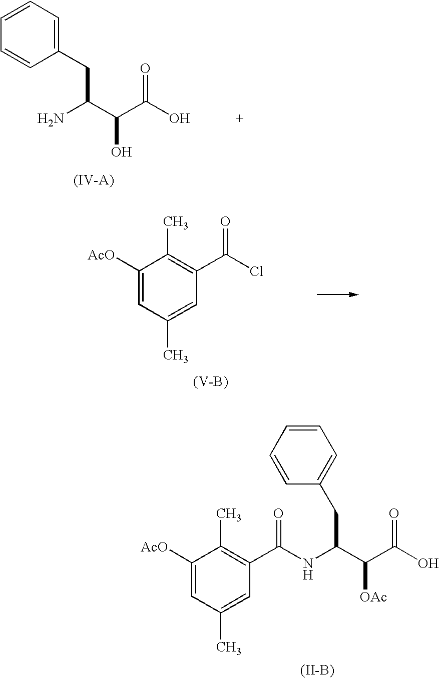
to afford a compound of formula (II-B);

(ii) treating the compound of formula (II-B) with an acetylating agent, 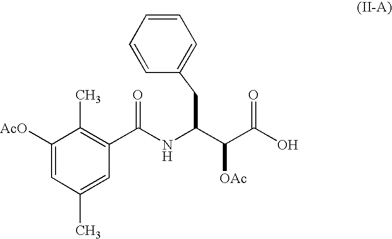
to afford a compound of formula (II-A);

(iii) reacting the compound of formula (II-A) with a compound of formula (III-A), 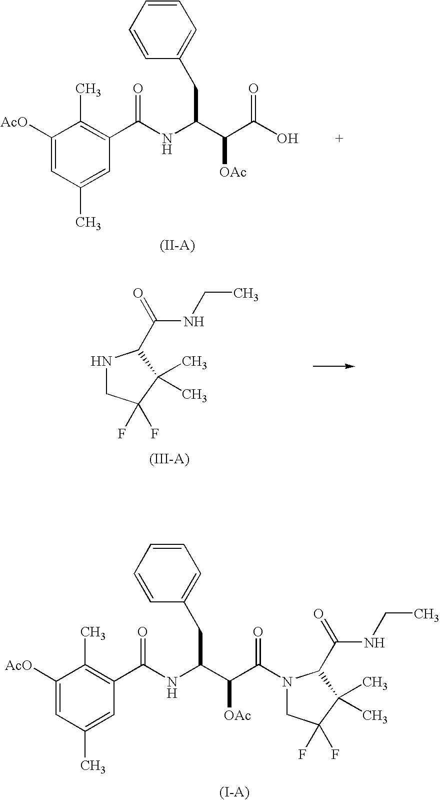
to afford a compound of formula (I-A); and

(iv) deprotecting the compound of formula (I-A).

In yet another aspect of the present invention are provided compounds of formula (II), 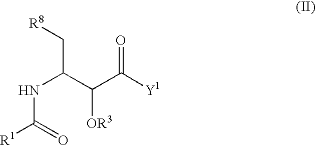
wherein:

R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;

R³ is hydrogen or a hydroxyl protecting group;

R⁸ is C₆₋₁₀ aryl optionally substituted with C₁₋₁₀ alkyl, —CF₃, halogen, hydroxyl, and —OC₁₋₁₀ alkyl; and

Y¹ is a leaving group or hydroxyl.

In yet another aspect of the present invention are provided compounds of formula (II), wherein:

R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy;

R³ is a hydroxyl protecting group; and

Y¹ is hydroxyl.

In still a further aspect of the present invention are provided compounds of formula (II), wherein:

R¹ is phenyl optionally substituted by at least one substituent independently chosen from methyl, hydroxyl, and C₁₋₆ alkylcarbonyloxy;

R³ is a hydroxyl protecting group; and

Y¹ is hydroxyl.

The present invention also provides compounds of formula (II), wherein:

R¹ is phenyl optionally substituted by at least one substituent independently chosen from methyl, hydroxyl, and methylcarbonyloxy;

R³ is a hydroxyl protecting group; and

Y¹ is hydroxyl.

Another aspect of the present invention also provides compounds of formula (II), wherein:

R¹ is phenyl substituted by methyl and methylcarbonyloxy;

R³ is a methylcarbonyl; and

Y¹ is hydroxyl.

Another aspect of the present invention features compounds of formulae (I-A), (I-B), (II-A), and (III-A): 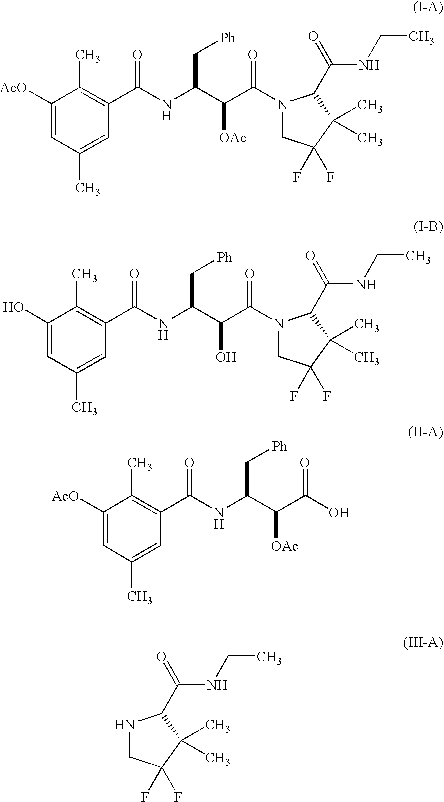
all of which are intermediates useful in the preparation of compounds of formula (I).

Also provided herein are compounds selected from:
(R)-3-((2S,3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
(S)-4,4-Difluoro-1-[4-(3-fluoro-phenyl)-(2S,3S)-hydroxy-(3-hydroxy-2-methyl-benzoylamino)-butyryl]-3,3-dimethyl-pyrrolidine-2-carboxylic acid (2-methoxy-(1S)-methyl-ethyl)-amide;
N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide;
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide;
(S)-4,4-Difluoro-1-[4-(3-fluoro-phenyl)-(2S)-hydroxy-(3S)-(3-hydroxy-2-methyl-benzoylamino)-butyryl]-3,3-dimethyl-pyrrolidine-2-carboxylic acid allylamide;
(S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (3-methyl-pyridin-4-ylmethyl)-amide;
2,3-Dihydro-1H-indole-4-carboxylic acid [(1S,2S)-1-benzyl-3-((S)-4,4-difluoro-2-isobutylcarbamoyl-3,3-dimethyl-pyrrolidin-1-yl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide;
2,3-Dihydro-1H-indole-4-carboxylic acid [(1S,2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin4-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide;
(S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide;
2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide;
(S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide;
(S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide;
1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
N-((1 S,2S)-3-{(2S)-2-[(allylamino)carbonyl]-4,4-difluoro-3,3-dimethylpyrrolidin-1-yl}-benzyl-2-hydroxy-3-oxopropyl)indoline-4-carboxamide;
(4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide;
(S)-3-((2S,3S)-2-Hydroxy-3-{[1-(3-hydroxy-2-methyl-phenyl)-methanoyl]-amino-}4-phenyl-butanoyl)-5,5-dimethyl-oxazolidine-4-carboxylic acid (S)-indan-1-ylamide;
(3R)-N-allyl-1-{(2S,3 S)-2-hydroxy-3-[(3-hydroxy-2-methylbenzoyl)amino]-4-phenylbutanoyl}-3-phenyl-L-prolinamide;
(4S)-N-(2-fluorobenzyl)-1-{(2S,3S)-2-hydroxy-3-[(3-hydroxy-2-methylbenzoyl)amino]-4-phenylbutanoyl}-4-methoxy-3,3-dimethyl-L-prolinamide; and
(4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide; or
   pharmaceutically acceptable salts or solvates thereof.

Also provided herein are compounds selected from:
(R)-3-((2S, 3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
(S)-4,4-Difluoro-1-[4-(3-fluoro-phenyl)-(2S,3S)-hydroxy-(3-hydroxy-2-methyl-benzoylamino)-butyryl]-3,3-dimethyl-pyrrolidine-2-carboxylic acid (2-methoxy-(1S)-methyl-ethyl)-amide;
N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide;
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide;
(S)-4,4-Difluoro-1-[4-(3-fluoro-phenyl)-(2S)-hydroxy-(3S)-(3-hydroxy-2-methyl-benzoylamino)-butyryl]-3,3-dimethyl-pyrrolidine-2-carboxylic acid allylamide;
(S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (3-methyl-pyridin-4-ylmethyl)-amide;
2,3-Dihydro-1H-indole-4-carboxylic acid [(1S,2S)-1-benzyl-3-((S)-4,4-difluoro-2-isobutylcarbamoyl-3,3-dimethyl-pyrrolidin-1-yl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide;
2,3-Dihydro-1H-indole-4-carboxylic acid [(1S,2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-4-ylmethyl)-amide; and
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide; or
   pharmaceutically acceptable salts or solvates thereof.

In still another aspect of the present invention are provided compounds selected from:
(R)-3-((2S,3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
(S)-4,4-Difluoro-1-[4-(3-fluoro-phenyl)-(2S,3S)-hydroxy-(3-hydroxy-2-methyl-benzoylamino)-butyryl]-3,3-dimethyl-pyrrolidine-2-carboxylic acid (2-methoxy-(1S)-methyl-ethyl)-amide; and
N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide; or
   pharmaceutically acceptable salts or solvates thereof.

In still a further aspect are provided compounds selected from:
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide;
(S)-4,4-Difluoro-1-(2S,3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide;
(S)-4,4-Difluoro-1-[4-(3-fluoro-phenyl)-(2S)-hydroxy-(3S)-(3-hydroxy-2-methyl-benzoylamino)-butyryl]-3,3-dimethyl-pyrrolidine-2-carboxylic acid allylamide;
(S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (3-methyl-pyridin-4-ylmethyl)-amide;
2,3-Dihydro-1H-indole-4-carboxylic acid [(1S,2S)-1-benzyl-3-((S)-4,4-difluoro-2-isobutylcarbamoyl-3,3-dimethyl-pyrrolidin-1-yl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide;
2,3-Dihydro-1H-indole-4-carboxylic acid [(1S,2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-4-ylmethyl)-amide; and
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide; or
   pharmaceutically acceptable salts or solvates thereof.

In yet another embodiment are compounds selected from:
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide;
(S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide;
2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide;
(S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide;
(S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide;
1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
N-((1S,2S)-3-{(2S)-2-[(allylamino)carbonyl]-4,4-difluoro-3,3-dimethylpyrrolidin-1-yl}-1-benzyl-2-hydroxy-3-oxopropyl)indoline-4-carboxamide;
(4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide;
(S)-3-((2S,3S)-2-Hydroxy-3-{[1-(3-hydroxy-2-methyl-phenyl)-methanoyl]-amino-}4-phenyl-butanoyl)-5,5-dimethyl-oxazolidine-4-carboxylic acid (S)-indan-1-ylamide;
(3R)-N-allyl-1-{(2S,3S)-2-hydroxy-3-[(3-hydroxy-2-methylbenzoyl)amino]-4-phenylbutanoyl}-3-phenyl-L-prolinamide;
(4S)-N-(2-fluorobenzyl)-1-{(2S,3S)-2-hydroxy-3-[(3-hydroxy-2-methylbenzoyl)amino]-4-phenylbutanoyl}-4-methoxy-3,3-dimethyl-L-prolinamide; and
(4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide; or
   pharmaceutically acceptable salts or solvates thereof.

Further still are provided compounds selected from:
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide;
(S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide;
2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide;
(S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide;
(S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide;
1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide; and
1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide; or
   pharmaceutically acceptable salts or solvates thereof.

In still a further aspect are provided compounds selected from:
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
N-((1S,2S)-3-{(2S)-2-[(allylamino)carbonyl]-4,4-difluoro-3,3-dimethylpyrrolidin-1-yl}-benzyl-2-hydroxy-3-oxopropyl)indoline-4-carboxamide;
(4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide;
(S)-3-((2S,3S)-2-Hydroxy-3-{[1-(3-hydroxy-2-methyl-phenyl)-methanoyl]-amino}4-phenyl-butanoyl)-5,5-dimethyl-oxazolidine-4-carboxylic acid (S)-indan-1-ylamide;
(3R)-N-allyl-1-{(2S,3S)-2-hydroxy-3-[(3-hydroxy-2-methylbenzoyl)amino]-4-phenylbutanoyl}3-phenyl-L-prolinamide;
(4S)-N-(2-fluorobenzyl)-1-{(2S,3S)-2-hydroxy-3-[(3-hydroxy-2-methylbenzoyl)amino]-4-phenylbutanoyl}-4-methoxy-3,3-dimethyl-L-prolinamide; and
(4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide; or
   pharmaceutically acceptable salts or solvates thereof.

A further aspect provides compounds selected from:
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide; and
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide; or
   pharmaceutically acceptable salts or solvates thereof.

A further aspect provides (S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide, or pharmaceutically acceptable salts or solvates thereof.

A further aspect provides (R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide or pharmaceutically acceptable salts or solvates thereof.

Also provided is (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide or pharmaceutically acceptable salts or solvates thereof.

Further provided is (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide or pharmaceutically acceptable salts or solvates thereof.

Also provided is (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide or pharmaceutically acceptable salts or solvates thereof.

Another aspect provides (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide or pharmaceutically acceptable salts or solvates thereof.

Further provided is (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide or pharmaceutically acceptable salts or solvates thereof.

In another aspect is provided (S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide or pharmaceutically acceptable salts or solvates thereof.

Further provided is (S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide or pharmaceutically acceptable salts or solvates thereof.

In a further aspect is provided (S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide or pharmaceutically acceptable salts or solvates thereof.

Further provided is (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide or pharmaceutically acceptable salts or solvates thereof.

The present invention also affords compounds selected from: 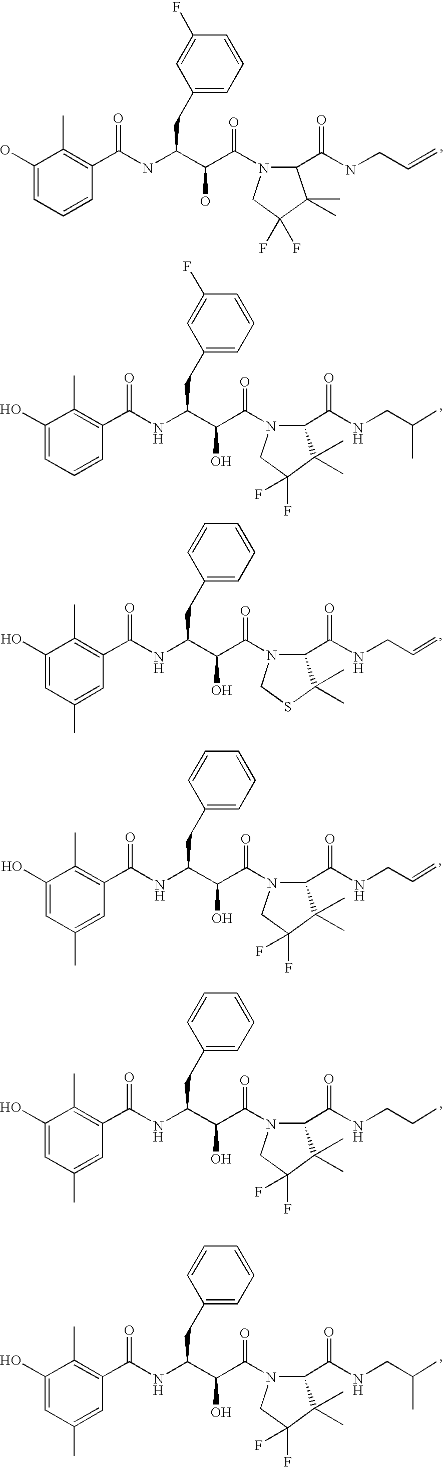 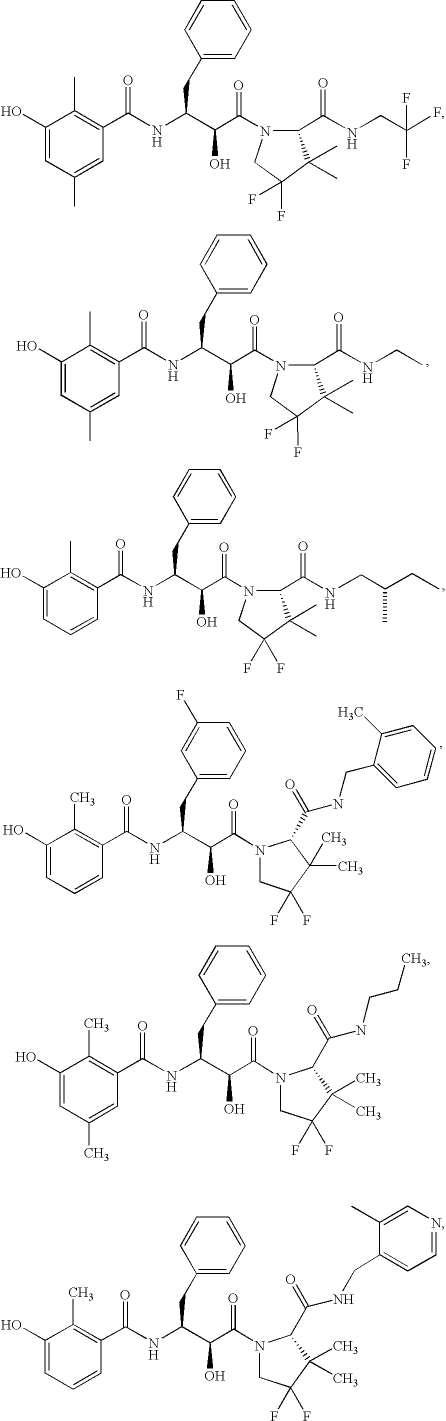 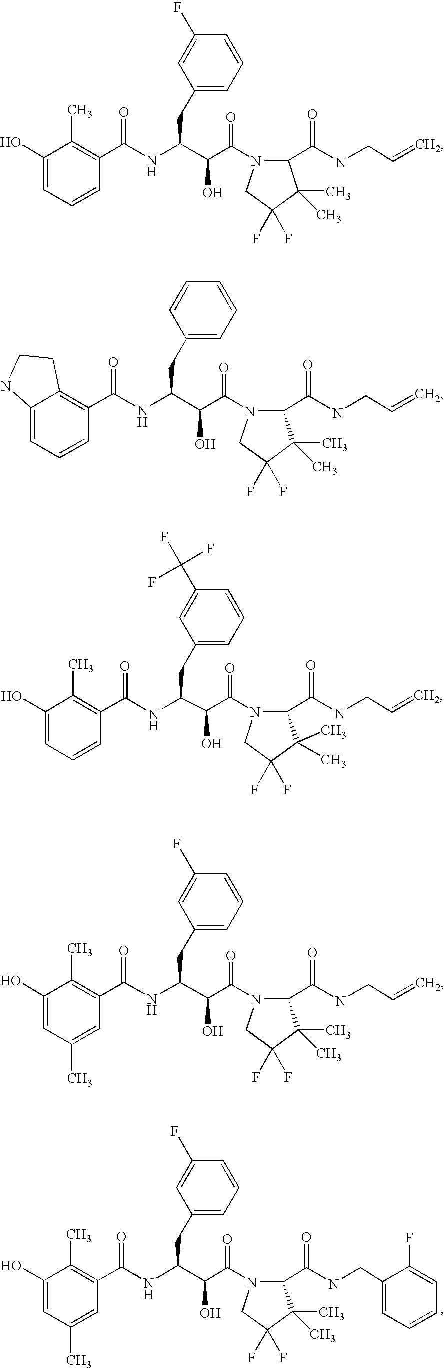 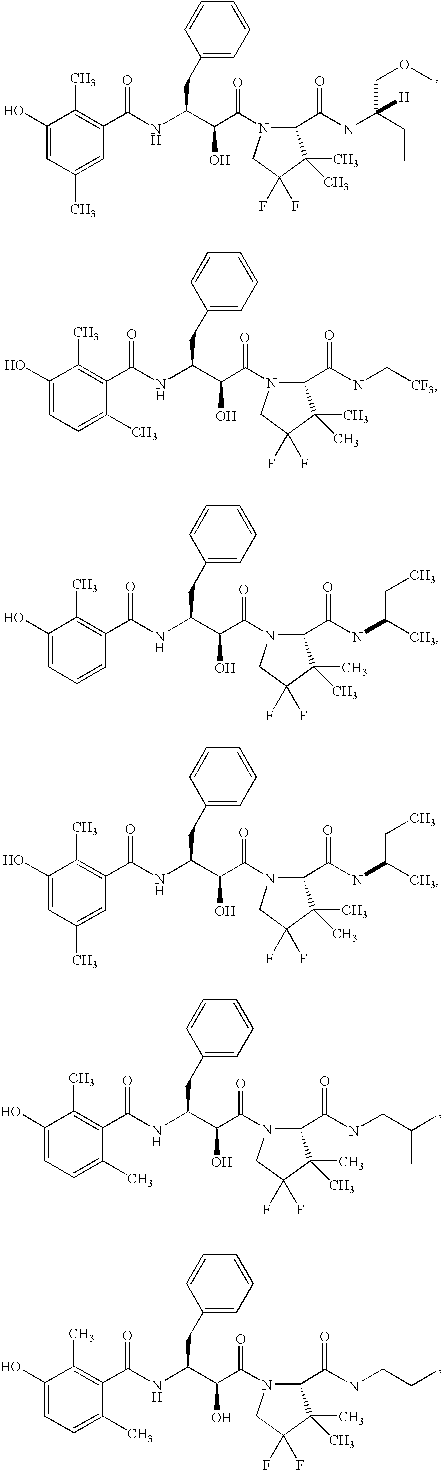 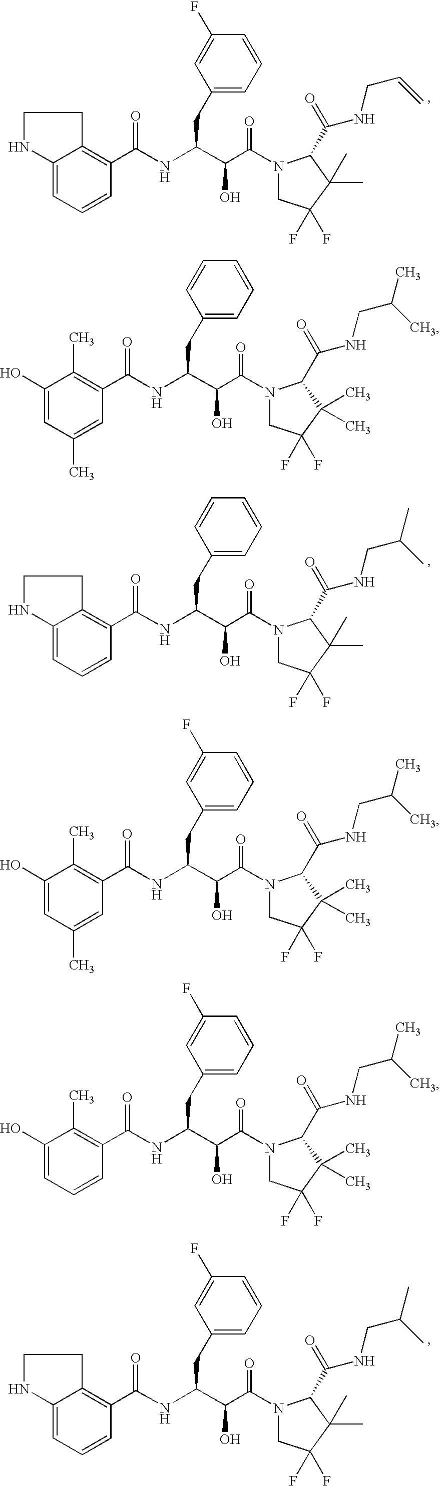 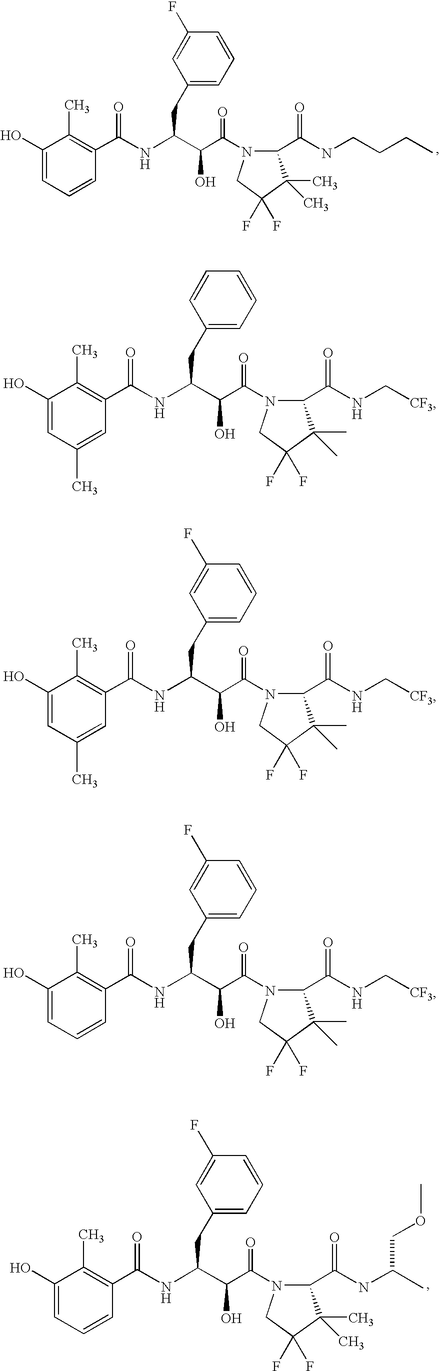 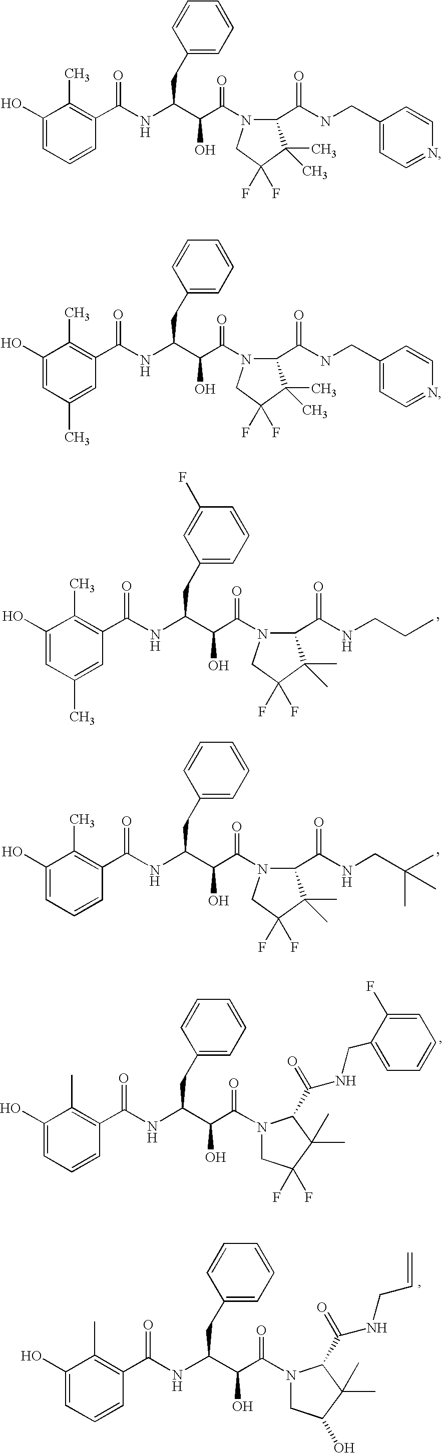 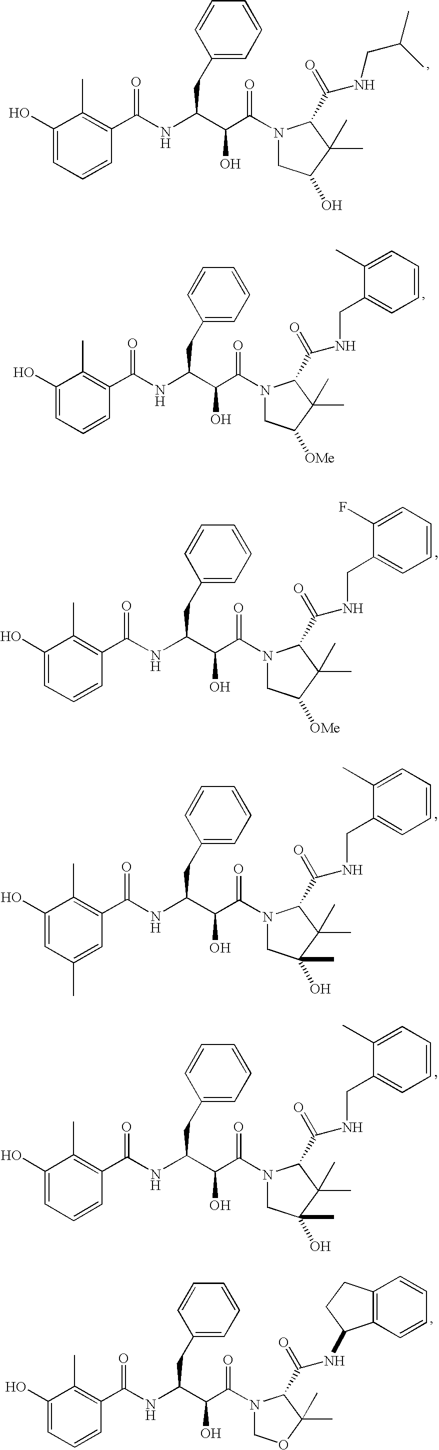 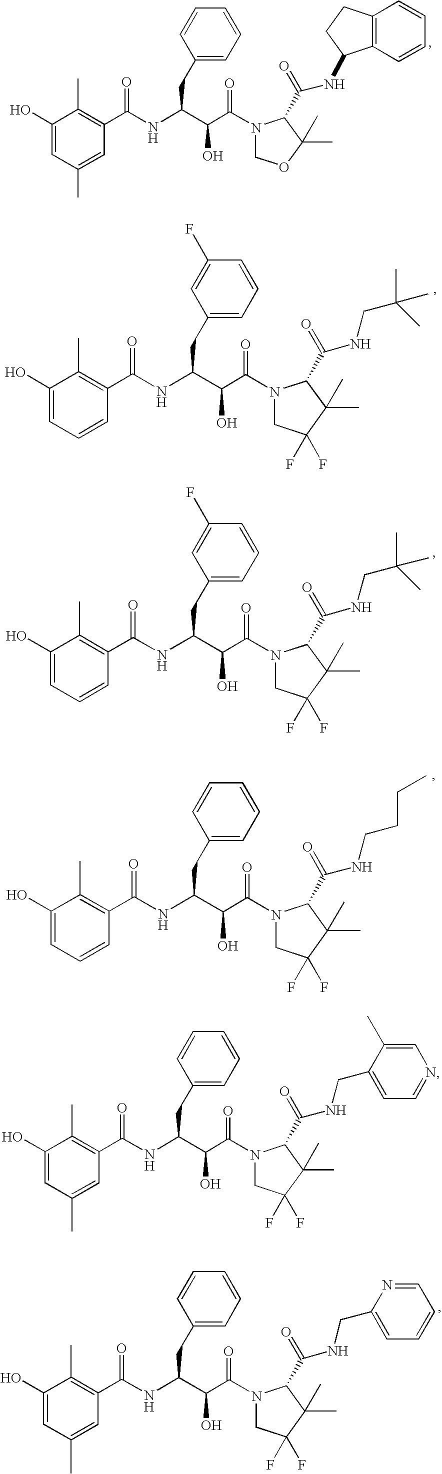 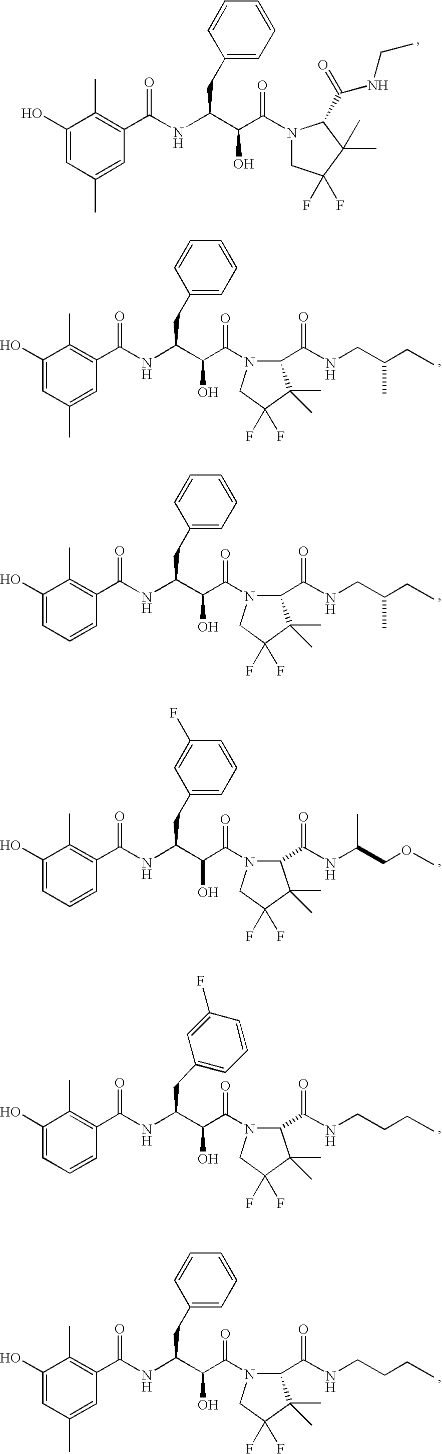 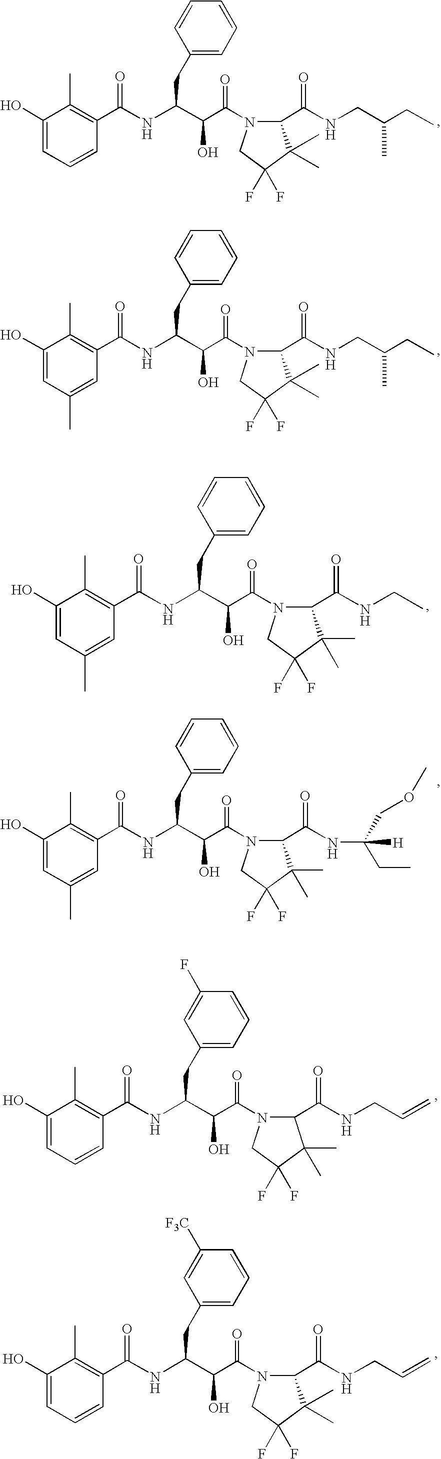 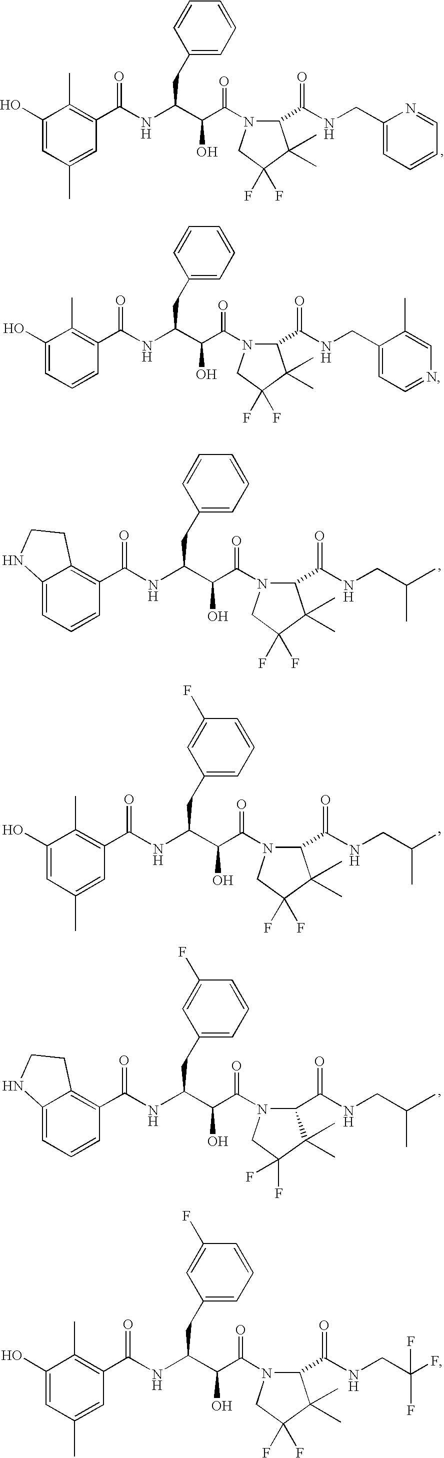 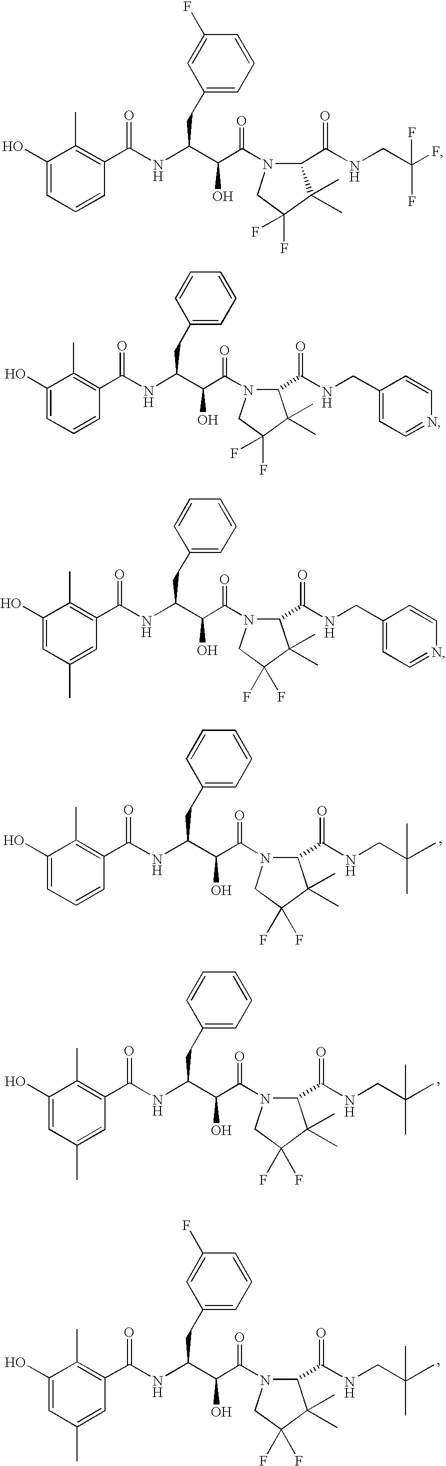 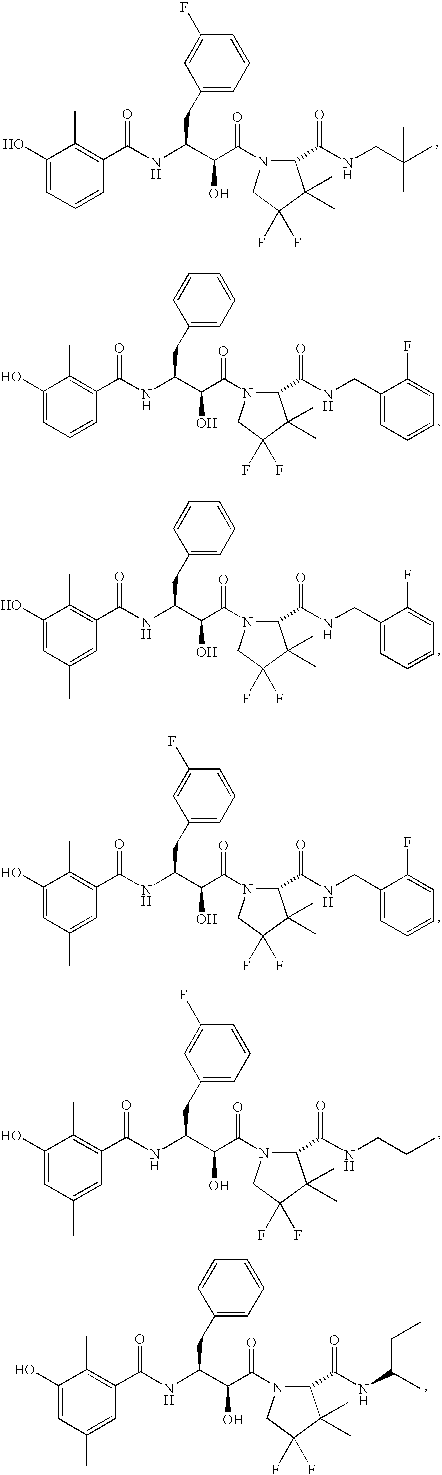 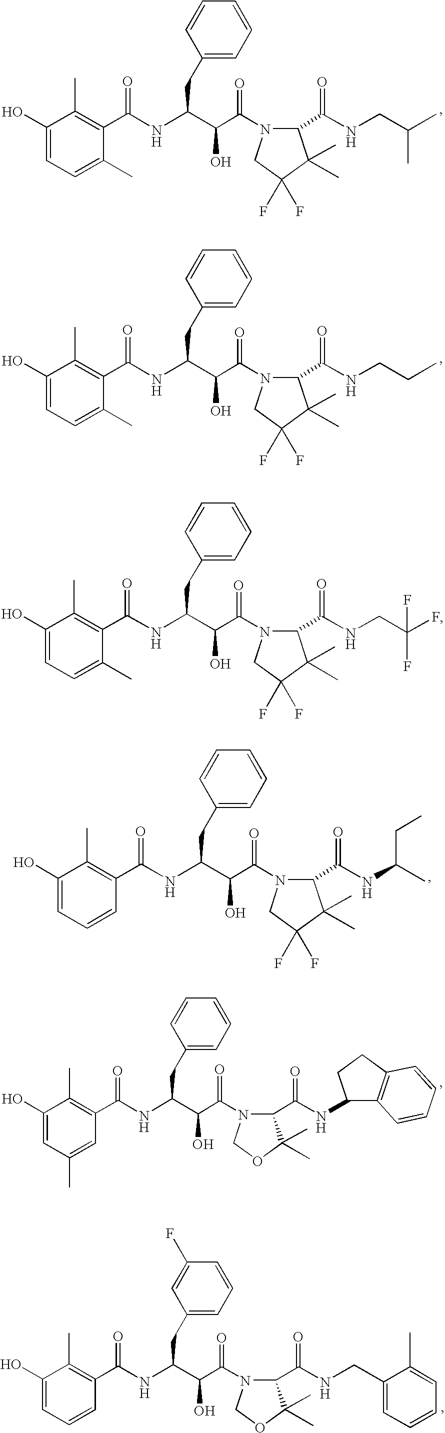 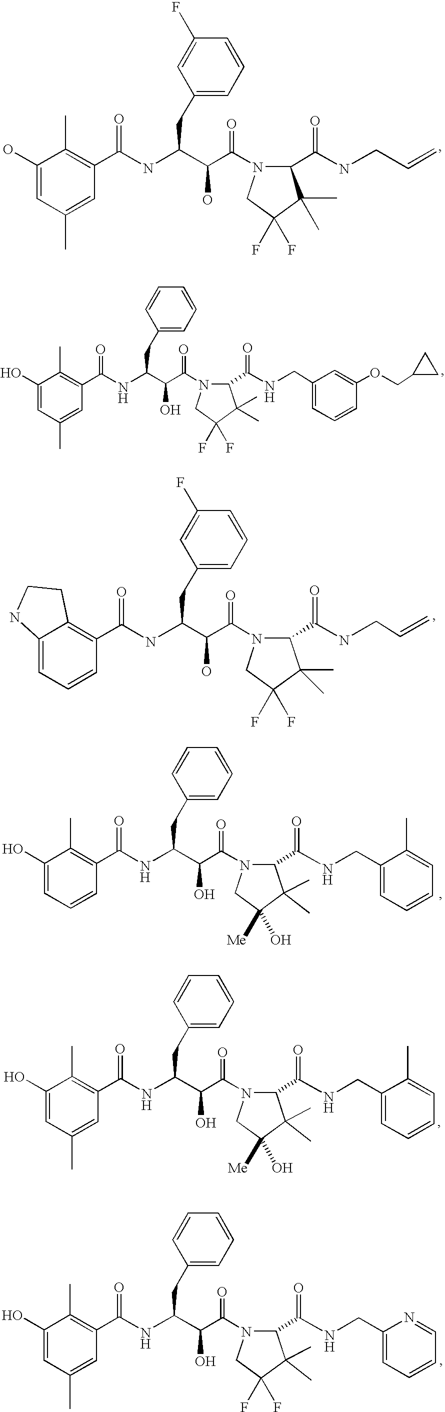 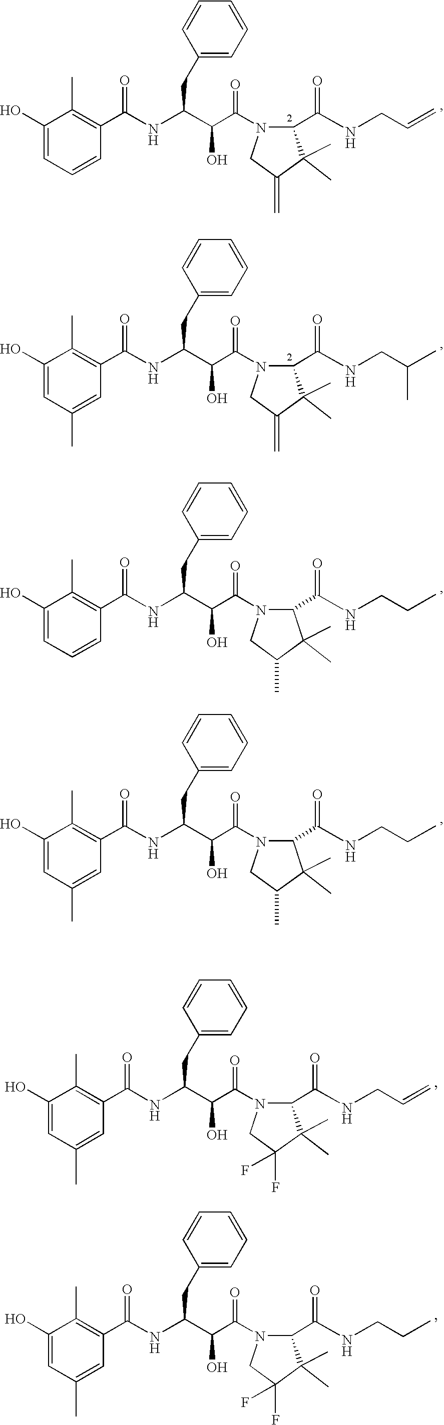 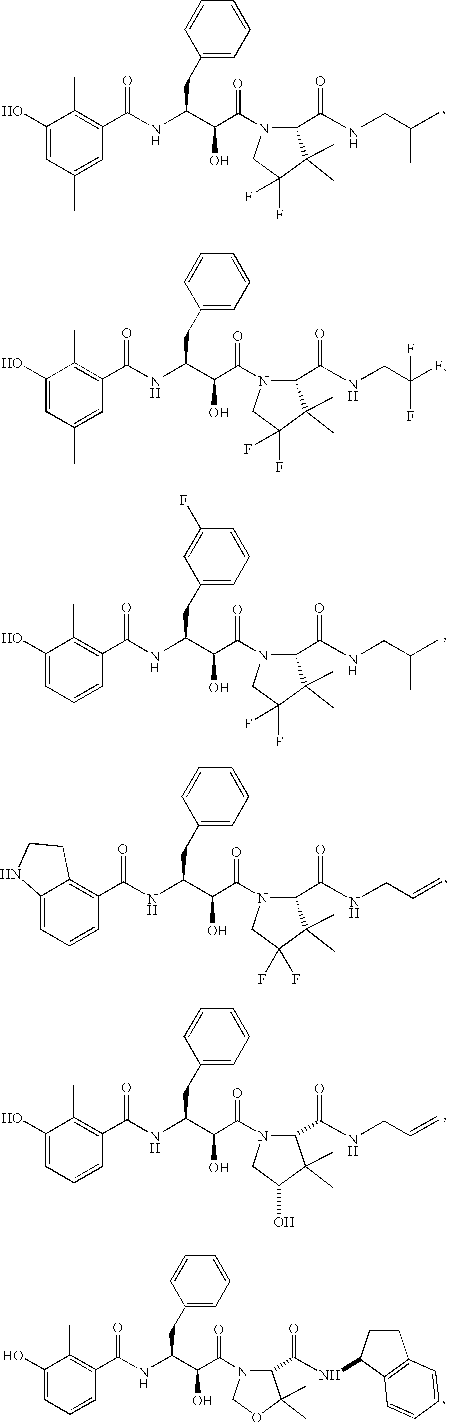 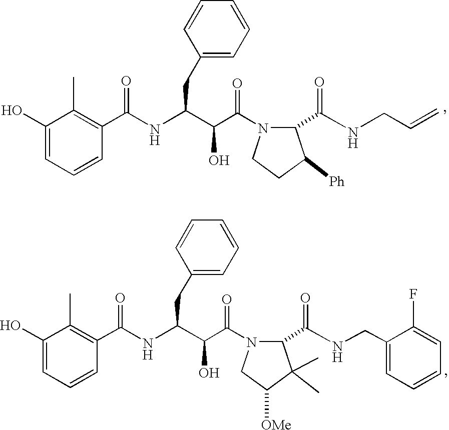 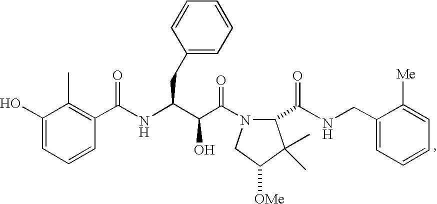
or pharmaceutically acceptable salts or solvates thereof.

### DETAILED DESCRIPTION

As used herein, the terms “comprising” and “including” are used in their open, non-limiting sense.

As used herein, the terms “compound(s) of formula (I),” or “compound(s) of the present invention,” or “compound(s) of the invention” should be understood to mean the compounds as described or depicted and any pharmaceutically acceptable salt or solvate thereof. Furthermore, such phrases are meant to include any compounds described herein that are specifically encompassed by the genus of Formula (I) or any subgenus included herein.

When the phrase, “substituted with at least one substituent” is used herein, it is meant to indicate that the group in question may be substituted by at least one of the substituents chosen. The number of substituents a group in the compounds of the invention may have depends on the number of positions available for substitution. For example, an aryl ring in the compounds of the invention may contain from 1 to 5 additional substituents, depending on the degree of substitution present on the ring. The maximum number of substituents that a group in the compounds of the invention may have can be determined by those of ordinary skill in the art.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above. The methods of treatment for mitigation of a disease condition include the use of the compounds in this invention in any conventionally acceptable manner, for example, as a prophylactic.

The term “HIV-inhibiting amount,” as used herein with reference to the compounds of the present invention means the amount of a compound of the present invention that is necessary to inhibit the replication of the HIV virus in a mammal, such as a human or in vivo, as in a cell culture. The amount of a compound of the present invention necessary to inhibit the replication of HIV in a mammal will vary depending on, for example, the condition of the mammal, the formulation in which the compounds of the invention are used, and any other retroviral compounds that may be administered in combination with the compounds of the present invention. The amount of a compound of the present invention that is required to inhibit HIV replication can be determined using methods known to those of ordinary skill in the art.

The terms “therapeutically effective amount” and “effective amount” are intended to mean the amount of a compound of the present invention that, when administered to a mammal in need of treatment, is sufficient to effect treatment for injury or disease conditions alleviated by the inhibition of HIV RNA replication such as for potentiation of anti-cancer therapies or inhibition of neurotoxicity consequent to stroke, head trauma, and neurodegenerative diseases. The amount of a given HIV-inhibiting agent used in the method of the invention that will be therapeutically effective will vary depending upon factors such as the particular HIV-inihibiting agent, the disease condition and the severity thereof, the identity and characteristics of the mammal in need thereof, which amount may be routinely determined by artisans.

The term “reacting,” as used herein, refers to a chemical process or processes in which two or more reactants are allowed to come into contact with each other to effect a chemical change or transformation. For example, when reactant A and reactant B are allowed to come into contact with each other to afford a new chemical compound(s) C, A is said to have “reacted” with B to produce C.

The term “protecting,” as used herein, refers to a process in which a functional group in a chemical compound is selectively masked by a non-reactive functional group in order to allow a selective reaction(s) to occur elsewhere on said chemical compound. Such non-reactive functional groups are herein termed “protecting groups.” For example, the term “hydroxyl protecting group,” as used herein refers to those groups that are capable of selectively masking the reactivity of a hydroxyl (—OH) group. The term “suitable protecting group,” as used herein, refers to those protecting groups that are useful in the preparation of the compounds of the present invention. Such groups are generally able to be selectively introduced and removed using mild reaction conditions that do not interfere with other portions of the subject compounds. Protecting groups that are suitable for use in the processes and methods of the present invention are known to those of ordinary skill in the art. The chemical properties of such protecting groups, methods for their introduction and their removal can be found, for example, in T. Greene and P. Wuts, Protective Groups in Organic Synthesis (3^{rd} ed.), John Wiley & Sons, NY (1999). The terms “deprotecting,” “deprotected,” or “deprotect,” as used herein, are meant to refer to the process of removing a protecting group from a compound.

The term “leaving group,” as used herein, refers to a chemical functional group that generally allows a nucleophilic substitution reaction to take place at the atom to which it is attached. For example, in acid chlorides of the formula Cl—C(O)R, wherein R is alkyl, aryl, or heterocyclic, the —Cl group is generally referred to as a leaving group because it allows nucleophilic substitution reactions to take place at the carbonyl carbon. Suitable leaving groups are known to those of ordinary skill in the art and can include halides, aromatic heterocycles, cyano, amino groups (generally under acidic conditions), ammonium groups, alkoxide groups, carbonate groups, formates, and hydroxy groups that have been activated by reaction with compounds such as carbodiimides. For example, suitable leaving groups can include, but are not limited to, chloride, bromide, iodide, cyano, imidazole, and hydroxy groups that have been allowed to react with a carbodiimide such as dicyclohexylcarbodiimide (optionally in the presence of an additive such as hydroxybenzotriazole) or a carbodiimide derivative.

The term “acetylating agent,” as used herein, refers to chemical compounds that are useful for the introduction of an acetyl group, —C(O)CH₃, onto a hydroxyl group in the compounds of the invention. The symbol “Ac—,” as used in chemical structures herein, is meant to represent an acyl group in the compounds of the invention. Useful acetylating agents include, but are not limited to, acetic anhydride, acetyl chloride, acetyl bromide, and acetyl iodide. In addition, such acetylating agents can be prepared in situ by reaction of an appropriate combination of compounds, such as the reaction of acetyl chloride with sodium iodide in acetone to afford an intermediate acetyl iodide agent. The term “acetic anhydride,” as used herein, is meant to represent a compound with the chemical formula CH₃C(O)OC(O)CH₃.

As used herein, the term “aliphatic” represents a saturated or unsaturated, straight- or branched-chain hydrocarbon, containing 1 to 10 carbon atoms which may be unsubstituted or substituted by one or more of the substituents described below. The term “aliphatic” is intended to encompass alkyl, alkenyl and alkynyl groups.

As used herein, the term “C₁₋₁₀ alkyl” represents a straight- or branched-chain saturated hydrocarbon, containing 1 to 10 carbon atoms which may be unsubstituted or substituted by one or more of the substituents described below. Exemplary alkyl substituents include, but are not limited to methyl (Me), ethyl (Et), propyl, isopropyl, butyl, isobutyl, t-butyl, and the like.

The term “C₂₋₁₀ alkenyl” represents a straight- or branched-chain hydrocarbon, containing one or more carbon-carbon double bonds and having 2 to 10 carbon atoms which may be unsubstituted or substituted by one or more of the substituents described below. Exemplary alkenyl substituents include, but are not limited to ethenyl, propenyl, butenyl, allyl, pentenyl and the like.

The term “C₂₋₁₀ alkynyl”, as used herein, unless otherwise indicated, includes C₂₋₁₀ alkyl moieties having at least one carbon-carbon triple bond wherein alkyl is as defined above. The triple bond may be located anywhere on the C₂₋₁₀ carbon chain that results in a stable structure.

The term “hydroxyl,” as used herein refers to a —OH group in the compounds of the present invention. Such a group may also be referred to herein as a “hydroxy” group.

As used herein, the terms “heterocyclic” and “heterocyclyl” refer to ring systems containing from 3 to 18 ring atoms, including 1 to 5 heteroatoms chosen from O, N, and S. Such ring systems may be attached to the remainder of the compounds of the present invention through any atom that will result in a stable structure, including any heteroatom or carbon atom.

The term “C₆₋₁₀ aryl,” as used herein, refers to carbocyclic, aromatic ring systems containing from 6 to 10 carbon atoms. Such ring systems may be mono-, bi-, or tri-cyclic. In addition, such ring systems may be attached to the remainder of the compounds of formula (I) at any atom that results in a stable structure. Examples of such ring systems include, but are not limited to, benzyl and napthyl. The term “phenyl,” as used herein refers to a fully unsaturated 6-membered carbocyclic group and is meant to be encompassed by the term “C₆₋₁₀ aryl.” A “phenyl” group may also be referred to herein as a benzene derivative. The symbol “Ph” may be used herein to denote a phenyl group.

The term “heteroaryl,” as used herein refers to a group comprising an aromatic monovalent monocyclic, bicyclic, or tricyclic group, containing 5 to 18 ring atoms, including 1 to 5 heteroatoms selected from nitrogen, oxygen and sulfur, which may be unsubstituted or substituted by one or more of the substituents described below. In addition, the term “heteroaryl” is meant to encompass those ring systems that are benzofused, such as indole, benzothiophene, benzofuran, and the like. The term is also meant to encompass those groups in which a heterocyclic ring, saturated, partially saturated, or unsaturated, is fused to a benzene ring. Examples of such ring systems are indoline, dihydrobenzofuran, and dihydrobenzothiophene. In such ring systems, it is specifically contemplated that such groups may contain a bond at any suitable atom, including carbon and any heteroatoms. For example, it is contemplated that in the compounds of the present invention, the indoline ring system may be contain a bond in either the 5-membered heterocyclic ring system or the 6-member carbocyclic ring system. As used herein, the term “heteroaryl” is also intended to encompass the N-oxide derivative (or N-oxide derivatives, if the heteroaryl group contains more than one nitrogen such that more than one N-oxide derivative may be formed) of the nitrogen-containing heteroaryl groups described herein. Illustrative examples of heteroaryl groups include, but are not limited to, thienyl, pyrrolyl, imidazolyl, pyrazolyl, furyl, isothiazolyl, furazanyl, isoxazolyl, thiazolyl, pyridyl, pyrazinyl, pyrimidinyl, pyridazinyl, triazinyl, benzo[b]thienyl, naphtho[2,3-b]thianthrenyl, isobenzofuranyl, chromenyl, xanthenyl, phenoxathienyl, indolizinyl, isoindolyl, indolyl, indazolyl, purinyl, isoquinolyl, quinolyl, phthalazinyl, naphthyridinyl, quinoxyalinyl, quinzolinyl, benzothiazolyl, benzimidazolyl, tetrahydroquinolinyl, cinnolinyl, pteridinyl, carbazolyl, beta-carbolinyl, phenanthridinyl, acridinyl, perimidinyl, phenanthrolinyl, phenazinyl, isothiazolyl, phenothiazinyl, and phenoxazinyl. Illustrative examples of N-oxide derivatives of heteroaryl groups include, but are not limited to, pyridyl N-oxide, pyrazinyl N-oxide, pyrimidinyl N-oxide, pyridazinyl N-oxide, triazinyl N-oxide, isoquinolyl N-oxide, and quinolyl N-oxide. Further examples of heteroaryl groups include the following moieties: 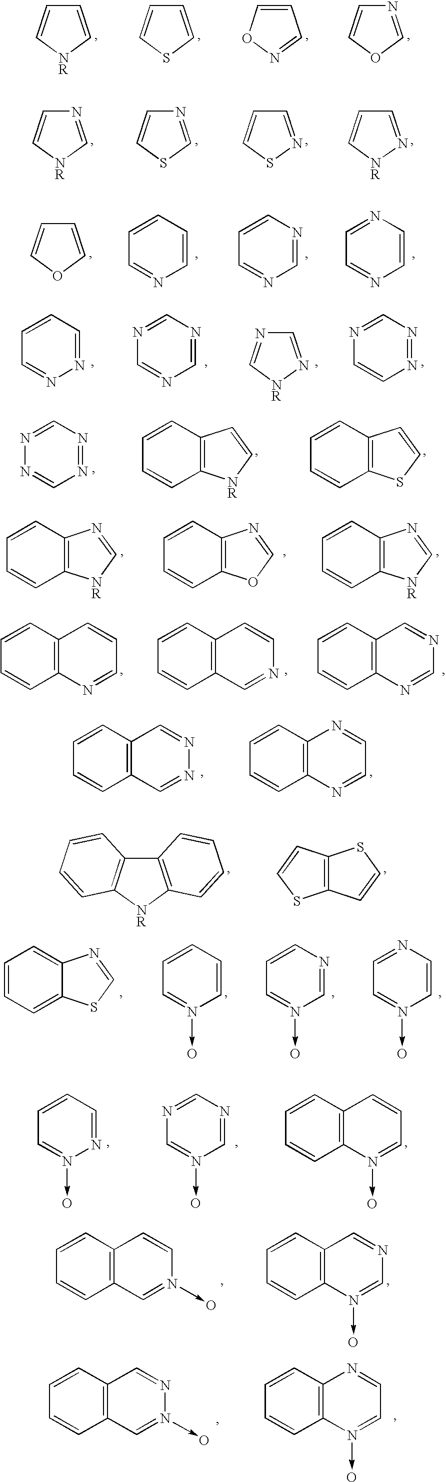 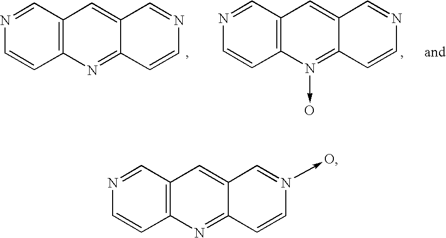
wherein R is H, alkyl, hydroxyl or represents a compound according to Formula I.

The terms “halogen” and “halo” represent chloro, fluoro, bromo or iodo substituents.

The term “C₁₋₆ alkylcarbonyloxy,” as used herein, refers to groups of the formula —OC(O)R, wherein R is an alkyl group comprising from 1 to 6 carbon atoms.

The term “C₆₋₁₀ arylcarbonyloxy,” as used herein, refers to a group of the formula —OC(O)R, wherein R is an aryl group comprising from 6 to 10 carbons.

The term “heteroarylcarbonyloxy,” as used herein, refers to a group of the formula —OC(O)R, wherein R is a heteroaromatic group as defined above.

The term “C₆₋₁₀arylC₁₋₁₀alkyl,” as used herein is meant to refer to a group in which a C₆₋₁₀ aryl group, as defined herein, is attached to a C₁₋₁₀ alkyl group as defined herein. The phenyl group may be attached at any point on the C₁₋₁₀alkyl group that will result in a stable structure. Examples of a C₆₋₁₀arylC₁₋₁₀alky group include, but are not limited to, PhCH₂—, PhCH₂CH₂—, (CH₃)PhCH—, and the like.

The term “heteroarylC₁₋₁₀alkyl,” as used herein, is meant to refer to a group in which a heteroaryl group, as defined herein, is attached to a C₁₋₁₀ alkyl group, as defined herein. The heteroaryl group may be attached at any point on the C₁₋₁₀alkyl group that will result in a stable structure. In addition, the heteroaryl group may be attached by any atom to the C₁₋₁₀ alkyl group at any point that will result in a stable structure. For example, a pyridyl group may be attached to the C₁₋₁₀ alkyl group at the 2, 3, or 4 position of the pyridyl ring system. Examples of a heteroarylC₁₋₁₀alkyl group include, but are not limited to (pyridyl)CH₂—, (imidazole)CH₂—, and the like.

The term “heterocyclylC₁₋₁₀alkyl,” as used herein is meant to refer to a group in which a heterocyclic group, as defined herein, is attached to a C₁₋₁₀ alkyl group, as defined herein. The heterocyclic group may be attached at any point on the C₁₋₁₀alkyl group that will result in a stable structure. In addition, the heterocyclic group may be attached by any atom to the C₁₋₁₀ alkyl group at any point that will result in a stable structure. For example, a piperazine moiety may be attached to the C₁₋₁₀ alkyl chain at either the 1, 2, 3, or 4 position of the piperazine moiety. Examples of heterocyclylC₁₋₁₀alkyl groups include, but are not limited to, (piperazinyl)CH₂—, (morpholine)CH₂—, and the like.

The terms “carbocycle” or “carbocyclic,” as used herein, refer to a saturated, partially saturated, unsaturated, or aromatic, monocyclic or fused or non-fused polycyclic, ring structure having only carbon ring atoms (no heteroatoms, i.e., non-carbon ring atoms). Exemplary carbocycles include cycloalkyl, aryl, and cycloalkyl-aryl groups.

A “C₃₋₁₀ cycloalkyl group” is intended to mean a saturated or partially saturated, monocyclic, or fused or spiro polycyclic, ring structure having a total of from 3 to 18 carbon ring atoms (but no heteroatoms). Exemplary cycloalkyls include cyclopropyl, cyclobutyl, cyclopentyl, cyclopentenyl, cyclohexyl, cycloheptyl, adamantyl, and like groups.

A “heterocycloalkyl group” is intended to mean a monocyclic, or fused or spiro polycyclic, ring structure that is saturated or partially saturated, and has a total of from 3 to 18 ring atoms, including 1 to 5 heteroatoms selected from nitrogen, oxygen, and sulfur. Illustrative Examples of heterocycloalkyl groups include pyrrolidinyl, tetrahydrofuryl, piperidinyl, piperazinyl, morpholinyl, thiomorpholinyl, aziridinyl, and like groups.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

The term “—OC₁₋₁₀ alkyl”, as used herein, unless otherwise indicated, includes O-alkyl of 1 to 10 carbons, wherein C₁₋₁₀ alkyl is as defined above. Examples of —OC₁₋₁₀alkyl groups include, but are not limited to, —OCH₃, —OCH(CH₃)CH₃, —OCH₂CH₂CH₃, and the like.

The term “amino” is intended to mean the —NH₂ radical.

In accordance with a convention used in the art, the symbol 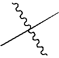
is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g., 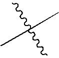
represents a methyl group, 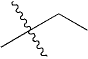
represents an ethyl group, 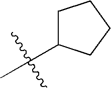
represents a cyclopentyl group, etc.

The term “substituted” means that the specified group or moiety bears one or more substituents. The term “unsubstituted” means that the specified group bears no substituents. The term “optionally substituted” means that the specified group is unsubstituted or substituted by one or more substituents. When the phrase, “substituted with at least one substituent” is used herein, it is meant to indicate that the group in question may be substituted by at least one of the substituents chosen. The number of substituents a group in the compounds of the invention may have depends on the number of positions available for substitution. For example, an aryl ring in the compounds of the invention may contain from 1 to 5 additional substituents, depending on the degree of substitution present on the ring. The maximum number of substituents that a group in the compounds of the invention may have can be determined by those of ordinary skill in the art.

An “HIV-inhibiting agent” means a compound represented by formula (I) or a pharmaceutically acceptable salt, hydrate, prodrug, active metabolite or solvate thereof.

A “prodrug” is a compound that may be converted under physiological conditions or by solvolysis to the specified compound or to a pharmaceutically acceptable salt of such compound. A prodrug may be a derivative of one of the compounds of the present invention that contains a moiety, such as for example —CO₂R,—PO(OR)₂ or —C═NR, that may be cleaved under physiological conditions or by solvolysis. Any suitable R substituent may be used that provides a pharmaceutically acceptable solvolysis or cleavage product. A prodrug containing such a moiety may be prepared according to conventional procedures by treatment of a hydroxamate derivative of this invention containing, for example, an amido, carboxylic acid, or hydroxyl moiety with a suitable reagent. An “active metabolite” is a pharmacologically active product produced through metabolism in the body of a specified hydroxamate derivative or salt thereof. Prodrugs and active metabolites of the hydroxamate derivative may be identified using routine techniques known in the art. See, e.g., Bertolini, et al., J. Med. Chem., 40:2011-2016 (1997); Shan et al., J. Pharm. Sci., 86 (7):765-767 (1997); Bagshawe, Drug Dev. Res., 34:220-230 (1995); Bodor, Advances in Drug Res., 13:224-331 (1984); Bundgaard, Design of Prodrugs (Elsevier Press, 1985); Larsen, Design and Application of Prodrugs, Drug Design and Development (Krogsgaard-Larsen et al. eds., Harwood Academic Publishers, 1991); Dear, et al., Chromatogr. B, 748:281-293 (2000); Spraul, et al., J. Pharmaceutical & Biomedical Analysis, 10 (8):601-605 (1992); and Prox, et al., Xenobiol, 3(2):103-112 (1992).

A “solvate” is intended to mean a pharmaceutically acceptable solvate form of a specified compound that retains the biological effectiveness of such compound. Examples of solvates include compounds of the invention in combination with water, isopropanol, ethanol, methanol, DMSO, ethyl acetate, acetic acid, or ethanolamine. A “pharmaceutically acceptable salt” is intended to mean a salt that retains the biological effectiveness of the free acids and bases of the specified derivative and that is not biologically or otherwise undesirable. Examples of pharmaceutically acceptable salts include sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

The phrase “pharmaceutically acceptable salt(s)”, as used herein, unless otherwise indicated, includes salts of acidic or basic groups, which may be present in the compounds herein described. The compounds that are basic in nature are capable of forming a wide variety of salts with various inorganic and organic acids. The acids that may be used to prepare pharmaceutically acceptable acid addition salts of such basic compounds herein described are those that form non-toxic acid addition salts, i.e., salts containing pharmacologically acceptable anions, such as the acetate, benzenesulfonate, benzoate, bicarbonate, bisulfate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts.

In particular, the compounds herein described that are basic in nature are capable of forming a wide variety of different salts with various inorganic and organic acids. Although such salts must be pharmaceutically acceptable for administration to animals, it is often desirable in practice to initially isolate the compound from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds herein described that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds herein described. Such non-toxic base salts include those derived from such pharmacologically acceptable cations as sodium, potassium calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantifies of reagents are preferably employed in order to ensure completeness of reaction and maximum yields of the desired final product.

If a derivative used in the method of the invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucuronic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of derivatives, prodrugs, salts, or solvates that are solids, it is understood by those skilled in the art that the derivatives, prodrugs, salts, and solvates used in the method of the invention, may exist in different polymorph or crystal forms, all of which are intended to be within the scope of the present invention and specified formulas. In addition, the derivative, salts, prodrugs and solvates used in the method of the invention may exist as tautomers, all of which are intended to be within the broad scope of the present invention.

The compounds of the present invention contain at least one chiral center and may exist as single stereoisomers (e.g., single enantiomers or single diastereomers), any mixture of stereoisomers (e.g., any mixture of enantiomers or diastereomers) or racemic mixtures thereof. It is specifically contemplated that, unless otherwise indicated, all stereoisomers, mixtures and racemates of the present compounds are encompassed within the scope of the present invention. Compounds identified herein as single stereoisomers are meant to describe compounds that are present in a form that contains from at least about 90% to at least about 99% of a single stereoisomer of each chiral center present in the compounds. Where the stereochemistry of the chiral carbons present in the chemical structures illustrated herein are not specified, it is specifically contemplated that all possible stereoisomers are encompassed therein. The compounds of the present invention may be prepared and used in stereoisomerically pure form or substantially stereoisomerically pure form. As used herein, the term “stereoisomeric” purity refers to the “enantiomeric” purity and/or “diastereomeric” purity of a compound. The term “stereoisomerically pure form,” as used herein, is meant to encompass those compounds that contain from at least about 95% to at least about 99%, and all values in between, of a single stereoisomer. The term “substantially enantiomerically pure,” as used herein is meant to encompass those compounds that contain from at least about 90% to at least about 95%, and all values in between, of a single stereoisomer. The term “diastereomerically pure,” as used herein, is meant to encompass those compounds that contain from at least about 95% to at least about 99%, and all values in between, of a single diastereoisomer. The term “substantially diastereomerically pure,” as used herein, is meant to encompass those compounds that contain from at least about 90% to at least about 95%, and all values in between, of a single diastereoisomer. The terms “racemic” or “racemic mixture,” as used herein, refer to a mixture containing equal amounts of stereoisomeric compounds of opposite configuration. For example, a racemic mixture of a compound containing one stereoisomeric center would comprise equal amount of that compound in which the stereoisomeric center is of the (S)- and (R)-configurations. The term “enantiomerically enriched,” as used herein, is meant to refer to those compositions wherein one stereoisomer of a compound is present in a greater amount than the opposite stereoisomer. Similarly, the term “diastereomerically enriched,” as used herein, refers to those compositions wherein one diastereomer of compound is present in amount greater than the opposite diastereomer. The compounds of the present invention may be obtained in stereoisomerically pure (i.e., enantiomerically and/or diastereomerically pure) or substantially stereoisomerically pure (i.e., substantially enantiomerically and/or diastereomerically pure) form. Such compounds may be obtained synthetically, according to the procedures described herein using stereoisomerically pure or substantially stereoisomerically pure materials. Alternatively, these compounds may be obtained by resolution/separation of mixtures of stereoisomers, including racemic and diastereomeric mixtures, using procedures known to those of ordinary skill in the art. Exemplary methods that may be useful for the resolution/separation of stereoisomeric mixtures include derivitation with stereochemically pure reagents to form diastereomeric mixtures, chromatographic separation of diastereomeric mixtures, chromatographic separation of enantiomeric mixtures using chiral stationary phases, enzymatic resolution of covalent derivatives, and crystallization/re-crystallization. Other useful methods may be found in Enantiomers, Racemates, and Resolutions, J. Jacques et al., 1981, John Wiley and Sons, New York, N.Y., the disclosure of which is incorporated herein by reference. Preferred stereoisomers of the compounds of this invention are described herein.

In one aspect of the present invention are provided compounds wherein the stereoisomeric centers (chiral carbons) have the following designated stereochemistry:

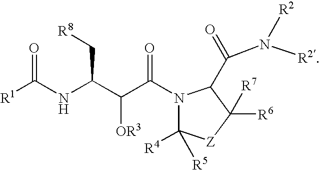

In still another aspect of the present invention are provided compounds wherein at least two of the stereoisomeric centers have the following stereochemistry:

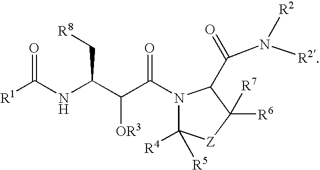

In yet another aspect of the present invention are provided compounds wherein three of the stereoisomeric centers have the following stereochemistry:

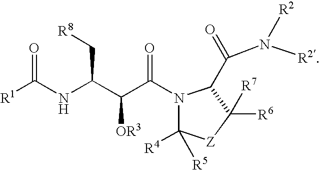

If the substituents themselves are not compatible with the synthetic methods of this invention, the substituent may be protected with a suitable protecting group that is stable to the reaction conditions used in these methods. The protecting group may be removed at a suitable point in the reaction sequence of the method to provide a desired intermediate or target compound. Suitable protecting groups and the methods for protecting and de-protecting different substituents using such suitable protecting groups are well known to those skilled in the art; examples of which may be found in T. Greene and P. Wuts, Protective Groups in Organic Synthesis (3^{rd} ed.), John Wiley & Sons, New York (1999), which is incorporated herein by reference in its entirety. In some instances, a substituent may be specifically selected to be reactive under the reaction conditions used in the methods of this invention. Under these circumstances, the reaction conditions convert the selected substituent into another substituent that is either useful in an intermediate compound in the methods of this invention or is a desired substituent in a target compound.

In the compounds of this invention, R² and R^{2′}, independently or taken together, may be a suitable nitrogen protecting group. As indicated above, suitable nitrogen protecting groups are known to those of ordinary skill in the art and any nitrogen protecting group that is useful in the methods of preparing the compounds of this invention or may be useful in the HIV protease inhibitory compounds of this invention may be used. Exemplary nitrogen protecting groups include alkyl, substituted alkyl, carbamate, urea, amide, imide, enamine, sulfenyl, sulfonyl, nitro, nitroso, oxide, phosphinyl, phosphoryl, silyl, organometallic, borinic acid and boronic acid groups. Examples of each of these groups, methods for protecting nitrogen moieties using these groups and methods for removing these groups from nitrogen moieties are disclosed in T. Greene and P. Wuts, supra. Preferably, when R² and/or R^{2′} are independently suitable nitrogen protecting groups, suitable R² and R^{2′} substituents include, but are not limited to, carbamate protecting groups such as alkyloxycarbonyl (e.g., Boc: t-butyloxycarbonyl) and aryloxycarbonyl (e.g., Cbz: benzyloxycarbonyl, or FMOC: fluorene-9-methyloxycarbonyl), alkyloxycarbonyls (e.g., methyloxycarbonyl), alkyl or arylcarbonyl, substituted alkyl, especially arylalkyl (e.g., trityl (triphenylmethyl), benzyl and substituted benzyl), and the like. When R² and R^{2′} taken together are a suitable nitrogen protecting group, suitable R²/R^{2′} substituents include phthalimido and a stabase (1,2-bis (dialkylsilyl)) ethylene).

The following processes illustrate the preparation of HIV protease inhibitors according to methods of the present invention. These compounds, prepared by the methods of the present invention, are potent inhibitors of HIV protease and thus are useful in the prevention and treatment of acquired immunodeficiency syndrome (AIDS) and AIDS related complex (“ARC”).

Unless otherwise indicated, variables according to the following processes are as defined above.

Starting materials, the synthesis of which are not specifically described herein or provided with reference to published references, are either commercially available or can be prepared using methods known to those of ordinary skill in the art. Certain synthetic modifications may be done according to methods familiar to those of ordinary skill in the art.

Compounds of formula (I), 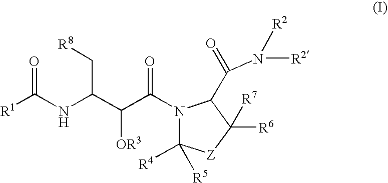
wherein R¹ is a 5- or 6-membered mono-cyclic carbocyclic or heterocyclic group, wherein said carbocyclic or heterocyclic group is saturated, partially unsaturated or fully unsaturated and is substituted by at least one hydroxyl, and Z, R², R^{2′}, R³, R⁴, R⁵, R⁶, R⁷, and R⁸ are as hereinbefore defined, may be prepared from compounds of formula (I) wherein R¹ is a 5- or 6-membered mono-cyclic carbocyclic or heterocyclic group, wherein said carbocyclic or heterocyclic group is saturated, partially unsaturated or fully unsaturated and is substituted by at least one substituent chosen from C₁₋₆ alkylcarbonyloxy, C₆₋₁₀ arylcarbonyloxy, and heteroarylcarbonyloxy. The C₁₋₆ alkylcarbonyloxy, C₆₋₁₀ arylcarbonyloxy, and heteroarylcarbonyloxy groups may be cleaved under conditions that directly provide the desired hydroxy substituted compounds of the invention. In general, the C₁₋₆ alkylcarbonyloxy, C₆₋₁₀ arylcarbonyloxy, and heteroarylcarbonyloxy groups may be cleaved under basic conditions, in a solvent that will not interfere with the desired transformation, and at a temperature that is compatible with the other reaction parameters, all of which are known to those of ordinary skill in the art. For example, appropriate bases include, but are not limited to, sodium bicarbonate, potassium bicarbonate, sodium carbonate, potassium carbonate, sodium hydroxide, potassium hydroxide, a sodium alkoxide such as sodium methoxide or sodium ethoxide, a potassium alkoxide such as potassium methoxide or potassium ethoxide, or a base formed in situ using an appropriate combination of reagents, such as a combination of a trialkyl or aryl amine in combination with an alkanol such as methanol. Or such a transformation may be accomplished using an acid that is known to those of skill in the art to be appropriate to cleave such a group without interfering with the desired transformation. Such acids include, but are not limited to, hydrogen halides such as hydrochloric acid or hydroiodic acid, an alkyl sulfonic acid such as methanesulfonic acid, an aryl sulfonic acid such as benzenesulfonic acid, nitric acid, sulfuric acid, perchloric acid, or chloric acid. Furthermore, appropriate solvents include those that are known to those of skill in the art to be compatible with the reaction conditions and include alkyl esters and aryl esters, alkyl, heterocyclic, and aryl ethers, hydrocarbons, alkyl and aryl alcohols, alkyl and aryl halogenated compounds, alkyl or aryl nitriles, alkyl and aryl ketones, and non-protic heterocyclic solvents. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, these transformations may be conducted at temperatures from −20° C. to 100° C., depending on the specific reactants and solvents and is within the skill of one of ordinary skill in the art. Further suitable reaction conditions may be found in T. Greene and P. Wuts, Protective Groups in Organic Synthesis (3^{rd} ed.), John Wiley & Sons, NY (1999).

Compounds of formula (I) wherein R³ is hydrogen and Z, R¹, R², R^{2′}, R⁴, R⁵, R⁶, R⁷, and R⁸ are as hereinbefore defined, may be prepared from compounds of formula (I) wherein R³ is a hydroxyl protecting group. The choice of a suitable hydroxy protecting group is within the knowledge of one of ordinary skill in the art. Suitable hydroxyl protecting groups that are useful in the present invention include, but are not limited to, alkyl or aryl esters, alkyl silanes, aryl silanes or alkylaryl silanes, alkyl or aryl carbonates, benzyl groups, substituted benzyl groups, ethers, or substituted ethers. The various hydroxy protecting groups can be suitably cleaved utilizing a number of reaction conditions known to those of ordinary skill in the art. The particular conditions used will depend on the particular protecting group as well as the other functional groups contained in the subject compound. Choice of suitable conditions is within the knowledge of those of ordinary skill in the art.

For example, if the hydroxy protecting group is an alkyl or aryl ester, cleavage of the protecting group may be accomplished using a suitable base, such as a carbonate, a bicarbonate, a hydroxide, an alkoxide, or a base formed in situ from an appropriate combination of agents. Furthermore, such reactions may be performed in a solvent that is compatible with the reaction conditions and will not interfere with the desired transformation. For example, suitable solvents may include alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the knowledge of one of ordinary skill in the art. Further suitable reaction conditions may be found in T. Greene and P. Wuts, Protective Groups in Organic Synthesis (3^{rd} ed.), John Wiley & Sons, NY (1999).

Additionally, if R³ is an alkyl silane, aryl silane or alkylaryl silane, such groups may be cleaved under conditions known to those of ordinary skill in the art. For example, such silane protecting groups may be cleaved by exposure of the subject compound to a source of fluoride ions, such as the use of an organic fluoride salt such as a tetraalkylammonium fluoride salt, or an inorganic fluoride salt. Suitable fluoride ion sources include, but are not limited to, tetramethylammonium fluoride, tetraethylammonium fluoride, tetrapropylammonium fluoride, tetrabutylammonium fluoride, sodium fluoride, and potassium fluoride. Alternatively, such silane protecting groups may be cleaved under acidic conditions using organic or mineral acids, with or without the use of a buffering agent. For example, suitable acids include, but are not limited to, hydrofluoric acid, hydrochloric acid, sulfuric acid, nitric acid, acetic acid, citric acid, and methanesulfonic acid. Such silane protecting groups may also be cleaved using appropriate Lewis acids. For example, suitable Lewis acids include, but are not limited to, dimethylbromo borane, triphenylmethyl tetrafluoroborate, and certain Pd (II) salts. Such silane protecting groups can also be cleaved under basic conditions that employ appropriate organic or inorganic basic compounds. For example, such basic compounds include, but are not limited to, sodium carbonate, potassium carbonate, sodium bicarbonate, potassium bicarbonate, sodium hydroxide, and potassium hydroxide. The cleavage of a silane protecting group may be conducted in an appropriate solvent that is compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the knowledge of one of ordinary skill in the art. Further suitable reaction conditions may be found in T. Greene and P. Wuts, Protective Groups in Organic Synthesis (3^{rd} ed.), John Wiley & Sons, NY (1999).

When R³ is a benzyl or substituted benzyl ether, cleavage of the protecting group may be accomplished by treating the subject compound with hydrogen in the presence of a suitable catalyst, oxidation with suitable compounds, exposure to light of particular wavelengths, electrolysis, treatment with protic acids, or treatment with Lewis acids. The choice of particular reagents to effect such a transformation will depend on the specific subject compound used and is within the skill of one of ordinary skill in the art. For example, such benzyl or substituted benzyl ethers may be cleaved using hydrogen gas in the presence of an appropriate catalyst. Suitable catalysts include, but are not limited to, 5% palladium on carbon, 10% palladium on carbon, 5% platinum on carbon, or 10% platinum on carbon. The choice of a particular catalyst and the amounts of catalyst, the amount of hydrogen gas, and the hydrogen gas pressure used to effect the desired transformation will depend upon the specific subject compound and the particular reaction conditions utilized. Such choices are within the skill of one of ordinary skill in the art. Furthermore, such benzyl and substituted benzyl ethers may be cleaved under oxidative conditions in which a suitable amount of an oxidizer is used. Such suitable oxidizers include, but are not limited to, dichlorodicyanoquinone (DDQ), ceric ammonium nitrate (CAN), ruthenium oxide in combination with sodium periodate, iron (III) chloride, or ozone. Additionally, such ethers may be cleaved using an appropriate Lewis acid. Such suitable Lewis acids include, but are not limited to, dimethylbromo borane, triphenylmethyl tetrafluoroborate, sodium iodide in combination with trifluoroborane-etherate, trichloroborane, or tin (IV) chloride. The cleavage of a benzyl or substituted benzyl ether protecting group may be conducted in an appropriate solvent that is compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the knowledge of one of ordinary skill in the art. Further suitable reaction conditions may be found in T. Greene and P. Wuts, Protective Groups in Organic Synthesis (3^{rd} ed.), John Wiley & Sons, NY (1999).

When R³ is methyl, cleavage of the protecting group may be accomplished by treating the subject compound with organic or inorganic acids or Lewis acids. The choice of a particular reagent will depend upon the type of methyl ether present as well as the other reaction conditions. The choice of a suitable reagent for cleaving a methyl ether is within the knowledge of one of ordinary skill in the art. Examples of suitable reagents include, but are not limited to, hydrochloric acid, sulfuric acid, nitric acid, para-toluenesulfonic acid, or Lewis acids such as boron trifluoride etherate. These reactions may be conducted in solvents that are compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the skill of one of ordinary skill in the art. Further suitable reaction conditions may be found in T. Greene and P. Wuts, Protective Groups in Organic Synthesis (3^{rd} ed.), John Wiley & Sons, NY (1999).

When R³ is a carbonate, cleavage of the protecting group may be accomplished by treating the subject compound with suitable basic compounds. Such suitable basic compounds may include, but are not limited to, sodium carbonate, sodium bicarbonate, potassium carbonate, potassium bicarbonate, sodium hydroxide, or potassium hydroxide. The choice of a particular reagent will depend upon the type of carbonate present as well as the other reaction conditions. These reactions may be conducted in solvents that are compatible with the specific reaction conditions chosen and will not interfere with the desired transformation. Among such suitable solvents are, for example, alkyl esters, alkylaryl esters, aryl esters, alkyl ethers, aryl ethers, alkylaryl esters, cyclic ethers, hydrocarbons, alcohols, halogenated solvents, alkyl nitriles, aryl nitriles, alkyl ketones, aryl ketones, alkylaryl ketones, or non-protic heterocyclic compounds. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Finally, such reactions may be performed at an appropriate temperature from −20° C. to 100° C., depending on the specific reactants used. The choice of a suitable temperature is within the knowledge of one of ordinary skill in the art. Further suitable reaction conditions may be found in T. Greene and P. Wuts, Protective Groups in Organic Synthesis (3^{rd} ed.), John Wiley & Sons, NY (1999).

Furthermore, compounds of formula (I) wherein R¹ is phenyl substituted by at least one group selected from hydroxy, and R³ is hydrogen, may be prepared from compounds of formula I wherein R¹ is phenyl optionally substituted by at least one substituent independently chosen from C₁₋₆ alkylcarbonyloxy, C₆₋₁₀ arylcarbonyloxy, and heteroarylcarbonyloxy; and R³ is a hydroxyl protecting group. In these compounds, the R¹ C₁₋₆ alkylcarbonyloxy, C₆₋₁₀ arylcarbonyloxy, and heteroarylcarbonyloxy group and the R³ hydroxyl protecting group may be removed using reactions conditions in which both groups are removed concomitantly or they may be removed in step-wise fashion. For example, when R¹ is phenyl substituted by alkylcarbonyloxy and R³ is an alkyl ester, both groups may be cleaved by reacting the subject compound with a base in an appropriate solvent and at an appropriate temperature. The choice of a suitable base, solvent, and temperature will depend on the particular subject compound and the particular protecting groups being utilized. These choices are within the skill of one of ordinary skill in the art.

Alternatively, in compounds of formula (I) wherein R¹ is phenyl substituted by at least one group selected from C₁₋₆ alkylcarbonyloxy, C₆₋₁₀ arylcarbonyloxy, and heteroarylcarbonyloxy, and R³ is a hydroxyl protecting group, the C₁₋₆ alkylcarbonyloxy, C₆₋₁₀ arylcarbonyloxy, and heteroarylcarbonyloxy group and the R³ hydroxyl protecting group may be cleaved in a stepwise manner to afford a compound of formula I wherein R¹ is phenyl substituted by hydroxy and R³ is hydrogen. The choice of the R³ hydroxyl protecting group and the conditions to affect its cleavage will depend upon the specific subject compound chosen and is within the knowledge of one of ordinary skill in the art. For example, in the compounds of formula (I) wherein R¹ is phenyl substituted by C₁₋₆ alkylcarbonyloxy and R³ is a silane protecting group, the R³ silane protecting group may be cleaved first by treatment of the subject compound with a fluoride source such as tetrabutylammonium fluoride in acetonitrile at room temperature, followed by cleavage of the C₁₋₆ alkylcarbonyloxy group in R¹ by treatment with a base such as potassium hydroxide in a mixture of methanol and acetonitrile at room temperature.

Compounds of formula (I) wherein Z, R¹, R², R^{2′}, R³, R⁴, R⁵, R⁶, R⁷, and R⁸ are as hereinbefore defined may be prepared by reacting a compound of formula (II), wherein Y¹ is a leaving group and R¹ and R³ are as hereinbefore defined, 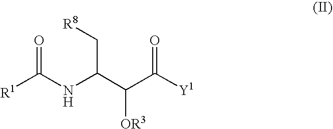
with a compound of formula (III), 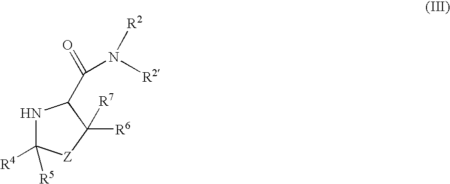
wherein Z, R², R^{2′}, R⁴, R⁵, R⁶, R⁷, and R⁸ are as hereinbefore defined, or a salt or solvate thereof, to afford a compound of formula (I).

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenztriazole (HOBt), hydroxyazabenzotriazole (HOAt), N-hydroxysuccinimide (HOSu), N-hydroxy-5-norbornene-endo-2,3-dicarboximide (HONB), 4-dimethylaminopyridine (DMAP). Whether these additives are necessary depends on the identity of the reactants, the solvent, and the temperature, and such a choice is within the knowledge of one of ordinary skill in the art.

In general, the leaving group Y¹ in the compounds of formula (II) should be such that it provides sufficient reactivity of the compounds of formula (II) with the compounds of formula (III). Compounds of formula (II) that contain such suitable leaving groups may be prepared, isolated and/or purified, and subsequently reacted with the compounds of formula (III). Alternatively, compounds of formula (II) with suitable leaving groups may be prepared and further reacted without isolation or further purification with the compounds of formula (III) to afford compounds of formula (I). Among suitable leaving groups, Y¹, are halides, aromatic heterocycles, sulfonic acid esters, anhydrides, or groups derived from the reaction of compounds of formula (II) wherein Y¹ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, —OPO(Oalkyl)₂, and those derived from the reaction of the compounds of formula (II) wherein Y¹ is —OH with carbodiimides. Other suitable leaving groups are known to those of ordinary skill in the art and may be found, for example, in Humphrey, J. M.; Chamberlin, A. R. Chem. Rev. 1997, 97, 2243; Comprehensive Organic Synthesis; Trost, B. M., Ed.; Pergamon: New York, 1991; Vol. 6, pp 301-434; and Comprehensive Organic Transformations; Larock, R. C.; VCH: New York, 1989, Chapter 9.

Compounds of formula (II) where in Y¹ is a halogen can be prepared from compounds of formula (II) wherein Y¹ is hydroxy by reaction with a suitable agent. For example, the compounds of formula (II) wherein Y¹ is chloro may be prepared from compounds of formula (II) wherein Y¹ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art. The present invention specifically contemplates that the compounds of formula (I-H) may be prepared by reacting compounds of formula (III) with compounds of formula (II), wherein R³ is hydrogen, an optionally substituted C₁₋₄ alkyl group, or a suitable protecting group, such as a C₁₋₆ alkylcarbonyl, C₆₋₁₀ arylcarbonyl, or heteroarylcarbonyl group.

Whether R³ in the compounds of formula (II) is hydrogen, an optionally substituted C₁₋₄ alkyl group, or a suitable protecting group is dependent on the specific product compounds desired and/or the specific reaction conditions used. Such choices are within the knowledge of one of ordinary skill in the art.

Compounds of formula (II) where in Y¹ is an aromatic heterocycle can be prepared from compounds of formula (II) wherein Y¹ is hydroxy by reaction with a suitable agent such as carbonyl diimidazole. These compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from <20° C. to 100° C. The specific reaction conditions chosen will depend on the specific subject compound and reagents chosen. Such knowledge is within the skill of one of ordinary skill in the art.

Compounds of formula (II) wherein Y¹ is —OC(O)alkyl or —OC(O)aryl may be prepared from compounds of formula (II) wherein Y¹ is hydroxy by reaction with suitable reagents such acyl halides, acyl imidazoles, or carboxylic acid under dehydrating conditions. Suitable reagents may include, but are not limited to, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (II) wherein Y¹ is —OC(O)Oalkyl, —OC(O)Oaryl can be prepared from compounds of formula (II) wherein Y¹ is hydroxy by reaction with a suitable agents such as chloroformates of the formula ClC(O)Oalkyl or ClC(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (II) wherein Y¹ is —OS(O₂)alkyl or —OS(O₂)aryl can be prepared from compounds of formula (II) wherein Y¹ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula (I) may be prepared by reaction of compounds of formula (II), wherein Y¹ is —OH, with compounds of formula (III) under dehydrating conditions, utilizing agents such as carbodiimides or carbodiimide derived species. Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N,N,N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenztriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature, and such choices are within the knowledge of one of ordinary skill in the art.

Compounds of formula (II), wherein R³ is a suitable protecting group and Y¹ and R¹ are as hereinbefore defined, may be prepared from compounds of formula (II) wherein R³ is hydrogen. The choice of a suitable protecting group is dependent upon the subject compound chosen and subsequent reaction conditions to which the compound of formula (II) will be subjected. Generally, R³ in the compounds of formula II can be chosen from alkyl or aryl esters, alkyl silanes, aryl silanes, alkylaryl silanes, carbonates, optionally substituted benzyl ethers, or other substituted ethers. Such protecting groups can be introduced into the compounds of formula (II) wherein R³ is hydrogen using methods known to those of ordinary skill in the art and as found in, for example, T. Greene and P. Wuts, Protective Groups in Organic Synthesis (3^{rd} ed.), John Wiley & Sons, NY (1999). For example, as shown below, compound (2) was allowed to react with acetic anhydride in ethyl acetate and methanesulfonic acid at about 70° C. to afford compound (5).

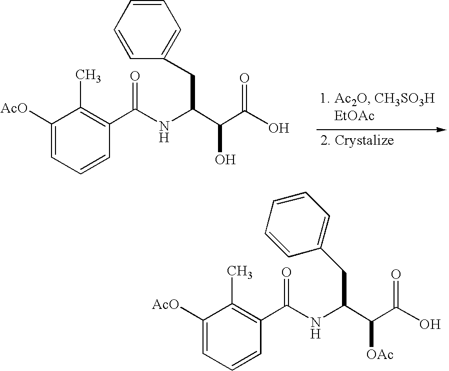

Compounds of formula (II), wherein Y¹ is hydroxy and R¹ and R³ are as hereinbefore defined, can be prepared by reaction of compounds of formula (IV), wherein Y¹ and R³ are as hereinbefore defined, with compounds of formula (V), wherein R¹ is as hereinbefore defined and Y² is hydroxy or a suitable leaving group, as shown below.

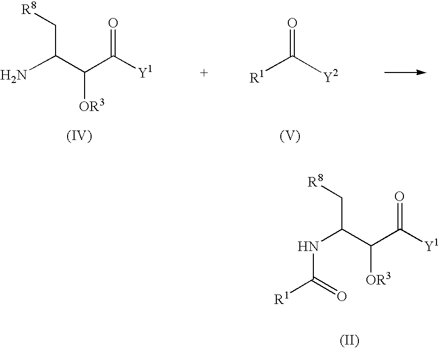

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenztriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

In general, the leaving group Y² in the compounds of formula (V) should be such that it provides sufficient reactivity with the amine in the compounds of formula (IV). Compounds of formula (V) that contain such suitable leaving groups may be prepared, isolated and/or purified, and subsequently reacted with the compounds of formula (IV). Alternatively, compounds of formula (V) with suitable leaving groups may be prepared and further reacted without isolation or further purification with the compounds of formula (IV) to afford compounds of formula (II). Among suitable leaving groups in the compounds of formula (V) are halides, aromatic heterocycles, sulfonic acid esters, anhydrides, or groups derived from the reaction of compounds of formula (V) wherein Y² is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, OPO(Oalkyl)₂, and those derived from the reaction of the compounds of formula V wherein Y² is —OH with carbodiimides. Other suitable leaving groups are known to those of ordinary skill in the art and may be found, for example, in Humphrey, J. M.; Chamberlin, A. R., Chem. Rev., 1997, 97, 2243; Comprehensive Organic Synthesis; Trost, B. M., Ed.; Pergamon: New York, 1991; Vol. 6, pp 301-434; and Comprehensive Organic Transformations; Larock, R. C.; VCH: New York, 1989, Chapter 9.

Compounds of formula (V) where in Y² is a halogen can be prepared from compounds of formula (V) wherein Y² is hydroxy by reaction with a suitable agent. For example, the compounds of formula (V) wherein Y² is chloro may be prepared from compounds of formula (V) wherein Y² is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IV) or they may be formed in situ and reacted with the compounds of formula (IV) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art. For example, as shown below, compound (7) was allowed to react with compound (8) in a mixture of tetrahydrofuran and water, in the presence of triethylamine, at room temperature to afford the desired compound (5).

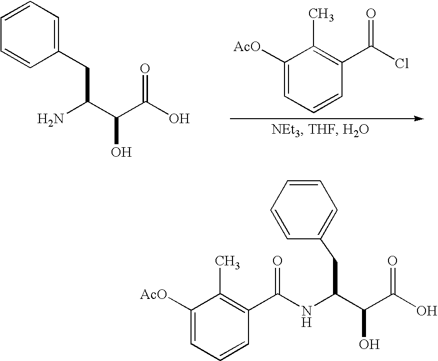

Compounds of formula (IV), wherein Y¹ is hydroxy and R³ is as defined above, are either commercially available or can be prepared by methods known to those of skill in the art.

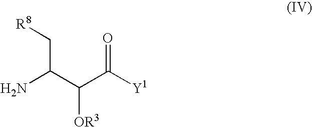

For example, the compounds of formula (IV) can be prepared as shown in the scheme below. In general, an N-protected amino acid derivative is reduced to an aldehyde using reducing agents that are suitable for such a transformation. For example, suitable reducing agents are dialkyl aluminum hydride agents, such as diisobutyl aluminum hydride for example. Another method of preparing the compounds of formula (IV) is to reduce an appropriate carboxylic acid to an alcohol with a suitable reducing agent such as LiAlH₄ or BH₃ or NaBH₄ for example, followed by oxidation of the alcohol to the corresponding aldehyde with PCC, under Swern conditions or using pyr.SO₃/DMSO/NEt₃ for example Another method of preparing the compounds of formula (IV) is to reduce an appropriate carboxylic acid derivative, such as a Weinreb amide or an acyl imidazole, using a suitable reducing agent such as LiAlH₄ or diisobutyl aluminum hydride for example. Alternatively, the compounds of formula (IV) can be prepared by the preparation of an appropriate aldehyde by reduction of the corresponding acid chloride. Next, a compound is added to the aldehyde that is the equivalent of adding a carboxylate CO₂ anion. For example, cyanide can be added to the aldehyde to afford a cyanohydrin that can then be hydrolyzed under either acidic or basic conditions to afford the desired compound, (d). Alternatively, nitromethane may be added to the aldehyde under basic conditions to afford an intermediate that is then converted into the desired compound. These compounds can be prepared according to the following procedures. In those compounds where Y³ is —CN, R. Pedrosa, et al., Tetrahedron Asymm. 2001, 12, 347. For those compounds in which Y³ is —CH₂NO₂, M. Shibasaki, et al., Tetrahedron Lett., 1994, 35, 6123.

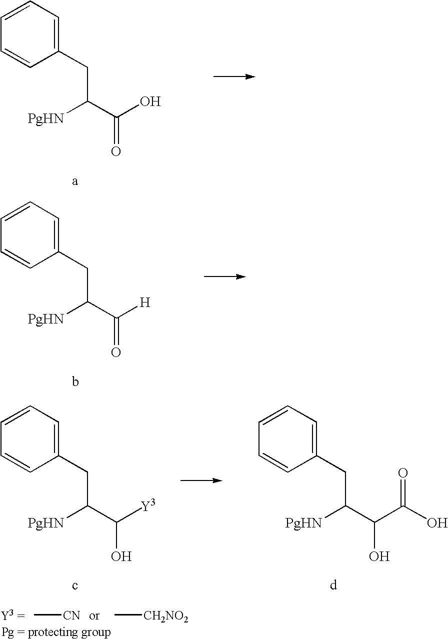

Compounds of formula (V), wherein Y² is hydroxy and R¹ is as hereinbefore defined, are either commercially available or can be prepared by methods known to those of skill in the art. For example, such compounds can be prepared from the corresponding alcohols by oxidation with suitable reagents. Such oxidation agents include, but are not limited to, KMnO₄, pyridinium dichromate (PDC), H₂Cr₂O₇ (Jones's reagent), and 2,2,6,6-tetramethylpiperidinyl-2-oxyl (TEMPO)NaClO₂.

Compounds of formula (III), wherein Z is S, O, SO, SO₂, CH₂, CFH, or CF₂, and R², R^{2′}, R⁴, R⁵, R⁶, and R⁷ are as hereinbefore defined, are either commercially available or can be prepared according to methods known to those of skill in the art. For example, see Mimoto, T., et al., J. Med. Chem., 1999, 42, 1789; EP 0751145; and U.S. Pat. Nos. 5,644,028, 5,932,550, 5,962,640, and 6,222,043, which are hereby incorporated by reference.

In addition, the compounds of formula (III), wherein Z is CF₂, R⁴ and R⁵ are hydrogen, R⁶, and R⁷ are methyl, and R² and R^{2′} are as hereinbefore defined, can be prepared according to the scheme below. The racemic material can be resolved according to methods known to those skilled in the art to provide compounds of formula (III) with an enantiomeric excess in the range of from 95% to 100%

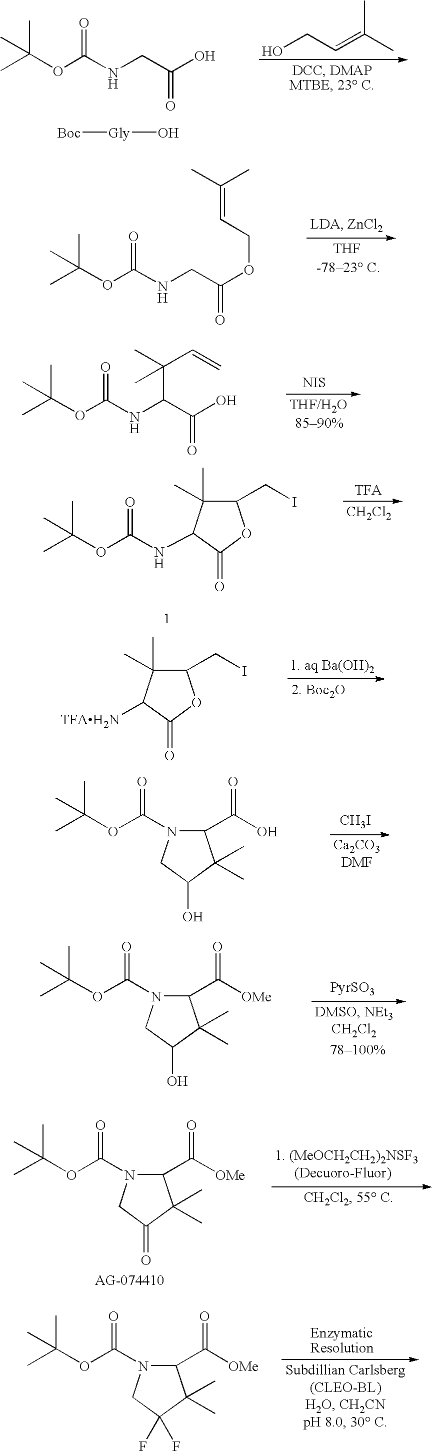

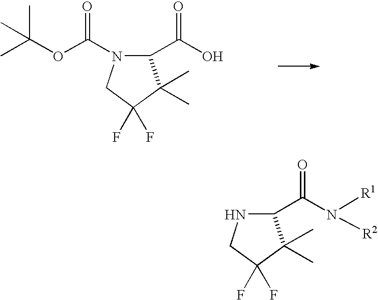

Alternatively, the compounds of formula (I), wherein R¹ is phenyl optionally substituted by at least one substituent independently chosen from C₁₋₆ alkyl, hydroxyl, C₁₋₆ alkylcarbonyloxy, C₆₋₁₀ arylcarbonyloxy, and heteroarylcarbonyloxy, and Z, R², R^{2′}, R³, R⁴, R⁵, R⁶, R⁷, and R⁸ are as hereinbefore defined, may be prepared by reaction of compounds of formula (VI), 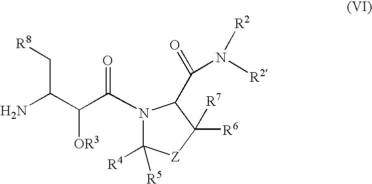
wherein Z, R², R^{2′}, R³, R⁴, R⁵, R⁶, R⁷, and R⁸ are as hereinbefore defined, with compounds of formula (V), wherein R¹ and Y² are as hereinbefore defined.

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used. Such reactions may also be promoted by the addition of optional additives. Examples of such additives include, but are not limited to, hydroxybenztriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

In general, the leaving group Y² in the compounds of formula (V) should be such that it provides sufficient reactivity with the amino group in the compounds of formula (VI). Compounds of formula (V) that contain such suitable leaving groups may be prepared, isolated and/or purified, and subsequently reacted with the compounds of formula (VI). Alternatively, compounds of formula (V) with suitable leaving groups may be prepared and further reacted without isolation or further purification with the compounds of formula (VI) to afford compounds of formula (I). Among suitable leaving groups in the compounds of formula (V) are halides, aromatic heterocycles, sulfonic acid esters, anhydrides, or groups derived from the reaction of compounds of formula (V) wherein Y² is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, —OPO(Oalkyl)₂, and those derived from the reaction of the compounds of formula (V), wherein Y² is —OH, with carbodiimides.

Compounds of formula (V) where in Y² is a halogen can be prepared from compounds of formula (V) wherein Y² is hydroxy by reaction with a suitable agent. For example, the compounds of formula (V) wherein Y² is chloro may be prepared from compounds of formula (V) wherein Y² is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (VI) or they may be formed in situ and reacted with the compounds of formula (VI) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VI), 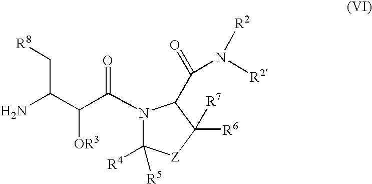
wherein Z, R², R^{2′}, R³, R⁴, R⁵, R⁶, R⁷, and R⁸ are as hereinbefore defined, may be prepared from reaction of compounds of formula (VII), 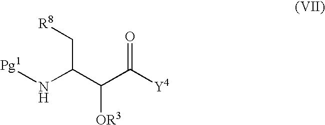
wherein Pg¹ is a suitable nitrogen protecting group, Y⁴ is hydroxy or a suitable leaving group, and R³ is as hereinbefore defined, with a compound of formula (III), wherein Z, R², R^{2′}, R⁴, R⁵, R⁶, R⁷, and R⁸ are as hereinbefore defined, or a salt or solvate thereof.

A suitable protecting group Pg¹ in the compounds of formula (VII) is one that is stable to subsequent reaction conditions in which the compounds of formula (VII) are allowed to react with the compounds of formula (III). Furthermore, such a protecting group should be chosen such that it can be removed after the compounds of formula (VII) have been allowed to react with the compounds of formula (III) to afford an intermediate compound that is subsequently deprotected to afford a compound of formula (VI). Suitable protecting groups include, but are not limited to, carbamates such as t-butyloxycarbonyl and benzyloxycarbonyl, imides such as phthaloyl, or suitable benzyl groups. Such protecting groups can be introduced into the compounds of formula (VII) and subsequently removed to provide compounds of formula (VI) according to methods known to those of ordinary skill in the art and as found in, for example, T. Greene and P. Wuts, Protective Groups in Organic Synthesis (3^{rd} ed.), John Wiley & Sons, NY (1999).

In general, the leaving group Y⁴ in the compounds of formula (VII) should be such that it provides sufficient reactivity with the amino group in the compounds of formula (III). Compounds of formula (VII) that contain such suitable leaving groups may be prepared, isolated and/or purified, and subsequently reacted with the compounds of formula (III). Alternatively, compounds of formula (VII) with suitable leaving groups may be prepared and further reacted without isolation or further purification with the compounds of formula (III) to afford compounds of formula (VI). Among suitable leaving groups in the compounds of formula (VII) are halides, aromatic heterocycles, sulfonic acid esters, anhydrides, or groups derived from the reaction of compounds of formula (VII) wherein Y⁴ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, —OPO(Oaryl)₂, —OPO(Oalkyl)₂, and those derived from the reaction of the compounds of formula (VII), wherein Y⁴ is —OH, with carbodiimides.

Compounds of formula (VII) where in Y⁴ is a halogen can be prepared from compounds of formula (VII) wherein Y⁴ is hydroxy by reaction with a suitable agent. For example, the compounds of formula (VII) wherein Y⁴ is chloro may be prepared from compounds of formula (VII) wherein Y⁴ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) where in Y⁴ is an aromatic heterocycle can be prepared from compounds of formula (VII) wherein Y⁴ is hydroxy by reaction with a suitable agent such as carbonyl diimidazole. These compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the skill of one of ordinary skill in the art.

Compounds of formula (VII) wherein Y⁴ is —OC(O)alkyl or —OC(O)aryl may be prepared from compounds of formula (VII) wherein Y⁴ is hydroxy by reaction with suitable reagents such acyl halides, acyl imidazoles, or carboxylic acid under dehydrating conditions. Suitable reagents may include, but are not limited to, pivaloyl chloride, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) wherein Y⁴ is —OC(O)Oalkyl, —OC(O)Oaryl can be prepared from compounds of formula (VII) wherein Y⁴ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VII) wherein Y⁴ is —OS(O₂)alkyl or —OS(O₂)aryl can be prepared from compounds of formula (VII) wherein Y⁴ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (III) or they may be formed in situ and reacted with the compounds of formula (III) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula (VI) may be prepared by reaction of compounds of formula (VII), wherein Y⁴ is —OH, with compounds of formula (III) under dehydrating conditions using agents such as carbodiimides or carbodiimide derived species. Suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N, N, N′, N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenztriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

Alternatively, the compounds of formula (I) may be prepared by reaction of a compound of formula (VII), 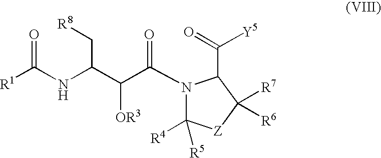
wherein Y⁵ is hydroxy or a suitable leaving group, and Z, R¹, R³, R⁴, R⁵, R⁶, R⁷and R⁸ are as hereinbefore defined, with a compound of formula (IX), 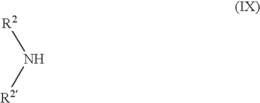
wherein R² and R^{2′} are hereinbefore defined, or a salt or solvate thereof.

In general, the leaving group Y⁵ in the compounds of formula (VIII) should be such that it provides sufficient reactivity with the amino group in the compounds of formula (IX). Compounds of formula (VIII) that contain such suitable leaving groups may be prepared, isolated and/or purified, and subsequently reacted with the compounds of formula (IX). Alternatively, compounds of formula (VIII) with suitable leaving groups may be prepared and further reacted without isolation or further purification with the compounds of formula (IX) to afford compounds of formula (I). Among suitable leaving groups in the compounds of formula (VIII) are halides, aromatic heterocycles, sulfonic acid esters, anhydrides, or groups derived from the reaction of compounds of formula (VIII) wherein Y⁵ is hydroxy with reagents such as carbodiimides or carbodiimide species. Examples of suitable leaving groups include, but are not limited to, chloride, iodide, imidazole, —OC(O)alkyl, —OC(O)aryl, —OC(O)Oalkyl, —OC(O)Oaryl, —OS(O₂)alkyl, —OS(O₂)aryl, and those derived from the reaction of the compounds of formula (VIII), wherein Y⁵ is —OH, with carbodiimides.

Compounds of formula (VIII) where in Y⁵ is a halogen can be prepared from compounds of formula (VIII) wherein Y⁵ is hydroxy by reaction with a suitable agent. For example, the compounds of formula (VIII) wherein Y⁵ is chloro may be prepared from compounds of formula (VIII) wherein Y⁵ is hydroxy by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) where in Y⁵ is an aromatic heterocycle can be prepared from compounds of formula (VIII) wherein Y⁵ is hydroxy by reaction with a suitable agent such as carbonyl diimidazole. These compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OC(O)alkyl or —OC(O)aryl may be prepared from compounds of formula (VIII) wherein Y⁵ is hydroxy by reaction with suitable reagents such acyl halides, acyl imidazoles, or carboxylic acid under dehydrating conditions. Suitable reagents may include, but are not limited to, pivaloyl chloride, acetyl chloride, acetyl iodide formed in situ from acetyl chloride and sodium iodide, acetyl imidazole, or acetic acid under dehydrating conditions. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OC(O)Oalkyl, —OC(O)Oaryl can be prepared from compounds of formula (VIII) wherein Y⁵ is hydroxy by reaction with a suitable agents such as chloroformates of the formula Cl—C(O)Oalkyl or Cl—C(O)Oaryl. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Compounds of formula (VIII) wherein Y⁵ is —OS(O₂)alkyl or —OS(O₂)aryl can be prepared from compounds of formula (VIII) wherein Y⁵ is hydroxy by reaction with a suitable agent such as an alkyl or aryl sulfonyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with the compounds of formula (IX) or they may be formed in situ and reacted with the compounds of formula (IX) without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

Alternatively, compounds of formula I may be prepared by reaction of compounds of formula (VIII), wherein Y⁵ is —OH, with compounds of formula (IX) under dehydrating conditions using agents such as carbodiimides or carbodiimide derived species Such suitable agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N,N,N′,N′-tetramethyluronium hexafluorophosphate (HATU), carbonyldiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrefluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenztriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature. Such choices are within the knowledge of one of ordinary skill in the art.

Compounds of formula (IX) are either commercially available or can be prepared by methods described herein or methods known to those of ordinary skill in the art.

Furthermore, the present invention relates to the treatment of mammals infected with HIV, suffering from acquired immunodeficiency syndrome (AIDS), AIDS-related complex (ARC), or other HIV- or AIDS-related diseases. The methods of the present invention comprise administering to a mammal an HIV-inhibiting amount of a compound of the present invention, or a pharmaceutically acceptable salt, prodrug, pharmaceutically active metabolite, or solvate thereof, in a pharmaceutically acceptable formulation, either alone or in combination with an effective amount of an additional agent, to treat said mammal suffering from infection with the HIV virus. The additional agent may be administered separately or may be administered as part of the same formulation as the compounds of the present invention. When administered separately, the compounds of the present invention and those of the additional agent may be administered sequentially or with a period of time in between administration.

The activity of the compounds as inhibitors of HIV activity may be measured by any of the suitable methods available in the art, including in vivo and in vitro assays. An Example of a suitable assay for activity measurements is the HIV assay described herein.

Administration of the compounds and their pharmaceutically acceptable prodrugs, salts, active metabolites, and solvates may be performed according to any of the accepted modes of administration available to those skilled in the art. Illustrative Examples of suitable modes of administration include oral, nasal, parenteral, topical, transdermal, and rectal. Oral and intravenous deliveries are preferred.

An HIV-inhibiting agent may be administered as a pharmaceutical composition in any suitable pharmaceutical form. Suitable pharmaceutical forms include solid, semisolid, liquid, or lyopholized formulations, such as tablets, powders, capsules, suppositories, suspensions, liposomes, and aerosols. The HIV-inhibiting agent may be prepared as a solution using any of a variety of methodologies. For Example, the HIV-inhibiting agent can be dissolved with acid (e.g., 1 M HCl) and diluted with a sufficient volume of a solution of 5% dextrose in water (D5W) to yield the desired final concentration of HIV-inhibiting agent (e.g., about 15 mM). Alternatively, a solution of D5W containing about 15 mM HCl can be used to provide a solution of the HIV-inhibiting agent at the appropriate concentration. Further, the HIV-inhibiting agent can be prepared as a suspension using, for example, a 1% solution of carboxymethylcellulose (CMC).

Acceptable methods of preparing suitable pharmaceutical forms of the pharmaceutical compositions are known or may be routinely determined by those skilled in the art. For Example, pharmaceutical preparations may be prepared following conventional techniques of the pharmaceutical chemist involving steps such as mixing, granulating, and compressing when necessary for tablet forms, or mixing, filling, and dissolving the ingredients as appropriate, to give the desired products for oral, parenteral, topical, intravaginal, intranasal, intrabronchial, intraocular, intraaural, and/or rectal administration.

Pharmaceutical compositions of the invention may also include suitable excipients, diluents, vehicles, and carriers, as well as other pharmaceutically active agents, depending upon the intended use. Solid or liquids, diluents, vehicles, or excipients may be employed in the pharmaceutical compositions. Illustrative solid carriers include starch, lactose, calcium sulfate dihydrate, terra alba, sucrose, talc, gelatin, pectin, acacia, magnesium stearate, and stearic acid. Illustrative liquid carriers include syrup, peanut oil, olive oil, saline solution, and water. The carrier or diluent may include a suitable prolonged-release material, such as glyceryl monostearate or glyceryl distearate, alone or with a wax. When a liquid carrier is used, the preparation may be in the form of a syrup, elixir, emulsion, soft gelatin capsule, sterile injectable liquid (e.g., solution), or a nonaqueous or aqueous liquid suspension.

A dose of the pharmaceutical composition may contain at least a therapeutically effective amount, or an HIV-inhibiting amount, of an HIV-inhibiting agent and preferably is made up of one or more pharmaceutical dosage units. The selected dose may be administered to a mammal, for example, a human patient, in need of treatment mediated by inhibition of HIV activity, by any known or suitable method of administering the dose, including topically, for example, as an ointment or cream; orally; rectally, for example, as a suppository; parenterally by injection; intravenously; or continuously by intravaginal, intranasal, intrabronchial, intraaural, or intraocular infusion. When the composition is administered in conjunction with a cytotoxic drug, the composition can be administered before, with, and/or after introduction of the cytotoxic drug. However, when the composition is administered in conjunction with radiotherapy, the composition is preferably introduced before radiotherapy is commenced.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15^{th} Edition (1975).

It will be appreciated that the actual dosages of the HIV-inhibiting agents used in the pharmaceutical compositions of this invention will be selected according to the properties of the particular agent being used, the particular composition formulated, the mode of administration and the particular site, and the host and condition being treated. Optimal dosages for a given set of conditions can be ascertained by those skilled in the art using conventional dosage-determination tests. For oral administration, e.g., a dose that may be employed is from about 0.001 to about 1000 mg/kg body weight, preferably from about 0.1 to about 100 mg/kg body weight, and even more preferably from about 1 to about 50 mg/kg body weight, with courses of treatment repeated at appropriate intervals. The dosage forms of the pharmaceutical formulations described herein may contain an amount of a compound of the present invention, or a pharmaceutically acceptable salt of solvate thereof, deemed appropriate by one of ordinary skill in the art. For example, such dosage forms may contain from about 1 mg to about 1500 mg of a compound of the present invention, or may contain from about 5 mg to about 1500 mg, or from about 5 mg to about 1250 mg, or from about 10 mg to about 1250 mg, or from about 25 mg to about 1250 mg, or from about 25 mg to about 1000 mg, or from about 50 mg to about 1000 mg, or from about 50 mg to about 750 mg, or from about 75 mg to about 750 mg, or from about 100 mg to about 750 mg, or from about 125 mg to about 750 mg, or from about 150 mg to about 750 mg, or from about 150 mg to about 500 mg of a compound of the present invention, or a pharmaceutically acceptable salt or solvate thereof.

Certain compounds may have asymmetric centers and therefore exist in different enantiomeric forms. All optical isomers and stereoisomers of the compounds, and mixtures thereof, are considered to be within the scope of the invention. With respect to the compounds herein described, the invention includes the use of a racemate, one or more enantiomeric forms, one or more diastereomeric forms, or mixtures thereof. The compounds may also exist as tautomers. This invention relates to the use of all such tautomers and mixtures thereof.

The subject invention also includes isotopically-labelled compounds, which are identical to those recited herein, but for the fact that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of this invention and prodrugs thereof can generally be prepared by carrying out the procedures disclosed in the Schemes and/or in the Examples and Preparations below, by substituting a readily available isotopically labelled reagent for a non-isotopically labelled reagent.

The compounds of the present invention may be administered in combination with an additional agent or agents for the treatment of a mammal, such as a human, that is suffering from an infection with the HIV virus, AIDS, AIDS-related complex (ARC), or any other disease or condition which is related to infection with the HIV virus. The agents that may be used in combination with the compounds of the present invention include, but are not limited to, those useful as HIV protease inhibitors, HIV reverse transcriptase inhibitors, non-nucleoside HIV reverse transcriptase inhibitors, inhibitors of HIV integrase, CCR5 inhibitors, HIV fusion inhibitors, compounds useful as immunomodulators, compounds that inhibit the HIV virus by an unknown mechanism, compounds useful for the treatment of herpes viruses, compounds useful as anti-infectives, and others as described below.

Compounds useful as HIV protease inhibitors that may be used in combination with the compounds of the present invention include, but are not limited to, 141 W94 (amprenavir), CGP-73547, CGP-61755, DMP-450, nelfinavir, ritonavir, saquinavir (invirase), lopinavir, TMC-126, BMS-232632 (atazanavir), palinavir, GS-3333, KN l413, KNl-272, LG-71350, CGP-61755, PD 173606, PD 177298, PD 178390, PD 178392, U-140690, ABT-378, DMP-450, AG-1776, MK-944, VX-478, indinavir, tipranavir, TMC-114, DPC-681, DPC-684, fosamprenavir calcium (Lexiva), benzenesulfonamide derivatives disclosed in WO 03053435, R-944, Ro-03-34649, VX-385, GS-224338, OPT-TL3, PL-100, SM-309515, AG-148, DG-35-VIII, DMP-850, GW-5950X, KNl-1039, L-756423, LB-71262, LP-130, RS-344, SE-063, UIC-94-003, Vb-19038, A-77003, BMS-182193, BMS-186318, SM-309515, and JE-2147.

Compounds useful as inhibitors of the HIV reverse transcriptase enzyme that may be used in combination with the compounds of the present invention include, but are not limited to, abacavir (1592U89), FTC, GS-840, lamivudine (3TC), adefovir dipivoxil, beta-fluoro-ddA, ddC (dideoxycytidine, zalcitabine), ddI (dideoxyinsine, didanosine), stavudine (d4T), zidovudine (AZT), tenofovir, amdoxovir, SPD-754, SPD-756, racivir, reverset (DPC-817), MIV-210 (FLG), beta-L-Fd4C (ACH-126443), MIV-310 (alovudine, FLT), dOTC, DAPD, and emtricitabine.

Compounds useful as non-nucleoside inhibitors of the HIV reverse transcriptase enzyme include, but are not limited to, efavirenz, HBY-097, nevirapine, TMC-120 (dapivirine), TMC-125, delaviradine, DPC-083, DPC-961, TMC-120, capravirine, and tricyclic pyrimidinone derivatives as disclosed in WO 03062238.

Compounds useful as CCR5 inhibitors that may be used in combination with the compounds of the present invention include, but are not limited to, TAK-779, SC-351125, SCH-D, UK-427857, PRO-140, and GW-873140 (Ono-4128, AK-602).

Compounds useful as inhibitors of HIV integrase enzyme that may be used in combination with the compounds of the present invention include, but are not limited to, 1,5-naphthyridine-3-carboxamide derivatives disclosed in WO 03062204, compounds disclosed in WO 03047564, compounds disclosed in WO 03049690, and 5-hydroxypyrimidine-4-carboxamide derivatives disclosed in WO 03035076.

Fusion inhibitors for the treatment of HIV that may be used in combination with the compounds of the present invention include, but are not limited to, T20, T-1249, AMD-3100, and fused tricyclic compounds disclosed in JP 2003171381.

Other compounds that are useful inhibitors of HIV that may be used in combination with the compounds of the present invention include, but are not limited to, Soluble CD4, TNX-355, PRO-542, BMS-806, tenofovir disoproxil fumarate, and compounds disclosed in JP 2003119137.

Compounds useful in the treatment or management of infection from viruses other than HIV that may be used in combination with the compounds of the present invention include, but are not limited to, acyclovir, penciclovir, HPMPC, oxetanocin G, AL-721, cidofovir, cytomegalovirus immune globin, cytovene, ganciclovir, famciclovir, Isis 2922, KNI-272, valaciclovir, and virazole ribavirin.

Compounds that act as immunomodulators and may be used in combination with the compounds of the present invention include, but are not limited to, AD-439, AD-519, Alpha Interferon, AS-101, bropirimine, acemannan, CL246,738, EL10, FP-21399, gamma interferon, granulocyte macrophage colony stimulating factor, IL-2, immune globulin intravenous, IMREG-1, IMREG-2, imuthiol diethyl dithio carbamate, alpha-2 interferon, methionine-enkephalin, MTP-PE, granulocyte colony stimulating sactor, remune, rCD4, recombinant soluble human CD4, interferon alfa-2, SK&F106528, soluble T4 yhymopentin, tumor necrosis factor (TNF), tucaresol, recombinant human interferon beta, and interferon alfa n-3.

Anti-infectives that may be used in combination with the compounds of the present invention include, but are not limited to, clindamycin with primaquine, fluconazole, pastill, ornidyl, eflornithine pentamidine, spiramycin, intraconazole-R51211, trimetrexate, daunorubicin, recombinant human erythropoietin, recombinant human growth hormone, megestrol acetate, testerone, and total enteral nutrition.

Other compounds that may be used in combination with the compounds of the present invention include, but are not limited to, acmannan, ansamycin, LM 427, AR177, BMS-232623, BMS-234475, Cl-1012, curdlan sulfate, dextran sulfate, STOCRINE EL10, hypericin, lobucavir, novapren, peptide T octabpeptide sequence, trisodium phosphonoformate, probucol, and RBC-CD4.

In addition, the compounds of the present invention may be used in combination with compounds that act as inhibitors of metallo-matrix proteases, so-called MMP inhibitors.

The particular choice of an additional agent or agents will depend on a number of factors that include, but are not limited to, the condition of the mammal being treated, the particular condition or conditions being treated, the identity of the compound or compounds of the present invention and the additional agent or agents, and the identity of any additional compounds that are being used to treat the mammal. The particular choice of the compound or compounds of the invention and the additional agent or agents is within the knowledge of one of ordinary skill in the art.

The compounds of the present invention may be administered in combination with any of the above additional agents for the treatment of a mammal, such as a human, that is suffering from an infection with the HIV virus, AIDS, AIDS-related complex (ARC), or any other disease or condition which is related to infection with the HIV virus. Such a combination may be administered to a mammal such that a compound or compounds of the present invention are present in the same formulation as the additional agents described above. Alternatively, such a combination may be administered to a mammal suffering from infection with the HIV virus such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

Additionally, the compounds of the present invention may be administered to a mammal, such as a human, in combination with an additional agent that has the effect of increasing the exposure of the mammal to a compound of the invention. The term “exposure,” as used herein, refers to the concentration of a compound of the invention in the plasma of a mammal as measured over a period of time. The exposure of a mammal to a particular compound can be measured by administering a compound of the invention to a mammal in an appropriate form, withdrawing plasma samples at predetermined times, and measuring the amount of a compound of the invention in the plasma using an appropriate analytical technique, such as liquid chromatography or liquid chromatography/mass spectroscopy. The amount of a compound of the invention present in the plasma at a certain time is determined and the concentration and time data from all the samples are plotted to afford a curve. The area under this curve is calculated and affords the exposure of the mammal to the compound. The terms “exposure,” “area under the curve,” and “area under the concentration/time curve” are intended to have the same meaning and may be used interchangeably throughout.

Among the agents that may be used to increase the exposure of a mammal to a compound of the present invention are those that can as inhibitors of at least one isoform of the cytochrome P450 (CYP450)enzymes. The isoforms of CYP450 that may be beneficially inhibited include, but are not limited to, CYP1A2, CYP2D6, CYP2C9, CYP2C19 and CYP3A4. Suitable agents that may be used to inhibit CYP 3A4 include, but are not limited to, ritonavir and delavirdine.

Such a combination may be administered to a mammal such that a compound or compounds of the present invention are present in the same formulation as the additional agents described above. Alternatively, such a combination may be administered such that the compound or compounds of the present invention are present in a formulation that is separate from the formulation in which the additional agent is found. If the compound or compounds of the present invention are administered separately from the additional agent, such administration may take place concomitantly or sequentially with an appropriate period of time in between. The choice of whether to include the compound or compounds of the present invention in the same formulation as the additional agent or agents is within the knowledge of one of ordinary skill in the art.

The present invention also includes the use of a compound of the present invention as described above in the preparation of a medicament for (a) inhibiting HIV protease, (b) preventing or treating infection by HIV, or (c) treating AIDS or ARC. Also provided are uses of a compound of the invention for the preparation of a medicament for the inhibition of HIV protease activity in an HIV-infected mammal.

The present invention further includes the use of any of the HIV protease inhibiting compounds of the present invention as described above in combination with one or more HIV infection/AIDS treatment agents selected from an HIV/AIDS antiviral agent, an anti-infective agent, and an immunomodulator for the manufacture of a medicament for (a) inhibiting HIV protease, (b) preventing or treating infection by HIV, or (c) treating AIDS or ARC, said medicament comprising an effective amount of the HIV protease inhibitor compound and an effective amount of the one or more treatment agents.

The compounds of this invention are also useful in the preparation and execution of screening assays for antiviral compounds. For example, the compounds of this invention are useful for isolating enzyme mutants that are excellent screening tools for more powerful antiviral compounds. Furthermore, the compounds of this invention are useful in establishing or determining the binding site of other antivirals to HIV protease, e.g., by competitive inhibition. Thus the compounds of this invention are commercial products to be sold for these purposes.

Specific Examples of various compounds according to the invention may be advantageously prepared as set out in the Examples below.

The structures of the compounds of the following Examples were confirmed by one or more of the following: proton magnetic resonance spectroscopy, infrared spectroscopy, elemental microanalysis, mass spectrometry, thin layer chromatography, melting point, boiling point, and HPLC.

Proton magnetic resonance (¹H NMR) spectra were determined using a 300 megahertz Tech-Mag, Bruker Avance 300DPX, or Bruker Avance 500 DRX spectrometer operating at a field strength of 300 or 500 megahertz (MHz). Chemical shifts are reported in parts per million (ppm, δ) downfield from an internal tetramethylsilane standard. Alternatively, ¹H NMR spectra were referenced to residual protic solvent signals as follows: CHCl₃=7.26 ppm; DMSO=2.49 ppm; C₆HD₅=7.15 ppm. Peak multiplicities are designated as follows: s=singlet; d=doublet; dd=doublet of doublets; t=triplet; q=quartet; br=broad resonance; and m=multiplet. Coupling constants are given in Hertz. Infrared absorption (IR) spectra were obtained using a Perkin-Elmer 1600 series FTIR spectrometer. Elemental microanalyses were performed by Atlantic Microlab Inc. (Norcross, Ga.) and gave results for the elements stated within ±0.4% of the theoretical values. Flash column chromatography was performed using Silica gel 60 (Merck Art 9385). Analytical thin layer chromatography (TLC) was performed using precoated sheets of Silica 60 F₂₅₄ (Merck Art 5719). HPLC chromatographs were run on a Hewlett Packard Model 1100 system fitted with a Zorbax SB-C18 4.6 mm×150 mm column having 3.5 micron packing material. Unless otherwise stated, a ramp of 5% CH₃CN/H₂O to 95% CH₃CN/H₂O over 7.5 minutes then holding at 95% CH₃CN/H₂O for 2.5 minutes (both solvents contained 0.1% v/v TFA) at a flow of 1 mL/min was used. Retention times (Rt) are given in minutes. Semi-preparative HPLC samples were run on a Gilson LC3D system fitted with a 21.2 mm×250 mm C8 column. Ramps were optimized for each compound with a CH₃CN/H₂O solvent system. Melting points were determined on a Mel-Temp apparatus and are uncorrected. All reactions were performed in septum-sealed flasks under a slight positive pressure of argon, unless otherwise noted. All commercial reagents were used as received from their respective suppliers with the following exceptions: tetrahydrofuran (THF) was distilled from sodium-benzophenone ketyl prior to use; dichloromethane (CH₂Cl₂) was distilled from calcium hydride prior to use; anhydrous lithium chloride was prepared by heating at 110° C. under vacuum overnight. Mass spectra, both low and high resolution, were measured using either electrospray (El) or fast atom bombardment (FAB) ionization techniques.

The following abbreviations are used herein: Et₂O (diethyl ether); DMF (N,N-dimethylformamide); DMSO (dimethylsulfoxide); MeOH (methanol); EtOH (ethanol); EtOAc (ethyl acetate); Ac (acetyl); Hex (hexane); Me (methyl); Et (ethyl); Ph (phenyl); DIEA (diisopropylethylamine); TFA (trifluoroacetic acid); DTT (dithiothreitol); and THF (tetrahydrofuran); and (precipitate); min. or min (minutes); h (hours).

Solid-phase syntheses were performed by immobilizing reagents with Rink amide linkers (Rink, Tetrahedron Letters (1987) 28:3787), which are standard acid-cleavable linkers that upon cleavage generate a free carboxamide group. Small-scale solid-phase syntheses, e.g., about 2-5 μmole, were performed using Chiron SynPhase® polystyrene O-Series crowns (pins) derivatized with Fmoc-protected Rink amide linkers. For larger scale (e.g., greater than about 100 μmole) syntheses, the Rink amide linkages were formed to Argonaut Technologies Argogel® resin, a grafted polystyrene-poly(ethylene glycol) copolymer. Any suitable resin may be used as the solid phase, selected from resins that are physically resilient and that, other than with regard to the linking and cleavage reactions, are inert to the synthetic reaction conditions.

### EXAMPLES

Biological Evaluation

Cells and Virus

T-cell lines, CEM-SS, and MT-2, and viruses HIV-1 RF and HIV-1 NL4-3 (pNL4-3) were obtained from the National Institutes of Health (AIDS Research and Reference Reagent Program, Bethesda, MD). HIV-1 NL4-3(I84V/L90M) was derived from a clinical isolate that exhibited the protease inhibitor-resistance associated substitutions I84V and L90M, by cloning of an reverse transcriptase-polymerase chain reaction amplified fragment into the unique Age I and Spe I restriction sites of pNL4-3.

Cytopathic Effect (CPE) Inhibition Assays

The ability of compounds to protect cells against HIV infection was measured by the MTT dye reduction method, essentially as described (See Pauwels, R. Balzarini, J. Baba, M. Snoeck, R. Schols, D. Herdewijn, P. Desmyter, J. and De Clercq, E. 1988, “Rapid and automated tetrazolium-based colorimetric assay for the detection of anti-HIV compounds,”. J Virol. Methods., 20: 309-321 and Weislow, O. S. Kiser, R. Fine, D. L. Bader, J. Shoemaker, R. H. and Boyd, M. R. 1989. “New soluble-formazan assay for HIV-1 cytopathic effects: application to high-flux screening of synthetic and natural products for AIDS-antiviral activity”. J. Natl. Cancer Inst. 81:577-586). Subject cells were infected with test virus at an moi of 0.025 to 0.819 or mock infected with medium only and added at 2×10⁴ cells per well into 96 well plates containing half-log dilutions of test compounds. Six days later, 50 μl of XTT (1 mg/ml XTT tetrazolium, 0.02 nM phenazine methosulfate) was added to the wells and the plate was reincubated for four hours. Viability, as determined by the amount of XTT formazan produced, was quantified spectrophotometrically by absorbance at 450 nm. Data from CPE assays were expressed as the percent of formazan produced in compound-treated cells compared to formazan produced in wells of uninfected, compound-free cells. The fifty percent effective concentration (EC₅₀) was calculated as the concentration of compound that effected an increase in the percentage of formazan production in infected, compound-treated cells to 50% of that produced by uninfected, compound-free cells. The 50% cytotoxicity concentration (CC₅₀) was calculated as the concentration of compound that decreased the percentage of formazan produced in uninfected, compound-treated cells to 50% of that produced in uninfected, compound-free cells. The therapeutic index was calculated by dividing the cytotoxicity (CC₅₀) by the antiviral activity (EC₅₀).

Susceptibility Assays

Compounds were tested in phenotypic susceptibility assays at Virologic, Inc., (See Petropoulos C. J., Parkin N. T., Limoli K. L., Lie Y. S., Wrin T., Huang W., Tian H., Smith D., Winslow G. A., Capon D J, Whitcomb J M. 2000, “A novel phenotypic drug susceptibility assay for human immunodeficiency virus type 1,” Antimicrob Agents Chemother 44(4):920-928) or using the assay described here. MT-2 cells were infected with either HIV-1 NL4-3 or HIV-1 NL4-3(I84V/L90M) and incubated in the presence of serial 0.5 log dilutions of test compounds. Three days later, culture supernatants were collected and virus production, as determined by p24 ELISA, was assayed. Percent inhibition was calculated as p24 concentration in compound-treated samples as compared to infected, compound-free controls. Inhibition of viral replication is determined by measuring reduction in HIV p24 present in the culture supernatant, using a Beckman-Coulter p24 HIV-1 Ag EIA kit and following the supplied protocol. Absorbance is read on a MRX microplate reader (Dynex Technologies). The EC₅₀ was calculated as the concentration of compound that effected a decrease in the p24 production by infected, compound-treated cells to 50% of that produced by infected, compound-free cells.

HIV-1 Protease RET Assay

Ki's for the inhibitors of HIV-1 protease were determined using a resonance energy transfer (RET) assay. A mutant form of this enzyme (Q7S) is used for this assay because it is more stable against auto-proteolysis than the wild-type protein. This enzyme is first partially purified as inclusion bodies from cell lysate. It is then solublized in 8M urea and passed through a Q-Sepharose column (Pharmacia) for further purification. To refold this protein, samples containing Q7S is dialyzed into 50 mM sodium phosphate pH 7.0, 50 mM NaCl, 10 mM DTT, and 10% glycerol.

The commercially available peptide substrate (Molecular Probes Cat. # H-2930) RE(EDANS)SQNYPIVQK(DABCYL)R is used to assess activity and Ki's. This peptide is cleaved quantitatively by HIV-1 Pr at the Tyr-Pro bond. The EDANS fluorophore absorbs at 340 nm and emits at 490 nm. The reaction is carried out in a 96 well plate in a total volume of 100□L and is run for 12 minutes at 37C under steady-state conditions with 5□M substrate and 2 nM active dimer enzyme concentration. The literature value Km for this substrate and enzyme is 103+/−8 μM (See Matayoshi, et al., “Novel Fluorogenic Substrates for Assaying Retroviral Proteases by Resonance Energy Transfer,” Science 247, 954 (1990)). The buffer for this reaction is 0.1M sodium acetate pH 4.8, 1M NaCl, 1 mM EDTA, 5 mM dithiothreitol, 10% dimethyl sulfoxide and 1 mg/ml bovine serum albumin. Inhibition curves are fit using the Morrison tight binding equation.

### Example D10

### (R)-3-((2S,3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide

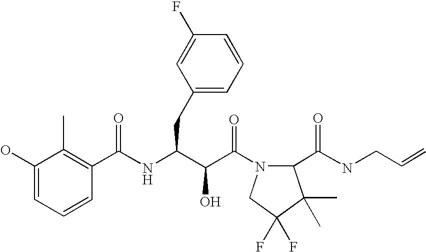

The following represents synthesis of key intermediates for the synthesis of the title compound. L-2-tert-Butoxycarbonylamino-3-(3-fluoro-phenyl)-propionic acid. A mixture of L-2-amino-3-(3-fluoro-phenyl)-propionic acid ( 20.0 g, 110 mmol, 1 eq) in H₂O (100 mL) was treated with Na₂CO₃ (16.2 g, 153 mmol, 1.4 eq) in H₂O (40 mL) followed by 1,4-dioxane (100 mL) and cooled to 0 C. The BOC₂O was added and the reaction mixture was stirred at ambient temperature for 5 h after which the dioxane was evaporated. H₂O (125 mL) was then added and the mixture then washed with Et₂O (2×100 mL). The aqueous phase was acidified with 10% citric acid followed by extraction with EtOAc (2×300 mL). The combined EtOAc layers were washed with H₂O (2×150 mL), brine (150 mL), dried (Na₂SO₄) and concentrated to give the acid as a colorless, viscous oil which slowly solidified upon standing (31 g, quant). ¹H NMR (CDCl₃) 7.33-7.26 (m, 1H), 7.00-6.91 (m, 3H), 4.96 (s, 1H), 4.62 (bs, 1H), 3.23 (dd, J=14,5.3 , 2H), 1.44 (s, 9H); Anal Calcd for C₁₄H₁₈NO₄F: C, 59.36; H, 6.40; N, 4.94. Found: C, 59.29; H, 6.34; N, 4.90.

L-[2-(3-Fluoro-phenyl)-1-(methoxy-methyl-carbamoyl)-ethyl]-carbamic acid -tert-butyl ester. To a solution of L-2-tert-butoxycarbonylamino-3-(3-fluoro-phenyl)-propionic acid (30.9 g, 109 mmol) in THF (180 mL) was added carbonyldiimidazole (21.2 g, 131 mmol, 1.2 eq). After stirring the solution at ambient temperature for 45 min was added DMF (64 mL), N,O-dimethylhydroxylamine hydrochloride (11.7 g, 120 mmol, 1.1 eq) and diisopropylethylamine (20 mL, 113 mmol, 1.04 eq). After stirring for a total time of 2 h, the solvents were evaporated in vacuo and the oily residue dissolved in EtOAc (300 mL). The organic phase was washed with H₂O (500 mL), 10% citric acid (2×150 mL), H₂O (500 mL), sat'd Na₂CO₃ (200 mL), brine (200 mL), dried (Na₂SO₄) and concentrated to give the product suitable for further use (31.6 g, 89%).

¹H NMR (CDCl₃) 7.29-7.22 (m, 1H), 6.98-6.89 (m, 3H), 5.20 (bs, 1H), 4.96 (bs, 1H), 3.72 (s, 3H), 3.19 (s, 3H), 3.07 (dd, J=13.6 , 5.9, 2H), 1.41 (s, 9H). Anal Calcd for C₁₆H₂₃N₂O₄F: C, 58.88; H, 7.10; N, 8.58. Found: C, 58.89; H, 7.19; N, 8.71.

L-[1-(3-Fluoro-benzyl)-2-oxo-ethyl]-carbamic acid tert-butyl ester. To a 3-neck flask which purged with argon was added a 1M solution of LAH in Et₂O (106 mL, 1.1 eq) and cooled to 0 C. A solution of L-[2-(3-fluoro-phenyl)-1-(methoxy-methyl-carbamoyl)-ethyl]-carbamic acid -tert-butyl ester(31.6 g, 97 mmol, 1 eq) in THF (150 mL) was added over a period of 1 h such that the temperature remained below 5 C. After stirring for an additional 30 min the reaction was quenched with EtOAc (60 mL) followed by 5% KHSO₄ (100 mL). EtOAc (500 mL) was added and the organic phase was washed with 1N HCl (3×100 mL), H₂O (500 mL), brine (200 mL), dried (Na₂SO₄) and concentrated to a white solid which was filtered and washed with heptane (200 mL). The aldehyde was suitable for further use (17.6 g, 68%). ¹H NMR (CDCl₃) 9.65 (s, 1H), 7.33-7.26 (m, 1H), 7.01-6.89 (m, 3H), 5.06 (bs, 1H), 4.43 (broad m, 2H), 1.45 (s, 9H). Anal Calcd for C₁₄H₁₈NO₃F: C, 62.91; H, 6.79; N, 5.24. Found: C, 62.73; H, 6.66; N, 5.21.

3-tert-Butoxycarbonylamino-4-(3-fluoro-phenyl)-2-hydroxy-butyric acid (diastereomeric). A solution of L-[1-(3-fluoro-benzyl)-2-oxo-ethyl]-carbamic acid tert-butyl ester(17.6 g, 66 mmol, 1 eq) in MeOH (104 mL) was cooled to 0 C. A solution of sodium bisulfite in H₂O (104 mL) was added and the mixture stirred for 5 h at 0 C after which it was placed in a freezer for 7 h. The reaction mixture was then charged with a solution of NaCN (3.87 g, 79 mmol, 1.2 eq) in H₂O (104 mL) followed by EtOAc (280 mL) and stirred at room temperature for 11 h after which the organic layer was separated, dried (Na₂SO₄) and concentrated to give the crude cyanohydrin as a waxy solid. This material was dissolved in 1,4 dioxane (265 mL), charged with anisole (11 mL) and cooled to 0 C. Concentrated HCl (265 mL) was added, with vigorous stirring, to the reaction mixture followed by heating at reflux for 1 h. The dioxane plus most of the water was evaporated in vacuo . The remaining residue was basified with 2N NaOH and washed with Et₂O (3×200 mL). The aqueous phase was then charged with 1,4 dioxane (120 mL) followed by BOC₂O (15.8 g, 1.1 eq). After stirring at ambient temperature for 3 h the dioxane was removed in vacuo and the remaining mixture acidified with 10% citric acid followed by extraction with EtOAc (2×300 ml). The combined organic layers were washed with H₂O (300 mL), brine (200 mL), dried (Na₂SO₄) and concentrated to give the acid as diastereomeric mixture (ca 1:1) and orange solid (10.56 g, 51%) ¹H NMR (DMSO) 7.35-7.25 (m, 2H), 7.06-6.96 (m, 6H), 6.76 (d, J=9.0, 1H), 6.43 (d, J=9.6, 1H), 4.02-3.89 (m, 4H), 3.57 (m, 2H), 2.83 (dd, J=13.4, 6.1, 2H), 1.28 (s, 9H), 1.26 (s, 9H).

(2S,3R)-3-tert-Butoxycarbonylamino-4-(3-fluoro-phenyl)-2-hydroxy-butyric acid methyl ester. To a solution of 3-tert-butoxycarbonylamino-4-(3-fluoro-phenyl)-2-hydroxy-butyric acid (diastereomeric) (10.56 g, 33.8 mmol., 1 eq) in DMF (130 mL) was suspended K₂CO₃ (6.07 g, 43 mmol, 1.3 eq) followed by CH₃I (4.2 mL, 68 mmol, 2 eq). After stirring for 2 h at ambient temperature the DMF was evaporated in vacuo. The remaining residue was dissolved in EtOAc (300 mL) and washed with H₂O (2×100 mL), sodium thiosulfate solution (100 mL), brine (200 mL) dried (Na₂SO₄) and concentrated to give a crude orange solid (9.55 g). Purification by column chromatography (1:1 EtOAc/hexanes) afforded 6.96 g total (63%); of which 3.28 g being the desired diastereomer (2S,3R)-3-tert-Butoxycarbonylamino-4-(3-fluoro-phenyl)-2-hydroxy-butyric acid methyl ester (cream colored solid), and 3.68 g being the undesired product (2R,3R)-3-tert-butoxycarbonylamino-4-(3-fluoro-phenyl)-2-hydroxy-butyric acid methyl ester. (2S,3R) product: ¹H NMR (CDCl₃) 7.30-7.22 (m, 1H), 7.01-6.90 (m, 3H), 4.88 (d, J=8.2, 1H), 4.32 (m, 2H), 3.67 (s, 3H), 2.79 (t, J=6.9, 2H), 1.40 (s, 9H). (2R,3R) product: ¹H NMR (CDCl₃) 7.32-7.25 (m, 1H), 7.09-6.91 (m, 3H), 4.82 (d, J=9.8, 1H), 4.27 (dd, J=16.9, 7.6, 1H), 4.08 (d, J=3.2, 1H), 3.78 (s, 3H), 3.17 (d, J=4.5, 1H), 2.93(d, J=4.5, 1H), 1.40 (s, 9H).

(2S,3R)-3-tert-Butoxycarbonylamino-4-(3-fluoro-phenyl)-2-hydroxy-butyric acid. A mixture of (2S,3R)-3-tert-Butoxycarbonylamino-4-(3-fluoro-phenyl)-2-hydroxy-butyric acid methyl ester (3.28 g, 10.05 mmol, 1 eq), 4N NaOH (4 mL, 16 mmol, 1.6 eq), MeOH (42 mL) and 1,4-dioxane (63 mL) was stirred at ambient temperature for 1.5 h after which the solvents were evaporated. To the residue was added 10% citric acid (100 mL) followed by extraction with EtOAc (100 mL). The organic layer was washed with H₂O (100 mL), brine (50 mL), dried (Na₂SO₄) and concentrated to give the desired product as a cream colored solid (3.06 g, 97%). ¹H NMR (DMSO) 7.33-7.26 (m, 1H), 7.02-6.97 (m, 3H), 6.78 (d, J=5.2, 1H), 3.98 (d, J=5.5, 1H), 3.99-3.86 (m, 2H), 2.77-2.82 (m, 2H), 1.27 (s, 9H).

Conversion of undesired (2R,3R) diastereomer-methylester to (2S,3R)-3-tert-butoxycarbonylamino-4-(3-fluoro-phenyl)-2-hydroxy-butyric acid. (2S,3R)-3-tert-Butoxycarbonylamino-2-(2-chloro-acetoxy)-4-(3-fluoro-phenyl)-butyric acid methyl ester. A solution of of the (2R,3R)-3-tert-butoxycarbonylamino-4-(3-fluoro-phenyl)-2-hydroxy-butyric acid methyl ester (8 g, 24.5 mmol, 1 eq), chloroacetic acid (5.79 g, 61.3 mmol, 2.5 eq), and PPh₃ (16 g, 61.3 mmol, 2.5 eq) in benzene (340 mL) was cooled to 0 C followed by the addition of diethylazodicarboxylate (9.7 mL, 61.3 mmol, 2.5 eq) over a 20 min period. After the addition, the reaction mixture was stirred at ambient temperature for 2 h after which the reaction mixture was concentrated and the residue purified by column chromatography with 30% EtOAc/hexanes as eluant. Appropriate fractions were combined and concentrated to give a yellow solid which was shaken with heptane and filtered to remove the yellow DEAD residues. The product was thus obtained as a white solid (4.25 g, 43%) ¹H NMR (CDCl₃) 7.32 (m, 1H), 7.03-6.96 (m, 3H), 5.34 (d, J=3.5, 1H), 4.26 (s, 2H), 4.75-4.5 (series of m, 2H), 3.77 (s, 3H), 2.92 (bd, J=7, 2H), 1.43 (s, 9H).

(2S,3R)-3-tert-butoxycarbonylamino-4-(3-fluoro-phenyl)-2-hydroxy-butyric acid. A mixture of (2S,3R)-3-tert-butoxycarbonylamino-2-(2-chloro-acetoxy)-4-(3-fluoro-phenyl)-butyric acid methyl ester (4.56 g, 11.3 mmol, 1 eq), 4N NaOH (6.5 mL, 25.9 mmol, 2.3 eq), MeOH (48 mL) and 1.4-dioxane (72 mL) was stirred at ambient temperature for 4 h after which the solvents were removed in vacuo and the residue was charged with H₂O (50 mL) and washed with Et₂O (100 mL). The aqueous layer was made acidic with 10% citric acid and extracted with EtOAc (2×75 mL). The combined EtOAc layers were washed with H₂O (3×50 mL) brine (50 mL), dried (Na₂SO₄), concentrated, shaken with heptane and filtered to give the desired acid as a white solid (3.3 g, 94%).

The title compound was prepared as described previously, D10. ¹H NMR (DMSO) 9.42 (s, 1H),8.26 (d, J=8.1, 1H), 8.17 (t, J=5.9, 1H), 7.32 (m, 1H), 7.18 (m, 2H), 7.00 (m, 2H), 6.79 (d, J=8.1, 1H), 6.56 (d, J=7.5, 1H), 5.79 (m, 1H), 5.51 (d, J=6.4, 1H), 5.24 (d, J=15.4, 1H),5.06 (d, J=10.4, 1H), 4.49-4.28 (series of m, 5H), 3.74 (broad m, 2H), 2.89-2.67 (m, 2H), 1.81 (s, 3H), 1.22 (s, 3H), 1.05 (s, 3H). Anal Calcd for C₂₈H₃₂N₃O₅F₃×0.25 H₂O: C, 60.91; H, 5.93; N, 7.61. Found: C, 60.96; H, 6.05; N, 7.20.

### Example D11

### (S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide

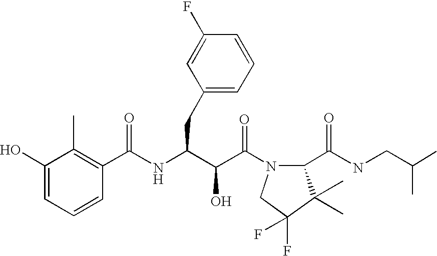

White solid: ¹H NMR (DMSO-d₆) δ 9.14 (s, 1H), 8.03 (d, 1H, J=8.3), 7.76 (t, 1H, J=5.8), 7.09 (dd, 1H, J=7.4, 14.4), 6.99 (d, 2H, J=7.6), 6.81-6.73 (m, 2H), 6.58 (d, 1H, J=8.1), 6.34 (d, 1H, J=6.8), 5.23 (d, 1H, J=6.6), 4.25 (dd, 1H, J=12.2, 25.0), 4.15-4.08 (m, 3H), 2.77-2.46 (m, 4H), 1.59 (s, 3H), 1.52-1.43 (m, 1H), 1.00 (s, 3H), 0.83 (s, 3H), 0.65 (d, 6H, J=6.4); HRMS (ESI) m/z calcd for C₃₀H₃₇F₃N₃O₅ (M+H)⁺564.6130, found: 564.2674; Anal. Calcd for C₃₀H₃₆F₃N₃O₅: C, 61.80; H, 6.44; N, 7.46. Found: C, 61.58; H, 6.45; N, 7.34.
REPRESENTATIVE PROCEDURE FOR THE HYDROXYLATION OF A SUBSTITUTED BENZOIC ACID

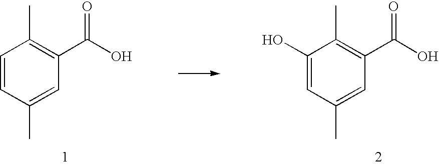

2,5-dimethyl-benzoic acid (1) (20 g, 133 mmol) was dissolved in concentrated H₂SO₄ (30 mL) and fuming H₂SO₄ (20% SO₃, 70 mL). The reaction mixture was heated to 110° C. for 2 hours. After cooling, the solution was poured carefully into a beaker of ice H₂O (400 mL) and was then neutralized with 20% aqueous NaOH (400 mL). The H₂O was partially removed in vacuo until a white salt mixture started to form. The solid was collected on a sintered-glass funnel and was then dried in a vacuum oven. The dried salt mixture was placed in a ceramic crucible with KOH (160 g) and was melted together using a butane torch for 0.5 h. After cooling, the fused solid was dissolved in H₂O (300 mL) and acidified with concentrated HCl (300 mL). The product was extracted from the aqueous solution with EtOAc (3×200 mL). The combined organic layers were washed with brine (100 mL) and dried over MgSO₄. The solvents were removed in vacuo and the solid residue was recrystallized with 20% EtOAc/CHCl₃ four times to afford 3-hydroxy-2,5-dimethyl-benzoic acid (2) as a light brown solid (9.8 g, 44%)

¹H NMR (Acetone-d₆) 10.93 (br s, 1H), 8.34 (br s, 1H), 7.20 (s, 1H), 6.86 (s, 1H), 2.37 (s, 3H), 2.24 (s, 3H).

References—Fujiwara, A. N.; Acton, E. M., Can. J. Chem., 1970, 48, 1346-1349.

Charlesworth, E. H.; Levene, L., Can. J. Chem., 1963, 41, 1071-1077.

### Example D12

### (R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide

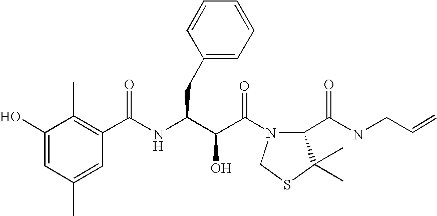

White solid: ¹H NMR (DMSO-d₆) δ 9.23 (s, 1H), 8.09 (m, 2H), 7.35-7.17 (m, 5H), 6.60 (s, 1H), 6.37 (s, 1H), 5.82-5.68 (m, 1H), 5.41 (br s, 1H), 5.20 (dd, 1H, J=1.6, 17.2), 5.11 (d, 1H, J=9.2), 5.02 (dd, 1H, J=1.5, 10.2), 5.00 (d, 1H, J=9.1), 4.46-4.37 (m, 3H), 3.79 (ddd, 1H, J=5.3, 5.5, 15.9), 3.63 (ddd, 1H, J=5.4, 5.3, 15.9), 2.82 (dd, 1H, J=0.3, 13.9), 2.71 (dd, 1H, J=10.7, 13.6), 2.16 (s, 3H), 1.76 (s, 3H), 1.51 (s, 3H), 1.36 (s, 3H); HRMS (ESI) m/z calcd for C₂₈H₃₆N₃O₅S (M+H)⁺526.6670, found 526.2376; Anal. Calcd for C₂₈H₃₅N₃O₅S.0.3 H₂O: C, 63.32; H, 6.76; N, 7.91, Found: C, 63.35; H, 6.70; N, 7.71.

### Example D13

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide

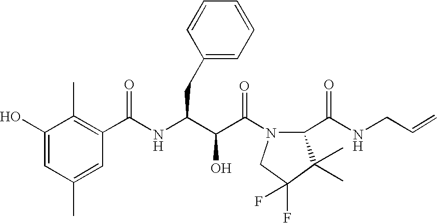

White solid: ¹H NMR (DMSO-d₆) δ 9.25 (s, 1H), 8.13-8.10 (m, 2H), 7.37-7.15 (m, 5H), 6.60 (s, 1H), 6.37 (s, 1H), 5.84-5.73 (m, 1H), 5.50 (d, 1H, J=6.1), 5.23 (dd, 1H, J=1.7, 17.5), 5.05 (dd, 1H, J=1.5, 10.4), 4.49-4.28 (m, 3H), 4.26 (s, 1H), 3.78-3.68 (m, 2H), 2.89,-2.66 (m, 2H), 2.16 (s, 3H), 1.75 (s, 3H), 1.21 (s, 3H), 1.05 (s, 3H); HRMS (ESI) m/z calcd for C₂₉H₃₆F₂N₃O₅ (M+H)⁺544.6070, found 544.2623; Anal. Calcd for C₂₉H₃₅F₂N₃O₅.0.5 H₂O: C, 63.05; H, 6.57; N, 7.60. Found: C, 63.05; H, 6.40; N, 7.39.

### Example D14

### (S)-4,4-Difluoro-1-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide

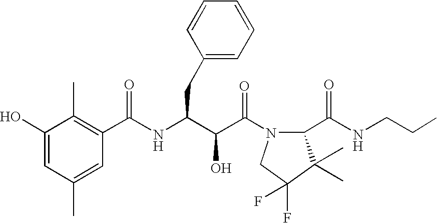

White solid: ¹H NMR (DMSO-d₆) δ 9.17 (s, 1H), 8.04 (d, 1H, J=8.1), 7.85 (t, 1H, J=5.1), 7.29-7.09 (m, 5H), 6.53 (s, 1H), 6.30 (s, 1H), 5.38 (d, 1H, J=6.1), 4.40-4.24 (m, 3H), 4.14 (s, 1H), 3.04-2.90 (m, 2H), 2.77 (d, 1H, J=2.2), 2.65-2.59 (m, 1H), 2.09 (s, 3H), 1.67 (3, 3H), 1.39-1.31 (m, 2H), 1.13 (s, 3H), 0.97 (s, 3H), 0.78 (s, 3H). HRMS (ESI) m/z calcd for C₂₉H₃₈F₂N₃O₅ (M+H)⁺546.6230, found 546.2780; Anal. Calcd for C₂₉H₃₇F₂N₃O₅: C, 63.84; H, 6.84; N, 7.70. Found: C, 63.44; H, 6.82; N, 7.52.

### Example D15

### (S)-4,4-Difluoro-1-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide

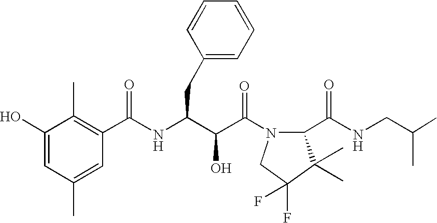

White solid: ¹H NMR (DMSO-d₆) δ 9.24 (s, 1H), 8.11 (d, 1H, J=8.3), 7.94 (t, 1H, J=5.8), 7.37-7.16(m, 5H), 6.60 (s, 1H), 6.38 (s, 1H), 5.44 (d, 1H, J=6.3), 4.48-4.29 (m, 3H), 4.25 (s, 1H), 2.94-2.83 (m, 3H), 2.73-2.64 (m, 1H), 2.16 (s, 3H), 1.75 (s, 3H), 1.74-1.65 (m, 1H), 1.21 (s, 3H), 1.05 (s, 3H), 0.86 (d, 6H, J=6.6); HRMS (ESI) m/z calcd for C₃₀H₄₀F₂N₃O₅ (M+H)⁺560.6500, found: 560.2928; Anal. Calcd for C₃₀H₃₉F₂N₃O₅: C, 64.38; H, 7.02; N, 7.51. Found: C, 64.09; H, 7.05; N, 7.29.

### Example D16

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide

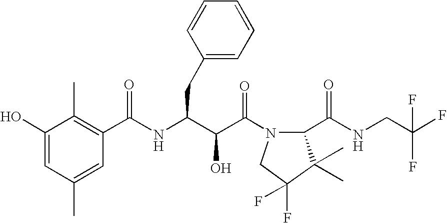

(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide

White solid: ¹H NMR (DMSO-d₆) δ 9.27 (s, 1H), 8.72 (t, 1H, J=6.2), 8.15 (d, 1H, J=8.1), 7.37-7.19 (m, 5H), 6.63 (s, 1H), 6.39 (s, 1H), 5.57 (d, 1H, J=6.3), 4.52-4.33 (m, 4H), 4.10-3.94 (m, 1H), 3.93-3.88 (m, 1H), 2.87 (d, 1H, J=7.3), 2.75-2.69 (m, 1H), 2.19 (s, 3H), 1.77 (s, 3H), 1.25 (s, 3H), 1.06 (s, 3H); HRMS (ESI) m/z calcd for C₂₈H₃₃F₃N₃O₅ (M+H)⁺ 586.5670, found 586.2340; Anal. Calcd for C₂₈H₃₂F₃N₃0₅.0.4 H₂O: C, 56.73; H, 5.58; N, 7.09. Found: C, 56.64; H, 5.41; N, 6.94.

### Example D17

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide (also named N-ethyl-4,4-difluoro-1-{(2S,3S)-2-hydroxy-3-[(3-hydroxy-2,5-dimethylbenzoyl)amino]-4-phenylbutanoyl}-3,3-dimethyl-L-prolinamide)

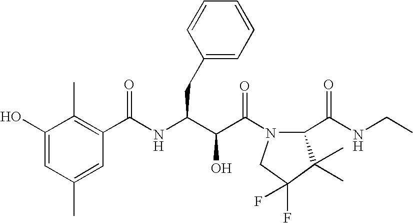

White solid: ¹H NMR (DMSO-d₆) δ 9.41 (s, 1H), 8.28 (d, 1H, J=8.3), 7.85 (t, 1H, J=5.6), 7.537.32 (m, 5H), 6.77 (s, 1H), 6.54 (s, 1H), 5.63 (d, 1H, J=6.2), 4.62-4.48 (m, 3H), 4.35 (s, 1H), 3.35-3.21 (m, 2H), 3.03 (d, 1H, J=12), 2.90-2.82 (m, 1H), 2.33 (s, 3H), 2.16 (s, 3H), 1.37 (s, 3H), 1.21 (s, 3H),1.19-1.16 (m, 3H), Anal. Calcd for C₂₈H₃₄F₂N₃O₅.0.5 H₂O C, 62.32; H, 6.54; N, 7.79. Found: C, 62.08; H, 6.79; N, 7.49.

### Example D18

### (S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide

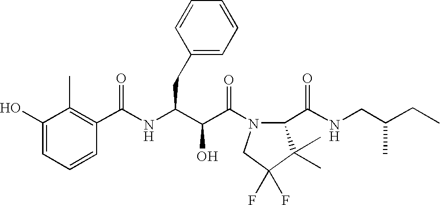

White solid: ¹H NMR (DMSO-d₆) δ 9.38(s, 1H), 8.17 (d, 1H, J=8.1), 7.90 (t, 1H, J=5.8), 7.37 (d, 1H, J=7.3), 7.26 (t, 1H, J=7.5), 7.17 (t, 1H, J=7.2), 6.95 (t, 1H, J=7.8), 6.78 (d, 1H, J=7.3), 6.54 (d, 1H, J=6.8), 5.48 (d, 1H, J=6.6), 4.46-4.26 (m, 4H), 3.08-2.66 (m, 4H), 1.81 (s, 3H), 1.53-1.31 (m, 3H), 1.21 (s, 3H), 1.18-1.06 (m, 2H), 1.04 (s, 3H), 0.85-0.82 (m, 6H); HRMS (ESI) m/z calcd for C₃₀H₄₀F₂N₃O₅ (M+H)⁺560.2936, found 560.2949; Calcd for C₃₀H₃₉F₂N₃O₅+0.1 eq of H₂O: C, 64.17; H, 7.04; N, 7.48. Found: C, 63.88; H, 7.22; N, 7.19.

### Example D19

### Preparation of 3-acetoxy-2,5-dimethyl-benzoic acid

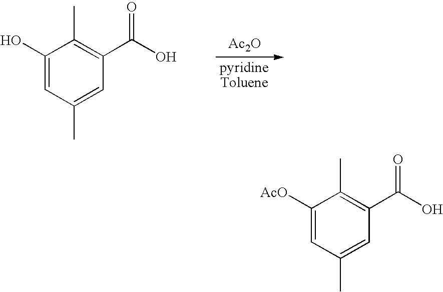

Pyridine (34.0 mL, 419 mmol) and acetic anhydride (150 mL, 1.59 mol) were sequentially added to a suspension of 3-hydroxy-2,5-dimethyl-benzoic acid (211 g, 1.27 mol) in toluene (1.05 L). The mixture was heated at 50° C. under argon for 6 h. Heating was discontinued and, while the mixture was still warm, n-heptane (2.10 L) was added. The mixture was allowed to cool and stir at ambient temperature overnight. The suspension was filtered, using n-heptane for rinsing, and the solid was dried in a vacuum oven at 50° C. to give 212 g (80.1%) of 3-acetoxy-2,5-dimethyl-benzoic acid as a pale yellow solid: m.p.=153-154 ° C.; ¹H NMR (300 MHz, CDCl₃) δ 11.5 (br s, 1H), 7.80 (s, 1H), 7.10 (s, 1H), 2.44 (s, 3H), 2.41 (s, 3H), 2.39 (s, 3H); ¹³C NMR (75 MHz, DMSO-d₆) δ 169.3, 168.8, 149.9, 136.3, 132.9, 128.4, 128.0, 126.3, 20.8, 20.5, 13.1; MS (Cl) m/z 209.0822 (209.0814 calcd for C₁₁H₁₃O₄, M+H⁺); elemental analysis calcd for C₁₁H₁₂O₄: C, 63.45; H, 5.81; found: C, 63.54; H, 5.88.

### Example D20

### Preparation of Acetic acid 3-chlorocarbonyl-2,5-dimethyl-phenyl ester

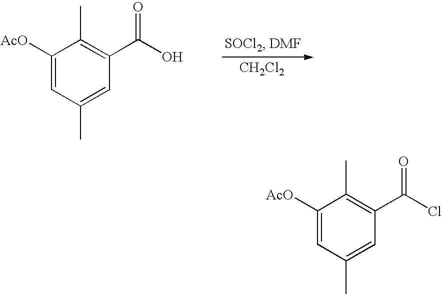

SOCl₂ (80.0 mL, 1.09 mol) was added to a suspension of 3-acetoxy-2,5-dimethyl-benzoic acid (206 g, 990 mmol), DMF (4.0 mL), and CH₂Cl₂ (1.03 L). The resulting mixture was stirred at ambient temperature for 1.5 h. n-Heptane (1.03 L) was added, followed by the slow addition of saturated aqueous NaHCO₃ (2.06 L), and the layers were then separated. The organic fraction was washed with saturated aqueous NaCl (1.00 L), dried over MgSO₄, filtered, and concentrated with a rotary evaporator to give 193 g (86.2%) of acetic acid 3-chlorocarbonyl-2,5-dimethyl-phenyl ester as a pale yellow solid: m.p.=52-54° C.; ¹H NMR (300 MHz, CDCl₃) δ 7.92 (s, 1H), 7.15 (s, 1H), 2.44 (s, 3H), 2.38 (s, 3H), 2.35 (s, 3H); ¹³C NMR (75 MHz, CDCl₃) δ 169.4, 167.7, 150.1, 137.3, 134.7, 132.0, 130.2, 129.1, 21.2, 21.1, 13.7; elemental analysis calcd for C₁₁H₁₁O₃Cl: C, 58.29; H, 4.89; found: C, 58.64; H, 4.89.

### Example D21

### Preparation of (2S,3S)-3-(3-Acetoxy-2,5-dimethyl-benzoylamino)-2-hydroxy-4-phenyl-butyric acid

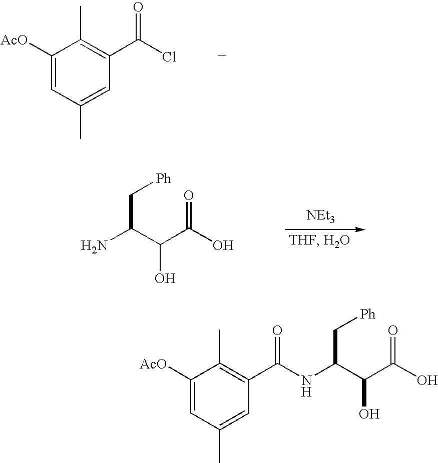

NEt₃ (265 mL, 1.88 mol) was added to a suspension of (2S,3S)-3-amino-2-hydroxy-4-phenyl-butyric acid (175 g, 896 mmol), tetrahydrofuran (875 mL), and H₂O (875 mL) at ambient temperature. The resulting solution was cooled to 0° C. A solution of acetic acid 3-chlorocarbonyl-2,5-dimethyl-phenyl ester (193 g, 854 mmol) and tetrahydrofuran (430 mL) was slowly added. One hour later, H₂O (225 mL) was added, followed by the slow addition of 3 N HCl (390 mL). The resulting mixture was allowed to slowly warm to ambient temperature with stirring overnight. The solid was filtered, using H₂O (430 mL) for rinsing. After drying in a vacuum oven at 50° C., 301 g (91.5%) of (2S,3S)-3-(3-acetoxy-2,5-dimethyl-benzoylamino)-2-hydroxy-4-phenyl-butyric acid was obtained as a white solid that was contaminated with ˜8 mol % Et₃N.HCl: m.p.=220-224° C.; ¹H NMR (300 MHz, DMSO-d₆) δ 12.65 (br s, 1H), 8.23 (d, J=9.0 Hz, 1H), 7.15-7.30 (m, 5H), 6.89 (s, 1H), 6.79 (s, 1H), 5.63 (br s, 1H), 4.39-4.50 (m, 1H), 4.07 (d, J=5.9 Hz, 1H), 2.91 (app dd, J=3.0, 14.0 Hz, 1H), 2.74 (app dd, J=11.1, 14.1 Hz, 1H), 2.27 (s, 3H), 1.24 (s, 3H), 1.72 (s, 3H) [characteristic resonances of Et₃N.HCl: δ 3.09 (q, J=7.3 Hz), 1.18 (t, J=7.3 Hz)]; ¹³C NMR (75 MHz, DMSO-d₆) δ 174.4, 169.2, 168.2, 149.4, 139.4, 135.9, 129.5, 128.3, 126.3, 125.6, 124.7, 123.5, 73.2, 53.5, 35.4, 20.8, 20.6, 12.2 [characteristic resonances of Et₃N.HCl: δ 45.9, 8.8]; MS (Cl) m/z 386.1600 (386.1604 calcd for C₂₁H₂₄NO₆, M+H⁺); elemental analysis calcd for C₂₁H₂₃NO₆.0.08 Et₃N.HCl: C, 65.08; H, 6.17; N, 3.82; found: C, 64.88; H, 6.10; N, 3.68.

### Example D22

### Preparation of (2S,3S)-2-Acetoxy-3-(3-acetoxy-2,5-dimethyl-benzoylamino)-4-phenyl-butyric acid

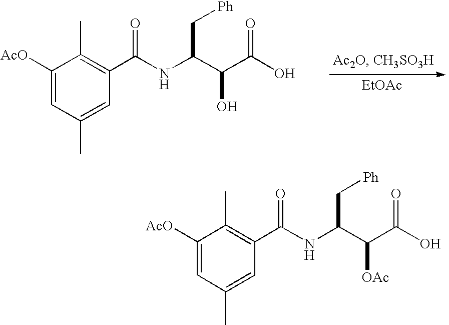

Methanesulfonic acid (16.5 mL, 253 mmol) and acetic anhydride (91.0 mL, 960 mmol) were sequentially added to a suspension of (2S,3S)-3-(3-acetoxy-2,5-dimethyl-benzoylamino)-2-hydroxy-4-phenyl-butyric acid (296 g, 768 mmol) in ethyl acetate (3.00 L) at ambient temperature. The mixture was heated at 75° C. for 2 h, and the resulting solution was then cooled to ambient temperature. The solution was sequentially washed with H₂O (2.0 L), half-saturated aqueous NaCl (2.0 L), and then with saturated aqueous NaCl (1.0 L). The resulting organic fraction was concentrated to approximately half volume by distillation at one atmosphere. Heating was discontinued and the solution was allowed to cool to ambient temperature to give a suspension. n-Heptane (3.0 L) was added and the suspension stirred at ambient temperature overnight. The solid was filtered, using 1:2 ethyl acetate/n-heptane (1.5 L) for rinsing. After drying in a vacuum oven at 50° C., 316 g (96.3%) of (2S,3S)-2-acetoxy-3-(3-acetoxy-2,5-dimethyl-benzoylamino)-4-phenyl-butyric acid was obtained as a white solid: m.p.=185-186° C.; ¹H NMR (300 MHz, DMSO-d₆) δ 13.3 (s, 1H), 8.49 (d, J=8.8 Hz, 1H), 7.19-7.34 (m, 5H), 6.91 (s, 1H), 6.71 (s, 1H), 5.11 (d, J=5.0 Hz, 1H), 4.61-4.72 (m, 1H), 2.79-2.90 (m, 2H), 2.27 (s, 3H), 2.24 (s, 3H), 2.14 (s, 3H), 1.73 (s, 3H); ¹³C NMR (75 MHz, DMSO-d₆) δ 170.3, 169.7, 169.2, 168.5, 149.4, 139.1, 138.5, 136.1, 129.4, 128.5, 126.6, 125.4, 124.7, 123.8, 73.9, 51.1, 35.2, 20.9, 20.8, 20.6, 12.1; MS (Cl) m/z 428.1713 (428.1709 calcd for C₂₃H₂₆NO₇, M+H⁺); elemental analysis calcd for C₂₃H₂₅NO₇: C, 64.63; H, 5.90; N, 3.28; found: C, 64.79; H, 5.96; N, 3.15.

### Example D23

### Preparation of (2S)-4,4-Difluoro-3,3-dimethyl-pyrrolidine-2-carboxylic acid ethylamide; hydrochloride

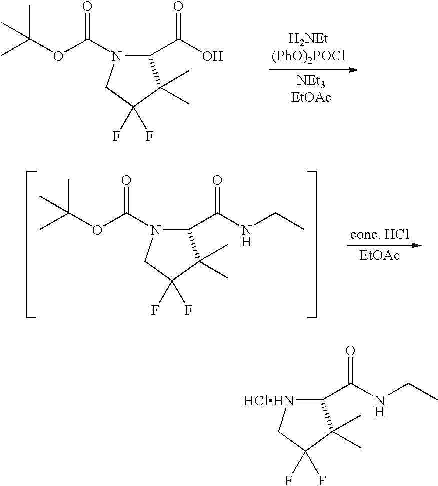

Chlorodiphenylphosphate (38.4 mL, 185 mmol) was added to a solution of (2S)-4,4-difluoro-3,3-dimethyl-pyrrolidine-1,2-dicarboxylic acid 1-tert-butyl ester (48.8 g, 175 mmol) in ethyl acetate (490 mL) at ambient temperature. The solution was cooled to 0° C., and NEt₃ (51.0 mL, 367 mmol) was added dropwise. Cooling was discontinued and the resulting suspension was allowed to warm to ambient temperature and stir for 1 h. The suspension was cooled to 0° C., and H₂NEt (96.0 mL of a 2.0 M solution in tetrahydrofuran, 192 mmol) was slowly added. The resulting mixture was allowed to warm to ambient temperature and stir for 2 h. 20% Aqueous citric acid (490 mL) was added and the layers were then separated. The aqueous fraction was extracted with ethyl acetate (125 mL). The combined organic fractions were washed with saturated aqueous NaHCO₃ (490 mL), and the layers were then separated. The aqueous fraction was extracted with ethyl acetate (125 mL). The combined organic fractions were washed with saturated aqueous NaCl (250 mL), dried over MgSO₄, and then concentrated to a volume of 500 mL using a rotary evaporator. Concentrated HCl (61.0 mL, 734 mmol) was added, and the solution was stirred at ambient temperature overnight. The resulting suspension was dried azeotropically with ethyl acetate (3×250 mL) by distillation at one atmosphere. The resulting suspension was cooled to ambient temperature, and was then filtered, using ethyl acetate (100 mL) for rinsing. After drying under vacuum at ambient temperature, 37.4 g (88.2%) of (2S)-4,4-difluoro-3,3-dimethyl-pyrrolidine-2-carboxylic acid ethylamide; hydrochloride was obtained as a white solid: m.p.=238-239° C. (decomp.); ¹H NMR (300 MHz, DMSO-d₆) δ 10.3 (br s, 2H), 8.70 (t, J=5.3 Hz, 1H), 4.08 (s, 1H), 3.71-3.80 (m, 2H), 3.08-3.34 (m, 2H), 1.21 (app d, J=2.2 Hz, 3H), 1.08 (t, J=7.2 Hz, 3H), 0.97 (app d, J=2.1Hz, 3H); ¹³C NMR (75 MHz, DMSO-d₆) δ 163.8, 128.1 (dd, J_{CF}=248.6, 255.5 Hz), 64.8, 48.1 (t, J_{CF}=33.7 Hz), 45.5 (t, J_{CF}=20.8 Hz), 34.3, 18.3 (d, J_{CF}=7.4 Hz), 17.4 (app dd, J_{CF}=2.1, 5.4 Hz), 14.8; MS (Cl) m/z 207.1317 (207.1309 calcd for C₉H₁₇N₂OF₂, M−HCl+H⁺); elemental analysis calcd for C₉H₁₇ClF₂N₂O: C, 44.54; H, 7.06; N, 11.54; F, 15.66; found: C, 44.40; H, 7.06; N, 11.65; F, 15.61.

### Example D24

### Preparation of Acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester

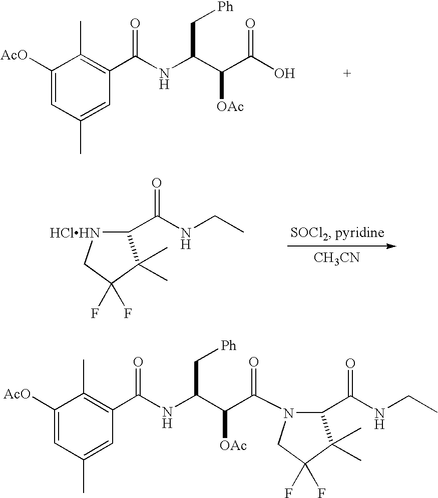

SOCl₂ (1.90 mL, 25.8 mmol) was added dropwise to a 0° C. solution of (2S,3S)-2-acetoxy-3-(3-acetoxy-2,5-dimethyl-benzoylamino)-4-phenyl-butyric acid (10.0 g, 23.5 mmol), pyridine (7.60 mL, 93.9 mmol), and CH₃CN (90.0 mL). The resulting solution was allowed to warm to ambient temperature for 1 h, then was cooled to 0° C. (2S)-4,4-Difluoro-3,3-dimethyl-pyrrolidine-2-carboxylic acid ethylamide; hydrochloride (5.71 g, 23.5 mmol) was added in one portion. The resulting solution was allowed to warm to ambient temperature and stir for 2.5 h. Saturated aqueous NaHCO₃ (110 mL) and methyl t-butyl ether (110 mL) were added, and the resulting layers were separated. The resulting organic fraction was sequentially washed with 20% aqueous citric acid (90 mL), saturated aqueous NaHCO₃ (70 mL), and saturated aqueous NaCl (70 mL). Activated charcoal (14 g) was added to the resulting organic fraction, and the mixture was stirred at ambient temperature overnight. The mixture was filtered on Celite, using methyl t-butyl ether for rinsing. The filtrate was dried over MgSO₄, filtered, and concentrated to a volume of ˜90 mL using a rotary evaporator. This solution of crude acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester was carried directly to the next step. Analytical data was obtained by concentrating a sample of this solution: m.p.=88-93° C.; ¹H NMR (300 MHz, DMSO-d₆) displayed a ˜10:1 mixture of rotamers. Major rotamer resonances: δ 8.58 (d, J=8.2 Hz, 1H), 8.02 (t, J=7.5 Hz, 1H), 7.18-7.42 (m, 5H), 6.92 (s, 1H), 6.84 (s, 1H), 5.34 (d, J=3.2 Hz, 1H), 4.41-4.66 (m, 2H), 4.19-4.32 (m, 2H), 3.03-3.26 (m, 2H), 2.95 (app dd, J=2.4, 13.8 Hz, 1H), 2.78 (app dd, J=11.7, 13.8 Hz, 1H), 2.27 (s, 3H), 2.25 (s, 3H), 1.73 (s, 3H), 1.22 (br s, 3H), 1.07 (br s, 3H), 1.04 (t, J=7.2 Hz, 3H) [characteristic minor rotamer resonances: δ 7.76-7.87 (m), 6.72 (s), 5.46 (d, J=3.7 Hz), 2.07 (s), 1.79 (s)]; ¹³C NMR (75 MHz, DMSO-d₆) displayed a ˜10:1 mixture of rotamers. Major rotamer resonances: δ 170.5, 169.2, 169.0, 166.8, 166.7, 149.4, 139.1, 138.8, 136.1, 129.7, 128.3, 127.8 (dd, J_{CF}=251.2, 254.9 Hz), 126.5, 125.7, 124.7, 123.9, 73.3, 68.2, 51.4, 43.9 (t, J_{CF}=20.5 Hz), 33.8, 33.4, 22.0 (d, J_{CF}=6.0 Hz), 20.8, 20.5, 17.6 (d, J_{CF}=7.0 Hz),15.0, 12.2 [characteristic minor rotamer resonances: δ 169.5, 168.9, 167.0, 149.5, 138.7, 129.3, 128.5, 125.4, 124.8, 124.2, 34.1, 21.2, 14.7]; MS (Cl) m/z 616.2859 (616.2834 calcd for C₃₂H40N₃O₇F₂, M+H⁺); elemental analysis calcd for C₃₂H₃₉F₂N₃O₇: C, 62.43; H, 6.38; N, 6.83; F, 6.17; found: C, 62.08; H, 6.68; N, 6.53; F, 5.85.

### Example D25

### Preparation of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide

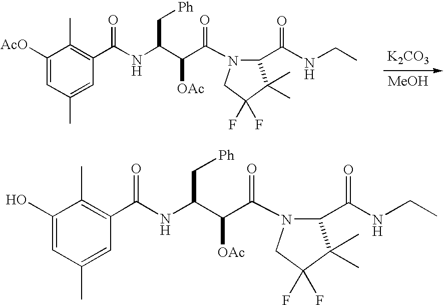

Methanol (30.0 mL) and K₂CO₃ (7.16 g, 51.7 mmol) were added to the methyl t-butyl ether solution of acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester (from above) at ambient temperature. After stirring for 2 h, the resulting yellow solution was diluted with ethyl acetate (140 mL), 1 N HCl (50 mL), and 0.5 N HCl (140 mL), and the layers were then separated. The resulting organic fraction was sequentially washed with saturated aqueous NaHCO₃ (90 mL), 0.5 N HCl (70 mL), H₂O (140 mL), and saturated aqueous NaCl (70 mL). The organic fraction was then concentrated to a volume of ˜100 mL by distillation at one atmosphere, and the resulting solution was then cooled to ambient temperature. Diisopropyl ether (190 mL) was slowly added, and the resulting crystalline suspension was stirred overnight at ambient temperature. The suspension was filtered, using diisopropyl ether (50 mL) for rinsing. After drying under vacuum, 9.88 g (79.1%) of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide was obtained as a white solid: m.p.=208-214° C.; ¹H NMR (300 MHz, DMSO-d₆) displayed a ˜9:1 mixture of rotamers. Major rotamer resonances: δ 9.21 (s, 1H), 8.07 (d, J=8.2 Hz, 1H), 7.90 (t, J=5.5 Hz, 1H), 7.15-7.39 (m, 5H), 6.62 (s, 1H), 6.40 (s, 1H), 5.45 (d, J=6.3 Hz, 1H), 3.95-4.50 (m, 5H), 3.02-3.22 (m, 2H), 2.89 (app dd, J=2.0, 13.5 Hz, 1H), 2.72 (app dd, J=10.4, 13.4 Hz, 1H), 2.17 (s, 3H), 1.78 (s, 3H), 1.22 (s, 3H), 1.05 (s, 3H), 1.03 (t, J=7.2 Hz, 3H) [characteristic minor rotamer resonances: δ 6.15 (d, J=8.7 Hz), 7.85 (t, J=5.7 Hz), 6.34 (s), 5.31 (d, J=7.6 Hz), 4.73 (s), 1.81 (s); ¹³C NMR (75 MHz, DMSO-d₆) displayed a ˜9:1 mixture of rotamers. Major rotamer resonances: δ 171.0, 169.6, 167.2, 155.5, 139.7, 139.1, 135.1, 129.8, 128.2, 128.1 (dd, J_{CF}=251.4, 254.0 Hz), 126.2, 118.7, 118.6, 116.2, 72.8, 68.5, 53.1, 51.5 (t, J_{CF}=32.0 Hz), 43.7 (t, J_{CF}=20.5 Hz), 34.2, 33.8, 22.5 (d, J_{CF}=4.7 Hz), 20.9, 17.4 (d, J_{CF}=7.3 Hz), 15.1, 12.2 [characteristic minor rotamer resonances: δ 171.8, 169.7, 168.0, 138.8, 129.5, 23.1, 14.9; MS (Cl) m/z 532.2614 (532.2623 calcd for C₂₈H₃₆N₃O₅F₂, M+H⁺); elemental analysis calcd for C₂₈H₃₅F₂N₃O₅: C, 63.26; H, 6.64; N, 7.90; F, 7.15; found: C, 63.20; H, 6.67; N, 7.87; F, 7.07. While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

| Example No. | Ave. Ki (nM) | Ave. EC50 |
|---|---|---|
| D10 | <0.1 | 0.016 |
| D11 | <0.1 | 0.013 |
| D12 | 0.7 | 0.016 |
| D13 | <0.1 | 0.017 |
| D14 | <0.1 | 0.009 |
| D15 | <0.1 | 0.011 |
| D16 | <0.1 | 0.018 |
| D17 | <0.1 | 0.066 |
| D18 | <0.1 | 0.010 |

The examples and preparations provided above further illustrate and exemplify the compounds of the present invention and methods of preparing such compounds. It is to be understood that the scope of the present invention is not limited in any way by the scope of the following examples and preparations.

### Synthesis of P2

### Synthesis of 2,5-dimethyl-3-hydroxybenzoic acid 4

The synthesis of 2,5-dimethyl-3-hydroxybenzoic acid 4 was prepared following the procedure published reports from Fujiwara A. N.; Anton, E. M. Can. J. Chem. 1970, 48, 1346-1349 and Charlesworth, E. H.; Levene, L. Can. J. Chem. 1963, 41, 1071-1077. Starting with 2,5-dimethylbenzoic acid 1, sulfonylation was done with with fuming H₂SO₄ at 110° C. A 1:1 mixture of regioisomers of the sodium sulfate salt 2:3 was observed, and this was taken on to the next step without further purification. Hydrolysis of this mixture with KOH followed by multiple recrystallizations with 4:1-CHCl₃: EtOAc afforded the desired product 4 in an overall yield over two steps of ˜30%.

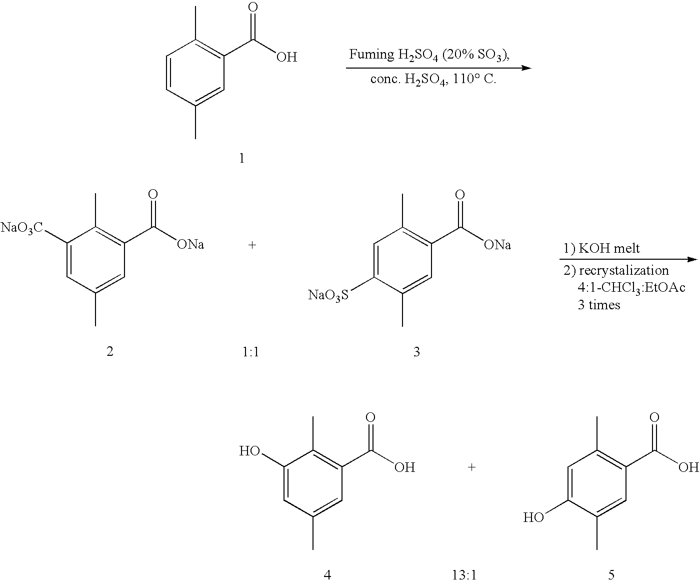

### Synthesis of acetic acid 3-chlorocarbonyl-2,4-dimethyl-phenyl ester 7

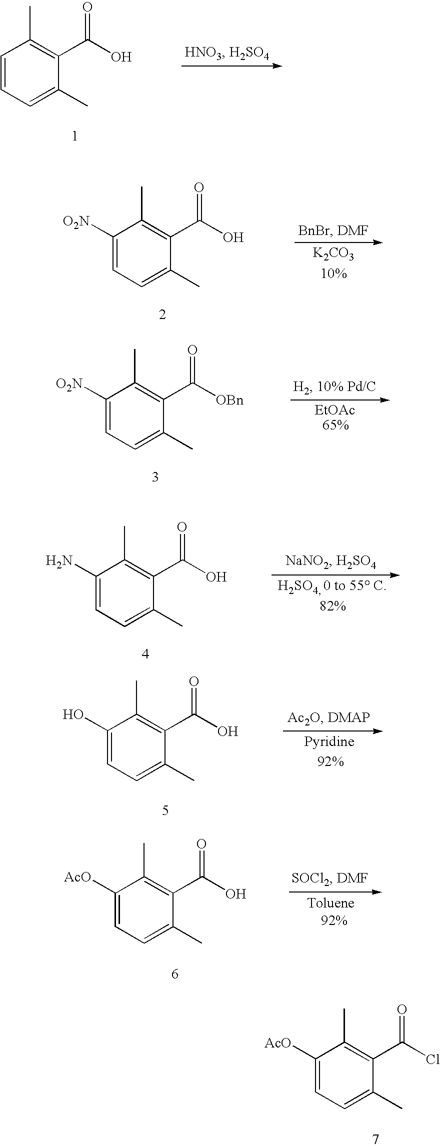

### 2,6-Dimethyl-3-nitro-benzoic acid 2

2,6-Dimethylbenzoic acid 1 (10.0 g, 66.6 mmol) was treated with H₂SO₄ (67 mL) and heated until the mixture became homogeneous. This was then cooled to 0° C. and then HNO₃ (42 mL, 66.6 mmol) was added dropwise over 5 min. The mixture was maintained at 0° C. for 15 min. The reaction mixture was added to ice-water (300 mL) and then extracted with EtOAc (2×100 mL). The organic layers were combined, washed with Brine (100 mL) and dried (MgSO₄). The product 2 was isolated as a mixture of regioisomers (crude ˜14.2 9) and was taken to the next step without further purification.

### 2,6-Dimethyl-3-nitro-benzoic acid benzyl ester 3

Crude regioisomers of acid 2 (13.0 g, 66.6 mmol) was treated with BnBr (9.5 mL, 79.9 mmol) and K₂CO₃ (18.4 g, 188 mmol), and DMF (100 mL) at 25° C. over 30 min. This was then diluted with EtOAc (100 mL), and the organic layer was washed with H₂O (3×100 mL), Brine (100 mL) and dried (MgSO₄). The crude product was purified by SiO₂ column chromatography (10:1-Hex:Et₂O) to give desired nitro benzyl ester 3 (4.20 g, 45% over two steps) which was purified by recrystallization with hexanes and ether.

### 3-Amino-2,6-dimethyl-benzoic acid 4

The nitro benzyl ester intermediate 3 (4.20 g, 14.7 mmol) was treated with 10% Pd/C (420 mg, 10% wt) and EtOAc (100 mL) under 35 psi of H₂ at 25° C. This heterogeneous mixture was filtered through Celite and washed with EtOAc (50 mL) and DCM (50 mL), followed by MeOH (1 L). The filtrate was concentrated to give the desired amino benzoic acid 4 (1.58 g, 65%). ¹HNMR (300 MHz, Acetone-d₆) δ 11.3 (bs, 1H), 6.89-6.87 (m, 1H), 6.78-6.76 (m, 1H), 2.82 (brs, 2H), 2.21 (s, 3H), 2.18 (s, 3H) ppm.

### 3-Hydroxy-2,6-dimethyl-benzoic acid 5

Amino benzoic acid 4 (1.58 g, 9.56 mmol) was treated with H₂O (65 mL) and concentrated H₂SO₄ (8 mL) at 0° C. A solution of NaNO₂ (8.58 g, 12.4 mmol) and H₂O (12 mL) was added to the amine solution dropwise over 10 min and was maintained at 0° C. for an additional 15 min. This was then added a pre-mix solution of H₂SO₄ (25 mL) and H₂O (120 mL), and the resulting mixture was heated to 55° C. for 30 min, 85° C. for 1.5 h, and allowed to cool to 25° C. over 12 h. This was then added EtOAc (100 mL) and H₂O (100 mL), and the layers were separated. The aqueous layer was extracted with EtOAc (2×100 mL), and the organic layers were combined, washed with Brine (100 mL) and dried (MgSO₄). The crude product was purified by SiO₂ column chromatography (0.5:5:94.5-AcOH:MeOH: DCM) to give the desired phenol 5 (1.30 g, 82%).

### 3-Acetoxy-2,6-dimethyl-benzoic acid 6

Phenol 5 (1.30 g, 7.82 mmol) was treated with Ac₂O (0.74 ml, 7.82 mmol), DMAP (96 mg, 0.78 mmol), pyridine (1.26 mL, 15.6 mmol) and DCM (26 mL) at 25°C. over 2 h. This was then added EtOAc (100 mL), and the organic layer was washed with aqueous HCl (1M, 3×100 mL), Brine (100 m) and dried (MgSO₄). This was then concentrated to give the acetyl product 6 (1.50 g, 92%), which was taken to the next step without further purification. ¹HNMR (300 MHz, Acetone-d₆) δ 11.6 (bs, 1H), 7.12-7.10 (m, 1H), 7.01-6.99 (m, 1H), 3.32 (s, 3H), 2.28 (s, 3H), 2.13 (s, 3H) ppm.

### Acetic acid 3-chlorocarbonyl-2,4-dimethyl-phenyl ester 7

The acid 6 (1.5 g, 7.20 mmol) was treated in DMF (0.011 mL, 0.144 mmol), toluene (20 mL) and SOCl₂ (0.59 mL, 8.07 mmol) at 25° C. and then heated to 65° C. for 3 h. This mixture was then allowed to cool to 25° C. and maintained over 12 h. The solution was then concentrated under vacuum to give the desired product acid chloride 7 (1.49 g, 92%) as a brown oil. This was taken on to the next step without further purification. ¹HNMR (300 MHz, Acetone-d₆) δ 7.25-7.14 (m, 2H), 2.38 (s, 3H), 2.31 (s, 3H), 2.19 (s, 3H) ppm.

### Synthesis of Indole-1,4-dicarboxylic acid 1-tert-butyl ester 3

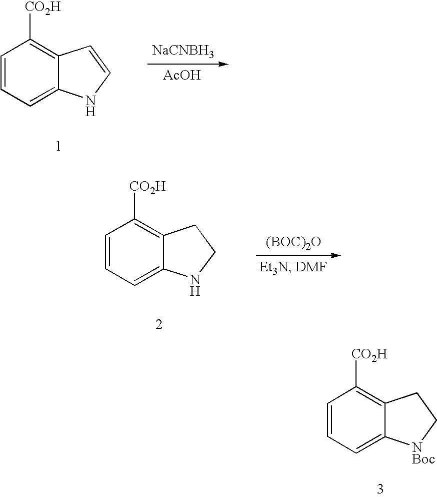

### 2,3-Dihydro-1H-indole-4-carboxylic acid 2

1H-indole-4-carboxylic acid 1 (1.5 g, 9.31 mmol) was added glacial AcOH (46 mL) and cooled to 0° C. Then NaCNBH₃ (5.85 g, 93.1 mmol) was added in five portions slowly over 30 min. This mixture was left to stir at 0° C. for 45 min and then allowed to warm to 25° C. over 5 h. Then H₂O (100 mL) was added and the aqueous layer was extracted with CH₂Cl₂ (2×50 mL), (4:1-CH₂Cl₂:i-Pr (4×50 mL), and dried (MgSO₄). This crude product was purified by SiO₂ column chromatography (2:1-EtOAc:Hex) to give pure reduce indole product 2 which was contaminated with excess AcOH but was pure enough to take on to the next step without further purification. ¹HNMR (300 MHz, CDCl₃) δ 7.39 (d, J=7.9 Hz, 1H), 7.09 (t, J=7.9 Hz, 1H), 6.82 (d, J=7.3 Hz, 1H), 3.60 (t, J=9.1 Hz, 1H), 3.47 (m, 1H), 3.40 (t, J=8.1Hz, 1H), 2.10 (AcOH, 3H), 1.20 (t, J=7.1 Hz, 1H) ppm.

### Indole-1,4-dicarboxylic acid 1-tert-butyl ester 3

2,3-Dihydro-1H-indole-4-carboxylic acid 2 (˜1.5 g, 9.2 mmol) was added DMF (11 mL), (BOC)₂O (2.0 g, 9.2 mmol), and Et₃N (3.8 mL, 27.6 mmol). This solution was left to stir at 25° C. over 12 h. This was then diluted with EtOAc (50 mL) and acidified with aqueous HCl (1M, 50 mL) and the layers were separated. The organic layer was washed with H₂O (2×50 mL), Brine (50 mL) and dried (MgSO₄). The crude product was purified by SiO₂ column chromatography (2:1-EtOAc:Hex) to give pure BOC protected indole product 3 (0.92 g, 38% over two step from X). ¹HNMR (300 MHz, CDCl₃) δ 8.12 (bs, 1H), 7.66 (d, J=7.9 Hz, 1H), 7.09 (m, 1H), 4.01 (d, J=8.6 Hz, 1H), 3.47 (t, J=5.8 Hz, 1H), 2.90 (m, 1H), 1.56 (s, 9H), 1.22 (t, J=7.1Hz, 1H) ppm.

### Synthesis of 3-Cyclopropylmethoxy-benzylamine 4

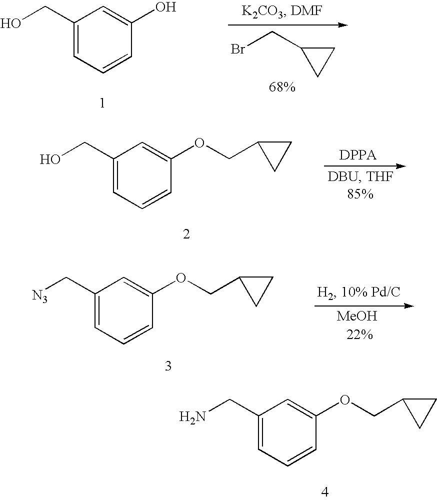

### (3-Cyclopropylmethoxy-phenyl)-methanol 2

3-Hydroxybenzyl alcohol 1 (4.6 g, 37.0 mmol) was added to DMF (200 mL) followed by cyclopropylmethyl bromide (5.00 g, 37.0 mmol), and K₂CO₃ (25.5 g, 185.2 mmol). This heterogeneous mixture was left to stir at 25° C. over 48 h. This was then diluted with H₂O (100 mL), washed with aqueous NaOH (1M, 100 mL), H₂O (3×100 mL), Brine (100 mL) and dried (MgSO₄). The desired cyclopropyl ether 2 (4.47 g , 68%) was taken on to the next step without further purification. ¹HNMR (300 MHz, DMSO-d₆) δ 7.19 (t, J=7.6 Hz, 1H), 6.85-6.74 (m, 3H), 5.14 (t, J=5.8 Hz, 1H), 4.45-4.38 (m, 2H), 3.78 (d, J=7.0 Hz, 1H), 1.20 (m, 1H), 0.57-0.52 (m, 2H), 0.31-0.28 (m, 2H) ppm.

### -Azidomethyl-3-cyclopropylmethoxy-benzene 3

Intermediate 2 (3.88 g, 21.8 mmol) was added to THF (73 mL) and the solution was cooled to 0° C. Then DPPA (5.63 mL, 26.1 mmol) and DBU (3.90 mL, 26.1 mmol) were added and the mixture was maintained at 0° C. for 30 min and allowed to warm to 25° C. over 12 h. The solvent was evaporated, and the mixture was purified by SiO₂ column chromatography (20:1-EtOAc:Hex) to give the product 3 (3.75 g, 85%) as a clear liquid.

¹HNMR (300 MHz, DMSO-D-d₆) δ 7.28 (t, J=7.8 Hz, 1H), 6.91-6.88 (m, 3H), 4.38 (s, 2H), 3.81 (d, J=7.1Hz, 2), 1.34-1.17 (m, 1H), 0.56-0.53 (m, 2H), 0.32-0.29 (m, 2H) ppm.

### 3-Cyclopropylmethoxy-benzylamine 4

The azide intermediate 3 (3.75 g, 21.2 mmol) was added to MeOH (70 mL) and 10% Pd/C (375 mg, 10% wt) and left under 35 psi of H₂ at 25° C. over 1.5 h in a Parr Shaker. The mixture was filtered through Celite and washed with EtOAc (2×˜50 mL), and the resulting filtrate was concentrated. This crude product was purified by SiO₂ column chromatography (1:5:94-NH₄OH:MeOH:CH₂Cl₂) to give the product 4 (0.83 g, 22%). ¹HNMR (300 MHz, DMSO-D-d₆) δ 7.16 (t, J=7.9 Hz, 1H), 6.89-6.83 (m, 2H), 6.73-6.70 (m, 1H), 3.78 (d, J=6.8 Hz, 2H), 3.64 (s, 2H), 2.22-1.92 (bs, 2H), 1.23-1.17 (m, 1H), 0.55-0.53 (m, 2H), 0.30-0.28 (m, 2H) ppm.

### Synthesis of 3-difluoromethyl-2-methyl-benzoic acid 5

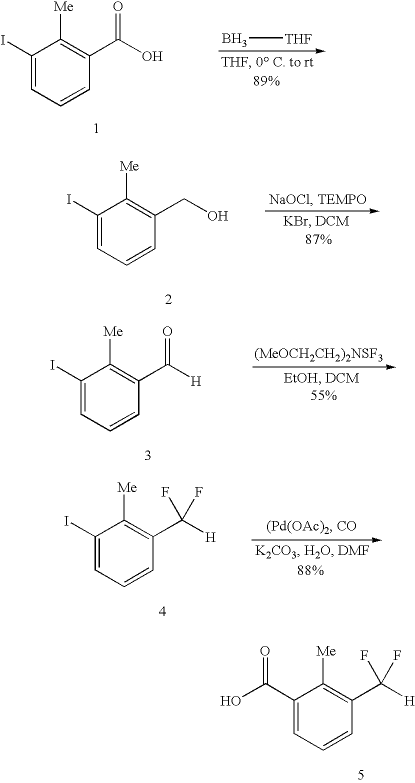

### (3-Iodo-2-methyl-phenyl)-methanol 2

3-iodo-2-methylbenzoic acid 1 (5.0 g, 19.1 mmol) was added to THF (25 mL) at 0° C. Then the BH₃-THF solution (1M, 48 mL, 47.7 mmol) was added. The solution was maintained at 0° C. and then allowed to warm to 25° C. over 12 h. The solvent was evaporated, and then the mixture was cooled to 0° C. The crude residue was partitioned with EtOAc (˜10 mL) and H₂O (˜10 mL). The layers were separated, and the organic layer was washed with aqueous HCl (1M, 20 mL), saturated NaHCO₃ (20 mL), Brine (20 mL), and dried (MgSO₄). The desired alcohol product 2 was isolated (4.23 g, 89%) as a foam and taken on to the next step without further purification.

### 3-iodo-2-methyl-benzaldehyde 3

Alcohol 2 was treated with DCM (12 mL), TEMPO (19 mg, 0.12 mmol), and KBr (0.71 g, 0.61 mmol) and cooled to 0° C. Then to this alcohol mixture was added a pre-mix solution of NaOCl (5 mL, 9.68 mmol), NaHCO₃ (0.60 g, 0.70 mmol), H₂O (5 mL) over 2 min. The heterogeneous mixture turned red and after stirring for 25 min, the layers were separated. The aqueous layer was extracted with DCM (3×50 mL), and the combined organic layers were washed with Brine (1×100 mL) and dried (MgSO₄). The crude product was purified by SiO₂ column chromatography (1:10-EtOAc:Hex) to give the aldehyde 3 (1.30 g, 87%).

¹HNMR (300 MHz, CDCl₃) δ 10.2 (s, 1H), 8.06 (d, J=7.9 Hz, 1H), 7.79 (d, J=6.6 Hz, 1H), 7.08 (t, J=7.9 Hz, 1H), 2.78 (s, 3H) ppm.

### C,C-Difluoro-C-(3-iodo-2-methyl-phenyl)-methylamine 4

Aldehyde 3 (1.40 g, 1.28 mmol) was added to a polypropylene tube with DCM (1.5 mL) at 25° C. To this solution was added a solution of bis-(methoxy ethyl)amino sulfurtrifluoride (1.8 mL, 9.67 mmol) in DCM (1 mL). Then EtOH (0.066 mL, 1.14 mmol) was added to the aldehyde mixture, and this was left to stir at 25° C. for 12 h. Additional bis-(methoxy ethyl)amino sulfurtrifluoride (0.50 mL) in DCM (0.5 mL) was added if starting aldehyde was still present by TLC analysis. This as then quenched with saturated NaHCO₃ (25 mL) slowly, and the aqueous layer was extracted with DCM (3×25 mL). The organic layers were combined and dried (MgSO₄). The crude product was then purified by SiO₂ column chromatography (3:97-EtOAc:Hex) to give the desired methylene difluoro product 4 (0.84 g, 55%). %). ¹HNMR (300 MHz, CDCl₃) δ 7.90-8.10 (m, 1H), 7.45-7.60 (m, 1H), 6.85-7.21 (m, 1H), 2.65 (s, 3H), 1.65 (s, 1H) ppm.

### 3-Difluoromethyl-2-methyl-benzoic acid 5

The difluoro intermediate 4 (100 mg, 0.37 mmol) was treaded with DMF (3 mL), H₂O (3 mL), and K₂CO₃ (309 mg, 2.24 mmol) under 1 atm of CO (balloon) at 25° C. Then Pd(OAc)₂ (0.8 mg, 0.0037 mmol) was added , and the mixture was left to stir over 12 h at 25° C. This was diluted with EtOAc (10 mL) and H₂O (10 mL), and the solution was acidified with aqueous HCl (1M) until pH˜3. The layers were separated, and the organic layer was extracted with Brine (20 mL) and dried (MgSO₄). The solvent was evaporated to give acid 5 (61 mg, 88%) which was taken on to the next step without further purification. (300 MHz, DMSO-d₆) δ 13.1 (bs, 1H), 7.84 (d, J=7.6 Hz, 1H), 7.68 (d, J=7.4 Hz, 1H), 7.41 (t, J=7.9 Hz, 1H), 7.38 (app s, 1H), 2.51 (s, 3H) ppm.

### Synthesis of P1

### Synthesis of (3S)-tert-Butoxycarbonylamino-4-(3-fluoro-phenyl)-(2S)-hydroxy-butyric acid 9

The protected amino acid 9 was prepared following the scheme below. The same procedure was used to prepare (3S)-tert-Butoxycarbonylamino-4-(3-trifluoromethyl-phenyl)-(2S)-hydroxy-butyric acid 10.

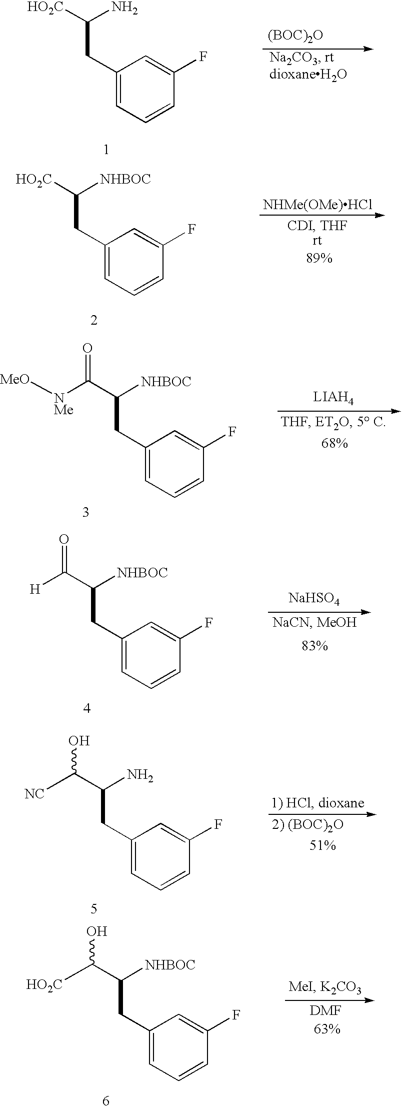

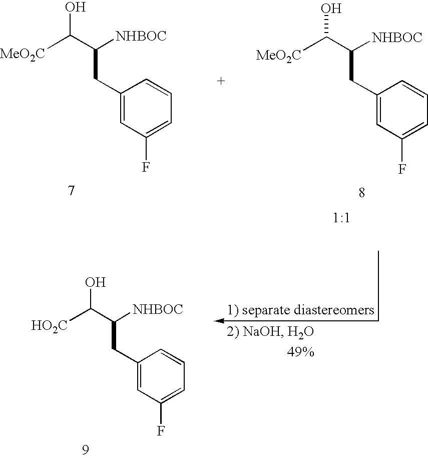

### (2S)-tert-Butoxycarbonylamino-3-(3-fluoro-phenyl)-propionic acid 2

A mixture of BOC-L-3-fluorophenylalanine 1 (20 g, 109 mmol) in water was treated with sodium carbonate (16.2 g, 15.3 mmol) in H₂O (40 mL). 1,4-Dioxane (100 mL) was added, and the mixture cooled to 0° C. The BOC₂O (28.6 g, 120 mmol) was added in one portion, and the mixture was maintained for 5 h at 25° C. The solvent was evaporated and H₂O (125 mL) was added. The aqueous layer was washed with diethyl ether (2×100 mL). The ether layers were discarded, and the aqueous layer was acidified with a 10% citric acid solution. The mixture was then extracted with EtOAc (2×150 mL). The organic layers were combined, washed with H₂O (2×150 mL), Brine (150 mL), dried (Na₂SO₄), filtered and evaporated to give the desire crude product 2 as a clear viscous oil 30.9 g, 100%,) which slowly solidified to a white solid at rt. ¹H NMR (300 MHz, CDCl₃) δ 7.33-7.26 (m, 1H), 7.00-6.91 (m, 3H), 4.96 (s, 1H), 4.62 (bs, 1H), 3.23 (dd, J=14, 5.3 Hz, 2H), 1.44 (s, 9H) ppm; Anal Calcd for C₁₄H₁₈NO₄F: C, 59.36; H, 6.40; N, 4.94. Found: C, 59.29; H, 6.34; N, 4.90.

### (1-(N-Methoxy-N-methyl)-carbamoyl)-(2S)-(3-fluoro-phenyl)-ethyl-carbamic acid tert-butyl ester 3

Amino acid 2 (30.9 g, 109 mmol) was added THF (180 mL) and stirred until the solution was homogeneous. Carbonyl diimidazole (21.2 g, 131 mmol) was added slowly. Gas evolution was observed and the solution became a yellow color. This solution was maintained at 25° C. for 45 min and DMF (64 mL), N,O-dimethylhydroxylamine hydrochloride (11.7 g, 120 mmol) and Hunig base (19.8 mL, 120 mmol) were added. The solution was left to stir at 25° C. for 2 h 15 min and the solvents were evaporated under vacuum. The oily residue was partitioned in EtOAc (300 mL) and washed with H₂O (500 mL), 10% aqueous citric acid (2×150 mL), H₂O (500 mL), saturated aqueous Na₂CO₃ (200 mL), and Brine (200 mL) followed by drying (Na₂SO₄). Filtration and evaporation of the organic solution provided the Weinreb amide intermediate 3 (31.6 g, 89%) which was taken on to the next step without further purification. ¹H NMR (300 MHz, CDCl₃) δ 7.29-7.22 (m,1H), 6.98-6.89 (m, 3H), 5.20 (bs, 1H), 4.96 (bs, 1H), 3.72 (s, 3H), 3.19 (s, 3H), 3.07 (dd, J=13.6 , 5.9 Hz, 2H,), 1.41 (s, 9H) ppm; Anal Calcd for C₁₆H₂₃N₂O₄F: C, 58.88; H, 7.10; N,8.58. Found: C, 58.89; H, 7.19; N, 8.71.

### [(2S)-(3-Fluoro-phenyl)-1-formyl-ethyl]-carbamic acid tert-butyl ester 4

In a 1-L 3-neck flask equipped with septum, stopper, and thermometer was added a solution of LiAlH₄ (1M in Et₂O, 106 mL) and cooled to 0° C. A solution of Weinreb amide 3 (31.6 g) in THF (150 mL) was cannulated into the reaction flask while the temperature was maintained below 5° C. during the addition. This took about 1 h to complete. The reaction mixture was then stirred for an additional 30 min, cooled back to 0° C., and then partitioned with EtOAc (60 mL) and 5% aqueous KHSO₄ (˜100 mL). Ethyl acetate (500 mL) was added, and the organic layer was extracted with 1N aqueous HCl (3×100 mL), H₂O (500 mL), Brine (200 mL), dried (Na₂SO₄). This was then filtered and evaporated to give a white solid which was shaken vigorously with n-heptane (200 mL), and filtered to give the pure aldehyde 4 as a white solid (17.5 g, 68%) which was used without further purification. ¹H NMR (300 MHz, CDCl₃) δ 9.65 (s, 1H), 7.33-7.26 (m, 1H), 7.01-6.89 (m, 3H), 5.06 (bs, 1H), 4.43 (broad m, 1H), 3.14 (m, 2H), 1.45 (s, 9H) ppm; Anal Calcd for C₁₄H₁₈NO₃F: C, 62.91; H, 6.79; N, 5.24. Found: C, 62.73; H, 6.66; N, 5.21.

### (3S)-Amino-(2R, 2S)-hydroxy-4-(3-fluoro-phenyl)-butyric acid 5

A solution of aldehyde 4 (17.5 g, 66 mmol) in MeOH (104 mL) was cooled to 0° C. followed by the addition of a solution of sodium bisulfite (7.6 g, 63.3 mmol) in H₂O (104 mL). The resulting mixture maintained for 5 h at 0° C. and was then left overnight in a freezer at 0° C. The solution was then treated with a solution of NaCN (3.9 g, 79.6 mmol) in H₂O (104 ml). Ethyl acetate was added (260 mL), and the mixture was stirred at 25° C. for 11 h. The organic layer was separated, dried (Na₂SO₄), filtered and evaporated to give the crude product cyanohydrins 5 as a 1:1 mixture of diastereomers that became a waxy solid (16.2 g, 83%) over time. This was taken on to the next step without further purification. LCMS (electrospray) m/z calcd for C₁₅H₂₀FN₂O₃(M+Na)⁺318.3, found 318.1.

### (3S)-tert-Butoxycarbonylamino-(2R,2S)-hydroxy-4-(3-fluoro-phenyl)-butyric acid 6

The cyanohydrin mixture 5 (16.2 g, 55 mmol) was dissolved in 1,4-dioxane (265 mL). Anisole (11 mL) was added and the mixture was cooled to 0° C. With vigorous stirring, concentrated HCl (˜12M, 265 mL) was added slowly. The mixture was then heated to reflux for 1 h followed by evaporation of the dioxane and most of the water under vacuum. Then aqueous NaOH (2M, 150 mL) was added, and the aqueous phase washed with Et₂O (3×200 mL). The organic layers were discarded, and the aqueous phase was treated with 1,4-dioxane (120 mL) followed by BOC₂O (15.8 g, 72 mmol) and stirred at room temperature for 3 h. The dioxane was evaporated, and the reaction mixture acidified with an aqueous solution of 10% citric acid followed by extraction with EtOAc (2×300 mL). The combined organic layers were washed with H₂O (300 mL), Brine (200 mL), dried (Na₂SO₄), filtered and evaporated to give the desired acid 6 as a mixture of diastereomers. The crude product became an orange solid (10.6 g, 61%) that was taken to the next step without further purification. ¹H NMR (300 MHz, DMSO-d₆) δ 7.35-7.25 (m, 2H), 7.06-6.96 (m, 6H), 6.76 (d, J=9.0 Hz, 1H), 6.43 (d, J=9.6 Hz, 1H), 4.02-3.89 (m, 4H), 3.57 (m, 2H), 2.83 (dd, J=13.4, 6.1, 2H), 1.28 (s, 9H), 1.26 (s, 9H) ppm.

### (3S)-tert-Butoxycarbonylamino-(2S)-hydroxy-4-(3-fluoro-phenyl)-butyric acid methyl ester 7 and (3S)-tert-Butoxycarbonylamino-(2R)-hydroxy-4-(3-fluoro-phenyl)-butyric acid methyl ester 8

To a solution of acid 6 (10.6 g, 33.8 mmol) in DMF (130 mL) was added K₂CO₃ (6.1 g, 44 mmol) followed by the addition of Mel (4.2 mL, 67.6 mmol). After stirring for 2 h at 25° C., the DMF was evaporated away in vacuo. EtOAc (300 mL) was then added to the mixture followed by extraction with H₂O (2×100 mL), saturated aqueous sodium thiosulfate solution (100 mL) and Brine (200 mL). The organic layer was then dried (Na₂SO₄), filtered and evaporated to afford the crude esters 7 and 8 as a diastereomeric mixture (9.55 g). Analysis by TLC (1:1 EtOAc-hexanes) shows the two diastereomers with the desired diastereomer 7 as the lower spot (lower Rf). The crude mixture of diastereomers were purified by column chromatography (2×); first using (1:1 EtOAc-hexanes), and then followed by (1:1 Et₂O-hexanes). The desired isomer 7 is a was isolated as a cream-colored solid (3.28 g), The undesired diastereomer 8 (3.68 g) was also isolated pure to give a total of 6.96 g recovery and 63% yield from methyl ester 6. (7) ¹H NMR (300 MHz, CDCl₃) □07.30-7.22 (m, 1H), 7.01-6.90 (m, 3H), 4.88 (d, J=8.2, 1H), 4.32 (m, 2H), 3.67 (s, 3H), 2.79 (t, J=6.9, 2H,), 1.40 (s, 9H). LCMS (electrospray) m/z calcd for C₁₆H₂₂FNO₅ (M+Na )⁺350.36, found 350.30. Compound (8) ¹H NMR (300 MHz, CDCl₃) δ 7.32-7.25 (m, 1H), 7.09-6.91 (m, 3H), 4.82 (d, J=9.8 Hz, 1H), 4.27 (dd, J=16.9, 7.6 Hz, 1H), 4.08 (d, J=3.2 Hz, 1H), 3.78 (s, 3H), 3.17 (d, J=4.5 Hz, 1H), 2.93(d, J=4.5 Hz, 1H), 1.40 (s, 9H) ppm. LCMS (electrospray) m/z calcd for C₁₆H₂₂FNO₅ (M+Na )⁺350.36, found 350.30.

### (3S)-tert-Butoxycarbonylamino-(2S)-hydroxy-4-(3-fluoro-phenyl)-butyric acid 9

A mixture of ester 7 (3.28 g, 10 mmol), 1,4-dioxane (63 mL), MeOH (42 mL) and a solution of aqueous NaOH (4M, 4 mL, 16 mmol) was stirred at 25° C. for 1.5 h. The solvents were evaporated in vacuo. Then 10% aqueous solution of citric acid (100 mL) and EtOAc (100 mL) was added, and the mixture left to stir. The layers were separated and the organic layer was washed with H₂O (100 mL), Brine (50 mL), dried (Na₂SO₄), filtered and evaporated to give the desired acid 9 as a cream-colored solid (3.06 g, 97%). This was taken to the next step without further purification. ¹H NMR (300 MHz, DMSO-d₆) δ 12.58 (br s, 1H), 7.33-7.26 (m, 1H), 7.02-6.97 (m, 3H), 6.78 (d, J=5.2 Hz, 1H,), 3.98 (d, J=5.5 Hz, 1H), 3.99-3.86 (m, 2H), 2.77-2.82 (m, 2H), 1.27 (s, 9H) ppm. LCMS (electrospray) m/z calcd for C₁₅H₂₀FNO₅(M+Na)⁺ 336.33, found 336.20.

### (3S)-tert-Butoxycarbonylamino-4-(trifluoromethyl-phenyl)-(2)-hydroxy-butyric acid 10

The synthesis was done the same manner as previously described.

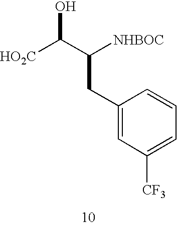

¹H NMR (300 MHz, DMSO-d₆) δ 12.58 (br s, 1H), 7.53-7.48 (m, 4H), 6.80 (d, J=8.9 Hz, 1H,), 5.61 (br s, 1H), 3.95-3.90 (m, 2H), 2.86-2.69 (m, 2H), 1.27 (s, 9H) ppm; Anal Calcd for C₁₆H₂₀NO₅F₃: C, 52.89; H, 5.55; N, 3.86. Found: C, 52.92; H, 5.50; N, 3.83.

### Synthesis of P1′

### Synthesis of 3,3-dimethyl-4-methylene-pyrrolidine-1,2-dicarboxylic acid 1-tert-butyl ester 2

Methyl triphenylphosphonium bromide (4.85 g, 13.6 mmol) in THF at 0° C. was added LHMDS (1 M solution in THF, 80 mL, 80 mmol) slowly. A light orange color was observed. This was maintained at 0° C. for 20 min and then a solution of racemic ketone F3 (1.2 g, 4.24 mmol) and THF (12 mL) was added. The solution remained orange in color. The ice-bath was removed and the solution was allowed to warm to 25° C. for 3 h and then heated at 35° C. for 1 h. This was then quenched with saturated aqueous NaHCO₃ (40 mL) and EtOAc (400 mL). The layers were separated, the organic phase was washed with saturated aqueous NaHCO₃ (3×100 mL) and dried (MgSO₄). The crude product was purified by SiO₂ column chromatography (1:4-EtOAc:Hex) to give the desired product 0.52 g of 1 in 46% yield:

¹H NMR (300 MHz, CDCl₃) δ 4.88 (m, 2H), 4.17 (m, 2H), 4.11 (s, 0.4H), 4.00 (s, 0.5H), 3.68 (s, 3H), 1.45 (s, 4H), 1.40 (s, 5H), 1.24 (s, 3H), 1.08 (s, 3H) ppm.

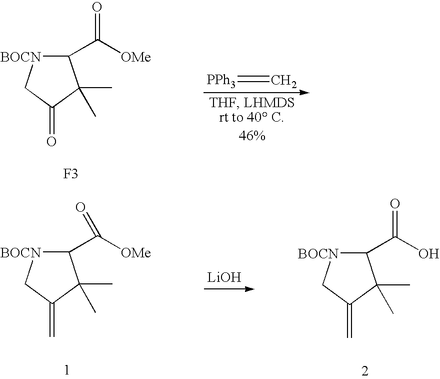

### 3,3-Dimethyl-4-methylene-pyrrolidine-1,2-dicarboxylic acid 1-tert-butyl ester 2-methyl ester 2

Ester 1 (380 mg, 1.41 mmol) in LiOH (68 mg, 2.82 mmol) in THF (10 mL) was left to stir over 12 h at 25° C. This was then quenched with 5% aqueous citric acid (50 mL) and extracted with EtOAc (50 mL). The layers were separated, and the organic layers was washed with Brine (50 mL) and dried (MgSO₄) and concentrated to an oil. This crude acid 2 (360 mg, quantitative) was taken to the next step without further purification: ¹H NMR (300 MHz, CDCl₃) δ 4.88-4.94 (m, 2H), 4.00-4.18 (m, 3H), 1.46 (s, 4H), 1.41 (s, 5H), 1.26 (s, 4H), 1.24 (s, 2H) ppm.

### Synthesis of 1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide: Starting with 1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide

(100 mg, 0.20 mmol) in EtOAc (30 mL) under Ar was added 10% Pd/C (˜20 mg) at 25° C. and left under a 1 atm of H₂ over 12 h. This was then filtered through Celite and rinsed with EtOAc. This was then concentrated to give a product as a white solid (65 mg, 64%).

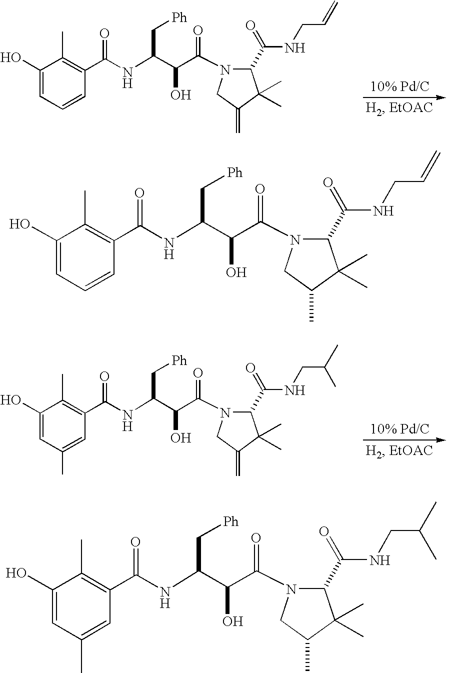

The synthesis of 1-[(2S,3S)-2-hydroxy-3-(3-hydroxy-2-methyl-benzoylamino)-4-phenyl-butyryl]-(4R)-3,3,4-trimethyl-pyrrolidine-2-carboxylic acid propylamide was synthesized in the same manner.

### Synthesis of (4R)-hydroxy-3,3,4-trimethyl-pyrrolidine-1,2-dicarboxylic acid 1-tert-butyl ester (2S)-methyl ester 1

Enantiopure ketone F3 (2.00 g, 7.30 mmol) in Et₂O (73 mL) at −78° C. was added a solution of MeMgBr (3 M in Et₂O, 3.70 mL, 10.9 mmol). A white slurry mixture was observed, and the progress of the reaction was followed by TLC (1:1-EtOAc-Hex). This was then allowed to warm to −40° C. after 15 min and then to 0° C. over an additional 30 min. This was then quenched with saturated aqueous ammonium chloride (30 mL), Brine (100 mL), and EtOAc (30 mL). The layers were separated and the aqueous layer was extracted with EtOAc (3×50 mL). The organic layers were combined and dried (Na₂SO₄). The crude product 1 was purified by SiO₂ column chromatography (1:4-EtOAc:Hex) to give the pure desired alcohol 1 (1.30 g, 62%): ¹H NMR (300 MHz, DMSO-d₆) δ 4.20 (s, 0.5H), 4.00 (s, 0.5H), 3.93 (s, 0.5H), 3.91 (s, 0.5H), 3.79 (s, 1.5H), 3.77 (s, 1.5H), 3.71 (d, J=11.6 Hz, 0.5H), 3.63 (d, J=11.4 Hz, 0.5H), 3.50 (d, J=5.3 Hz, 0.5H), 3.47 (d, J=5.1Hz, 0.5H), 1.44 (s, 4.5H), 1.39 (s, 4.5H), 1.15 (s, 1.5H), 1.14 (s, 1.5H), 1.10 (s, 3H), 0.99 (s, 1.5H), 0.98 (s, 1.5H) ppm.

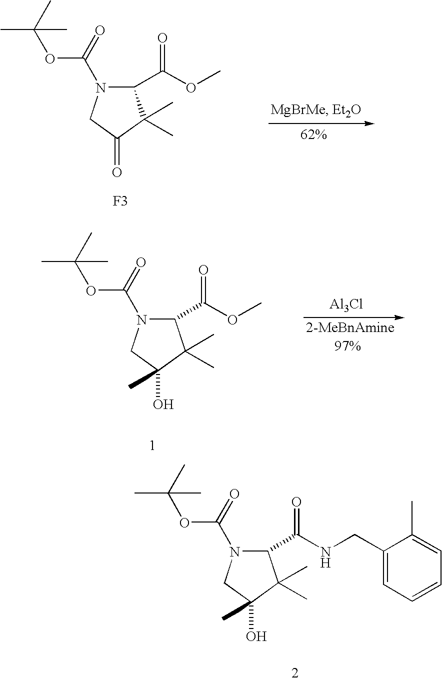

### Synthesis of (4R)-Hydroxy-3,3,4-trimethyl-2-(2-methyl-benzylcarbamoyl)-pyrrolidine-(1S)-carboxylic acid tert-butyl ester 2

Trimethyl aluminum (2.30 mL, 4.61 mmol), toluene (4 mL) at 0° C. was added slowly 2-methyl benzyl amine (0.58 mL, 4.43 mmol). This solution was warmed to 25° C. and a solution of ester 1 (0.51 g, 1.77 mmol) and toluene (6 mL) was added to the amide solution at 25° C. This was then allowed to heat at 50° C. over 12 h. The progress of the reaction was followed by TLC (3:2-EtOAc-Hex). This was then quenched with 20% aqueous Rochelle's salt solution (15 mL) and hexanes (50 mL) and left to stir vigorously for 20 min until the organic layer became a clear solution. The layers were separated, and the aqueous layer was extracted with EtOAc (2×40 mL). The organic layers were combined and dried (Na₂SO₄). The crude product 2 was purified by SiO₂ column chromatography (3:2-EtOAc:Hex) to give pure amide 2 (648 mg, 97%). ¹H NMR (300 MHz, DMSO-d₆) δ 7.15-7.29 (m, 4H), 6.29 (t, J=4.8 Hz, 1H), 5.89 (s, 1H), 4.54 (m, 1H), 4.39 (m, 1H), 3.77 (s, 1H), 3.66 (m, 1H), 3.49 (t, J=11.0 Hz, 1H), 2.31 (s, 3H), 1.43 (s, 9H), 1.15 (s, 3H), 1.04 (s, 3H), 1.03 (s, 3H) ppm.

### Synthesis of 4R-hydroxy-3,3-dimethyl-pyrrolidine-(1S)-2-dicarboxylic acid 1-tert-butyl ester 5

Synthesis of racemic compound 1 was done following the prep from Zook, S. E.; Dagnino, R., Jr.; Deason, M. E.; Bender, S. L.; Melnick, M. J. U.S. (2000), 45 pp., Cont.-in-part of U.S. Pat. No. 5,753,653 and PCT Int. Appl. (1997), 150 pp. WO 9720824 A1; Bartlett, P. A.; Barstow, J. F., J. Org. Chem., 1982, 47, 3933-3941; Kazmaier, U., Angew., Int. Ed. Engl., 1994, 33, 998-999.

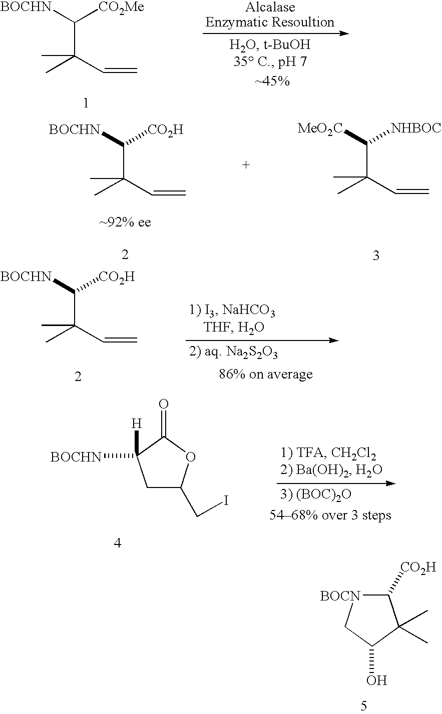

### Synthesis of (2S)-tert-butoxycarbonylamino-3,3-dimethyl-pent-4-enoic acid 2

To a 50-L three-neck flask equipped with a pH electrode, an overhead stirrer a heating mantle and a base addition line, was added the racemic ester 1 (78 g, 0.30 mol) in CH₃CN (280 mL). A mixture of Alcalase (350 mL from a 5× concentrated crude solution- Alcalase was passed through the tangential filtration system and concentrated to 1/5 of the original volume before us) and distilled H₂O (2.80 L) was then prepared at pH=7.0. The enzyme solution was added to the reaction flask. The suspension was then stirred at 30° C. for 51 h. The pH of the solution was maintained at 7.0 by adding 1N NaOH. Reaction was followed by RP-HPLC looking at both conversion and ee of the product, and stopped after 45% starting material had been consumed (after 51 h under these conditions, 95.8 mL of 1N NaOH added). The mixture was extracted MTBE (3×1.75 L), and the combined organic layers dried (MgSO₄) and concentrated under vacuum to afford 50.8 g of crude scalemic ester 3, (R)-enriched (>55% yield, approx. 56% ee). This crude mixture contained some carboxylic acid <7%, which was recovered later by acid-base extraction. The remaining aqueous solution was passed through a Pellicon 2 tangential flow filtration equipped with an Ultracel cellulose membrane. During this step most of the enzyme is removed from the aqueous solution. The remaining solution was acidified to pH 4.0 with concentrated HCl and extracted with MTBE (3×1.75 L). The acid fractions were combined and dried (Na₂SO₄) and concentrated under vacuo. A pale yellow oil acid 2 was obtained (31 g, 91.4% ee, 42% yield, >98% HPLC pure). ¹HNMR (300 MHz, CDCl₃): δ 10.69 (s, 1H 5.78 (dd, 2H5.02 (m, 2H4.96 (s, 1H4.09 (d, 1H1.36 (s, 9H1.06 (s, 6HppmThe RP-HPLC conditions to detect racemic acid 2: Detector wavelength: 200 nm; Column: Chiralcel OJ-R, 3 μm, C-184.6×100 mm; Flow rate 0.5 ml/min; Injection volume: 10 μL; Mobil Phases: A: 25 mM NaH₂PO₄ pH 2.0; B: Acetonitrile; Run: Isocratic: 25% B for 55 min, 3 min post run; Retention times: (R)-Acid 2-16.33 min and (S)-Acid 2-17.97 min; (S)-Ester 3-50.40 min and (R)-Ester 3-51.30 min.

### Synthesis of (5-iodomethyl-2-oxo-tetrahydro-furan-3-yl)-carbamic acid tert-butyl ester 4

Intermediate 2 (45 g, 18.52 mmol), and THF: H₂O (4:1 ratio, 625 mL) was cooled to 0° C. Iodine (141 g, 556 mmol) was added in portions. After 15 minutes, the reaction was warmed to 25° C. After stirring for 2 h at 25° C., saturated solution of NaHCO₃ (200 mL) was added. The reaction was left to stir for additional 30 min. The reaction mixture was poured into a 10% solution of Na₂S₂O₃ and the aqueous solution was extracted with EtOAc (3×250 mL). The organic layer was washed with a saturated solution of NaHCO₃ (2×200 mL) and dried (MgSO₄). The solvents were evaporated under vacuo to obtain product 4. Isolated yield: 70-79%. ¹H-NMR (400 MHz, DMSO-d₆) δ 7.38 (d, 1H), 4.57 (d, 1H), 4.52 (dd, 1H), 3.57 (m, 1H), 3.17 (t, 1H), 1.38 (s, 9H), 1.04 (s, 3H), 0.65 (s, 3H) ppm; MS (APCl, m/z): 314, 270, 142.

### Synthesis of 4R-hydroxy-3,3-dimethyl-pyrrolidine-(1S)-2-dicarboxylic acid 1-tert-butyl ester 5

Intermediate 4 (47.8 g, 129.64 mmol) and CH₂Cl₂ (150 mL) at 25° C. was added TFA (147.8 g, 1296 mmol) slowly. This reaction mixture was then left to reflux for 3 h. The reaction was cooled to 25° C., and the solvents and excess trifluoroacetic acid was removed under vacuum. ¹H-NMR (400 MHz, DMSO-d₆) δ 8.8 (brs, 2H), 4.61 (d, 1H), 4.35 (s, 1H), 3.65 (d, 1H), 3.26 (t, 1H), 1.26 (s, 9H), 0.8 (s, 6H) ppm; MS (APCl, m/z): 270. Aqueous solution of barium hydroxide (1 M, 204.5 g) and THF (1:1 ratio) was added to this residue. The reaction was kept under stirring at 25° C. for 4 h. Then, (BOC)₂O (31.1 g, 143 mmol) was added. The reaction was maintained at 25° C. for 16 h and then diluted with EtOAc (500 mL). This was then acidified to pH 2-3 with dilute HCl or citric acid. The organic layer was separated and dried (Na₂SO₄), and the solvents were evaporated. The residue was triturated with Et₂O/hexanes to obtain a solid compound 5, which was filtered and dried under vacuum. Isolated yield: 58-90%. ¹HNMR (400 MHz, DMSO-d₆) δ 3.70 (brm, 2H), 3.52 (m, 1H), 3.0 (m, 1H), 1.30 (s, 9H), 1.04 (s, 3H), 0.78 (s, 3H) ppm; MS (APCl, m/z): 204,160.

### Synthesis of 4R-methoxy-3,3-dimethyl-pyrrolidine-(1S)-2-dicarboxylic acid 1-tert-butyl ester 8

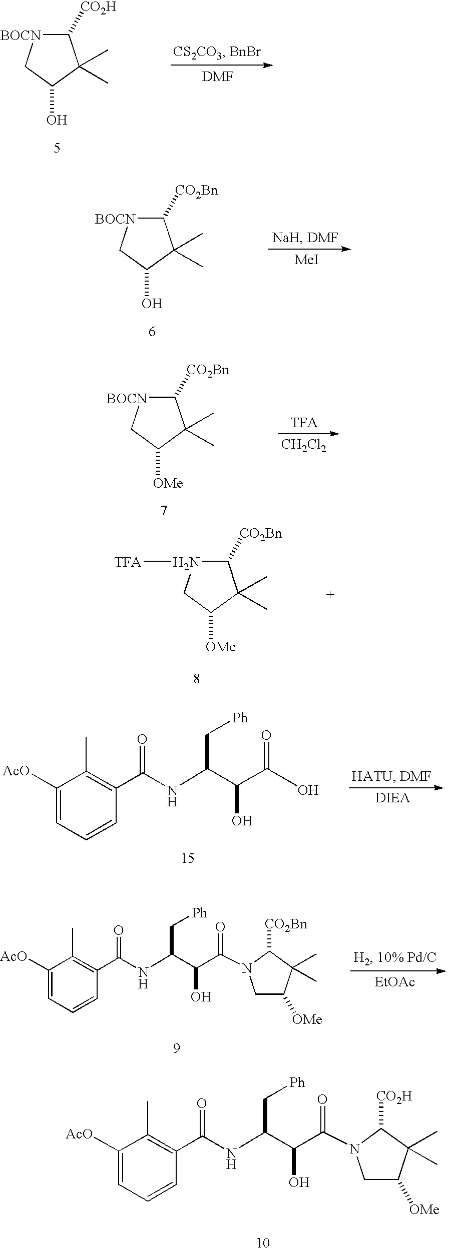

### Synthesis of 4R-hydroxy-3,3-dimethyl-pyrrolidine-(1S)-2-dicarboxylic acid 2-benzyl ester 1-tert-butyl ester 6

Hydroxy acid 5 (1.00 g, 3.86 mmol), DMF (10 mL), Cs₂CO₃ (3.44 g, 11.6 mmol), was added Nal (67 mg, 0.45 mmol) and BnBr (1.00 mL, 8.40 mmol). The heterogeneous mixture was left to stir at 25° C. for 12 h. This was then diluted with EtOAc (75 mL) and solution of 1M HCl (2×75 mL). The layers were separated and the organic layer was dried (Na₂SO₄), filetered and concentrated to a semi-solid oil. Benzyl ester 6 was collected (1.4 g, ˜100%) and taken on to the next step without further purification. ¹H NMR (400 MHz, DMSO-d₆) δ 7.42-7.31 (m, 5H), 5.14 (m, 1H), 3.90 (s, 1H), 3.77-3.76 (m, 1H), 3.59-3.56 (m, 1H), 3.04-3.02 (m, 1H), 1.39 (m, 3H), 1.26 (m, 6H), 1.07 (m, 3H), 0.73 (m, 3H) ppm.

### Synthesis of 4R-methoxy-3,3-dimethyl-pyrrolidine-(1S)-2-dicarboxylic acid 2-benzyl ester 1-tert-butyl ester 7

Benzyl ester 6 (0.70 g, 2.00 mmol) in DMF (7 mL) was added NaH (60% dispersion, 0.16 g, 4.00 mmol) all at once at 25° C. followed by addition of Mel (0.50 mL, 8.02 mmol) slowly. This was left to stir for 1 h and then quenched with Brine (50 mL), and EtOAc (2×50 mL). The organic layers were combined, dried (MgSO₄) and concentrated to give 7 (0.52 g, 98%) as a clear oil which was taken to the next step without further purification. ¹H NMR (400 MHz, DMSO-d₆) δ 7.18-7.15 (m, 5H), 4.94-4.84 (m, 2H), 3.68 (s, 1H), 3.50-3.44 (m, 1H), 3.29-3.26 (m, 1H), 3.06 (s, 3H), 2.90 (m, 1H), 1.18 (s, 3H), 1.05 (m, 6H), 0.92 (m, 3H), 0.57 (s, 3H) ppm; LCMS (electrospray) m/z calcd for C₂₀H₃₉NO₅ (M+H)⁺364.45, found 364.4.

### Synthesis of 4R-methoxy-3,3-dimethyl-pyrrolidinium-2S-carboxylic acid benzyl ester trifluoroacetate 8

Intermediate 7 (0.52 g, 1.98 mmol), CH₂Cl₂ (10 mL) and TFA (5 mL) was left to stir at 25° C. for 5 h. The solution was then concentrated under vacuo and taken to the next step without further purification.

### Synthesis of I-[3-(3-Acetoxy-2-methyl-benzoylamino)-2S-hydroxy-4S-phenyl-butyryl]-4R-methoxy-3,3-dimethyl-pyrrolidine-2S-carboxylic acid benzyl ester 9

The general procedure using HATU as the coupling agent was used to generate intermediate 9. The crude oil 9 was purified by SiO₂ column chromatography (1:4-EtOAc:Hex) to (1:2-EtOAc:Hex to (1:1-EtOAc:Hex) to give pure 9. LCMS (electrospray) m/z calcd for C₃₅H₄₁N₂O₈(M+H)⁺617.70, found 617.7.

### Synthesis of 1-[3-(3-Acetoxy-2-methyl-benzoylamino)-2S-hydroxy-4S-phenyl-butyryl]-4R-methoxy-3,3-dimethyl-pyrrolidine-2S-carboxylic acid 10

Benzyl ester 9 (0.50 g, 0.81 mmol) in EtOAc (10 mL) was evacuated and refilled with Ar (3×) and added 10% Pd/C (0.30 g). This heterogeneous mixture was left under a 1 atm of H₂ for 9 h and filtered through Celite. The pad of Celite was washed with EtOAc (30 mL) and the filtrate was concentrated to give acid 10 (0.41 g, 96%). This was used without further purification. ¹H NMR (400 MHz, DMSO-d₆) δ 8.53 (d, J=8.6 Hz, 1H), 7.35-7.01 (m, 8H), 4.44 (bs, 1H), 4.38-4.30 (m, 1H), 4.21-4.07 (m, 1H), 3.98 (s, 1H), 3.63-3.60 (m 2H), 2.89-2.83 (m, 1H), 2.72-2.69 (m 1H) ppm; LCMS (electrospray) m/z calcd for C₂₈H₃₅N₂O₈ (M+H)⁺527.6, found 527.6 and (M+Na)⁺549.6, found 549.6.

### Synthesis of 3R-phenyl -pyrrolidine-2S-carboxylic acid allylamide 4

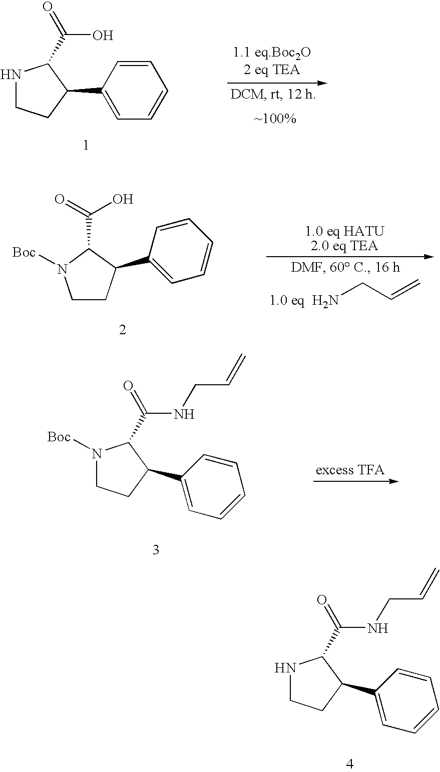

### Synthesis of 3R-Phenyl-pyrrolidine-1,2S-dicarboxylic acid 1-tert-butyl ester 2

To a solution of 1 (0.10 g, 0.50 mmol) in CH₂Cl₂ (5 mL) was added HATU (0.19 g, 0.50 mmol) and Et₃N (0.14 mL, 1.0 mmol). The resulting reaction mixture was maintained at 25° C. over 12 h. The solvent was then removed in vacuo, and the residue was purified by SiO₂ column chromatography with (99:1-EtOAc:AcOH) to give acid 2 (˜145 mg, ˜100%). ¹HNMR (300 MHz, CDCl₃) δ 12.46 (s, 1H), 7.27-7.10 (m, 5H), 3.95 (d, J=6.0 Hz, 0.35H), 3.92(d, J=9.0 Hz, 0.63H), 3.49-3.38 (m, 1H), 3.35-3.19 (m, 2H), 2.15-2.00 (m, 1H), 1.94-1.78 (m, 1H), 1.30 (s, 3.18H), 1.23 (s, 5.94H) ppm; HRMS (ESI) m/z calcd for C₁₆H₂₁NO₄: 291.1471, found for C₁₆H₂₁NO₄Na (M+Na): 314.1368.

### Synthesis of 2S-allylcarbamoyl-3R-phenyl-pyrrolidine-1-carboxylic acid tert-butyl ester 3

To a solution of acid 2 (0.29 g, 1.0 mmol) in anhydrous DMF (4 mL) was added a solution of HATU (0.38 g, 1.0 mmol) and DMF (4 mL), followed by a solution of allylamine (0.07 g, 1.0 mmol) and DMF (4 mL), and Et₃N (0.28 mL, 2.0 mmol). The resulting reaction mixture was stirred at 60° C. for 16 h. The solvent and volatiles were removed in vacuo to afford 3 as dark residue that was used without further purification.

### Synthesis of 3R-phenyl-pyrrolidine-2S-carboxylic acid allylamide 4

Amide 3 and trifluoracetic acid (3 mL) was maintained at 25° C. for 3-5 h. The excess acid was removed in vacuo, and the resulting residue was redissolved EtOAc (20 mL). The organic layer was then washed with saturated aqueous NaHCO₃ (2×20 mL), brine (1×20 mL), and dried (MgSO₄). This was then concentrated to afford 4 which was used in the next step without further purification.

### Specific Case-Modified General Method C

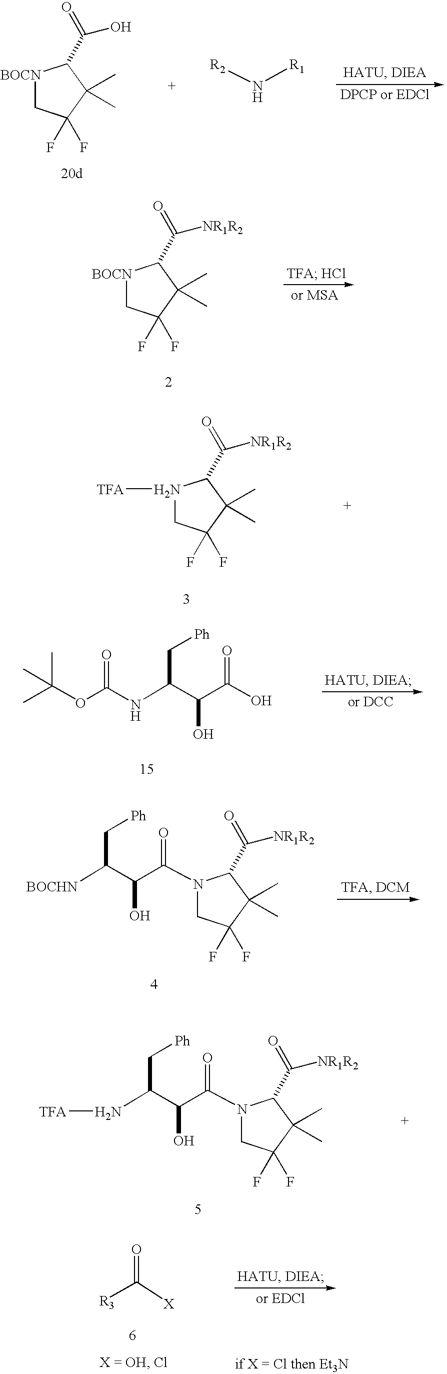

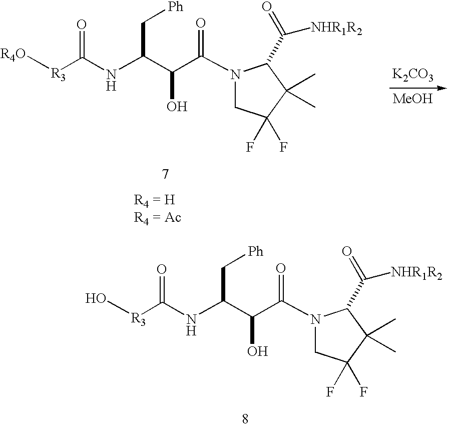

The synthesis of the compound where the P1′ has the functionality of the difluorodimethyl proline was synthesized following the general Method C procedure.

HATU coupling—To a solution of acid 20d (1 eq) and amine 1 (1.2 eq) or acid 15 (1 eq) and amine 3 (1.2 eq) or acid/acid chloride 6 and amine 5, was added DMF (1M) and DIEA until pH˜7-8. This was then followed by HPLC until reaction is completed and no more starting material was detected. The reaction mixture was quenched with aqueous HCl (1 M) and extracted with EtOAc. The layers were separated, and the organic layer was washed with saturated aqueous NaHCO₃, Brine and dried (MgSO₄). After solvent evaporation, the crude products 2, 4, or 7 respectively were purified by SiO₂ column chromatography.

Coupling of P2 piece by acylation with acid chloride R₃COCl—To a 0° C. solution of amine 5 (1 eq) in THF:DCM (1:1, 0.4 M), was added acid chloride 6 (1 eq) followed by addition of Et₃N (1.2 eq). This was left to warm to 25° C. over 12 h and then quenched with DCM and H₂O. The layers were separated, and the organic layer was washed with saturated aqueous NaHCO₃, and dried (MgSO₄). The product 7 was then purified by SiO₂ column chromatography.

In cases where P2 piece is protected as the acetate prior to coupling to intermediate 5, the final product was obtained by deprotection of the acetate group using the following procedure. Acetate intermediate 7 (R₄=Ac) (1 eq) and MeOH (1 M) was added K₂CO₃ (3-5 eq) at 25° C. This was then concentrated and partitioned with EtOAc and aqueous 1M HCl. The layers were separated, and the organic layer was dried (MgSO₄) and concentrated.

The following compounds have been prepared according to the procedures described herein (General Method C) and have demonstrated the noted activity.

| | | EC₅₀ |
|---|---|---|
| | Ki | (wt) |
| Molecular Structure | (nm) | (nm) |
| 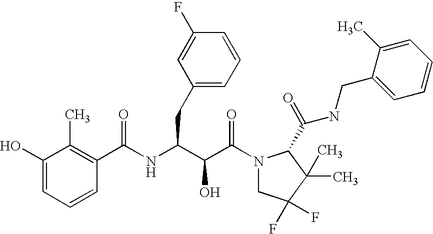 | <0.1 | 17 |
| 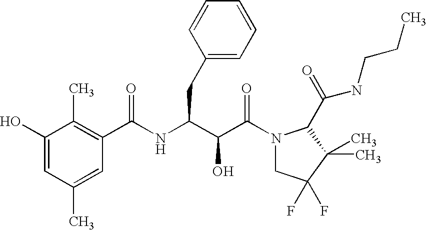 | <0.1 | 9 |
| 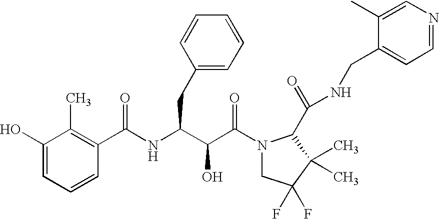 | <0.1 | 8 |
| 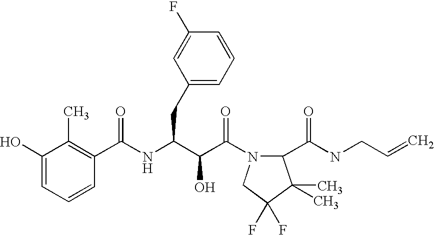 | <0.1 | 16 |
| 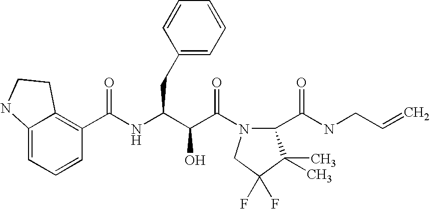 | <0.1 | 16 |
| 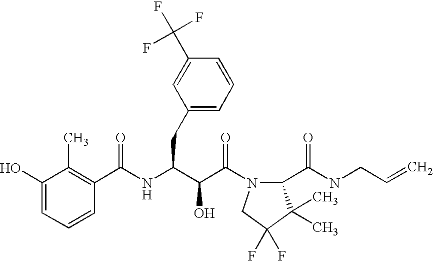 | <0.1 | 42 |
| 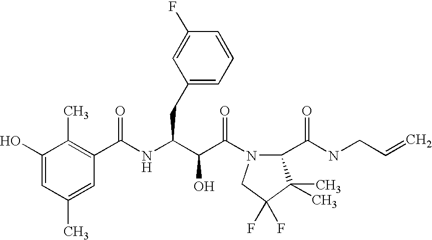 | <0.1 | 25 |
| 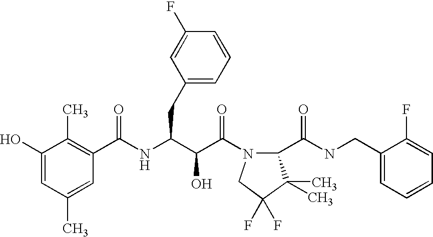 | <0.1 | 6.7 |
| 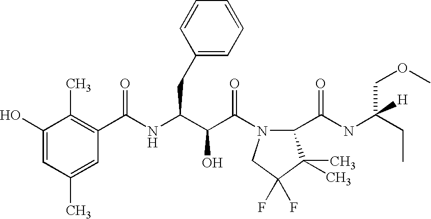 | <0.1 | 30 |
| 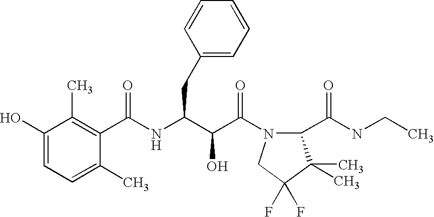 | <0.1 | 35 |
| 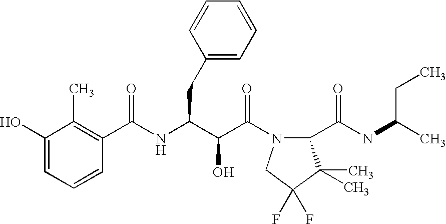 | <0.1 | 31 |
| 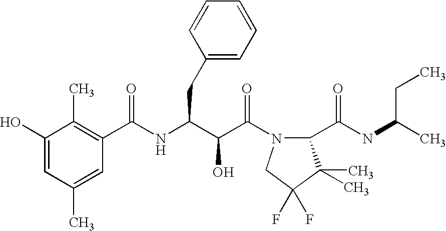 | <0.1 | 16 |
| 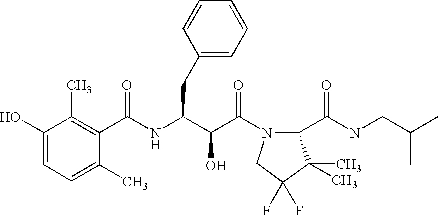 | <0.1 | 44 |
| 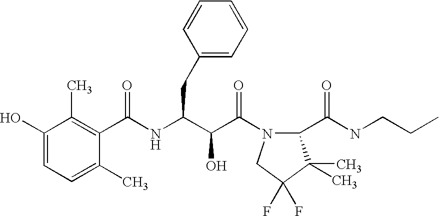 | <0.1 | 33 |
| 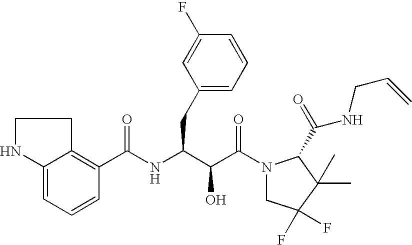 | <0.1 | 35 |
| 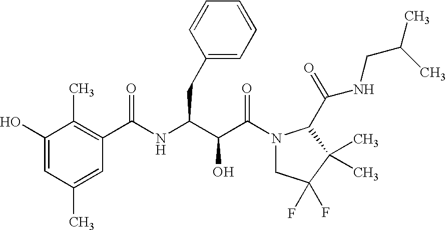 | <0.1 | 31 |
| 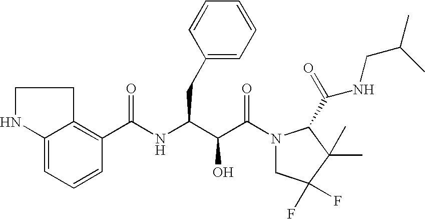 | <0.1 | 35 |
| 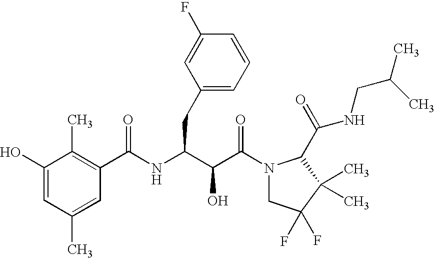 | <0.1 | 11 |
| 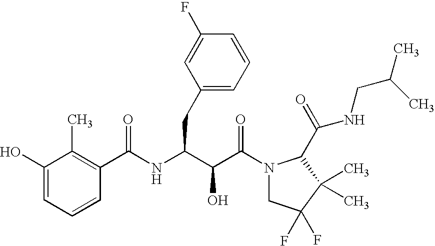 | <0.1 | 13 |
| 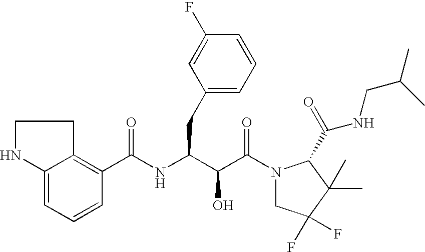 | <0.1 | 42 |
| 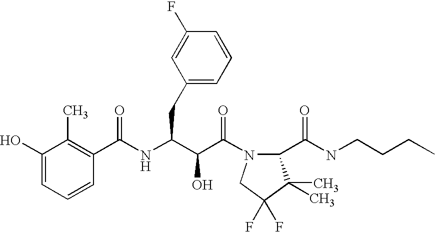 | <0.1 | 32 |
| 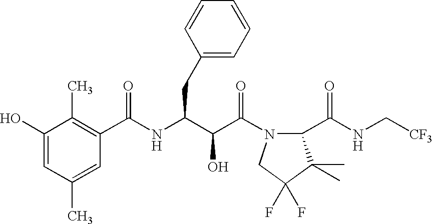 | <0.1 | 18 |
| 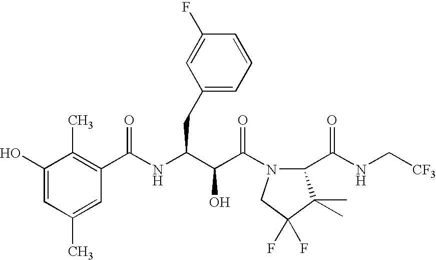 | <0.1 | 14 |
| 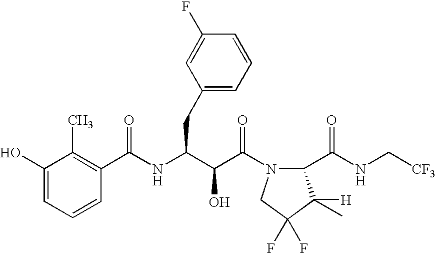 | <0.1 | 10 |
| 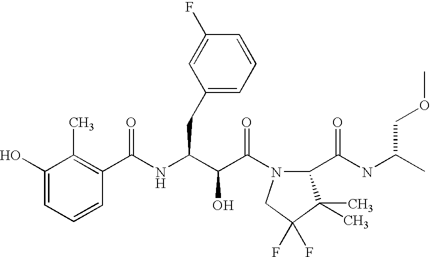 | <0.1 | 100 |
| 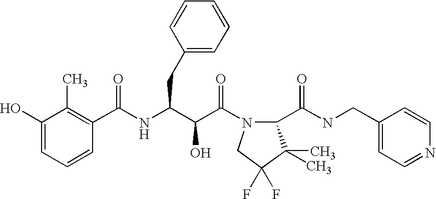 | <0.1 | 130 |
| 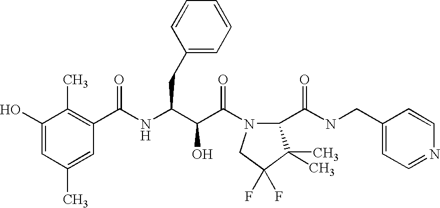 | 0.26 | 110 |
| 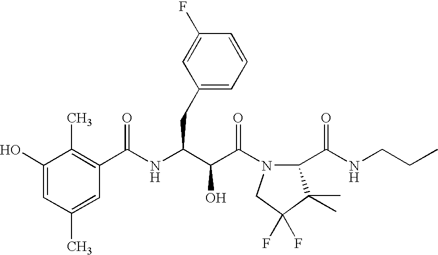 | <0.1 | 14 |
| 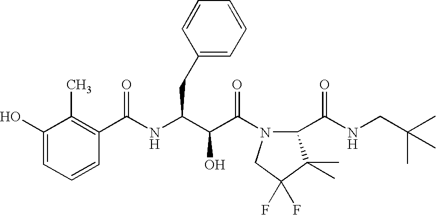 | <0.1 | 4.7 |
| 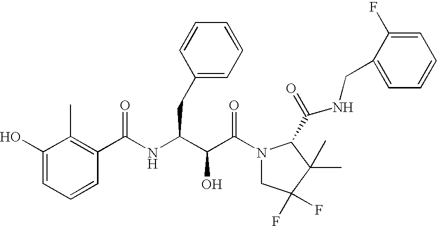 | <0.1 | 16 |
| 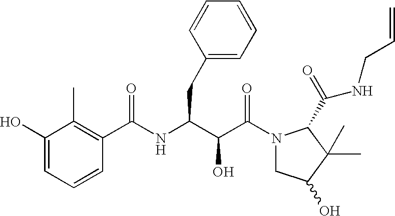 | 3.6 | 470 |
| 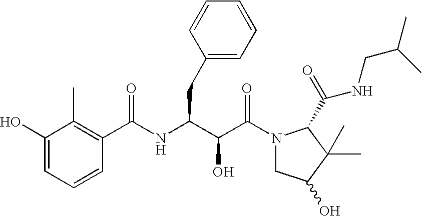 | 0.7 | 450 |
| 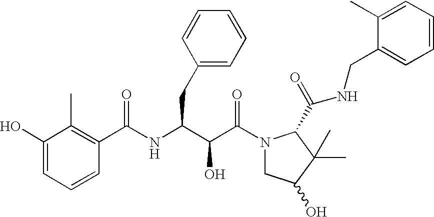 | <0.1 | 140 |
| 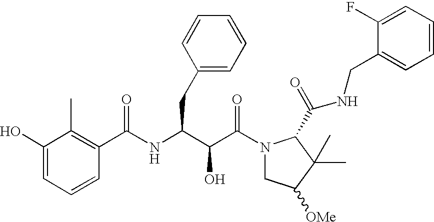 | 0.34 | 1000 |
| 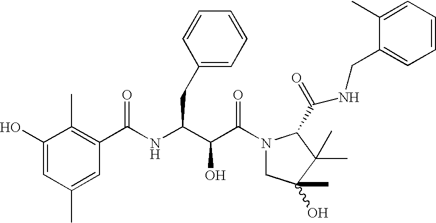 | <0.1 | 12 |
| 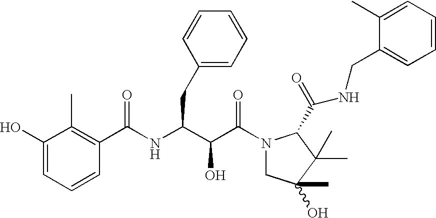 | <0.1 | 33 |
| 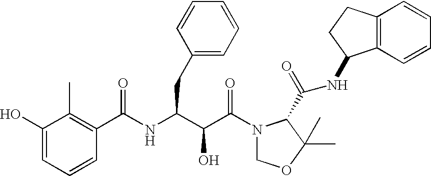 | <0.1 | 33 |
| 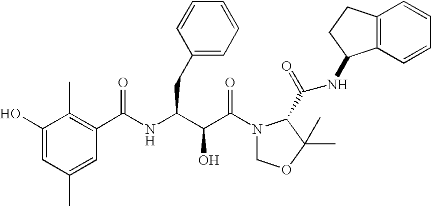 | <0.1 | 61 |
| 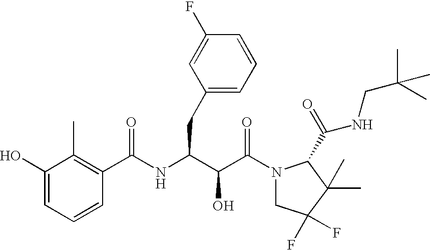 | <0.1 | 13 |
| 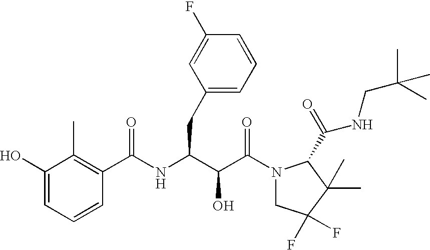 | <0.1 | 8.8 |
| 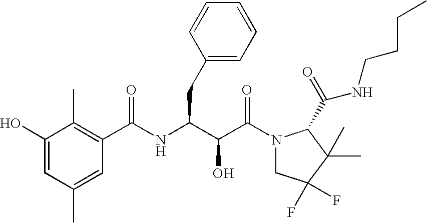 | <0.1 | 23 |
| 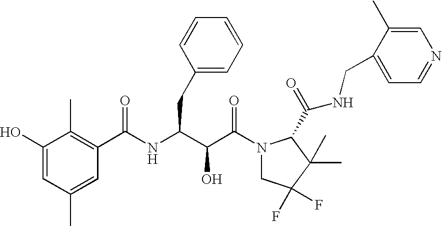 | <0.1 | 34 |
| 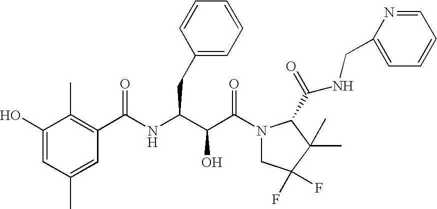 | <0.1 | 61 |
| 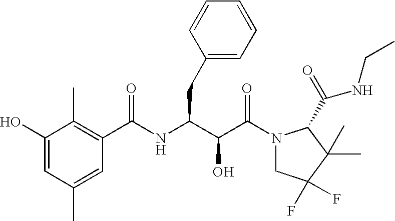 | <0.1 | 37 |
| 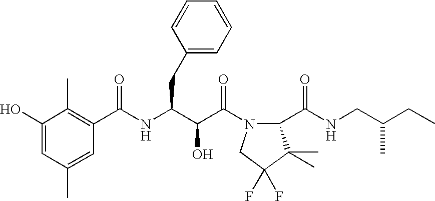 | <0.1 | 7.5 |
| 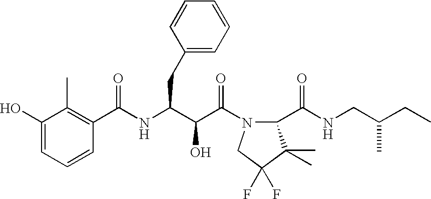 | <0.1 | 11 |

### Example E1

### (S)-4,4-Difluoro-1-[4-(3-fluoro-phenyl)-(2S,3S)-hydroxy-(3-hydroxy-2-methyl-benzoylamino) -butyryl]-3,3-dimethyl-pyrrolidine-2-carboxylic acid (2-methoxy-(1S)-methyl-ethyl)-amide

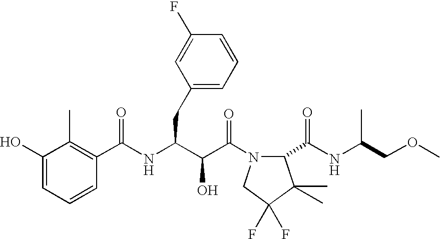

White solid: ¹H NMR (300 MHz, DMSO-d₆) δ 9.49(s, 1H), 8.38 (d, J=8.5 Hz, 1H), 8.00 (d, J=8.1 Hz, 1H), 7.42-7.29 (m, 3H), 7.11-7.06 (m, 2H), 6.88 (d, J=7.6, 1H), 6.65 (d, J=7.6 Hz 1H), 5.45 (d, J=6.8, 1H), 4.55-4.36 (m, 4H), 4.11 (m, 1H), 3.33 (s, 3H), 2.93-2.74 (m, 2H), 1.89 (s, 3H), 1.53-1.31 (m, 3H), 1.37-1.24 (m, 3H), 1.17-1.15 (m, 6H) ppm; LCMS (electrospray) m/z calcd for C₂₉H₃₆F₃N₃O₆ (M+H)⁺580.63, found 580.15.

### Example E2

### N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide

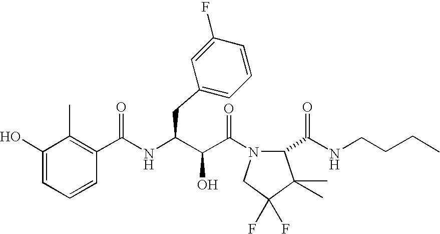

White solid: ¹H NMR (300 MHz, DMSO-d₆) δ 9.40(s, 1H), 8.24 (d, J=8.3 Hz, 1H), 7.93 (t, J=5.6 Hz, 1H), 7.34-7.19 (m, 3H), 7.06-6.94 (m, 2H), 6.79 (d, J=8.1 Hz, 1H), 6.55 (d, J=7.5 Hz, 1H,), 5.45 (d, J=6.4 Hz, 1H), 4.55-4.36 (m, 5H), 3.08 (m, 2H), 2.88-2.62 (m, 2H), 1.80 (s, 3H), 1.43-1.13 (m, 4H), 1.20 (s, 3H), 1.03 (s, 3H), 0.85 (t, J=7.2 Hz, 3H) ppm; Calcd for C₂₉H₃₆F₃N₃O₅+2.0 eq of H₂O+0.5 eq of EtOAc: C, 64.17; H, 7.04; N, 7.48. Found: C, 63.88; H, 7.22; N, 7.19; LCMS (electrospray) m/z calcd for C₂₉H₃₆F₃N₃O₅ (M+H)⁺564.63, found 564.20.

### Example E3

### (S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide

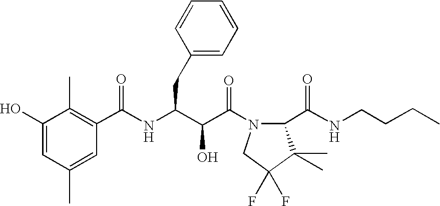

Beige solid: ¹H NMR (300 MHz, DMSO-d₆) δ 9.12(s, 1H), 7.98 (d, J=8.1Hz, 1H), 7.75 (t, J=5.6 Hz, 1H), 7.23-7.02 (m, 5H), 6.47 (s, 1H), 6.24 (s, 1H), 5.32 (d, J=6.4 Hz, 1H,), 4.32-4.07 (m, 5H), 2.94 (m, 2H), 2.74-2.52 (m, 2H), 2.03 (s, 3H), 1.61 (s, 3H), 1.30-1.02 (m, 4H), 1.07 (s, 3H), 0.90 (s, 3H), 0.72 (t, J=7.2 Hz, 3H) ppm; Calcd for C₃₀H₃₉F₂N₃O₅: C, 64.38; H, 7.02; N, 7.51. Found: C, 64.27; H, 7.23; N, 7.34; LCMS (electrospray) m/z calcd for C₃₀H₃₉F₂N-₃O₅(M+H)⁺560.67, found 560.15.

### Example E4

### (S)-4,4-Difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide

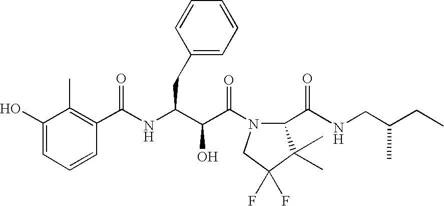

White solid: ¹H NMR (300 MHz, DMSO-d₆) δ 9.38 (s, 1H), 8.17 (d, J=8.1 Hz, 1H), 7.90 (t, J=5.8 Hz, 1H,), 7.37 (d, J=7.3 Hz, 1H,), 7.26 (t, J=7.5 Hz, 1H,), 7.17 (t, J=7.2 Hz, 1H), 6.95 (t, J=7.8 Hz, 1H), 6.78 (d, J=7.3 Hz, 1H), 6.54 (d, J=6.8 Hz, 1H), 5.48 (d, J=6.6 Hz, 1H), 4.46-4.26 (m, 4H), 3.08-2.66 (m, 4H), 1.81 (s, 3H), 1.53-1.31 (m, 3H), 1.21 (s, 3H), 1.18-1.06 (m, 2H), 1.04 (s, 3H), 0.85-0.82 (m, 6H) ppm; HRMS (ESI) m/z calcd for C₃₀H₄₀F₂N₃O₅ (M+H)⁺560.2936, found 560.2949; Calcd for C₃₀H39F₂N₃O₅+0.1 eq of H₂O: C, 64.17; H, 7.04; N, 7.48. Found: C, 63.88; H, 7.22; N, 7.19.

### Example E5

### (S)-4,4-Difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide

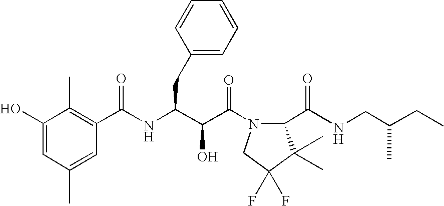

White solid: ¹H NMR (300 MHz, DMSO-d₆) δ 9.29 (s, 1H), 8.17 (d, J=8.4 Hz, 1H), 7.95 (t, J=5.5 Hz, 1H,), 7.41 (d, J=7.4 Hz, 1H,), 7.31 (t, J=7.6 Hz, 2H,), 7.23 (t, J=7.3 Hz, 1H), 6.65 (s, 1H), 6.43 (s, 1H), 5.51 (br s, 1H), 4.50-4.31 (m, 4H), 3.08-2.75 (m, 4H), 2.56 (s, 3H), 1.81 (s, 3H), 1.55-1.43 (m, 2H), 1.27 (s, 3H), 1.25-1.12 (m, 2H), 1.10 (s, 3H), 0.90-0.87 (m, 6H) ppm; HRMS (ESI) m/z calcd for C₃₁H₄₂F₂N₃O₅ (M+H)⁺574.3093, found 574.3100; Calcd for C₃₁H₄₁F₂N₃O₅+0.5 eq of EtOAc: C, 64.90; H, 7.53; N, 6.68. Found: C, 63.38; H, 7.492; N, 6.63.

### Example E6

### (S)-4,4-Difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid ethylamide

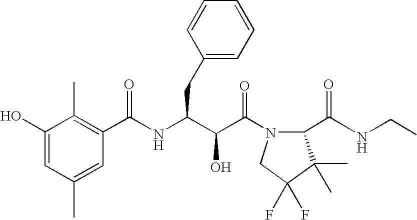

Beige solid: ¹H NMR (300 MHz, DMSO-d₆) δ 9.41 (s, 1H), 8.28 (d, J=8.3 Hz, 1H), 8.09 (t, J=5.6, 1H), 7.56-7.29 (m, 5H), 6.77 (s, 1H), 6.54 (s, 1H), 5.63 (d, J=6.2 Hz, 1H), 4.66-4.35 (m, 5H), 3.29 (m, 2H), 3.05-2.81 (m, 2H), 2.33 (s, 3H), 1.91 (s, 3H), 1.37 (s, 3H), 1.21 (s, 3H), 1.19 (t, J=7.2 Hz, 3H) ppm; Calcd for C₂₈H₃₅F₂N₃O₅+0.5 eq of H₂O: C, 62.21; H, 6.71; N, 7.77. Found: C, 62.13; H, 6.67; N, 7.39; LCMS (electrospray) m/z calcd for C₂₈H₃₅F₂N₃O₅ (M+H)⁺532.61, found 532.10.

### Example E7

### (S)-4,4-Difluoro-1-(2S, 3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide

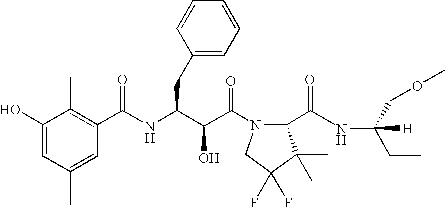

White solid: ¹H NMR (300 MHz, DMSO-d₆) δ 9.28(s, 1H), 8.20 (d, J=8.5 Hz, 1H), 7.82 (d, J=8.7, 1H), 7.41-7.17 (m, 5H), 6.63 (s, 1H), 6.42 (s, 1H), 5.35 (d, J=7.0 Hz, 1H), 4.50-4.30 (m, 5H), 3.89 (m, 1H), 3.27 (s, 3H), 2.85-2.66 (m, 2H), 2.19 (s, 3H), 1.78 (s, 3H), 1.64-1.49 (m, 2H), 1.42-1.18 (m, 2H), 1.24 (s, 3H), 1.09 (s, 3H), 0.89 (t, J=7.4 Hz, 3H) ppm; Calcd for C₃₁H₄₁F₂N₃O₆: C, 63.14; H, 7.01; N, 7.12. Found: C, 62.98; H, 6.89; N, 7.05; LCMS (electrospray) m/z calcd for C₃₁H₄₁F₂N₃O₆ (M+H)⁺590.69, found 590.15.

### Example E8

### (S)-4,4-Difluoro-1-[4-(3-fluoro-phenyl)-42S)-hydroxy-(3S)-(3-hydroxy-2-methyl-benzoylamino)-butyryl]-3,3-dimethyl-pyrrolidine-2-carboxylic acid allylamide

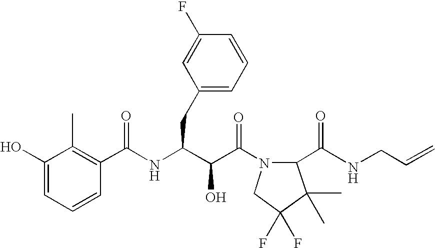

White solid: ¹H NMR (300 MHz, DMSO-d₆) δ 9.42(s, 1H), 8.26 (d, J=7.9 Hz, 1H), 8.17 (t, J=5.1 Hz, 1H), 7.33-7.18 (m, 3H), 7.04-6.96 (m, 2H), 6.80 (d, J=7.8 Hz, 1H), 6.57 (d, J=7.4 Hz, 1H), 5.87-5.74 (m, 1H), 5.52 (d, J=6.6 Hz, 1H), 5.24 (d, J=18.9 Hz, 1H), 5.07 (d, J=10.4 Hz, 1H) 4.50-4.29 (m, 5H), 3.75 (m, 2H), 2.90-2.66 (m, 2H), 1.82 (s, 3H), 1.22 (s, 3H), 1.06 (s, 3H) ppm; Calcd for C₂₈H₃₂F₃N₃O₅+0.25 eq of H₂0: C, 60.91; H, 5.93; N, 7.61. Found: C, 60.96; H, 6.05; N, 7.20; LCMS (electrospray) m/z calcd for C₂₈H₃₂F₃N₃O₅ (M+H)⁺548.59, found 548.2.

### Example E9

### (S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide

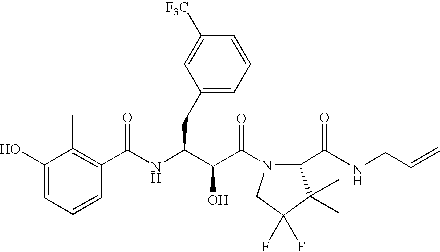

White solid: ¹H NMR (300 MHz, DMSO-d₆) δ 9.47(s, 1H), 8.36 (d, J=8.3 Hz, 1H), 8.23 (t, J=5.7 Hz, 1H), 7.82-7.54 (m, 4H), 7.01 (m, 1H), 6.85 (d, J=7.7 Hz, 1H), 6.60 (d, J=7.5 Hz, 1H), 5.89-5.79 (m, 1H), 5.58 (d, J=6.0 Hz, 1H), 5.28 (d, J=15.8 Hz, 1H), 5.12 (d, J=10.4 Hz, 1H) 4.54-4.31 (m, 5H), 3.79 (m, 2H), 3.02-2.79 (m, 2H) 1.84 (s, 3H), 1.27 (s, 3H), 1.10 (s, 3H) ppm; Calcd for C₂₉H₃₂F₅N₃O₅+0.25 eq of H₂O: C, 58.29; H, 5.40; N, 7.03. Found: C, +H)⁺557.2939, found 557.2910; Calcd for C₃₀H₃₈F₂N₄O₄: C, 64.73; H, 6.88; N, 10.07. Found: C, 64.33; H, 6.86; N, 9.81.

### Example E13

### (S)-4,4-Difluoro-1-[(2S, 3S)-4-(3-fluoro-phenyl)-2-hydroxy-3-(3-hydroxy-2,5-dimethyl-benzoylamino)-butyryl]3,3-dimethyl-pyrrolidine-2-carboxylic acid isobutyl-amide

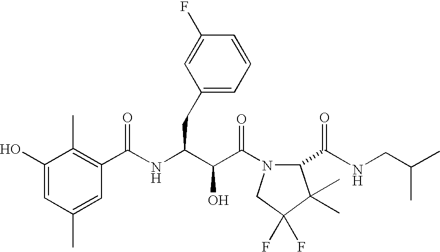

White solid: ¹H NMR (300 MHz, DMSO-d₆) δ 9.25 (s, 1H), 8.18 (d, J=8.6 Hz, 1H), 7.96 (t, J=5.7 Hz, 1H), 7.29 (m, 1H), 7.19 (d, J=7.8 Hz, 2H), 6.99 (m, 1H), 6.60 (s, 1H), 6.37 (s, 1H), 5.38 (d, J=5.8 Hz, 1H), 4.43 (q, J=12.7 Hz, 1H), 4.24-4.34 (m, 4H), 2.85-2.96 (m, 2H), 2.82 (m, 1H), 2.68 (m, 1H), 2.15 (s, 3H), 1.74 (s, 3H), 1.68 (m, 1H), 1.20 (s, 3H), 1.04 (s, 3H), 0.84 (d, J=6.8 Hz, 6H) ppm; HRMS (ESI) m/z calcd for C₃₀H₃₉F₃N₃O₅ (M+H)⁺578.2842, found 578.2833; Calcd for C₃₀H₃₈F₃N₃O₅: C, 62.38; H, 6.63; N, 7.27. Found: C, 62.30; H, 6.67; N, 7.03.

### Example E14

### 2,3-Dihydro-1H-indole-4-carboxylic acid [(1S, 2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide

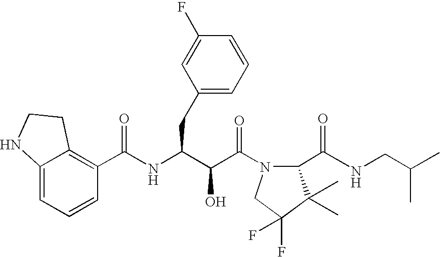

White solid: ¹H NMR (300 MHz, DMSO-d₆) δ 8.10 (d, J=8.3 Hz, 1H), 7.95 (t, J=5.8 Hz, 1H), 7.26 (m, 1H), 7.20 (d, J=7.1 Hz, 2H), 6.90-6.98 (m, 2H), 6.70 (d, J=7.6 Hz, 1H), 6.54 (d, J=7.6 Hz, 1H), 5.71 (s, 1H), 5.42 (d, J=5.6 Hz, 1H), 4.24-4.42 (m, 5H), 2.75-2.96 (m, 8H), 1.68 (m, 1H), 1.21 (s, 3H), 1.03 (s, 3H), 0.84 (d, J=6.8 Hz, 6H) ppm; HRMS (ESI) m/z calcd for C₃₀H₃₈F₃N₄O₄ (M+H)⁺575.2845, found 575.2850; Calcd for C₃₀H₃₇F₃N₄O₄: C, 62.70; H, 6.49; N, 9.75. Found: C, 62.50; H, 6.51; N, 9.62.

### Example E15

### (S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide

White solid: ¹H NMR (300 MHz, DMSO-d₆) δ 8.02 (d, J=8.3 Hz, 1H), 7.91 (t, J=5.8 Hz, 1H), 7.35 (d, J=7.1 Hz, 2H), 7.23 (t, J=7.5 Hz, 2H), 7.13 (t, J=7.3 Hz, 1H), 6.91 (t, J=7.7 Hz, 1H), 6.68 (d, J=7.3 Hz, 1H), 6.53 (d, J=7.6 Hz, 1H), 5.62 (s, 1H), 5.46 (d, J=6.1 Hz, 1H), 4.26-4.42 (m, 4H), 4.22 (s, 1H), 3.29-3.34 (m, 2H), 2.74-2.98 (m, 6H), 1.67 (m, 1H), 1.20 (s, 3H), 1.03 (s, 3H), 0.83 (d, J=6.8 Hz, 6H) ppm; HRMS (ESI) m/z calcd for C₃₀H₃₉F₂N₄O₄ (M +H)+557.2939, found 557.2910; Calcd for C₃₀H₃₈F₂N₄O₄: C, 64.73; H, 6.88; N, 10.07. Found: C, 64.33; H, 6.86; N, 9.81.

### Example E13

### (S)-4,4-Difluoro-1-[(2S, 3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide

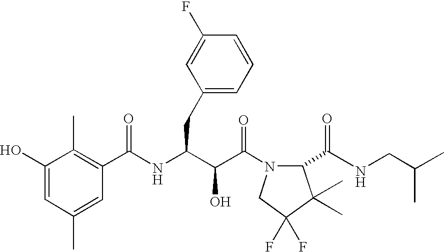

White solid: ¹H NMR (300 MHz, DMSO-d₆) δ 9.25 (s, 1H), 8.18 (d, J=8.6 Hz, 1H), 7.96 (t, J=5.7 Hz, 1H), 7.29 (m, 1H), 7.19 (d, J=7.8 Hz, 2H), 6.99 (m, 1H), 6.60 (s, 1H), 6.37 (s, 1H), 5.38 (d, J=5.8 Hz, 1H), 4.43 (q, J=12.7 Hz, 1H), 4.24-4.34 (m, 4H), 2.85-2.96 (m, 2H), 2.82 (m, 1H), 2.68 (m, 1H), 2.15 (s, 3H), 1.74 (s, 3H), 1.68 (m, 1H), 1.20 (s, 3H), 1.04 (s, 3H), 0.84 (d, J=6.8 Hz, 6H) ppm; HRMS (ESI) m/z calcd for C₃₀H₃₉F₃N₃O₅ (M+H)⁺578.2842, found 578.2833; Calcd for C₃₀H₃₈F₃N₃O₅: C, 62.38; H, 6.63; N, 7.27. Found: C, 62.30; H, 6.67; N, 7.03.

### Example E14

### 2,3-Dihydro-1H-indole-4-carboxylic acid [(1S, 2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide

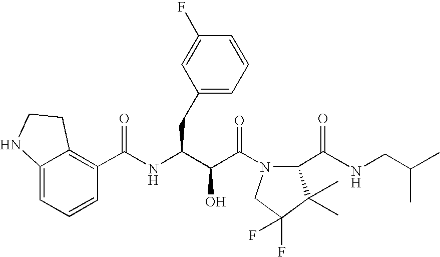

White solid: ¹H NMR (300 MHz, DMSO-d₆) δ 8.10 (d, J=8.3 Hz, 1H), 7.95 (t, J=5.8 Hz, 1H), 7.26 (m, 1H), 7.20 (d, J=7.1 Hz, 2H), 6.90-6.98 (m, 2H), 6.70 (d, J=7.6 Hz, 1H), 6.54 (d, J=7.6 Hz, 1H), 5.71 (s, 1H), 5.42 (d, J=5.6 Hz, 1H), 4.24-4.42 (m, 5H), 2.75-2.96 (m, 8H), 1.68 (m, 1H), 1.21 (s, 3H), 1.03 (s, 3H), 0.84 (d, J=6.8 Hz, 6H) ppm; HRMS (ESI) m/z calcd for C₃₀H₃₈F₃N₄O₄ (M+H)⁺575.2845, found 575.2850; Calcd for C₃₀H₃₇F₃N₄O₄: C, 62.70; H, 6.49; N, 9.75. Found: C, 62.50; H, 6.51; N, 9.62.

### Example E15

### (S)-4,4-Difluoro-1-[(2S, 3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide

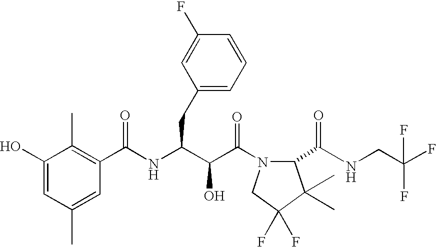

White solid: ¹H NMR (300 MHz, DMSO-d₆) δ 9.26 (s, 1H), 8.71 (t, J=6.1 Hz, 1H), 8.18 (d, J=8.3 Hz, 1H), 7.29 (m, 1H), 7.15-7.17 (m, 2H), 6.99 (t, J=8.5 Hz, 1H), 6.60 (s, 1H), 6.37 (s, 1H), 5.50 (d, J=6.3 Hz, 1H), 4.47 (q, J=11.5 Hz, 1H), 4.26-4.32 (m, 4H), 4.01 (m, 1H), 3.88 (m, 1H), 2.83 (m, 1H), 2.69 (dd, J=12.8 Hz, 11.2, 1H), 2.15 (s, 3H), 1.74 (s, 3H), 1.21 (s, 3H), 1.03 (s, 3H) ppm; HRMS (ESI) m/z calcd for C₂₈H₃₂F₆N₃O₅ (M+H)⁺604.2244, found 604.2245; Calcd for C₂₈H₃₁F₆N₃O₅: C, 55.72; H, 5.18; N, 6.96. Found: C, 55.42; H, 5.31; N, 6.75.

### Example E16

### (S)-4,4-Difluoro-1-[(2S, 3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)amide

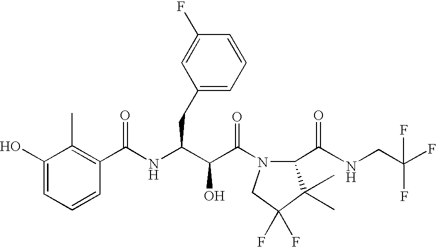

White solid: ¹H NMR (300 MHz, DMSO-d₆) δ 9.39 (s, 1H), 8.71 (t, J=6.3 Hz, 1H), 8.23 (d, J=8.3 Hz, 1H), 7.29 (m, 1H), 7.14-7.17 (m, 2H), 6.99 (m, 1H), 6.96 (t, J=7.6 Hz, 1H), 6.77 (d, J=7.3 Hz, 1H), 6.53 (d, J=6.6 Hz, 1H), 5.55 (d, J=5.1 Hz, 1H), 4.47 (m, 1H), 4.27-4.37 (m, 4H), 4.03 (m, 1H), 3.86 (m, 1H), 2.85 (m, 1H), 2.68 (dd, J=13.4, 11.1 Hz, 1H), 1.78 (s, 3H), 1.20 (s, 3H), 1.02 (s, 3H) ppm; HRMS (ESI) m/z calcd for C₂₇H₃₀F₆N₃O₅ (M+H)⁺590.2090, found 590.2103; Calcd for C₂₇H₂₉F₆N₃O₅: C, 55.01; H, 4.96; N, 7.13. Found: C, 54.80; H, 5.04; N, 7.06.

### Example E17

### (S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide

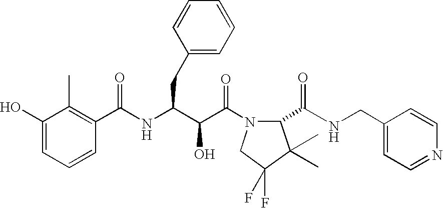

White solid: ¹H NMR (300 MHz, DMSO-d₆) δ 9.37 (s, 1H), 8.57 (t, J=5.9 Hz, 1H), 8.45 (d, J=5.8 Hz, 2H), 8.16 (d, J=8.3 Hz, 1H), 7.30 (d, J=5.8 Hz, 2H), 7.28 (d, J=7.3 Hz, 2H), 7.21 (t, J=7.5 Hz, 2H), 7.14 (t, J=7.0 Hz, 1H), 6.93 (t, J=7.7 Hz, 1H), 6.76 (d, J=8.3 Hz, 1H), 6.53 (d, J=7.6 Hz, 1H), 5.61 (d, J=6.1 Hz, 1H), 4.42-4.52 (m, 2H), 4.29-4.35 (m, 4H), 4.21 (dd, J=16.6, 5.7 Hz, 1H), 2.88 (dd, J=13.1, 1.3 Hz, 1H), 2.69 (dd, J=13.5, 10.2 Hz, 1H), 1.79 (s, 3H), 1.21 (s, 3H), 1.03 (s, 3H) ppm; HRMS (ESI) m/z calcd for C₃₁H₃₅F₂N₄O₅ (M+H)⁺581.2576, found 581.2587; Calcd for C₃₁H₃₄F₂N₄O₅+0.7 eq of H₂O: C, 62.76; H, 6.02; N, 9.44. Found: C, 62.68; H, 6.07; N, 9.42.

### Example E18

### (S)-4,4-Difluoro-1-[(2S, 3S)-2-hydroxy-3-(3-hydroxy-2,5-dimethyl-benzoylamino)-4-phenyl-butyryl]-3,3-dimethyl-pyrrolidine-2-carboxylic acid (pyridin-4-ylmethyl)-amide

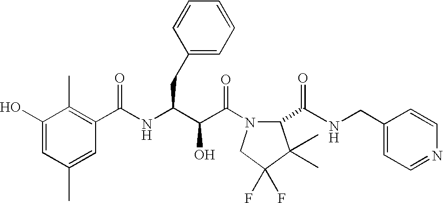

White solid: ¹H NMR (300 MHz, DMSO-d₆) δ 9.23 (s, 1H), 8.57 (t, J=5.6 Hz, 1H), 8.44 (d, J=5.1 Hz, 2H), 8.11 (d, J=7.8 Hz, 1H), 7.14-7.31 (m, 7H), 6.58 (s, 1H), 6.36 (s, 1H), 5.57 (d, J=5.6 Hz, 1H), 4.45 (dd, J=16.2, 7.1 Hz, 1H), 4.30-4.32 (m, 4H), 4.21 (dd, J=16.3, 5.4 Hz, 1H), 4.02 (q, J=7.2 Hz, 1H), 2.87 (dd, J=13.6, 1.3 Hz, 1H), 2.68 (dd, J=13.6, 10.1 Hz, 1H), 2.13 (s, 3H), 1.73 (s, 3H), 1.22 (s, 3H), 1.03 (s, 3H) ppm; HRMS (ESI) m/z calcd for C₃₂H₃₇F₂N₄O₅ (M+H)⁺595.2732, found 595.2732; Calcd for C₃₂H₃₆F₂N₄O₅ +0.8 eq of H₂O: C, 63.10; H, 6.22; N, 9.20. Found: C, 63.12; H, 6.40; N, 8.93.

### Example E19

### (S)-4,4-Difluoro-1-[(2S, 3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide

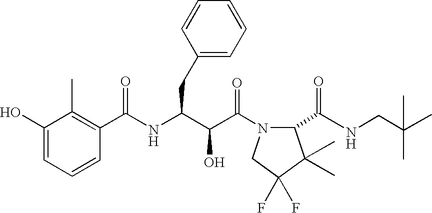

White solid: ¹H NMR (300 MHz, DMSO-d₆) δ 9.36 (s, 1H), 8.19 (d, J=8.6 Hz, 1H), 7.84 (t, J=6.2 Hz, 1H), 7.35 (d, J=7.3 Hz, 2H), 7.24 (t, J=7.5 Hz, 1H), 7.15 (t, J=7.3 Hz, 1H), 6.94 (t, J=7.7 Hz, 1H), 6.77 (d, J=7.8 Hz, 1H), 6.53 (d, J=7.3 Hz, 1H), 5.39 (d, J=4.0 Hz, 1H), 4.44 (q, J=12.5 Hz, 1H), 4.25-4.34 (m, 4H), 4.00 (m,1H), 2.95 (dd, J=13.4, 6.6 Hz, 1H), 2.88 (dd, J=13.1, 6.3 Hz, 1H), 2.80 (m, 1H), 2.66 (dd, J=13.4, 11.4 Hz, 1H), 1.79 (s, 3H), 1.21 (s, 3H), 1.05 (s, 3H), 0.85 (s, 9H) ppm; HRMS (ESI) m/z calcd for C₃₀H₄₀F₂N₃O₅ (M+H)⁺560.2936, found 560.2934; Calcd for C₃₀H₄₀F₂N₃O₅+0.4 eq of H₂O: C, 63.56; H, 7.08; N, 7.41. Found: C, 63.53; H, 7.14; N, 7.31.

### Example E20

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide

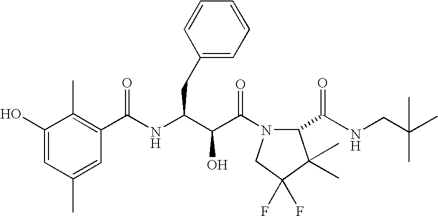

White solid: ¹H NMR (300 MHz, DMSO-d₆) δ 9.22 (s, 1H), 8.12 (d, J=8.6 Hz, 1H), 7.85 (t, J=6.2 Hz, 1H), 7.34 (d, J=7.3 Hz, 2H), 7.23 (t, J=7.3 Hz, 2H), 7.15 (t, J=7.3 Hz, 1H), 6.58 (s, 1H), 6.37 (s, 1H), 5.35 (s, 1H), 4.43 (m, 1H), 4.23-4.33 (m, 4H), 2.95 (dd, J=12.9, 5.8 Hz, 1H), 2.88 (dd, J=13.1, 6.3 Hz, 1H), 2.79 (dd, J=13.1, 2.3 Hz, 1H), 2.66 (dd, J=12.9, 11.4 Hz, 1H), 2.14 (s, 3H), 1.74 (s, 3H), 1.21 (s, 3H), 1.05 (s, 3H), 0.85 (s, 9H) ppm; HRMS (ESI) m/z calcd for C₃₁H₄₂F₂N₃O₅ (M+H)⁺574.3093, found 574.3096; Calcd for C₃₁H₄₂F₂N₃O₅+0.2 eq of H₂O: C, 64.50; H, 7.23; N, 7.28. Found: C, 64.50; H, 7.26; N, 7.30.

### Example E21

### (S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide

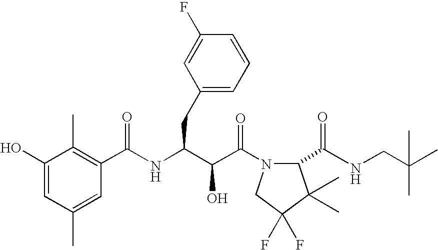

White solid: ¹H NMR (300 MHz, DMSO-d₆) δ 9.26 (s, 1H), 8.21 (d, J=8.6 Hz, 1H), 7.89 (t, J=6.2 Hz, 1H), 7.27 (m, 1H), 7.19-7.22 (m, 2H), 6.98 (t, J=9.4 Hz, 1H), 6.60 (s, 1H), 6.38 (s, 1H), 5.31 (s, 1H), 4.45 (m, 1H), 4.35 (m, 2H), 4.23-4.30 (m, 2H), 2.97 (dd, J=12.9, 6.1 Hz, 1H), 2.88 (dd, J=13.0, 5.9 Hz, 1H), 2.79 (m, 1H), 2.67 (dd, J=13.3, 11.8 Hz, 1H), 2.15 (s, 3H), 1.74 (s, 3H), 1.22 (s, 3H), 1.06 (s, 3H), 0.85 (s, 9H) ppm; HRMS (ESI) m/z calcd for C₃₁H₄₁F₃N₃O₅ (M+H)⁺592.2998, found 592.2998; Calcd for C₃₁H₄₁F₃N₃O₅+0.1 eq of H₂O: C, 62.74; H, 6.83; N, 7.08. Found: C, 62.48; H, 6.82; N, 6.87.

### Example E22

### (S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide

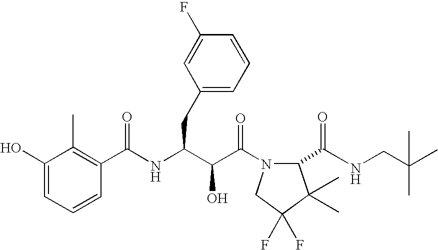

White solid: ¹H NMR (300 MHz, DMSO-d₆) δ 9.39 (s, 1H), 8.27 (d, J=8.6 Hz, 1H), 7.89 (t, J=5.2 Hz, 1H), 7.28 (m, 1H), 7.20 (d, J=4.3 Hz, 2H), 6.93-6.98 (m, 2H), 6.77 (d, J=7.8 Hz, 1H), 6.54 (d, J=7.6 Hz, 1H), 5.35 (s, 1H), 4.41-4.50 (m, 1H), 4.35 (m, 2H), 4.24-4.31 (m, 2H), 2.97 (dd, J=12.9, 5.8 Hz, 1H), 2.88 (dd, J=13.0, 6.4 Hz, 1H), 2.79 (m, 1H), 2.67 (m, 1H), 1.76-1.81 (m, 3H), 1.21 (s, 3H), 1.06 (s, 3H), 0.84 (s, 9H) ppm; HRMS (ESI) m/z calcd for C₃₀H₃₉F₃N₃O₅ (M+H)⁺578.2842, found 578.2859; Calcd for C₃₀H₃₈F₃N₃O₅+0.1 eq of H₂O: C, 62.18; H, 6.65; N, 7.25. Found: C, 61.93; H, 6.69; N, 7.22.

### Example E23

### (S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide

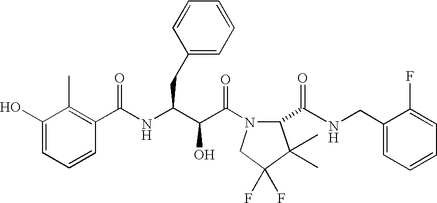

White solid: ¹H NMR (300 MHz, DMSO-d₆) δ 9.37 (s, 1H), 8.46 (t, J=5.9 Hz, 1H), 8.17 (d, J=8.6 Hz, 1H), 7.42 (t, J=7.6 Hz, 1H), 7.21-7.32 (m, 5H), 7.09-7.17 (m, 3H), 6.93 (t, J=7.8 Hz, 1H), 6.76 (d, J=8.1 Hz, 1H), 6.53 (d, J=7.6 Hz, 1H), 5.56 (d, J=7.3 Hz, 1H), 4.45 (m, 1H), 4.28-4.38 (m, 6H), 2.87 (dd, J=12.6, 1.3 Hz, 1H), 2.68 (m, 1H), 1.79 (s, 3H), 1.18 (s, 3H), 0.98 (s, 3H) ppm; HRMS (ESI) m/z calcd for C₃₂H35F₃N₃O₅ (M+H)⁺598.2529, found 598.2511; Calcd for C₃₂H₃₄F₃N₃O₅: C, 64.31; H, 5.73; N, 7.03. Found: C, 64.45; H, 5.85; N, 6.79.

### Example E24

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide

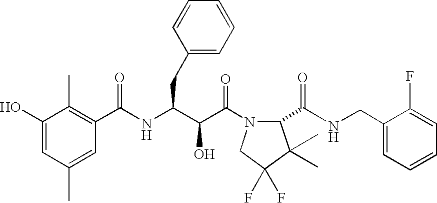

White solid: ¹H NMR (300 MHz, DMSO-d₆) δ 9.23 (s, 1H), 8.47 (t, J=5.8 Hz, 1H), 8.12 (d, J=8.6 Hz, 1H), 7.42 (t, J=8.0 Hz, 1H), 7.21-7.32 (m, 5H), 7.09-7.17 (m, 3H), 6.58 (s, 1H), 6.36 (s, 1H), 5.51 (s, 1H), 4.28-4.49 (m, 7H), 2.85 (dd, J=13.9, 2.0 Hz, 1H), 2.67 (m, 1H), 2.14 (s, 3H), 1.73 (s, 3H), 1.19 (s, 3H), 0.99 (s, 3H) ppm; HRMS (ESI) m/z calcd for C₃₃H₃₇F₃N₃O₅ (M+H)⁺612.2685, found 612.2657; Calcd for C33H36F₃N₃O₅: C, 64.80; H, 5.93; N, 6.87. Found: C, 64.47; H, 5.95; N, 6.77.

### Example E25

### (S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide

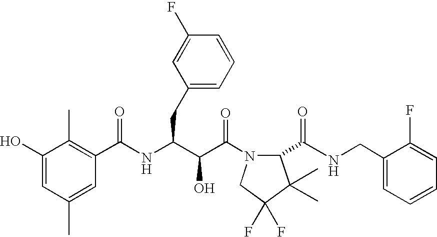

White solid: ¹H NMR (300 MHz, DMSO-d₆) δ 9.26 (s, 1H), 8.51 (t, J=5.7 Hz, 1H), 8.20 (d, J=8.3 Hz, 1H), 7.42 (t, J=7.6 Hz, 1H), 7.23-7.29 (m, 2H), 7.09-7.17 (m, 4H), 6.97 (t, J=7.8 Hz, 1H), 6.60 (s, 1H), 6.38 (s, 1H), 5.46 (s, 1H), 4.46 (m, 1H), 4.25-4.35 (m, 6H), 2.84 (m, 1H), 2.68 (dd, J=13.1, 11.4 Hz, 1H), 2.15 (s, 3H), 1.74 (s, 3H), 1.19 (s, 3H), 0.99 (s, 3H) ppm; HRMS (ESI) m/z calcd for C₃₃H₃₆F₄N₃O₅ (M+H)⁺630.2591, found 630.2570; Calcd for C₃₃H₃₅F₄N₃O₅+0.5 eq of H₂O: C, 62.06; H, 5.68; N, 6.58. Found: C, 62.09; H, 5.55; N, 6.49.

### Example E26

### (S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid propylamide

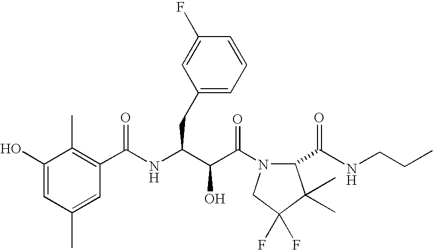

White solid: ¹H NMR (300 MHz, DMSO-d₆) δ 9.25 (s, 1H), 8.18 (d, J=8.3 Hz, 1H), 7.94 (t, J=5.7 Hz, 1H), 7.29 (m, 1H), 7.19 (d, J=7.6 Hz, 2H), 6.99 (t, J=7.8 Hz, 1H), 6.59 (s, 1H), 6.37 (s, 1H), 5.40 (d, J=6.6 Hz, 1H), 4.42 (m, 1H), 4.27-4.33 (m, 3H), 4.21 (s, 1H), 2.96-3.11 (m, 2H), 2.83 (m, 1H), 2.68 (dd, J=13.1, 11.1 Hz, 1H), 2.15 (s, 3H), 1.74 (s, 3H), 1.36-1.44 (m, 2H), 1.19 (s, 3H), 1.03 (s, 3H), 0.84 (t, J=7.5 Hz, 3H) ppm; HRMS (ESI) m/z calcd for C₂₉H₃₇F₃N₃O₅ (M+H)⁺564.2685, found 564.2709; Calcd for C₂₉H₃₇F₃N₃O₅+0.3 eq of H₂O: C, 61.21; H, 6.48; N, 7.39. Found: C, 61.13; H, 6.35; N, 7.31.

### Example E27

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide

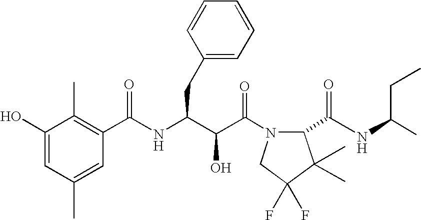

White solid: ¹H NMR (300 MHz, DMSO-d₆) δ 9.23 (s, 1H), 8.15 (d, J=8.3 Hz, 1H), 7.72 (d, J=8.3 Hz, 1H), 7.35 (d, J=7.1 Hz, 2H), 7.23 (t, J=7.5 Hz, 2H), 7.15 (t, J=7.2 Hz, 1H), 6.59 (s, 1H), 6.37 (s, 1H), 5.35 (d, J=6.6 Hz, 1H), 4.44 (m, 1H), 4.26-4.35 (m, 3H), 4.24 (s, 1H), 3.72 (m, 1H), 2.81 (m, 1H), 2.66 (dd, J=13.5, 11.2 Hz, 1H), 2.15 (s, 3H), 1.73 (s, 3H), 1.30-1.45 (m, 2H), 1.20 (s, 3H), 1.04 (s, 3H), 1.02 (d, J=6.6 Hz, 3H), 0.83 (t, J=7.3 Hz, 3H) ppm; HRMS (ESI) m/z calcd for C₃₀H₄₀F₂N₃O₅ (M+H)⁺560.2936, found 560.2919; Calcd for C₃₀H₃₉F₂N₃O₅: C, 64.08; H, 7.02; N, 7.51. Found: C, 64.08; H, 7.02; N, 7.35.

### Example E28

### (S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide

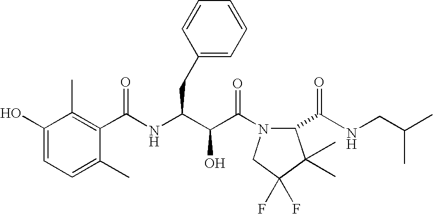

White solid: ¹H NMR (300 MHz, DMSO-d₆) δ 9.06 (s, 1H), 8.36 (d, J=8.3 Hz, 1H), 8.02 (t, J=5.8 Hz, 1H), 7.37 (d, J=7.1 Hz, 2H), 7.22 (t, J=7.3 Hz, 2H), 7.14 (t, J=7.3 Hz, 1H), 6.71 (d, J=8.1 Hz, 1H), 6.61 (d, J=8.1 Hz, 1H), 5.06 (s, 1H), 4.44-4.53 (m, 2H), 4.31-4.37 (m, 3H), 2.87-2.99 (m, 2H), 2.70 (dd, J=13.6, 2.8 Hz, 1H), 2.63 (dd, J=13.6, 11.1 Hz, 1H), 1.66-1.79 (m, 7H), 1.23 (s, 3H), 1.06 (s, 3H), 0.85 (d, J=6.8 Hz, 6H) ppm; HRMS (ESI) m/z calcd for C₃₀H₄₀F₂N₃O₅ (M+H)⁺560.2936, found 560.2945; Calcd for C₃₀H₃₉F₂N₃O₅+0.1 eq of H₂O: C, 64.17; H, 7.04; N, 7.48. Found: C, 63.96; H, 7.05; N, 7.40.

### Example E29

### (S)-4,4-Difluoro-1[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide

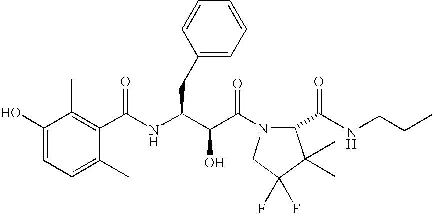

White solid: ¹H NMR (300 MHz, DMSO-d₆) δ 9.06 (s, 1H), 8.36 (d, J=8.1 Hz, 1H), 8.00 (t, J=5.7 Hz, 1H), 7.37 (d, J=7.1 Hz, 2H), 7.22 (t, J=7.5 Hz, 2H), 7.14 (t, J=7.3 Hz, 1H), 6.71 (d, J=8.1 Hz, 1H), 6.61 (d, J=8.1 Hz, 1H), 5.08 (s, 1H), 4.43-4.52 (m, 2H), 4.30-4.36 (m, 2H), 4.27 (s, 1H), 2.98-3.14 (m, 2H), 2.71 (dd, J=13.9, 3.0 Hz, 1H), 2.65 (m, 1H), 1.79 (bs, 3H), 1.68 (bs, 3H), 1.37-1.45 (m, 2H), 1.22 (s, 3H), 1.05 (s, 3H), 0.85 (t, J=7.5 Hz, 3H) ppm; HRMS (ESI) m/z calcd for C₂₉H₃₈F₂N₃O₅ (M+H)⁺546.2780, found 546.2798; Calcd for C₂₉H₃₇F₂N₃O₅+0.3 eq of H₂O: C, 63.21; H, 6.88; N, 7.63. Found: C, 63.21; H, 6.76; N, 7.54.

### Example E30

### (S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide

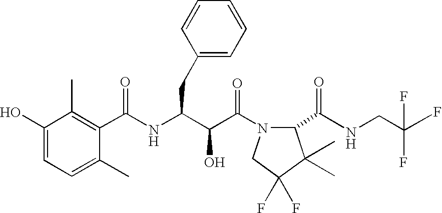

White solid: ¹H NMR (300 MHz, DMSO-d₆) δ 9.07 (s, 1H), 8.75 (t, J=5.9 Hz, 1H), 8.36 (d, J=8.5 Hz, 1H), 7.34 (d, J=7.5 Hz, 2H), 7.23 (t, J=7.2 Hz, 2H), 7.14 (t, J=7.4 Hz, 1H), 6.71 (d, J=8.1 Hz, 1H), 6.60 (d, J=8.1 Hz, 1H), 5.16 (d, J=6.8 Hz, 1H), 4.32-4.57 (m, 5H), 4.06 (m, 1H), 3.88 (m, 1H), 2.64-2.72 (m, 2H), 1.78 (bs, 3H), 1.68 (bs, 3H), 1.23 (s, 3H), 1.05 (s, 3H) ppm; HRMS (ESI) m/z calcd for C₂₈H₃₃F₅N₃O₅ (M+H)⁺586.2340, found 586.2340; Calcd for C₂₈H₃₂F₅N₃O₅+0.3 eq of H₂O: C, 56.91; H, 5.56; N, 7.11. Found: C, 56.76; H, 5.48; N, 6.99.

### Example E31

### (S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide

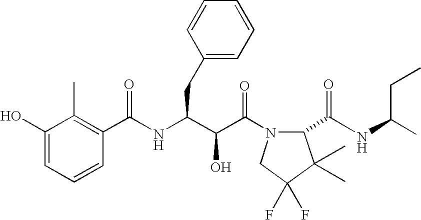

White solid: ¹H NMR (300 MHz, DMSO-d₆), δ 9.36 (s, 1H), 8.20 (d, J=8.3 Hz, 1H), 7.71 (d, J=8.0 Hz, 1H), 7.36 (s, 1H), 7.34 (s, 1H), 7.25-7.15 (m, 3H), 6.94 (t, J=7.8 Hz, 1H), 6.76 (d, J=7.6 Hz, 1H), 6.53 (d, J=7.3 Hz, 1H), 5.39 (d, J=6.9 Hz, 1H), 4.34-4.27 (m, 2H), 4.24 (s, 1H), 4.02 (q, J=7.1 Hz, 1H), 2.79 (s, 1H), 2.66 (s, 1H), 1.98 (s, 1H), 1.79 (s, 3H), 1.43-1.33 (m, 2H), 1.19-1.14 (m, 4H), 1.04-1.01 (m, 6H), 0.83 (t, J=7.4 Hz, 3H) ppm; HRMS m/z calcd for C₂₉H₃₈F₂N₃O₅ (M+H)⁺546.2775, found 546.2780; Anal. Calcd for C₂₉H₃₇F₂N₃O₅+0.63 eq of H₂O: C, 63.84; H, 6.84; N, 7.70. Found: C, 62.90; H, 6.89; N, 7.14.

### Example E32

### (S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide

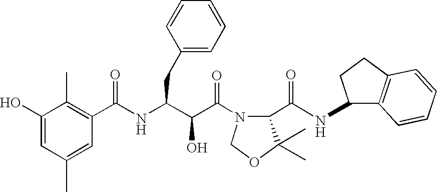

White solid: ¹H NMR (DMSO-d₆) δ 9.22 (s, 1H), 8.32 (d, J=8.3 Hz, 1H), 8.07 (d, J=8.6, 1H), 7.29-7.18 (m, 9H), 6.58 (s, 1H), 6.37 (s, 1H), 5.67 (d, J=6.1 Hz, 1H), 5.45 (d, J=4.0 Hz, 1H), 5.33-5.23 (m, 2H), 4.36 (m, 1H), 4.19 (d, 1H), 4.17 (s, 1H), 2.91-2.66 (m, 6H), 2.15 (s, 3H), 1.72 (s, 3H), 1.29 (s, 3H), 1.27 (s, 3H) ppm; HRMS m/z calcd for C₃₄H₄₀N₃₀O₆ (M+H)⁺586.2900, found 586.2917. Anal Calcd for C₃₄H₃₉N₃O₆+H₂O: C, 69.72; H, 6.71; N, 7.17. Found: C, 69.77; H, 6.87; N, 6.95.

### Example E33

### (S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide

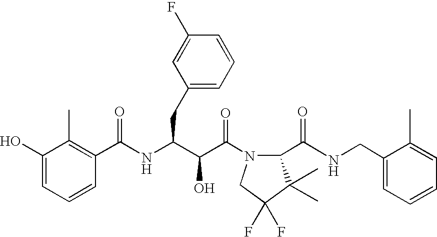

White solid: ¹H NMR (DMSO-d₆) δ 9.38(s, 1H), 8.35 (t, J=5.7 Hz, 1H,), 8.25 (d, J=8.4 Hz, 1H,), 7.31-6.92 (m, 2H), 7.19-7.11 (m, 5H), 7.00-6.92 (m, 2H), 6.78 (d, J=8.1 Hz, 1H,), 6.55 (d, J=7.3 Hz, 1H,), 5.47 (d, J=5.5 Hz, 1H,), 4.50-4.24 (m, 6H), 4.17 (dd, J=15.2, 5.1 Hz, 1H), 2.87-2.65 (m, 2H), 2.27 (s, 3H), 1.79 (s, 3H), 1.19 (s, 3H), 1.02 (s, 3H) ppm; HRMS (ESI) m/z calcd for C₃₃H₃₇F₃N₃O₅ (M+H)⁺612.2685, found 612.2701.

### Example E34

### (S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide

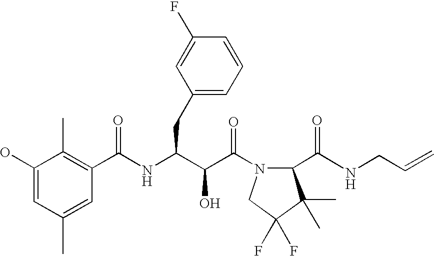

White solid: ¹H NMR (DMSO-d₆) δ 9.25 (s, 1H), 8.18 (d, J=8.3 Hz, 1H), 8.14 (t, J=5.8 Hz, 1H), 7.25-7.31 (m, 1H), 7.18 (d, J=7.6 Hz, 2H), 6.99 (t, J=7.1 Hz, 1H), 6.60 (s, 1H), 6.37 (s, 1H), 5.73-5.82 (m, 1H), 5.43 (bs, 1H), 5.21 (dd, J=1.8, 17.2 Hz, 1H), 5.04 (dd, J=1.4, 10.4 Hz, 1H), 4.44 (q, J=12.4 Hz, 1H), 4.26-4.32 (m, 4H), 3.70-3.75 (m, 2H), 2.81-2.85 (m, 1H), 2.69 (dd, J=11.2, 13.8 Hz, 1H), 2.15 (s, 3H), 1.74 (s, 3H), 1.20 (s, 3H), 1.03 (s, 3H) ppm; HRMS (ESI) m/z calcd for C₂₉H₃₅F₃N₃O₅ (M+H)⁺562.2529, found 562.2534; Calcd for C₂₉H₃₄F₃N₃O₅: C, 62.02; H, 6.10; N, 7.48. Found: C, 61.94; H, 6.31; N, 7.29.

### Example E35

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide

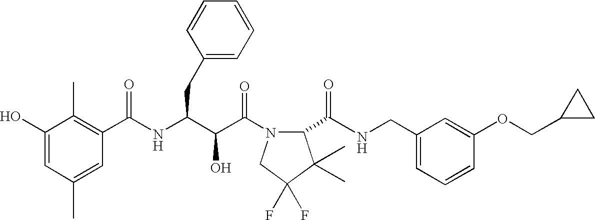

White solid: ¹H NMR (300 MHz, DMSO-d₆) δ 9.23 (s, 1H), 8.41 (t, J=5.9 Hz, 1H), 8.10 (d, J=7.8 Hz, 1H), 7.32 (d, J=7.3 Hz, 2H), 7.23 (t, J=7.5 Hz, 2H), 7.16 (t, J=8.1 Hz, 2H), 6.83 (d, J=7.3 Hz, 2H), 6.74 (d, J=8.8 Hz, 1H), 6.59 (s, 1H), 6.36 (s, 1H), 5.47 (d, J=6.0 Hz, 1H), 4.28-4.46 (m, 6H), 4.14 (dd, J=5.3, 15.2 Hz, 1H), 3.72-3.80 (m, 2H), 2.85-2.96 (m, 1H), 2.69 (dd, J=9.6, 13.6 Hz, 1H), 2.14 (s, 3H), 1.74 (s, 3H), 1.20 (s, 3H), 1.02 (s, 3H), 0.53 (m, 2H), 0.27 (m, 2H) ppm; HRMS (ESI) m/z calcd for C₃₇H₄₄F₂N₃O₆ (M+H)⁺664.3198, found 664.3225; Calcd for C₃₇H₄₄F₂N₃O₆: C, 66.95; H, 6.53; N, 6.33. Found: C, 66.86; H, 6.50; N, 6.07.

### Example E36

### 2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide

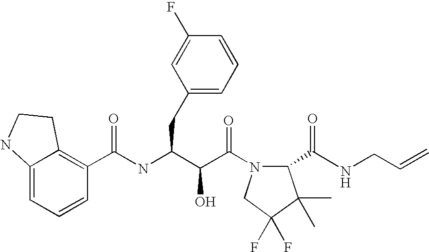

White solid: ¹H NMR (300 MHz, DMSO-d₆) δ 8.14 (t, J=5.7 Hz, 1H), 8.09 (d, J=8.3 Hz, 1H), 7.24-7.29 (m, 1H), 7.19 (d, J=7.6 Hz, 2H), 6.90 (m, 2H), 6.69 (d, J=7.3 Hz, 1H), 6.53 (d, J=7.5 Hz, 1H), 5.73-5.82 (m, 1H), 5.61 (bs, 1H), 5.47 (d, J=6.6 Hz, 1H), 5.21 (dd, J=1.8, 17.2 Hz, 1H), 5.04 (dd, J=1.6, 10.2 Hz, 1H), 4.25-4.43 (m, 5H), 3.72-3.73 (m, 2H), 3.30-3.35 (m, 2H), 2.79-2.97 (m, 4H), 1.21 (s, 3H), 1.03 (s, 3H) ppm; HRMS (ESI) m/z calcd for C₂₉H₃₄F₃N₄O₄ (M+H)⁺559.2532, found 559.2560; Calcd for C₂₉H₃₃F₃N₄O₄: C, 62.36; H, 5.95; N, 10.03. Found: C, 62.27; H, 5.96; N, 9.83.

### Example E37

### (S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide

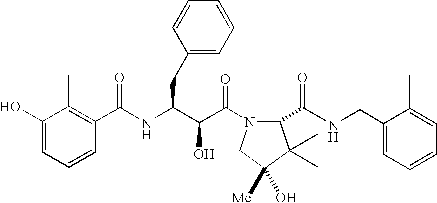

White solid: ¹H NMR (300 MHz, DMSO-d₆) δ 9.36 (s, 1H), 8.89 (t, J=5.6 Hz, 1H), 8.19 (d, J=8.3 Hz, 1H), 7.35 (d, J=6.8 Hz, 1H), 7.28 (d, J=7.3 Hz, 2H), 7.22 (t, J=7.5 Hz, 2H), 7.07-7.16 (m, 4H), 6.94 (t, J=7.7 Hz, 1H), 6.77 (d, J=8.1 Hz, 1H), 6.54 (d, J=7.3, 1H), 5.77 (s, 1H), 5.22 (d, J=6.8 Hz, 1H), 4.44 (dd, J=15.0 Hz, 6.19, 1H), 4.26-4.34 (m, 2H), 4.25 (s, 1H), 4.18 (dd, J=15.4, 5.1 Hz, 1H), 4.13 (d, J=11.1 Hz, 1H), 3.81 (d, J=10.9 Hz, 1H), 2.76-2.79 (m, 1H), 2.66 (dd, J=13.0 Hz, 11.24, 1H), 2.26 (s, 3H), 1.80 (s, 3H), 1.06 (s, 3H), 1.01 (s, 3H), 0.91 (s, 3H) ppm; HRMS (ESI) m/z calcd for C₃₄H₄₂N₃O₆ (M+H)⁺588.3074, found 588.3068; Calcd for C₃₄H₄₁N₃O₆+1.1 eq of H₂O: C, 67.22; H, 7.17; N, 6.93. Found: C, 67.16; H, 6.81; N, 6.92.

### Example E38

### (S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide

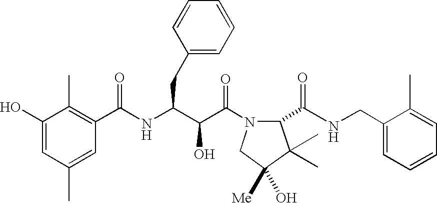

White solid: ¹H NMR (300 MHz, DMSO-d₆) δ 9.22 (s, 1H), 8.89 (t, J=5.6 Hz, 1H), 8.13 (d, J=8.3 Hz, 1H), 7.35 (d, J=6.8 Hz, 1H), 7.28 (d, J=7.1 Hz, 2H), 7.21 (t, J=7.5 Hz, 2H), 7.08-7.16 (m, 4H), 6.59 (s, 1H), 6.38 (s, 1H), 5.77 (s, 1H), 5.17 (d, J=6.8 Hz, 1H), 4.44 (dd, J=15.2, 6.1 Hz, 1H), 4.28-4.34 (m, 2H), 4.25 (s, 1H), 4.18 (dd, J=15.0, 4.7 Hz, 1H), 4.12 (d, J=10.9 Hz, 1H), 3.80 (d, J=10.9 Hz, 1H), 2.76 (m, 1H), 2.66 (dd, J=13.1, 11.1 Hz, 1H), 2.25 (s, 3H), 2.15 (s, 3H), 1.75 (s, 3H), 1.07 (s, 3H), 1.02 (s, 3H), 0.91 (s, 3H) ppm; HRMS (ESI) m/z calcd for C₃₅H₄₄N₃O₆ (M+H)⁺602.3230, found 602.3264; Calcd for C₃₅H₄₄N₃O₆+1 eq of H₂O: C, 67.83; H, 7.32; N, 6.78. Found: C, 67.79; H, 7.11; N, 6.76.

### Example E39

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide

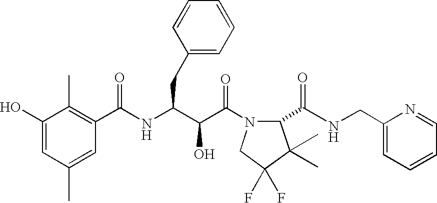

White solid: ¹H NMR (300 MHz, DMSO-d₆) δ 9.08(s, 1H), 8.35 (t, J=8.1 Hz, 1H), 8.17 (d, J=8.1 Hz, 1H), 7.91 (d, J=5.8 Hz, 1H), 7.48 (t, J=7.3 Hz, 1H), 7.20 (d, J=7.4 Hz, 1H), 7.17-6.90 (m, 5H), 6.40 (s, 1H), 6.22 (s, 1H), 6.47 (s, 1H), 4.46-4.26 (m, 4H), 2.68 (d, J=8.2 Hz, 2H), 2.51-2.34 (m, 2H), 1.81 (s, 3H), 1.51 (s, 3H), 1.05 (s, 3H), 0.83 (s, 3H) ppm; HRMS (ESI) m/z calcd for C₃₂H₃₆F₂N₄O₅ (M+H)⁺595.2662, found 595.2654; Calcd for C₃₂H₃₆F₂N₄O₅+2.5 eq of H₂O: C, 60.02; H, 6.46; N, 8.76. Found: C, 60.02; H, 6.08; N, 8.36.

### Example E40:

### 1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide

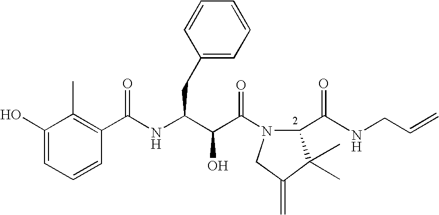

The diastereomers at C₂ were separated by SiO₂ using (1:1-Hex:EtOAc) to provide the desired Example E40: ¹H NMR (300 MHz, DMSO-d₆) δ 9.35 (s, 1H), 8.17 (t, J=5.9 Hz, 1H), 8.10 (d, J=7.7 Hz, 1H), 7.15-7.30 (m, 6H), 6.94 (t, J=7.8 Hz, 1H), 6.76 (d, J=7.3 Hz, 1H), 6.54 (d, J=7.1 Hz, 1H), 5.73 (m, 1H), 5.18 (d, J=17.5 Hz, 1H), 4.87-5.02 (m, 3H), 4.58 (d, J=10.7 Hz, 1H), 4.33 (m, 3H), 4.01 (m, 1H), 3.63-3.72 (m, 2H), 2.80 (m, 1H), 2.66 (m, 1H), 1.79 (s, 3H), 1.13 (s, 3H), 1.08 (s, 3H) ppm; Calcd for C₂₉H₃₅N₃O₅+0.5 eq of H₂O: C, 67.68; H, 7.05; N, 8.17. Found: C, 67.40; H, 7.16; N, 7.93.

### Example E41

### 1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide

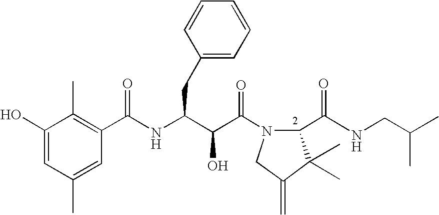

The diastereomers at C₂ were separated by SiO₂ using (1:1-Hex:EtOAc) to provide the desired Example E41: ¹H NMR (300 MHz, DMSO-d₆) δ 9.21 (s, 1H), 8.06 (d, J=8.5 Hz, 1H), 8.01 (t, J=5.9 Hz, 1H), 7.29 (d, J=7.1 Hz, 1H), 7.22 (dd, J=7.6, 7.1 Hz, 2H), 7.15 (d, J=7.1 Hz, 1H), 6.58 (s, 1H), 6.37 (s, 1H), 5.21 (d, J=6.3 Hz, 1H), 4.87 (s, 1H), 4.33-4.37 (m, 3H), 4.50-4.60 (m, 2H), 2.87 (t, J=6.4 Hz, 2H), 2.76 (m, 1H), 2.65 (m, 1H), 2.15 (s, 3H), 1.73 (s, 3H), 1.66 (m, 1H), 1.14 (s, 3H), 1.08 (s, 3H), 0.81 (d, J=6.8 Hz, 6H) ppm; Calcd for C₃₁H₄₁N₃O₅+0.5 eq of H₂O: C, 68.36; H, 7.77; N, 7.71. Found: C, 68.34; H, 7.91; N, 7.41.

### Example E42

### 1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide

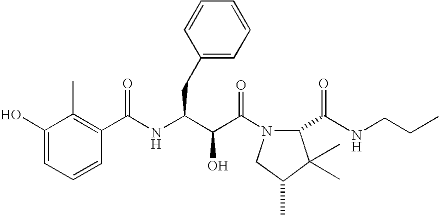

¹H NMR (300 MHz, DMSO-d₆) δ 9.36 (s, 1H), 8.14 (d, J=8.1 Hz, 1H), 7.73 (t, J=5.6 Hz, 1H), 7.37 (d, J=7.3 Hz, 2H), 7.24 (t, J=7.5 Hz, 1H), 7.15 (t, J=7.3 Hz, 1H), 6.94 (t, J=7.8 Hz, 1H), 6.77 (d, J=7.8 Hz, 1H), 6.55 (d, J=6.8 Hz, 1H), 5.56 (d, J=7.8 Hz, 1H), 4.30-4.37 (m, 2H), 3.91-3.95 (m, 2H), 3.26-3.32 (m, 3H), 2.89-3.14 (m, 2H), 2.80 (dd, J=13.4, 2.5 Hz, 1H), 2.66 (dd, J=12.9, 10.9 Hz, 1H), 1.82 (s, 3H), 1.37-1.42 (m, 2H), 1.12 (s, 3H), 0.89 (d, J=6.8 Hz, 3H), 0.84 (t, J=7.6 Hz, 3H), 0.73 (s, 3H) ppm; Calcd for C₂₉H₃₉N₃O₅: C, 68.35; H, 7.71; N, 8.24. Found: C, 68.35; H, 8.01; N, 8.55.

### Example E43

### 1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide

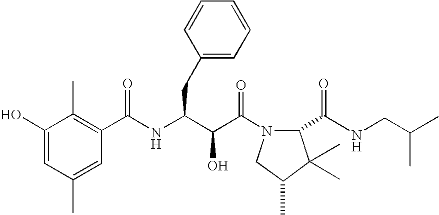

¹H NMR (300 MHz, DMSO-d₆) δ 9.23 (s, 1H), 8.09 (d, J=8.5 Hz, 1H), 7.77 (t, J=5.8 Hz, 1H), 7.37 (d, J=6.9 Hz, 2H), 7.24 (t, J=7.3 Hz, 2H), 7.15 (t, J=7.2 Hz, 1H), 6.59 (s, 1H), 6.38 (s, 1H), 4.82 (d, J=5.5 Hz, 1H), 4.34 (m, 2H), 3.95 (s, 1H), 3.92 (m,1H), 3.01 (m,1H), 2.76 (m, 3H), 2.64 (m, 1H), 2.15 (s, 3H), 1.93 (m, 1H), 1.76 (s, 3H), 1.68 (m, 1H), 1.13 (s, 3H), 0.89 (d, J=6.8 Hz, 3H), 0.85 (d, J=6.8 Hz, 3H), 0.84 (d, J=6.8 Hz, 3H), 0.74 (s, 3H) ppm; HRMS (ESI) m/z calcd for C₃₁H₄₄N₃O₅ (M+H)⁺538.3281, found 538.3297.

### Example E44

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide

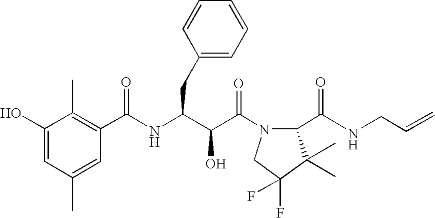

White solid: ¹H NMR (300 MHz, DMSO-d₆) δ 9.25 (s, 1H), 8.13-8.10 (m, 2H), 7.37-7.15 (m, 5H), 6.60 (s, 1H), 6.37 (s, 1H), 5.84-5.73 (m, 1H), 5.50 (d, J=6.1, 1H), 5.23 (dd, J=1.7, 17.5 Hz, 1H), 5.05 (dd, J=1.5, 10.4 Hz, 1H), 4.49-4.28 (m, 3H), 4.26 (s, 1H), 3.78-3.68 (m, 2H), 2.89-2.66 (m, 2H), 2.16 (s, 3H), 1.75 (s, 3H), 1.21 (s, 3H), 1.05 (s, 3H) ppm; HRMS (ESI) m/z calcd for C₂₉H₃₆F₂N₃O₅ (M+H)⁺544.6070, found 544.2623; Calcd for C₂₉H₃₅F₂N₃O₅+0.5 eq of H₂O: C, 63.05; H, 6.57; N, 7.60. Found: C, 63.05; H, 6.40; N, 7.39.

### Example E45

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide

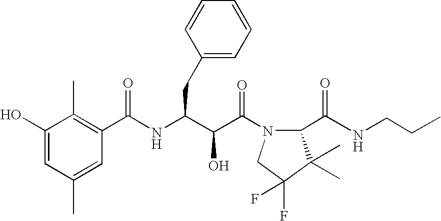

White solid: ¹H NMR (300 MHz, DMSO-d₆) δ 9.17 (s, 1H), 8.04 (d, J=8.1 Hz, 1H), 7.85 (t, J=5.1 Hz, 1H), 7.29-7.09 (m, 5H), 6.53 (s, 1H), 6.30 (s, 1H), 5.38 (d, J=6.1 Hz, 1H), 4.40-4.24 (m, 3H), 4.14 (s, 1H), 3.04-2.90 (m, 2H), 2.77 (d, J=2.2 Hz, 1H), 2.65-2.59 (m, 1H), 2.09 (s, 3H), 1.67 (3, 3H), 1.39-1.31 (m, 2H), 1.13 (s, 3H), 0.97 (s, 3H), 0.78 (s, 3H) ppm; HRMS (ESI) m/z calcd for C₂₉H₃₈F₂N₃O₅ (M+H)⁺546.6230, found 546.2780; Calcd for C₂₉H₃₇F₂N₃O₅: C, 63.84; H, 6.84; N, 7.70. Found: C, 63.44; H, 6.82; N, 7.52.

### Example E46

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide

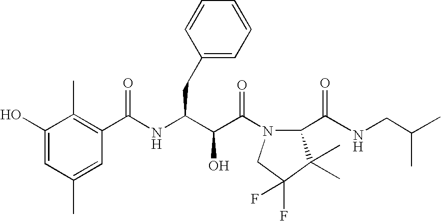

White solid: ¹H NMR (300 MHz, DMSO-d₆) δ 9.24 (s, 1H), 8.11 (d, J=8.3 Hz, 1H), 7.94 (t, J=5.8 Hz, 1H), 7.37-7.16 (m, 5H), 6.60 (s, 1H), 6.38 (s, 1H), 5.44 (d, J=6.3 Hz, 1H), 4.48-4.29 (m, 3H), 4.25 (s, 1H), 2.94-2.83 (m, 3H), 2.73-2.64 (m, 1H), 2.16 (s, 3H), 1.75 (s, 3H), 1.74-1.65 (m, 1H), 1.21 (s, 3H), 1.05 (s, 3H), 0.86 (d, J=6.6 Hz, 6H) ppm; HRMS (ESI) m/z calcd for C₃₀H₄₀F₂N₃O₅ (M+H)⁺560.6500, found: 560.2928; Calcd for C₃₀H₃₉F₂N₃O₅: C, 64.38; H, 7.02; N, 7.51. Found: C, 64.09; H, 7.05; N, 7.29.

### Example E47

### (S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide

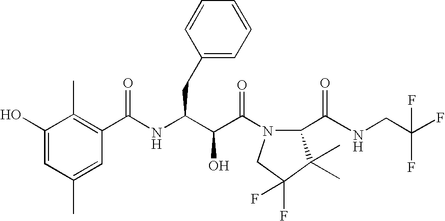

White solid: ¹H NMR (300 MHz, DMSO-d₆) δ 9.27 (s, 1H), 8.72 (t, J=6.2 Hz, 1H), 8.15 (d, J=8.1Hz, 1H), 7.37-7.19 (m, 5H), 6.63 (s, 1H), 6.39 (s, 1H), 5.57 (d, J=6.3 Hz, 1H), 4.52-4.33 (m, 4H), 4.10-3.94 (m, 1H), 3.93-3.88 (m, 1H), 2.87 (d, J=7.3 Hz, 1H), 2.75-2.69 (m, 1H), 2.19 (s, 3H), 1.77 (s, 3H), 1.25 (s, 3H), 1.06 (s, 3H) ppm; HRMS (ESI) m/z calcd for C₂₈H₃₃F₃N₃O₅ (M+H)⁺586.5670, found 586.2340; Calcd for C₂₈H₃₂F₃N₃O₅+0.4 eq. of H₂O: C, 56.73; H, 5.58; N, 7.09. Found: C, 56.64; H, 5.41; N, 6.94.

### Example E48

### (S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide

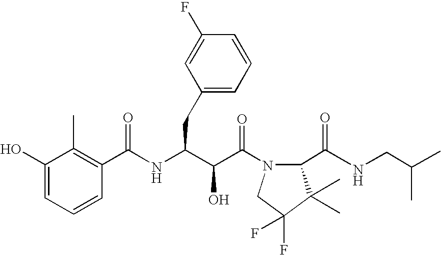

White solid: ¹H NMR (300 MHz, DMSO-d₆) δ 9.14 (s, 1H), 8.03 (d, J=8.3 Hz, 1H), 7.76 (t, J=5.8 Hz, 1H), 7.09 (dd, J=7.4, 14.4 Hz, 1H), 6.99 (d, J=7.6 Hz, 2H), 6.81-6.73 (m, 2H), 6.58 (d, J=8.1Hz, 1H), 6.34 (d, J=6.8Hz, 1H), 5.23 (d, J=6.6Hz, 1H), 4.25 (dd, J=12.2, 25.0 Hz, 1H), 4.15-4.08 (m, 3H), 2.77-2.46 (m, 4H), 1.59 (s, 3H), 1.52-1.43 (m, 1H), 1.00 (s, 3H), 0.83 (s, 3H), 0.65 (d, J=6.4, 6H) ppm; HRMS (ESI) m/z calcd for C₃₀H₃₇F₃N₃O₅ (M+H)⁺564.6130, found: 564.2674; Calcd for C₃₀H₃₆F₃N₃O₅: C, 61.80; H, 6.44; N, 7.46. Found: C, 61.58; H, 6.45; N, 7.34.

### Example E49

### N-((1S,2S)-3-{(2S)-2-[(allylamino)carbonyl]-4,4-difluoro-3,3-dimethylpyrrolidin-1-yl}-1-benzyl-2-hydroxy-3-oxopropyl)indoline-4-carboxamide

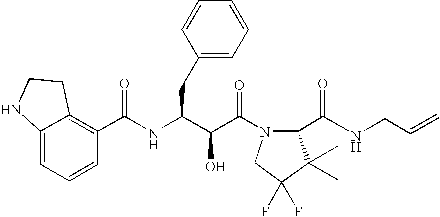

White solid: ¹H NMR (300 MHz, DMSO-d₆) δ 8.10 (t, J=5.8 Hz, 1H), 8.01 (d, J=8.1 Hz, 1H), 7.35 (d, J=7.1 Hz, 2H), 7.23 (t, J=7.3 Hz, 2H), 7.13 (t, J=7.3 Hz, 1H), 6.90 (t, J=7.8 Hz, 1H), 6.67 (d, J=6.8 Hz, 1H), 6.51 (d, J=7.1 Hz, 1H), 5.72-5.82 (m, 1H), 5.59 (brs, 1H), 5.50 (d, J=6.3 Hz, 1H), 5.18-5.24 (m, 1H), 5.02-5.06 (m, 1H), 4.27-4.40 (m, 3H), 4.23 (s, 1H), 3.70-3.73 (m, 2H), 3.27-3.36 (m, 3H), 2.77-2.97 (m, 4H), 1.21 (s, 3H), 1.02 (s, 3H) ppm; HRMS (ESI) m/z calcd for C₂₉H₃₅F₂N₄O₄ (M+H)⁺541.2640, found 541.2626; Calcd for C₂₉H₃₄F₂N₄O₄+0.1 eq of H₂O: C, 64.43; H, 6.34; N, 10.36. Found: C, 4.21; H, 6.36; N, 10.33.

### Example E50

### (4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide

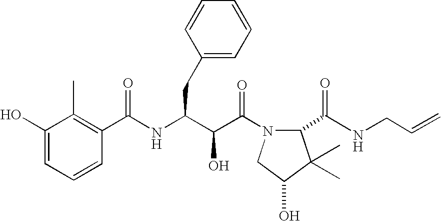

¹H NMR (300 MHz, DMSO-d₆) δ 9.36 (s, 1H), 8.40 (t, J=5.8 Hz, 1H), 8.16 (d, J=7.8 Hz, 1H), 7.30 (d, J=7.1 Hz, 2H), 7.23 (t, J=7.5 Hz, 2H), 7.15 (t, J=7.2 Hz, 1H), 6.94 (t, J=7.7 Hz, 1H), 6.77 (d, J=8.1 Hz, 1H), 6.54 (d, J=7.1 Hz, 1H), 5.76 (m, 1H), 5.35 (d, J=9.1 Hz, 1H), 5.24 (dd, J=17.2, 1.8 Hz, 1H), 5.18 (d, J=4.8 Hz, 1H), 5.02 (dd, J=10.2, 1.6 Hz, 1H), 4.31 (m, 2H), 4.06 (s, 1H), 3.99 (d, J=4.0 Hz, 1H), 3.85 (dd, J=11.1, 3.5 Hz, 1H), 3.68-3.73 (m, 3H), 2.80 (m, 1H), 2.66 (dd, J=13.1, 11.1 Hz, 1H), 1.81 (s, 3H), 1.06 (s, 3H), 0.92 (s, 3H) ppm; Calcd for C₂₈H₃₅N₃O₆+0.7 eq of H₂O: C, 64.40; H, 7.03; N, 8.05. Found: C, 64.38; H, 6.98; N, 7.66.

### Example E51

### (S)-3-((2S,3S)-2-Hydroxy-3-{[1-(3-hydroxy-2-methyl-phenyl)-methanoyl]-amino}-4-phenyl-butanoyl)-5,5-dimethyl-oxazolidine-4-carboxylic acid (S)-indan-1-ylamide

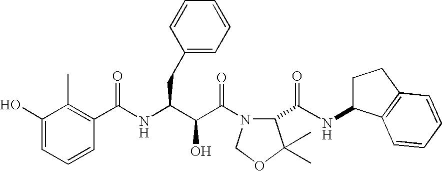

¹H NMR (300 MHz, DMSO-d₆) δ 9.34 (s, 1H), 8.31 (d, J=8.1 Hz, 1H), 8.13 (d, J=8.9 Hz, 1H), 7.22 (m, 9H), 6.94 (t, J=7.7 Hz, 1H), 6.76 (d, J=8.1 Hz, 1H), 6.55 (d, J=7.5 Hz, 1H), 5.71 (d, J=5.8 Hz, 1H), 5.46 (d, J=3.6 Hz, 1H), 5.24-5.34 (m, 2H), 4.55 (s, 1H), 4.37 (m, 1H), 4.18 (m, 1H), 2.71-2.96 (m, 4H), 2.33-2.41 (m, 2H), 1.77 (s, 3H), 1.28 (s, 3H), 1.27 (s, 3H) ppm; Calcd for C₃₃H₃₇N₃O₆+1.9 eq of H₂O+0.2 eq of EtOAc: C, 65.11; H, 6.85; N, 6.74. Found: C, 65.36; H, 6.99; N, 6.42.

### Example E52

### (3R)-N-allyl-1-{(2S,3S)-2-hydroxy-3-[(3-hydroxy-2-methylbenzoyl)amino]- 4-phenylbutanoyl}-3-phenyl-L-prolinamide

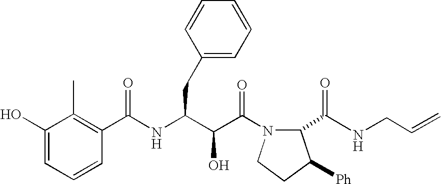

¹HNMR (300 MHz, CD₃OD): δ 7.32-7.06 (m, 1OH), 6.86 (t, J=7.8 Hz, 1H), 6.68 (d, J=8.0 Hz, 1H), 6.53 (d, J=7.6 Hz, 1H), 5.70-5.54 (m, 1H), 5.01-4.81 (m, 1H), 4.63 (m, 1H), 4.54-4.49 (m, 1H), 4.33(d, J=9.0 Hz, 1H), 4.12-4.01 (m, 1H), 3.95-3.80 (m, 1H), 3.76-3.66 (m, 1H), 3.65-3.53 (m, 1H), 3.46-3.33 (m, 1H), 2.95-2.84 (m, 1H), 2.78-2.62 (m, 1H), 2.36-2.22 (m, 1H), 2.20-2.04 (m, 1H), 1.81(s, 3H) ppm. HRMS (ESI) m/z calcd for C₃₂H₃₅N₃O₅Na (M+Na): 564.2475, found for C₃₂H₃₅N₃O₅ Na (M+Na): 564.2474.

### Example E53

### (4S)-N-(2-fluorobenzyl)-1-{(2S,3S)-2-hydroxy-3-[(3-hydroxy-2-methylbenzoyl)amino]-4-phenylbutanoyl}-4-methoxy-3,3-dimethyl-L-prolinamide

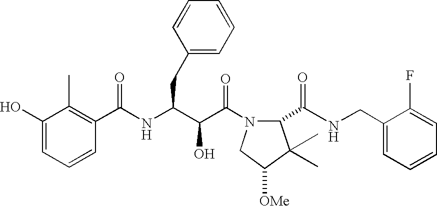

¹HNMR (300 MHz, CD₃OD): δ 7.52-7.41 (m, 1H), 7.36-7.16 (m, 6H), 7.15-7.00 (m, 2H), 6.96 (t, J=9.0 Hz, 1H), 6.77 (d, J=9.0 Hz, 1H), 6.62 (d, J=9.0 Hz, 1H), 4.69-4.55 (m, 1H), 4.54-4.47 (m, 2H), 4.44-4.39 (m, 1H), 4.22-4.13 (m, 1H), 4.11-4.08 (m, 1H), 4.07-3.98 (m, 1H), 3.63-3.56 (m, 1H), 3.43 (s, 3H), 3.13-3.02 (m, 1H), 2.85-2.72 (m, 1H), 1.89 (s, 3H), 1.22 (s, 3H), 0.97 (s, 3H) ppm; HRMS (ESI) m/z calcd for C₃₃H₃₉FN₃O₆ (M+H): 592.2823, found for C₃₃H₃₉FN₃O₆ (M+H): 592.2823. HPLC purity: 99%.

### Example E54

### (4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide

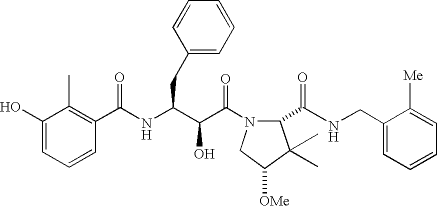

¹HNMR (300 MHz, CD₃OD): δ 7.38-7.07 (m, 9H), 6.95 (t, J=9.0 Hz, 1H), 6.77 (d, J=9.0 Hz, 1H), 6.62 (d, J=9.0 Hz, 1H), 4.69-4.55 (m, 1H), 4.54-4.47 (m, 1H), 4.44 (s, 1H), 4.34-4.26 (m, 1H), 4.22-4.13 (m, 1H), 4.11 (s, 1H), 4.07-3.98 (m, 1H), 3.63-3.56 (m, 1H), 3.43 (s, 3H), 3.13-3.02 (m, 1H), 2.85-2.72 (m, 1H), 2.32 (s, 3H), 1.89 (s, 3H), 1.22 (s, 3H), 1.02 (s, 3H). HRMS (ESI) m/z calcd for C₃₄H₄₂N₃O₆ (M+H): 588.3073, found for C₃₄H₄₂N₃O₆ 588.3065. HPLC purity: 99%.

The examples and preparations provided above further illustrate and exemplify the compounds of the present invention and methods of preparing such compounds. It is to be understood that the scope of the present invention is not limited in any way by the scope of these examples and preparations.

### Example E55

### Preparation of 3acetoxy-2,5-dimethyl-benzoic acid

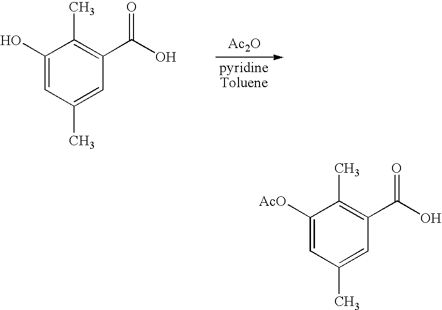

Pyridine (34.0 mL, 419 mmol) and acetic anhydride (150 mL, 1.59 mol) were sequentially added to a suspension of 3-hydroxy-2,5-dimethyl-benzoic acid (211 g, 1.27 mol) in toluene (1.05 L). The mixture was heated at 50° C. under argon for 6 h. Heating was discontinued and, while the mixture was still warm, n-heptane (2.10 L) was added. The mixture was allowed to cool and stir at ambient temperature overnight. The suspension was filtered, using n-heptane for rinsing, and the solid was dried in a vacuum oven at 50° C. to give 212 g (80.1%) of 3-acetoxy-2,5-dimethyl-benzoic acid as a pale yellow solid: m.p.=153-154° C.; ¹H NMR (300 MHz, CDCl₃) δ 11.5 (brs, 1H), 7.80 (s, 1H), 7.10 (s, 1H), 2.44 (s, 3H), 2.41 (s, 3H), 2.39 (s, 3H); ¹³C NMR (75 MHz, DMSO-d₆) δ 169.3, 168.8, 149.9, 136.3, 132.9, 128.4, 128.0, 126.3, 20.8, 20.5, 13.1; MS (Cl) m/z 209.0822 (209.0814 calcd for C₁₁H₁₃O₄, M+H⁺); elemental analysis calcd for C₁₁H₁₂O₄: C, 63.45; H, 5.81; found: C, 63.54; H, 5.88.

### Example E56

### Preparation of Acetic acid 3-chlorocarbonyl-2,5-dimethyl-phenyl ester

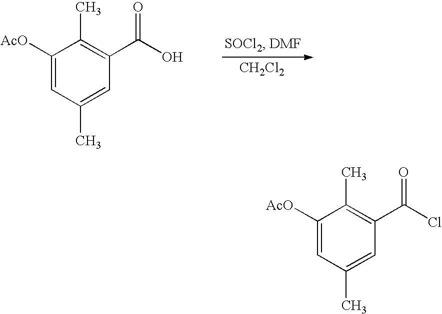

SOCl₂ (80.0 mL, 1.09 mol) was added to a suspension of 3-acetoxy-2,5-dimethyl-benzoic acid (206 g, 990 mmol), DMF (4.0 mL), and CH₂Cl₂ (1.03 L). The resulting mixture was stirred at ambient temperature for 1.5 h. n-Heptane (1.03 L) was added, followed by the slow addition of saturated aqueous NaHCO₃ (2.06 L), and the layers were then separated. The organic fraction was washed with saturated aqueous NaCl (1.00 L), dried over MgSO₄, filtered, and concentrated with a rotary evaporator to give 193 g (86.2%) of acetic acid 3-chlorocarbonyl-2,5-dimethyl-phenyl ester as a pale yellow solid: m.p.=52-54° C; ¹H NMR (300 MHz, CDCl₃) δ 7.92 (s, 1H), 7.15 (s, 1H), 2.44 (s, 3H), 2.38 (s, 3H), 2.35 (s, 3H); ¹³C NMR (75 MHz, CDCl₃) δ 169.4, 167.7, 150.1, 137.3, 134.7, 132.0, 130.2, 129.1, 21.2, 21.1, 13.7; elemental analysis calcd for C₁₁H₁₁O₃Cl: C, 58.29; H, 4.89; found: C, 58.64; H, 4.89.

### Example E57

### Preparation of (2S,3S)-3-(3-Acetoxy-2,5-dimethyl-benzoylamino)-2-hydroxy-4-phenyl-butyric acid

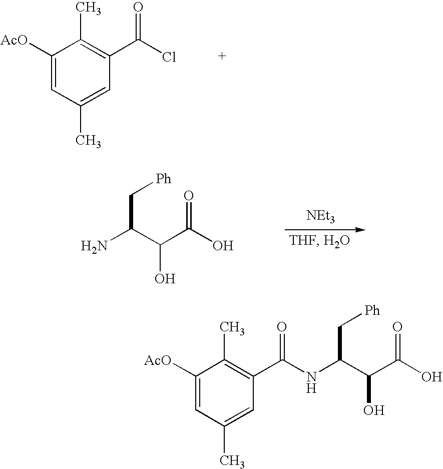

NEt₃ (265 mL, 1.88 mol) was added to a suspension of (2S,3S)-3-amino-2-hydroxy-4-phenyl-butyric acid (175 g, 896 mmol), tetrahydrofuran (875 mL), and H₂O (875 mL) at ambient temperature. The resulting solution was cooled to 0° C. A solution of acetic acid 3-chlorocarbonyl-2,5-dimethyl-phenyl ester (193 g, 854 mmol) and tetrahydrofuran (430 mL) was slowly added. One hour later, H₂O (225 mL) was added, followed by the slow addition of 3 N HCl (390 mL). The resulting mixture was allowed to slowly warm to ambient temperature with stirring overnight. The solid was filtered, using H₂O (430 mL) for rinsing. After drying in a vacuum oven at 50° C., 301 g (91.5%) of (2S,3S)-3-(3-acetoxy-2,5-dimethyl-benzoylamino)-2-hydroxy-4-phenyl-butyric acid was obtained as a white solid that was contaminated with ˜8 mol % Et₃N.HCl: m.p.=220-224° C.; ¹H NMR (300 MHz, DMSO-d₆) δ 12.65 (br s, 1H), 8.23 (d, J=9.0 Hz, 1H), 7.15-7.30 (m, 5H), 6.89 (s, 1H), 6.79 (s, 1H), 5.63 (br s, 1H), 4.39-4.50 (m, 1H), 4.07 (d, J=5.9 Hz, 1H), 2.91 (app dd, J=3.0, 14.0 Hz, 1H), 2.74 (app dd, J=11.1, 14.1 Hz, 1H), 2.27 (s, 3H), 1.24 (s, 3H), 1.72 (s, 3H) [characteristic resonances of Et₃N.HCl: δ 3.09 (q, J=7.3 Hz), 1.18 (t, J=7.3 Hz)]; ¹³C NMR (75 MHz, DMSO-d₆) δ 174.4, 169.2, 168.2, 149.4, 139.4, 135.9, 129.5, 128.3, 126.3, 125.6, 124.7, 123.5, 73.2, 53.5, 35.4, 20.8, 20.6, 12.2 [characteristic resonances of Et₃N.HCl: δ 45.9, 8.8]; MS (Cl) m/z 386.1600 (386.1604 calcd for C₂₁H₂₄NO₆, M+H⁺); elemental analysis calcd for C₂₁H₂₃NO₆.0.08 Et₃N.HCl: C, 65.08; H, 6.17; N, 3.82; found: C, 64.88; H, 6.10; N, 3.68.

### Example E58

### Preparation of (2S,3S)-2-Acetoxy-3-(3-acetoxy-2,5-dimethyl-benzoylamino)-4-phenyl-butyric acid

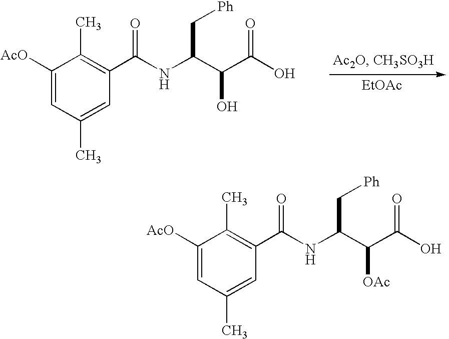

Methanesulfonic acid (16.5 mL, 253 mmol) and acetic anhydride (91.0 mL, 960 mmol) were sequentially added to a suspension of (2S,3S)-3-(3-acetoxy-2,5-dimethyl-benzoylamino)-2-hydroxy-4-phenyl-butyric acid (296 g, 768 mmol) in ethyl acetate (3.00 L) at ambient temperature. The mixture was heated at 75° C. for 2 h, and the resulting solution was then cooled to ambient temperature. The solution was sequentially washed with H₂O (2.0 L), half-saturated aqueous NaCl (2.0 L), and then with saturated aqueous NaCl (1.0 L). The resulting organic fraction was concentrated to approximately half volume by distillation at one atmosphere. Heating was discontinued and the solution was allowed to cool to ambient temperature to give a suspension. n-Heptane (3.0 L) was added and the suspension stirred at ambient temperature overnight. The solid was filtered, using 1:2 ethyl acetaten-heptane (1.5 L) for rinsing. After drying in a vacuum oven at 50° C., 316 g (96.3%) of (2S,3S)-2-acetoxy-3-(3-acetoxy-2,5-dimethyl-benzoylamino)-4-phenyl-butyric acid was obtained as a white solid: m.p.=185-186° C.; ¹H NMR (300 MHz, DMSO-d₆) δ 13.3 (s, 1H), 8.49 (d, J=8.8 Hz, 1H), 7.19-7.34 (m, 5H), 6.91 (s, 1H), 6.71 (s, 1H), 5.11 (d, J=5.0 Hz, 1H), 4.61-4.72 (m, 1H), 2.79-2.90 (m, 2H), 2.27 (s, 3H), 2.24 (s, 3H), 2.14 (s, 3H), 1.73 (s, 3H); ¹³C NMR (75 MHz, DMSO-d₆) δ 170.3, 169.7, 169.2, 168.5, 149.4, 139.1, 138.5, 136.1, 129.4, 128.5, 126.6, 125.4, 124.7, 123.8, 73.9, 51.1, 35.2, 20.9, 20.8, 20.6, 12.1; MS (Cl) m/z 428.1713 (428.1709 calcd for C₂₃H₂₆NO₇, M+H⁺); elemental analysis calcd for C₂₃H₂₅NO₇: C, 64.63; H, 5.90; N, 3.28; found: C, 64.79; H, 5.96; N, 3.15.

### Example E59

### Preparation of (2S)-4,4-Difluoro-3,3-dimethyl-pyrrolidine-2-carboxylic acid ethylamide; hydrochloride

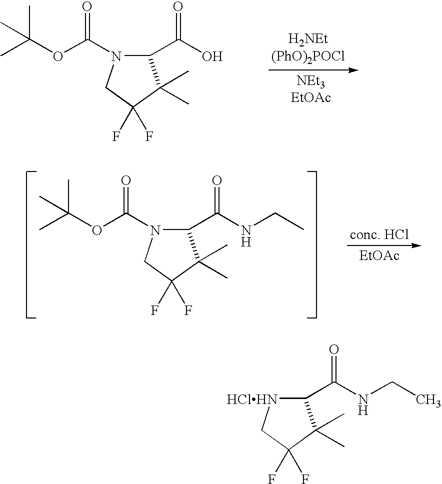

Chlorodiphenylphosphate (38.4 mL, 185 mmol) was added to a solution of (2S)-4,4-difluoro-3,3-dimethyl-pyrrolidine-1,2-dicarboxylic acid 1-tert-butyl ester (48.8 g, 175 mmol) in ethyl acetate (490 mL) at ambient temperature. The solution was cooled to 0° C., and NEt₃ (51.0 mL, 367 mmol) was added dropwise. Cooling was discontinued and the resulting suspension was allowed to warm to ambient temperature and stir for 1 h. The suspension was cooled to 0° C., and H₂NEt (96.0 mL of a 2.0 M solution in tetrahydrofuran, 192 mmol) was slowly added. The resulting mixture was allowed to warm to ambient temperature and stir for 2 h. 20% Aqueous citric acid (490 mL) was added and the layers were then separated. The aqueous fraction was extracted with ethyl acetate (125 mL). The combined organic fractions were washed with saturated aqueous NaHCO₃ (490 mL), and the layers were then separated. The aqueous fraction was extracted with ethyl acetate (125 mL). The combined organic fractions were washed with saturated aqueous NaCl (250 mL), dried over MgSO₄, and then concentrated to a volume of ˜500 mL using a rotary evaporator. Concentrated HCl (61.0 mL, 734 mmol) was added, and the solution was stirred at ambient temperature overnight. The resulting suspension was dried azeotropically with ethyl acetate (3×250 mL) by distillation at one atmosphere. The resulting suspension was cooled to ambient temperature, and was then filtered, using ethyl acetate (100 mL) for rinsing. After drying under vacuum at ambient temperature, 37.4 g (88.2%) of (2S)-4,4-difluoro-3,3-dimethyl-pyrrolidine-2-carboxylic acid ethylamide; hydrochloride was obtained as a white solid: m.p.=238-239° C. (decomp.); ¹H NMR (300 MHz, DMSO-d₆) δ 10.3 (br s, 2H), 8.70 (t, J=5.3 Hz, 1H), 4.08 (s, 1H), 3.71-3.80 (m, 2H), 3.08-3.34 (m, 2H), 1.21 (app d, J=2.2 Hz, 3H), 1.08 (t, J=7.2 Hz, 3H), 0.97 (app d, J=2.1 Hz, 3H); ¹³C NMR (75 MHz, DMSO-d₆) δ 163.8, 128.1 (dd, J_{CF}=248.6, 255.5 Hz), 64.8, 48.1 (t, J_{CF}=33.7 Hz), 45.5 (t, J_{CF}=20.8 Hz), 34.3, 18.3 (d, J_{CF}=7.4 Hz), 17.4 (app dd, J_{CF}=2.1, 5.4 Hz), 14.8; MS (Cl) m/z 207.1317 (207.1309 calcd for C₉H₁₇N₂OF₂, M−HCl+H⁺); elemental analysis calcd for C₉H₁₇ClF₂N₂O: C, 44.54; H, 7.06; N, 11.54; F, 15.66; found: C, 44.40; H, 7.06; N, 11.65; F, 15.61.

### Example E60

### Preparation of Acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester

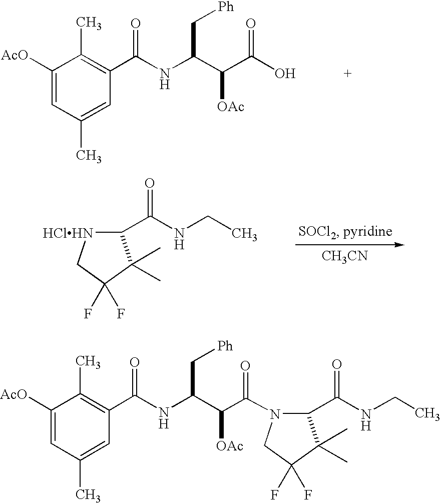

SOCl₂(1.90 mL, 25.8 mmol) was added dropwise to a 0° C. solution of (2S,3S)-2-acetoxy-3-(3-acetoxy-2,5-dimethyl-benzoylamino)-4-phenyl-butyric acid (10.0 g, 23.5 mmol), pyridine (7.60 mL, 93.9 mmol), and CH₃CN (90.0 mL). The resulting solution was allowed to warm to ambient temperature for 1 h, then was cooled to 0° C. (2S)-4,4-Difluoro-3,3-dimethyl-pyrrolidine-2-carboxylic acid ethylamide; hydrochloride (5.71 g, 23.5 mmol) was added in one portion. The resulting solution was allowed to warm to ambient temperature and stir for 2.5 h. Saturated aqueous NaHCO₃ (110 mL) and methyl t-butyl ether (110 mL) were added, and the resulting layers were separated. The resulting organic fraction was sequentially washed with 20% aqueous citric acid (90 mL), saturated aqueous NaHCO₃ (70 mL), and saturated aqueous NaCl (70 mL). Activated charcoal (14 g) was added to the resulting organic fraction, and the mixture was stirred at ambient temperature overnight. The mixture was filtered on Celite, using methyl t-butyl ether for rinsing. The filtrate was dried over MgSO₄, filtered, and concentrated to a volume of ˜90 mL using a rotary evaporator. This solution of crude acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester was carried directly to the next step. Analytical data was obtained by concentrating a sample of this solution: m.p.=88-93° C.; ¹H NMR (300 MHz, DMSO-d₆) displayed a ˜10:1 mixture of rotamers. Major rotamer resonances: δ 8.58 (d, J=8.2 Hz, 1H), 8.02 (t, J=7.5 Hz, 1H), 7.18-7.42 (m, 5H), 6.92 (s, 1H), 6.84 (s, 1H), 5.34 (d, J=3.2 Hz, 1H), 4.41-4.66 (m, 2H), 4.19-4.32 (m, 2H), 3.03-3.26 (m, 2H), 2.95 (app dd, J=2.4, 13.8 Hz, 1H), 2.78 (app dd, J=11.7, 13.8 Hz, 1H), 2.27 (s, 3H), 2.25 (s, 3H), 1.73 (s, 3H), 1.22 (br s, 3H), 1.07 (br s, 3H), 1.04 (t, J=7.2 Hz, 3H) [characteristic minor rotamer resonances: 6 7.76-7.87 (m), 6.72 (s), 5.46 (d, J=3.7 Hz), 2.07 (s), 1.79 (s)]; ¹³C NMR (75 MHz, DMSO-d₆) displayed a ˜10:1 mixture of rotamers. Major rotamer resonances: δ 170.5, 169.2, 169.0, 166.8, 166.7, 149.4, 139.1, 138.8, 136.1, 129.7, 128.3, 127.8 (dd, J_{CF}=251.2, 254.9 Hz), 126.5, 125.7, 124.7, 123.9, 73.3, 68.2, 51.4, 43.9 (t, J_{CF}=20.5 Hz), 33.8, 33.4, 22.0 (d, J_{CF}=6.0 Hz), 20.8, 20.5, 17.6 (d, J_{CF}=7.0 Hz),15.0, 12.2 [characteristic minor rotamer resonances: δ 169.5, 168.9, 167.0, 149.5, 138.7, 129.3, 128.5, 125.4, 124.8, 124.2, 34.1, 21.2, 14.7]; MS (Cl) m/z 616.2859 (616.2834 calcd for C₃₂H₄₀N₃O₇F₂, M+H⁺); elemental analysis calcd for C₃₂H₃₉F₂N₃O₇: C, 62.43; H, 6.38; N, 6.83; F, 6.17; found: C, 62.08; H, 6.68; N, 6.53; F, 5.85.

### Example E61

### Preparation of (2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide

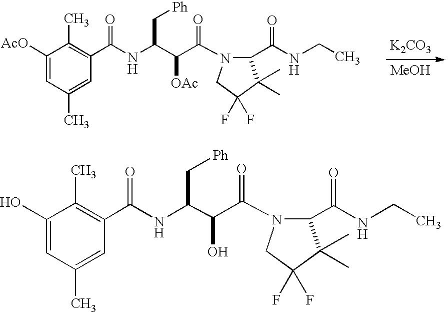

Methanol (30.0 mL) and K₂CO₃ (7.16 g, 51.7 mmol) were added to the methyl t-butyl ether solution of acetic acid 3-{(1S,2S)-2-acetoxy-1-benzyl-3-[(2S)-2-ethylcarbamoyl-4,4-difluoro-3,3-dimethyl-pyrrolidin-1-yl]-3-oxo-propylcarbamoyl}-2,5-dimethyl-phenyl ester (from above) at ambient temperature. After stirring for 2 h, the resulting yellow solution was diluted with ethyl acetate (140 mL), 1 N HCl (50 mL), and 0.5 N HCl (140 mL), and the layers were then separated. The resulting organic fraction was sequentially washed with saturated aqueous NaHCO₃ (90 mL), 0.5 N HCl (70 mL), H₂O (140 mL), and saturated aqueous NaCl (70 mL). The organic fraction was then concentrated to a volume of ˜100 mL by distillation at one atmosphere, and the resulting solution was then cooled to ambient temperature. Diisopropyl ether (190 mL) was slowly added, and the resulting crystalline suspension was stirred overnight at ambient temperature. The suspension was filtered, using diisopropyl ether (50 mL) for rinsing. After drying under vacuum, 9.88 g (79.1%) of (2S)-4,4-difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide was obtained as a white solid: m.p.=208-214° C.; ¹H NMR (300 MHz, DMSO-d₆) displayed a ˜9:1 mixture of rotamers. Major rotamer resonances: δ 9.21 (s, 1H), 8.07 (d, J=8.2 Hz, 1H), 7.90 (t, J=5.5 Hz, 1H), 7.15-7.39 (m, 5H), 6.62 (s, 1H), 6.40 (s, 1H), 5.45 (d, J=6.3 Hz, 1H), 3.95-4.50 (m, 5H), 3.02-3.22 (m, 2H), 2.89 (app dd, J=2.0, 13.5 Hz, 1H), 2.72 (app dd, J=10.4, 13.4 Hz, 1H), 2.17 (s, 3H), 1.78 (s, 3H), 1.22 (s, 3H), 1.05 (s, 3H), 1.03 (t, J=7.2 Hz, 3H) [characteristic minor rotamer resonances: δ 6.15 (d, J=8.7 Hz), 7.85 (t, J=5.7 Hz), 6.34 (s), 5.31 (d, J=7.6 Hz), 4.73 (s), 1.81 (s); ¹³C NMR (75 MHz, DMSO-d₆) displayed a 9:1 mixture of rotamers. Major rotamer resonances: δ 171.0, 169.6, 167.2, 155.5, 139.7, 139.1, 135.1, 129.8, 128.2, 128.1 (dd, J_{CF}=251.4, 254.0 Hz), 126.2, 118.7, 118.6, 116.2, 72.8, 68.5, 53.1, 51.5 (t, J_{CF}=32.0 Hz), 43.7 (t, J_{CF}=20.5 Hz), 34.2, 33.8, 22.5 (d, J_{CF}=4.7 Hz), 20.9, 17.4 (d, J_{CF}=7.3 Hz), 15.1, 12.2 [characteristic minor rotamer resonances: δ 171.8, 169.7, 168.0, 138.8, 129.5, 23.1, 14.9; MS (Cl) m/z 532.2614 (532.2623 calcd for C₂₈H₃₆N₃O₅F₂, M+H⁺); elemental analysis calc for C₂₈H₃₅F₂N₃O₅: C, 63.26; H, 6.64; N, 7.90; F, 7.15; found: C, 63.20; H, 6.67; N, 7.87; F, 7.07.

1. A compound selected from:
(R)-3-((2S,3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
(S)-4,4-Difluoro-1-[4-(3-fluoro-phenyl)-(2S,3S)-hydroxy-(3-hydroxy-2-methyl-benzoylamino)-butyryl]-3,3-dimethyl-pyrrolidine-2-carboxylic acid (2-methoxy-(1S)-methyl-ethyl)-amide;
N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide;
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide;
(S)-4,4-Difluoro-1-(2S,3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide;
(S)-4,4-Difluoro-1-[4-(3-fluoro-phenyl)-(2S)-hydroxy-(3S)-(3-hydroxy-2-methyl-benzoylamino)-butyryl]-3,3-dimethyl-pyrrolidine-2-carboxylic acid allylamide;
(S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (3-methyl-pyridin-4-ylmethyl)-amide;
2,3-Dihydro-1H-indole-4-carboxylic acid [(1S,2S)-1-benzyl-3-((S)-4,4-difluoro-2-isobutylcarbamoyl-3,3-dimethyl-pyrrolidin-1-yl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide;
2,3-Dihydro-1H-indole-4-carboxylic acid [(1S,2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide;
(S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide;
2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide;
(S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide;
(S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide;
1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
N-((1S,2S)-3-{(2S)-2-[(allylamino)carbonyl]-4,4-difluoro-3,3-dimethylpyrrolidin-1-yl}-1-benzyl-2-hydroxy-3-oxopropyl)indoline-4-carboxamide;
(4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide;
(S)-3-((2S,3S)-2-Hydroxy-3-{[1-(3-hydroxy-2-methyl-phenyl)-methanoyl]-amino}-4-phenyl-butanoyl)-5,5-dimethyl-oxazolidine-4-carboxylic acid (S)-indan-1-ylamide;
(3R)-N-allyl-1-{(2S,3S)-2-hydroxy-3-[(3-hydroxy-2-methylbenzoyl)amino]-4-phenylbutanoyl}-3-phenyl-L-prolinamide;
(4S)-N-(2-fluorobenzyl)-1-{(2S,3S)-2-hydroxy-3-[(3-hydroxy-2-methylbenzoyl)amino]-4-phenylbutanoyl}4-methoxy-3,3-dimethyl-L-prolinamide; and
(4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide.; or
a pharmaceutically acceptable salt or solvate thereof.

2. A compound according to claim 1 selected from:
(R)-3-((2S,3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
(S)-4,4-Difluoro-1-[4-(3-fluoro-phenyl)-(2S,3S)-hydroxy-(3-hydroxy-2-methyl-benzoylamino)-butyryl]-3,3-dimethyl-pyrrolidine-2-carboxylic acid (2-methoxy-(1S)-methyl-ethyl)-amide;
N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide;
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide;
(S)-4,4-Difluoro-1-(2S,3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide;
(S)-4,4-Difluoro-1-[4-(3-fluoro-phenyl)-(2S)-hydroxy-(3S)-(3-hydroxy-2-methyl-benzoylamino)-butyryl]-3,3-dimethyl-pyrrolidine-2-carboxylic acid allylamide;
(S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (3-methyl-pyridin-4-ylmethyl)-amide;
2,3-Dihydro-1H-indole-4-carboxylic acid [(1S,2S)-1-benzyl-3-((S)-4,4-difluoro-2-isobutylcarbamoyl-3,3-dimethyl-pyrrolidin-1-yl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide;
2,3-Dihydro-1H-indole-4-carboxylic acid [(1S,2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-4-ylmethyl)-amide; and
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide; or
a pharmaceutically acceptable salt or solvate thereof.

3. A compound according to claim 1 selected from:
(R)-3-((2S,3R)-4,4-Difluoro-1-[4-(3-fluoro-phenyl)-2-hydroxy-3-(3-hydroxy-2-methyl-benzoylamino)-butyryl])-3,3-dimethyl-pyrrolidine-2-carboxylic acid allylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(2S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
(S)-4,4-Difluoro-1-[4-(3-fluoro-phenyl)-(2S,3S)-hydroxy-(3-hydroxy-2-methyl-benzoylamino)-butyryl]-3,3-dimethyl-pyrrolidine-2-carboxylic acid (2-methoxy-(1S)-methyl-ethyl)-amide; and
N-[3-(2S)-Butylcarbamoyl-4,4-difluoro-3,3-dimethyl-cyclopentyl)-(1S,2S)-(3-fluoro-benzyl)-2-hydroxy-3-oxo-propyl]-3-hydroxy-2-methyl-benzamide; or
a pharmaceutically acceptable salt or solvate thereof.

4. A compound according to claim 1 selected from:
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide;
(S)-4,4-Difluoro-1-(2S,3S)-2-hydroxy-3-(3-hydroxy-2,5-dimethyl-benzoylamino)-4-phenyl-butyryl]-3,3-dimethyl-pyrrolidine-2-carboxylic acid (1-methoxymethyl-2(S)-propyl)-amide;
(S)-4,4-Difluoro-1-[4-(3-fluoro-phenyl)-(2S)-hydroxy-(3S)-(3-hydroxy-2-methyl-benzoylamino)-butyryl]-3,3-dimethyl-pyrrolidine-2-carboxylic acid allylamide;
(S)-4,4-Difluoro-1-[(2S,3S)-hydroxy-3-(3-hydroxy-2-methyl-benzoylamino)-4-(3-trifluoromethyl-phenyl)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (3-methyl-pyridin-4-ylmethyl)-amide;
2,3-Dihydro-1H-indole-4-carboxylic acid [(1S,2S)-1-benzyl-3-((S)-4,4-difluoro-2-isobutylcarbamoyl-3,3-dimethyl-pyrrolidin-1-yl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid isobutyl-amide;
2,3-Dihydro-1H-indole-4-carboxylic acid [(1S,2S)-3-((S)-4,4-difluoro-2-isobutylcarbamoyl-3,3-dimethyl-pyrrolidin-1-yl)-1-(3-fluoro-benzyl)-2-hydroxy-3-oxo-propyl]-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (pyridin-4-ylmethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-4-ylmethyl)-amide; and
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid (2,2-dimethyl-propyl)-amide; or
a pharmaceutically acceptable salt or solvate thereof.

5. A compound according to claim 1 selected from:
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide;
(S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide;
2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide;
(S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide;
(S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide;
1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide;
1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
N-((1S,2S)-3(2S)-2-[(allylamino)carbonyl]-4,4-difluoro-3,3-dimethylpyrrolidin-1-benzyl-2-hydroxy-3-oxopropyl)indoline-4-carboxamide;
(4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide;
(S)-3-((2S,3S)-2-Hydroxy-3-{[1-(3-hydroxy-2-methyl-phenyl)-methanoyl]-amino}4-phenyl-butanoyl)-5,5-dimethyl-oxazolidine-4-carboxylic acid (S)-indan-1-ylamide;
(3R)-N-allyl-1-{(2S,3S)-2-hydroxy-3-[(3-hydroxy-2-methylbenzoyl)amino]-4-phenylbutanoyl}-3-phenyl-L-prolinamide;
(4S)-N-(2-fluorobenzyl)-1-{(2S,3S)-2-hydroxy-3-[(3-hydroxy-2-methylbenzoyl)amino]-4-phenylbutanoyl}-4-methoxy-3,3-dimethyl-L-prolinamide; and
(4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide; or
a pharmaceutically acceptable salt or solvate thereof.

6. A compound according to claim 1 selected from:
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid (2,2-dimethyl-propyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid (2,2-dimethyl-propyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid 2-fluoro-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (R)-sec-butylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,6-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((R)-sec-butyl)-amide;
(S)-3-[(2S,3S)-2-Hydroxy-3-(3-hydroxy-2,5-dimethyl-benzoylamino)-4-phenyl-butyryl]-5,5-dimethyl-oxazolidine-4-carboxylic acid (S)-indan-1-ylamide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid 2-methyl-benzylamide;
(S)-4,4-Difluoro-1-[(2S,3S)-4-(3-fluoro-phenyl)-2-hydroxy-3-(3-hydroxy-2,5-dimethyl-benzoylamino)-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid 3-cyclopropylmethoxy-benzylamide;
2,3-Dihydro-1-H-indole-4-carboxylic acid [3S-(2-allylcarbamoyl-4,4-difluoro-3,3-dimethyl-pyrrolidin-1-yl)-1S-(3-fluoro-benzyl)-2S-hydroxy-3-oxo-propyl]-amide;
(S)-4R-Hydroxy-1-[(2S,3S)-2-hydroxy-3-(3-hydroxy-2-methyl-benzoylamino)-4-phenyl-butyryl]-3,3,4-trimethyl-pyrrolidine-2-carboxylic acid 2-methyl-benzylamide;
(S)-4R-Hydroxy-1-[(2S,3S)-2-hydroxy-3-(3-hydroxy-2,5-dimethyl-benzoylamino)-4-phenyl-butyryl]-3,3,4-trimethyl-pyrrolidine-2-carboxylic acid 2-methyl-benzylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (pyridin-2-ylmethyl)-amide;
1-[(2S,3S)-2-Hydroxy-3-(3-hydroxy-2-methyl-benzoylamino)-4-phenyl-butyryl]-3,3-dimethyl-4-methylene-pyrrolidine-2-carboxylic acid allylamide;
1-[(2S,3S)-2-Hydroxy-3-(3-hydroxy-2,5-dimethyl-benzoylamino)-4-phenyl-butyryl]-3,3-dimethyl-4-methylene-pyrrolidine-2-carboxylic acid isobutyl-amide;
1-[(2S,3S)-2-Hydroxy-3-(3-hydroxy-2-methyl-benzoylamino)-4-phenyl-butyryl]-(4R)-3,3,4-trimethyl-pyrrolidine-2-carboxylic acid propylamide; and
1-[(2S,3S)-2-Hydroxy-3-(3-hydroxy-2,5-dimethyl-benzoylamino)-4-phenyl-butyryl]-(4R)-3,3,4-trimethyl-pyrrolidine-2-carboxylic acid isobutyl-amide; or
a pharmaceutically acceptable salt or solvate thereof.

7. A compound according to claim 1 selected from:
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
N-((1S,2S)-3-{(2S)-2-[(allylamino)carbonyl]-4,4-difluoro-3,3-dimethylpyrrolidin-1-yl}-1-benzyl-2-hydroxy-3-oxopropyl)indoline4-carboxamide;
(4S)-Hydroxy-1-[(2S,3S)-2-hydroxy-3-(3-hydroxy-2-methyl-benzoylamino)-4-phenyl-butyryl]-3,3-dimethyl-pyrrolidine-2-carboxylic acid allylamide;
(S)-3-((2S,3S)-2-Hydroxy-3-{[1-(3-hydroxy-2-methyl-phenyl)-methanoyl]-amino}4-phenyl-butanoyl)-5,5-dimethyl-oxazolidine-4-carboxylic acid (S)-indan-1-ylamide;
(3R)-N-allyl-1-{(2S,3S)-2-hydroxy-3-[(3-hydroxy-2-methylbenzoyl)amino]-4-phenylbutanoyl}-3-phenyl-L-prolinamide;
(4S)-N-(2-fluorobenzyl)-1-{(2S,3S)-2-hydroxy-3-[(3-hydroxy-2-methylbenzoyl)amino]-4-phenylbutanoyl}-4-methoxy-3,3-dimethyl-L-prolinamide; and
(4S)-1-{(2S,3S)-2-hydroxy-3-[(3-hydroxy-2-methylbenzoyl)amino]-4-phenylbutanoyl}-4-methoxy-3,3-dimethyl-N-(2-methylbenzyl)-L-prolinamide; or
a pharmaceutically acceptable salt or solvate thereof.

8. A compound according to claim 1 selected from:
(S)-4,4-Difluoro-1-[(2S,3S)-4-(3-fluoro-phenyl)-2-hydroxy-3-(3-hydroxy-2-methyl-benzoylamino)-butyryl]-3,3-dimethyl-pyrrolidine-2-carboxylic acid isobutyl-amide;
(R)-3-[(2S,3S)-2-Hydroxy-3-(3-hydroxy-2,5-dimethyl-benzoylamino)-4-phenyl-butyryl]-5,5-dimethyl-thiazolidine-4-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid allylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid propylamide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid isobutyl-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid (2,2,2-trifluoro-ethyl)-amide;
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ethylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide;
(S)-4,4-Difluoro-1-[(2S,3S)-hydroxy-(3-hydroxy-2,5-dimethyl-benzoylamino)-4-phenyl-butyryl]-3,3-dimethyl-pyrrolidine-2-carboxylic acid butylamide;
(S)-4,4-Difluoro-1-[(2S,3S)-2-hydroxy-3-(3-hydroxy-2-methyl-benzoylamino)-4-phenyl-butyryl]-3,3-dimethyl-pyrrolidine-2-carboxylic acid ((2S)-2-methyl-butyl)-amide; and
(S)-4,4-Difluoro-1-[(2S,3S)-2-hydroxy-3-(3-hydroxy-2,5-dimethyl-benzoylamino)-4-phenyl-butyryl]-3,3-dimethyl-pyrrolidine-2-carboxylic acid ((2S)-2-methyl-butyl)-amide; or
a pharmaceutically acceptable salt or solvate thereof.

9. A method of inhibiting HIV protease activity, comprising contacting said HIV protease with an effective amount of a compound according to claim 1.

10. A method of inhibiting HIV protease activity in an HIV-infected mammal, comprising administering to said mammal an HIV-inhibiting amount of a compound according to claim 1.

11. A pharmaceutical composition, comprising an HIV-protease inhibiting amount of a compound according to claim 1 and at least one pharmaceutically acceptable carrier.
